(19) United States
(12) Patent Application Publication
(10) Pub. No.: US 2018/0127748 A1
(43) Pub. Date: May 10, 2018

(54) METHODS RELATING TO THE PREVENTION AND TREATMENT OF DRUG RESISTANCE

(71) Applicant: THE GENERAL HOSPITAL CORPORATION, Boston, MA (US)

(72) Inventor: Johnathan R. WHETSTINE, Winchester, MA (US)

(73) Assignee: THE GENERAL HOSPITAL CORPORATION, Boston, MA (US)

(21) Appl. No.: 15/574,165

(22) PCT Filed: May 12, 2016
(86) PCT No.: PCT/US2016/032043

§ 371 Date: Nov. 15, 2017

Related U.S. Application Data
(60) Provisional application No. 62/162,141, filed on May 15, 2015.

(51) Int. Cl. C12N 15/113; A61K 31/194; A61K 31/4375; A61K 31/443; A61K 31/4436; A61K 31/444; A61K 31/7105; A61K 45/06; C12N 15/52; A61P 35/00; A61P 31/00

(52) U.S. Cl. CPC C12N 15/113; C12N 2310/16; A61K 31/4375; A61K 31/443; A61K 31/4436; A61K 31/444; A61K 31/7105; A61K 45/06; C12N 15/52; C12Y 114/11; A61P 35/00; A61P 31/00; C12N 2310/11; C12N 2310/141; A61K 31/194

(57) ABSTRACT

Described herein are methods, assays, and compositions relating to the treatment and/or prevention of drug-resistance, e.g, by inhibiting the activity of KDM4A-like enzymes.

### CROSS-REFERENCE TO RELATED APPLICATIONS

This application claims benefit under 35 U.S.C. § 119(e) of U.S. Provisional Application No. 62/162,141 filed May 15, 2015, the contents of which are incorporated herein by reference in their entirety.

### GOVERNMENT SUPPORT

This invention was made with government support under Grant Nos. CA059267 and R01GM097360 awarded by the National Institutes of Health. The U.S. government has certain rights in the invention.

### TECHNICAL FIELD

The technology described herein relates to methods of preventing and/or reducing drug resistance in, e.g. infections or cancer.

### BACKGROUND

Cancer is often characterized by copy gains or losses of chromosome arms, whole chromosomes, and/or amplifications/deletions of smaller genomic fragments. Traditionally, somatic copy number alterations (SCNA) and copy number variations (CNV) have been thought of as heritable genetic events in cancer cells that emerge through an adaptive advantage; however, recent work suggests that at least some copy gains may be transient and could arise given the correct genetic, therapeutic or environmental conditions (Black et al. 2013; Nathanson et al. 2014).

### SUMMARY

As described herein, the inventors have found that the activity of KDM4A, and related enzymes, promotes copy number gain at specific locations, particularly those that promote drug resistance. Accordingly, provided herein are methods of preventing and/or reducing drug resistance by administering inhibitors of KDM4A-like proteins, thereby preventing gene amplification of drug resistance-related genes. The methods described herein are applicable to the treatment of, e.g. cancer or pathogenic infections.

The inventors have further discovered that the enzymatic domain of KDM4A is conserved, e.g. in bacteria. Accordingly, the methods of preventing and/or reducing drug resistance are also applicable to the treatment of infections, e.g. bacterial or fungal infections.

In one aspect, described herein is a method of reducing and/or preventing the development of drug resistance in a cell, the method comprising contacting the cell with an inhibitor of a KDM4A-like enzyme or an inhibitor of an enzyme that hydroxylates nucleic acids and/or histones or histone-like proteins. In some embodiments, the cell is a prokaryotic cell. In some embodiments, the drug resistance is antibiotic resistance. In some embodiments, the cell is a eukaryotic cell. In some embodiments, the cell is selected from the group consisting of: a yeast cell and a mammalian cell. In some embodiments, the cell is a cancer cell. In some embodiments, the drug resistance is chemotherapeutic resistance. In some embodiments, the cell is contacted with an inhibitor of a KDM4A-like enzyme. In some embodiments, the KDM4A-like enzyme comprises a cupin β barrel domain. In some embodiments, the KDM4A-like enzyme is selected from the group consisting of KDM4A; KDM5A; KDM6B; KDM4B; KDM4C; a member of the JmjC enzyme family (e.g., KDM2A, KDM2B, KDM3A, KDM3B, KDM4A, KDM4B, KDM4C, KDM5C, KDM6B, and KDM7); a Cupin protein; and the proteins listed in Tables 1 and 2 and/or homologs thereof. In some embodiments, the inhibitor of a KDM4A-like enzyme is selected from the group consisting of: an inhibitory nucleic acid; an aptamer; a miRNA; Suv39H1; HP1; increased oxygen levels; an inhibitor of a KDM4A-targeting KMT (agonist or antagonists); an inhibitor of Tudor or PHD domain interaction; succinate; and JIB-04 or additional drugs targeting the enzymatic domain. In some embodiments, the cell is a cell determined to be experiencing hypoxic conditions. In some embodiments, the prokaryotic cell comprises a gene encoding a KDM4A-like enzyme. In some embodiments, the method further comprises the step of determining that the prokaryotic cell comprises a gene encoding a KDM4A-like enzyme.

In one aspect, described herein is a method of treating an infection in a subject, the method comprising administering inhibitor of a KDM4A-like enzyme or an inhibitor of an enzyme that hydroxylates nucleic acids and/or histones or histone-like proteins. In one aspect, described herein is a method of treating an infection in a subject, the method comprising administering: a) an antibiotic and b) an inhibitor of a KDM4A-like enzyme or an inhibitor of an enzyme that hydroxylates nucleic acids and/or histones or histone-like proteins. In some embodiments, the antibiotic is a DNA damage inducing agent or an antibiotic used to treat an anaerobe infection. In some embodiments, the infection is selected from the group consisting of: a fungal infection; a yeast infection; a eurkaryotic infection; a prokaryotic infection; and a bacterial infection. In some embodiments, the infection comprises an organism comprising a gene encoding a KDM4A-like enzyme. In some embodiments, the method further comprises the step of determining that the infection comprises an organism comprising a gene encoding a KDM4A-like enzyme. The inhibition of KDM4A-like enyzmes in the infectious microbe can reduce resistance in these microbes to human.

In one aspect, described herein is a method of reducing and/or preventing the development of drug resistance in a subject in need of treatment for cancer, the method comprising administering a) a chemotherapeutic agent and b) an inhibitor of a KDM4A-like enzyme or an inhibitor of an enzyme that hydroxylates nucleic acids and/or histones or histone-like proteins. In some embodiments, the chemotherapeutic agent is selected from the group consisting of DNA-damaging agents (e.g. doxorubicin); S-phase chemotherapeutics; mTOR inhibitors; protein synthesis inhibitors; Braf inhibitors; PI3K inhibitors; Cdk inhibitors; Aurora B inhibitors; FLT3 inhibitors; PLK1/2/3 inhibitors; Eg5 inhibitors; β-tubulin inhibitors; BMP inhibitors; HDAC inhibitors; Akt inhibitors; IGF1R inhibitors; p53 inhibitors; hdm2 inhibitors; STAT3 inhibitors; VEGFR inhibitors; angiogenesis inhibitors; proteasomal inhibitors; ubiquitin-targeting drugs; and bortezomib.

In one aspect, described herein is a method of reducing and/or preventing the development of drug resistance in a subject in need of treatment with an angiogenesis inhibitor, the method comprising administering: a) the angiogeneisis inhibitor and b) an inhibitor of a KDM4A-like enzyme or an inhibitor of an enzyme that hydroxylates nucleic acids and/or histones or histone-like proteins. In one aspect, described herein is a method comprising administering: a) an angiogenesis inhibitor and b) an inhibitor of a KDM4A-like enzyme or an inhibitor of an enzyme that hydroxylates nucleic acids and/or histones or histone-like proteins to a subject in need of anti-angiogenic therapy. The foregoing methods, combining the administration of an angiogenesis inhibitor and an inhibitor of a KDM4A-like enzyme or an inhibitor of an enzyme that hydroxylates nucleic acids and/or histones or histone-like proteins reduces and/or prevents drug resistance in the subject.

In some embodiments, the angiogenesis inhibitor is selected from the group consisting of: bevacizumab; sorefenib; sunitinib; pazopanib; and everolimus. In some embodiments, the subject is administered an inhibitor of a KDM4A-like enzyme. In some embodiments, the KDM4A-like enzyme comprises a cupin β barrel domain. In some embodiments, the KDM4A-like enzyme is selected from the group consisting of KDM4A; KDM5A; KDM6B; KDM4B; KDM4C; a member of the JmjC enzyme family (e.g., KDM2A, KDM2B, KDM3A, KDM3B, KDM4A, KDM4B, KDM4C, KDM5C, KDM6B, and KDM7); a Cupin protein; and the proteins listed in Tables 1 and 2 and/or homologs thereof. In some embodiments, the inhibitor of a KDM4A-like enzyme is selected from the group consisting of: an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; and JIB-04. In some embodiments, the inhibitor of KDM4A can be a nucleic acid comprising the sequence of hsa-mir-23a-3p, hsa-mir-23b-3p and/or hsa-mir-137.

In one aspect, described herein is a method of detecting a drug-resistance promoting state in a subject, the method comprising: detecting the presence of a copy-gained region in a sample of cell-free DNA obtained from the subject. In some embodiments, the copy-gained region comprises the 1q12h (hsat2), 1q12h/21 (e.g., ANK) CKS1B, DHFR BCL9, Xp13.1 gene. In some embodiments, the copy-gained region is a region of the genome that is subject to copy number variation in cancer cells. In some embodiments, the copy-gained region is selected from the group consisting of: 1q12-1q25;1q12h; 1q21.2; and Xq31.1. In some embodiments, the copy-gained region comprises the 1q21-23 locus. In some embodiments, the sample is a tissue sample, urine sample, or plasma sample. In some embodiments, the presence of a copy-gained regions is detected by FISH, a cytological approach, DNA sequencing, or PCR-based analysis. In some embodiments, the method further comprises the step of treating the subject with an inhibitor of a KDM4A-like enzyme or an inhibitor of an enzyme that hydroxylates nucleic acids and/or histones or histone-like proteins.

### BRIEF DESCRIPTION OF THE DRAWINGS

FIG. 1 presents a summary of gene amplification phenomena.

FIG. 2 depicts a three-dimensional model of the beta-sheet coiling pattern of JMJD2A.

FIG. 3 depicts an alignment of 13 hits from the beta-sheet coiling pattern search.

FIG. 4 depicts an alignment of the top 3 hits shown in FIG. 3.

FIG. 5 depicts a sequence alignment of the iron-containing structure proteins.

FIG. 6 depicts a distance tree of the proteins depicted in FIG. 5.

FIG. 7 depicts the results of an HMM profile search for the structurally aligned regions of the iron containing structures.

FIGS. 8A-8G demonstrate that hypoxia, but not other physiological stresses promote transient site-specific copy gain. (FIG. 8A) Schematic detailing the approach used in the screen of physiological stresses. RPE cells were exposed to the indicated stress for 24 hours prior to collection for FISH and FACS analysis. (FIG. 8B) Hypoxia promotes site-specific copy gain of 1q12h and 1q21.2 by FISH analysis. (FIG. 8C) Hypoxia amplified regions are not contiguous. Table summarizing co-amplification of 1q12h, and 1q21.2. Data are presented as percent of all amplified cells (sum of all replicates) having 2 or 3 or more (3+) copies of the indicated FISH probes. (FIG. 8D) Hypoxia induced copy gain of 1q12h is reversible. Quantification of FISH for 1q12h and Chr 8 after 24-72 hours of 21% O₂ (normoxia), 1% O₂ (hypoxia), or return to normoxia from 1% O₂ for 24 hours (Rescue). † indicates significant difference from 1% O₂ for 72 hours by two-tailed Student's t-test (p<0.05). (FIG. 8E) Hypoxia-dependent copy gains are removed within four hours of return to normoxia. Quantification of FISH probes for the indicated times after 48 hours of normoxia or hypoxia treatment. † indicates significant difference from zero hour release from 1% O₂ by two-tailed Student's t-test (p<0.05). (FIG. 8F) Hypoxia-induced copy gains occur during S phase. Quantification of FISH for 1q12h, 1q21.2 and 8c in RPE cells following HU arrest in normoxia or 1% O₂ (time 0) or the indicated time after HU release. † indicates significant difference from Asynchronous (−) 1% O₂ by two-tailed Student's t-test (p<0.05). (FIG. 8G) Regions with hypoxia-dependent copy gain are rereplicated. CsCl density gradient purification of rereplicated DNA was analyzed by qPCR for regions amplified in hypoxia. Error bars represent the S.E.M. * indicates significant difference from normoxia by two-tailed Student's t-test (p<0.05).

FIGS. 9A-9B demonstrate that hypoxia induces site-specific copy in primary human T cells. (FIG. 9A) Schematic illustrating collection, isolation and stimulation of primary human T cells. (FIG. 9B) Hypoxia induces site-specific copy gain only in stimulated primary human T cells. Error bars represent the S.E.M. * indicates significant difference from normoxia by two-tailed Student's t-test (p<0.05).

FIGS. 10A-10J demonstrate that hypoxia induced site-specific copy gains are KDM4A-dependent. (FIG. 10A) KDM4B-D are not required for copy gain in hypoxia. Quantification of FISH for 1q12h and 8c in RPE cells depleted of KDM4B, C or D and maintained in normoxia or hypoxia. Data presented are an average of two independent experiments, each performed with two independent siRNAs. (FIG. 10B) Hypoxia-induced 1q12h and 1q21.2 copy gains require KDM4A. Quantification of FISH for 1q12h, and 8c in RPE cells after 24 hours of normoxia or hypoxia and with or without depletion of KDM4A. Data presented are an average of two independent siRNA. (FIG. 10C) Genomic deletion of KDM4A using CRISPR/Cas9 abrogates hypoxia-driven copy gain. Quantification of FISH for 1q12h and 8c in 293T CRISPR cell lines stably expressing either GFP or GFP-KDM4A, following 24 hours of normoxia or hypoxia. Data represents an average of two independent experiments for two independently derived single cell clones of GFP (GFP8 and GFP14) or GFP-KDM4A (WT19 and WT28). (FIG. 10D) Hypoxia stabilizes KDM4A protein levels. Western blot indicates KDM4A protein levels after 24 and 48 hours of hypoxic treatment in RPE cells (left panel), and in primary human T cells with or without stimulation (right panel). (FIG. 10E) Hypoxia increases the half-life of KDM4A protein in RPE cells. (Top panel) Western blot of half-life experiment demonstrates that KDM4A is stabilized in hypoxia following cycloheximide treatment. (Bottom panel) Graphical representation of KDM4A half-life in RPE cells. Quantification of half-life indicates a half-life of 1 hr 49 min±3 min in normoxia and 4 hr 56 min±37 min in hypoxia. * indicates significant difference from normoxia at the same time point by two-tailed Student's t-test (p<0.05). (FIG. 10F) Hypoxia abrogates the interaction of the SCF complex with KDM4A. KDM4A was immunoprecipitated from RPE cells maintained in normoxia or hypoxia, and the interaction with components of the SCF complex was analyzed by western blot. (FIG. 10G) KDM4A levels are increased on chromatin during hypoxia (lane 5 and 6 respectively; 1% O₂; Cyto=cytoplasm; NE=nuclear extract; Chrom=chromatin fraction). (FIG. 10H) KDM4A demethylase activity is retained after 24 hours in hypoxia. RPE cells expressing 3×HA-WT-KDM4A were maintained in normoxia or hypoxia for 24 hours and H3K9 and H3K36 demethylation was assessed by immunofluorescence. The graph represents an average of two independent experiments with demethylase activity in hypoxia normalized to activity in normoxia. (FIG. 10I) Demethylase inhibition with JIB-04 blocks hypoxia-dependent copy gain. Quantification of FISH for 1q12h and Chr 8 in RPE cells upon JIB-04 treatment. (FIG. 10J) Hypoxia-dependent copy gains can be suppressed by treatment with 2 mM succinate. In all panels: error bars indicate S.E.M., * indicates significant difference from normoxia (FIGS. 10B,10C), and significant difference from vehicle treated normoxia samples (FIGS. 10I, 10J) by two-tailed Student's t-test (p<0.05). † indicates significant difference from siCTRL (1% O₂) (FIG. 10B) and significant difference from Vehicle (1% O₂) (FIGS. 10I, 10J) by two-tailed Student's t-test (p<0.05).

FIGS. 11A-11I demonstrate that hypoxia induced copy gains are conserved in zebrafish. (FIG. 11A) Schematic depicting homology of huKDM4A and zfKDM4A. Table depicts the H3K9 and H3K36 demethylase activity of zebrafish KDM4A expressed in RPE cells as determine by immunofluorescence. (FIG. 11B) Expression levels of zebrafish and human KDM4A proteins in RPE cells expressing wild-type (WT) and catalytically mutant (H185A) zebrafish KDM4A. (FIG. 11C) Zebrafish KDM4A promotes copy gain in human cells. Quantification of FISH for 1q12h, 1q21.2 and 8c for RPE cells expressing zfKDM4A or catalytically inactive, zfKDM4A CAT. (FIG. 11D) Quantification of H3K9 and H3K36 demethylase activity by immunofluorescence in normoxia and hypoxia for RPE cells ectopically expressing zebrafish KDM4A (zfKDM4A). (FIG. 11E) Hypoxia stabilizes zfKDM4A in RPE cells. (FIG. 11F) Schematic depicting syntenic region of 1q21.2 in zebrafish used for FISH analysis. Green bars indicate the location of the human (stick figure) and zebrafish (fish icon) probes used. (FIG. 11G) Hypoxia promotes copy gain of BCL9 in zebrafish AB.9 cells. Quantification of FISH for BCL9 after 72 hours of normoxia or 1% O₂. (FIG. 11H) Schematic of IGBP1 homologous region in zebrafish. Green bars indicate the location of the human (stick figure) and zebrafish (fish icon) probes used. (FIG. 11I) Hypoxia does not induce copy gain of IGBP1 in zebrafish. Quantification of FISH for IGBP1 after 72 hours of normoxia or 1% O₂. Error bars represent the S.E.M. * indicates significant difference from control samples by two-tailed Student's t-test (p<0.05).

FIGS. 12A-12J demonstrate that tumors with a hypoxic signature have copy gains of regions observed in hypoxic cell culture. (FIG. 12A) TCGA Breast Cancer samples with a hypoxic gene signature have a faster time to death. (FIG. 12B) TCGA Lung Adenocarcinoma samples with a hypoxic gene signature have a faster time to death. (FIG. 12C) TCGA Breast Cancer samples with a hypoxic gene signature have increased focal copy number variation. (FIG. 12D) TCGA Lung Adenocarcinoma samples with a hypoxic gene signature have increased focal copy number variation. (FIG. 12E) TCGA Breast Cancer samples with a hypoxic gene signature have an enrichment of copy gain of 1p11.2 through 1q23.3. (FIG. 12F) TCGA Breast Cancer samples without a hypoxic gene signature do not have enrichment of copy gain of 1p11.2 through 1q23.3. (FIG. 12G) Mean copy number of hypoxic (red) and non-hypoxic (blue) breast cancer samples. (FIG. 12H) TCGA Lung Adenocarcinoma samples with a hypoxic gene signature have enriched copy gain of 1p11.2 through 1q23.3. (FIG. 12I) TCGA Lung Adenocarcinoma samples without a hypoxic gene signature do not have enriched copy gain of 1p11.2 through 1q23.3. (FIG. 12J) Mean copy number of hypoxic (red) and non-hypoxic (blue) lung adenocarcinoma samples. For each co-amplification plot, blue shaded regions indicate 1p11.2 through 1q23.3.

FIGS. 13A-13D demonstrate that CKS1B exhibits site-specific copy gain and increased expression in hypoxic cells. (FIGS. 13A, 13B) CKS1B is copy-gained and overexpressed in hypoxic breast cancer cell lines. Quantification of FISH (FIG. 13A) and CKS1B mRNA expression (FIG. 13B) in MDA-MB 231 cells maintained in hypoxia for 24-72 hours, or maintained in hypoxia for 48 hrs prior to return to normoxia for 24 hours (rescue). † indicates significant difference from 1% O₂ at 24 hours by two-tailed Student's t-test (p<0.05). (FIG. 13C) Hypoxia-dependent CKS1B copy gain requires KDM4A. Quantification of FISH for 1q12h and 8c for MDA-MB-231 cells maintained in normoxia or hypoxia, with or without siRNA depletion of KDM4A. † indicates significant difference from 1% O₂ siCTRL by two-tailed Student's t-test. (FIG. 13D) Hypoxia-dependent CKS1B transcript induction requires KDM4A. Circled * indicates significant difference from siCTRL in hypoxia by two-tailed Student's t-test (p<0.05). In all panels, * indicates significant difference from normoxia by two-tailed Student's t-test (p<0.05).

FIG. 14 depicts a model depicting how site-specific copy gains could explain intra-tumoral heterogeneity.

FIGS. 15A-15R demonstrate that treatment with chemical and metabolic stresses does not promote copy gain. (FIG. 15A) Hypoxic conditions increase HIF1α and CAIX levels in RPE cells. Western blot indicating protein levels of HIF1α and CAIX in normoxia or following 24 hours in hypoxia (1% O₂). (FIG. 15B-FIG. 15F) Treatment with chemical and metabolic stresses does not promote copy gain. Quantification of FISH for 1q12h, Chr 8, 1q23.3 and 1q21.2 after 24 hours of ROS (H₂O₂) (FIG. 15B), 43° C. heat shock (HS) (FIG. 15C), reduced serum (0.1% FBS) (FIG. 15D), Tunicamycin (TU) (FIG. 15E), and glucose deprivation (FIG. 15F). (FIG. 15G-FIG. 15L) Cell cycle analysis following 24 hours exposure to the indicated stresses. (FIG. 15M-FIG. 15R) Oxidants and reducing reagents do not induce site-specific copy gains. Quantification of FISH for 1q12h and 8c (FIG. 15M-FIG. 150) and cell cycle analysis (FIG. 15P-FIG. 15R) in RPE cells following 24 hours of treatment with 2 mM DTT, 5 mM N-acetyl Cysteine (NAC), and 1 μM DMNQ. In all panels, error bars represent the S.E.M. * indicates significant difference from control samples by two-tailed Student's t-test (p<0.05). * adjacent to bar graphs for cell cycle distribution indicate p<0.05 compared to control samples for that cell cycle phase.

FIGS. 16A-16S demonstrate that hypoxia promotes site-specific copy gains in diverse cancer cell types. (FIG. 16A-16D) Hypoxia promotes site-specific gains in breast cancer cell lines. Western blots depict the hypoxic response of MDA-MB 468 (FIG. 16A) and MDA-MB 231 (FIG. 16C) cells following 24 hours of hypoxic exposure. Quantification of FISH indicates amplification of 1q12h but not 8c in hypoxic MDA-MB 468 (FIG. 16B) and MDA-MB 231 (FIG. 16D) cells. (FIG. 16E-16J) SK-N-AS neuroblastoma (FIG. 16E,16F), 293T kidney (FIGS. 16G,16H), and MM.1S multiple myeloma (FIG. 161,16J) cells are hypoxic and exhibit copy gain of 1q12h following 24 hours of 1% O₂. (FIG. 16K-16M) Hypoxia promotes site-specific gain in renal cancer cells independent of activated HIF1/2a (UMRC2—lack VHL and have constitutively active HIF). (FIG. 16K) Western blot indicating the hypoxic response of UMRC2 cells lacking (−) or expressing (+) VHL following 24 hours in hypoxia. (FIG. 16L,16M) Quantification of FISH for 1q12h and 8c (FIG. 16L) or 1q23.3 and 1qte1 (FIG. 16M) after 24 hours of normoxia or 1% O₂. (FIG. 16N) Hypoxia-induced copy gains are not dependent on HIF1α. Quantification of FISH for 1q12h and Chr 8 in RPE cells maintained in either in normoxia or 1% O₂, with or without depletion of HIF1α. (FIG. 16O) Western blot demonstrating abrogation of CAIX induction upon HIF1α depletion. (FIG. 16P) Hypoxia-driven copy gains are not dependent on HIF2a. Quantification of FISH for 1q12h and Chr 8 in RPE cells maintained in either in normoxia or 1% O₂, with or without depletion of HIF2a. (FIG. 16Q) Western blot demonstrating CAIX induction upon HIF2a depletion. (FIG. 16R) Representative FACS analysis demonstrating cell cycle progression through HU release in normoxia and hypoxia. Cell cycle profiles are provided for asynchronous (ASYN), HU arrested (0 hr), and released (4 hr and 10 hr) cells at normoxia or 1% O₂. (FIG. 16S) A graph of the CsCl density gradient profile from the normoxia and hypoxia triplicate samples used in the rereplication experiment. Positions of the light:light (L:L; no replication), heavy:light (H:L; normal replication) and heavy:heavy (H:H; rereplicated) are indicated. Error bars represent the S.E.M. * indicates significant difference from control samples by two-tailed Student's t-test (p<0.05).

FIGS. 17A-17M demonstrate that hypoxia stabilizes KDM4A protein levels. (FIG. 17A,17B) Overexpression of KDM3A does not promote copy gain. Western blot depicting overexpression of Halo-KDM3A for 24 or 72 hours (FIG. 17A), which is insufficient to promote copy gain of 1q12h (FIG. 17B). (FIG. 17C-17F) Depletion of KDM4B-C does not impede hypoxia-mediated copy gain. (FIG. 17C) siRNA-directed depletion of KDM4B,C or D in normoxic and hypoxic RPE cells was verified by qRT-PCR analyses. (FIG. 17D-17F) Western blot confirming depletion of KDM4B (FIG. 17D), KDM4C (FIG. 17E) and KDM4D (FIG. 17F) in RPE cells maintained in normoxia and hypoxia. (FIG. 17G) Western blot depicting siRNA-mediated depletion of KDM4A under normoxic and hypoxic conditions. (FIG. 17H) Cell cycle profile following siRNA depletion of KDM4A in normoxia and hypoxia. (FIGS. 17I-17K) Genomic deletion of KDM4A using CRISPR/Cas9 abrogates KDM4A expression. (FIG. 17I) Western blot indicating relative KDM4A protein levels in 293T parental (293T) and 293T CRISPR cell lines expressing GFP-KDM4A (WT19 and WT28). “Endo” indicates endogenous KDM4A in parental 293T cells, while “GFP” indicates exogenous GFP-KDM4A reintroduced in to WT19 and WT28. (FIG. 17J) A western blot demonstrating KDM4A protein levels in 293T CRISPR cell lines stably expressing GFP and GFP-KDM4A upon normoxic and hypoxic exposure. Lanes were spliced together from different regions of the same exposure of the same blot. (FIG. 17K) Cell cycle profiles of 293T CRISPR GFP and GFP-KDM4A cell lines in normoxia or hypoxia. (FIG. 17L) KDM4A transcript levels do not correlate with increased protein observed in hypoxia. KDM4A mRNA levels were analyzed by qRT-PCR and normalized to β-actin. (FIG. 17M) Hypoxia increases KDM4A protein levels in breast (MDA-MB-468 and MDA-MB-231), neuroblastoma (SK-NAS and SK-N-DZ), and myeloma (MM. 1S) cell lines. For all panels, error bars represent the S.E.M. * indicates significant difference from control samples by two-tailed Student's t-test (p<0.05).

FIGS. 18A-18O demonstrate that KDM4A protein levels are dynamic and correlate with hypoxia treatment. (FIG. 18A) KDM4A levels are increased in hypoxia but return to baseline when cells are returned to normoxia (Rescue). (FIG. 18B) KDM4A levels return to baseline within four hours of return to normoxia. KDM4A levels were analyzed by western blot at the indicated times after a 48 hour 1% O₂ treatment. (FIG. 18C) Western blot depicting KDM4A levels in asynchronous (−) and HU arrested and released cells in hypoxic and normoxic conditions. (FIG. 18D) Hypoxia increases the half-life of KDM4A in 293T cells. Quantification of half-life indicates a half-life of 1 hr 51 min 28 min in normoxia and 6 hr 13 min±10 min in hypoxia. * indicates significant difference from control samples at the same time point by two-tailed Student's t-test (p<0.05). (FIG. 18E,18F) KDM4A ubiquitination is decreased in hypoxic conditions. (FIG. 18E) KDM4A was immunoprecipitated from 293T cells maintained in normoxia or hypoxia using KDM4A-P006 (D4) and KDM4A-P014 (D5) (Van Rechem et al. 2015). IPs were washed under denaturing conditions and analyzed by western blotting. (FIG. 18F) Graphical representation of KDM4A ubiquitination in normoxia and hypoxia. Quantification of ubiquitination indicates an approximately 2.2-fold reduction in ubiquitination upon exposure to hypoxia. Data represents the average of seven independent experiments. (FIG. 18G) KDM4A demethylase activity is retained following prolonged hypoxic exposure. RPE cells expressing 3×HA-WT-KDM4A were maintained in normoxia or hypoxia for 48 hours and H3K9 and H3K36 demethylation was assessed by immunofluorescence. The graph represents an average of two independent experiments with demethylase activity in hypoxia normalized to activity in normoxia. (FIG. 18H) Western blot depicting that JIB-04 treatment does not alter KDM4A protein levels upon hypoxia treatment. Lanes were spliced together from different regions of the same exposure of the same blot. (FIG. 18I) Cell cycle analysis following JIB-04 treatment demonstrating no difference in cell cycle phases. (FIG. 18J-18M) Depletion of KDM5A and KDM6B does not rescue hypoxia-dependent copy gains. (FIG. 18J) Quantification of FISH for 1q12h and 8c in RPE cells maintained in normoxia or hypoxia with or without depletion of KDM5A or KDM6B. Data represents the average of two independent experiments performed with two independent siRNAs. (FIG. 18K) Western blot demonstrating siRNA depletion of KDM5A and CAIX induction in hypoxia. Lanes were spliced together from different regions of the same exposure of the same blot. (FIG. 18L,18M) Quantification of siRNA-mediated depletion of KDM6B (FIG. 18L) and induction of CAIX (FIG. 18M) in normoxic or hypoxic RPE cells using qRT-PCR. Expression was normalized to β-actin and siCTRL in normoxia. (FIG. 18N) Western blot depicting that succinate does not alter KDM4A protein levels upon hypoxia treatment. Lanes were spliced together from different regions of the same exposure of the same blot. (FIG. 18O) Cell cycle analysis following succinate treatment demonstrating no difference in cell cycle phases. For all panels, error bars represent the S.E.M. and * indicates significant difference from control samples by two-tailed Student's t-test (p<0.05).

FIGS. 19A-19E demonstrate that hypoxic tumor samples have copy gains of regions amplified in hypoxic cell culture. (FIG. 19A) TCGA Breast Cancer samples with a hypoxic gene signature have increased focal copy number gain. (FIG. 19B) TCGA Breast Cancer samples with a hypoxic gene signature have increased focal copy number loss. (FIG. 19C) TCGA lung adenocarcinoma samples with a hypoxic gene signature have increased focal copy number gain. (FIG. 19E) TCGA lung adenocarcinoma samples with a hypoxic gene signature have increased focal copy number loss. (FIG. 19E) Western blot depicting siRNA-directed depletion of KDM4A in normoxia and hypoxia.

FIGS. 20A-20E depict graphs of experiments in which E. coli (Top10) were subjected to hypoxia (1%) and normoxia and genomic DNA was isolated and sequenced. The data demonstrates that altered DNA levels are occurring with hypoxic stress as observed with the KDM4-related regions in mammalian cells.

FIGS. 21A-21D demonstrate the regulation of KDM4A by miRNA. FIG. 21A depicts a schematic of KDM4A 3′UTR. The length in base pairs and the positions of TARGETSCAN 6.2 predicted seed sequences are indicated. The seed sequences are indicated as are the mutations performed to generate the mutant 3′-UTR (MT) in the schematic. FIG. 21B depicts western blot analysis of KDM4A protein levels following treatment with the indicated miRNA mimics. Representative western from one of two biological replicates. FIG. 21C depicts Western blot analysis of KDM4A protein levels following treatment with the indicated miRNA inhibitors (anti-miRs). Representative western from one of two biological replicates. FIG. 21D depicts luciferase analysis of KDM4A WT and KDM4A MT 3′-UTR response to miRNA mimics. Data were normalized to the co-transfected β-galactosidase levels for relative light units. Data represent average of two biological replicates assayed in technical triplicates. Error bars represent the S.E.M. * indicates significant difference from CTRL by two-tailed Student's t-test (p<0.05).

FIGS. 22A-22L demonstrate regulation of KDM4A by miRNAs promotes copy gain. FIG. 22A depicts Western blot analysis of KDM4A levels in response to miRNA inhibitors in RPE cells. Representative western from one of two biological replicates. FIG. 22B demonstrates that treatment of RPE cells with the indicated anti-miRs does not affect cell cycle distribution. Representative cell cycle distribution from one of two biological replicates. FIG. 22C depicts representative images of FISH for 1q12h and 8c in anti-miR treated RPE cells. Note the increased number of 1q12h foci (indicated by green chevrons), but not chromosome 8c foci (indicated by red chevrons) in the anti-miR treated cells. Scale bars represent 5 am. FIG. 22D demonstrates that treatment of RPE cells with anti-miRs induces copy gain of 1q12-21. Quantification of FISH analysis. Data represent the average of two biological replicates. Error bars represent the S.E.M. * indicates significant difference from CTRL by two-tailed Student's t-test (p<0.05). FIG. 22E depicts Western blot analysis of KDM4A levels in response to miRNA inhibitors in MDA-MB-231 cells. Representative western from one of two biological replicates. FIG. 22F demonstrates that steady state KDM4A transcript levels do not change in response to miRNA inhibitors in MDA-MB-231 cells. Data represent the average of two biological replicates. Error bars represent the S.E.M. * indicates significant difference from CTRL by two-tailed Student's t-test (p<0.05). FIG. 22G depicts cell cycle distribution of MDA-MB-231 cells treated with anti-mirs. Representative distribution from one of two biological replicates. FIG. 22H demonstrates that treatment of MDA-MB-231 cells with anti-miRs induces copy gain of 1q12h. Quantification of FISH analysis. Data represent the average of two biological replicates. Error bars represent the S.E.M. * indicates significant difference from CTRL by two-tailed Student's t-test (p<0.05). FIG. 22I depicts Western blot analysis of KDM4A levels in response to miRNA inhibitors in SK-N-AS neuroblastoma cells. Representative western from one of two biological replicates. FIG. 22J demonstrates that treatment of SK-N-AS cells with anti-miRs induces copy gain of 1q12h. Quantification of FISH analysis. Data represent the average of two biological replicates. Error bars represent the S.E.M. * indicates significant difference from CTRL by two-tailed Student's t-test (p<0.05). FIG. 22K depicts Western blot analysis of KDM4A levels in response to miRNA inhibitors in H2591 lung cancer cells. Representative western from one of two biological replicates. FIG. 22L demonstrates that treatment of H2591 cells with anti-miRs induces copy gain of 1q12h. Quantification of FISH analysis. Data represent the average of two biological replicates. Error bars represent the S.E.M. * indicates significant difference from CTRL by two-tailed Student's t-test (p<0.05).

FIGS. 23A-23E demonstrate that microRNA-dependent regulation of KDM4A promotes TSSG. FIG. 23A depicts a Western blot depicting KDM4A levels in asynchronous or hydroxyurea (HU) arrested and released cells treated with miRNA inhibitors. Representative western from one of two biological replicates. FIG. 23B demonstrates that copy gain induced by miRNA inhibitors is transient. Quantification of FISH analysis from of asynchronous RPE cells (Asyn), or HU arrested (HU 0) or HU released for four hours (HU 4). Data represent the average of two biological replicates. Error bars represent the S.E.M. * indicates significant difference from untreated CTRL by two-tailed Student's t-test (p<0.05). FIGS. 23C-23E demonstrate that treatment of RPE cells with the indicated anti-miRs does not affect cell cycle distribution (FIG. 23C) or HU arrest (FIG. 23D) or HU release (FIG. 23E). Representative cell cycle profiles from one of two biological replicates.

FIGS. 24A-24C demonstrate that regulation of TSSG by miRNA is KDM4A-dependent. FIG. 24A depicts a Western blot depicting KDM4A levels from combined anti-miR and KDM4A depletion. Representative western from one of two biological replicates. FIG. 24B demonstrates that treatment of RPE cells with the indicated anti-miRs and siRNAs does not affect cell cycle distribution. Representative cell cycle distribution from one of two biological replicates. FIG. 24C demonstrates that TSSG induced by miRNA inhibitor treatment is KDM4A-dependent. Quantification of FISH analysis. Data represent the average of two biological replicates. Error bars represent the S.E.M. * indicates significant difference from CTRL by two-tailed Student's t-test (p<0.05). † indicates significant difference from corresponding anti-miR treated with siCTRL by two-tailed Student's t-test (p<0.05) but not significantly different from anti-miR CTRL/siCTRL.

FIGS. 25A-25C demonstrate that increased MicroRNA expression can ablate hypoxia-dependent TSSG. FIG. 25A depicts a Western blot depicting inhibition of hypoxia-dependent KDM4A induction using miRNA mimics. Representative western from one of two biological replicates. FIG. 25B demonstrates that cell cycle distribution of RPE cells treated with anti-mirs. Representative cell cycle distribution from one of two biological replicates. FIG. 25C depicts quantification of FISH analysis of TSSG in hypoxia-treated cells following miRNA mimic treatment. Data represent the average of two biological replicates. Error bars represent the S.E.M. * indicates significant difference from Normoxia CTRL by two-tailed Student's t-test (p<0.05). † indicates significant difference from Hypoxia CTRL by two-tailed Student's t-test (p<0.05) but not significantly different from the Normoxia CTRL.

FIGS. 26A-26D demonstrate that hsa-mir-23a loss in breast cancer correlates with 1q12-21 copy gain and CKS1B expression. FIG. 26A demonstrates that TCGA primary breast tumor samples with loss of hsa-mir-23a have an enrichment for copy gain of 1p11.2 through 1q23.3 (shaded region). Dashed line indicates genomic location of the indicated miRNA. FIG. 26B demonstrates that TCGA primary breast tumor samples with loss of hsa-mir-137 have enrichment for copy gain of 1p11.2 through 1q23.3 (shaded region). Dashed line indicates genomic location of the indicated miRNA. FIGS. 26C-26D demonstrate that expression of the drug resistance oncogene CKS1B is increased in tumors with loss of hsa-mir-23a (FIG. 26C) or gain of KDM4A (FIG. 26D). The wilcoxon p-value is indicated in each box-plot.

FIGS. 27A-27D demonstrate that regulation of CKS1B copy number and expression by miRNAs correlates with a reduced response to cisplatin. FIG. 27A demonstrates that treatment of MDA-MB-231 cells with anti-miRs induces copy gain of CKS1B, but not the control region CDKN2C. Quantification of FISH analysis. Data represent the average of two biological replicates. Error bars represent the S.E.M. * indicates significant difference from CTRL by two-tailed Student's t-test (p<0.05). FIG. 27B demonstrates that treatment of MDA-MB-231 cells with anti-miRs induces expression of CKS1B. Data represent the average of two biological replicates. Error bars represent the S.E.M. * indicates significant difference from CTRL by two-tailed Student's t-test (p<0.05). FIG. 27C demonstrates that treatment of MDA-MB-231 cells with anti-miRs reduced the response to 300 μM cisplatin. Cells were plated and transfected with the indicated anti-miRs. 24 hours later vehicle (0.9% NaCl) or 300 μM cisplatin was added. Cell survival was measured 48 hours later by MTT assay. Data represent the average of eight biological replicates measured in technical quadruplicate. Error bars represent the S.E.M. * indicates significant difference from CTRL by two-tailed Student's t-test (p<0.05). FIG. 27D depicts a Targetscan 7.0 UTR schematic depicting reduced read count at KDM4A 3′-UTR, which would remove hsa-mir-137 seed sequence in some transcripts. Adapted from TARGETSCAN 7.0.

### DETAILED DESCRIPTION

In one aspect, described herein is a method of reducing and/or preventing the development of drug resistance in a cell, the method comprising contacting the cell with an inhibitor of a KDM4A-like enzyme or an inhibitor of an enzyme that hydroxylates nucleic acids and/or histones or histone-like proteins. In one aspect, described herein is a method of reducing and/or preventing an increase in the expression, activity and/or copy number of a drug resistance gene, the method comprising contacting the cell with an inhibitor of a KDM4A-like enzyme or an inhibitor of an enzyme that hydroxylates nucleic acids and/or histones or histone-like proteins.

In some embodiments, the cell can be a prokaryotic cell, e.g. a bacterial cell. In some embodiments the drug resistance can be antibiotic resistance. In some embodiments, the cell can be a eurkaryotic cell, e.g. a yeast, fungal, or mammalian cell. In some embodiments, the cell can be a cancer cell. In some embodiments, the drug resistance can be chemotherapeutic resistance.

As used herein, “drug resistance” refers to a lack of sensitivity of a cell to a cytotoxic and/or cytostatic agent or the lack of responsiveness of a disease to a treatment drug. Drug resistance can be associated with and/or caused by, e.g., mutations in a drug target, expression/overexpression/gene amplification of a drug transporter protein (e.g. MDR1, ABC transporter proteins). Drug resistance can be resistance to a specific compound, class of compounds, or resistance to multiple compounds and/or classes of compounds. Drug resistance can refer to, e.g., resistance of a cancer cell to a chemotherapeutic agent or resistance of a microbe to an antibiotic or antifungal agent.

In some embodiments, the cell is a cell determined to be experiencing hypoxic conditions. In some embodiments, the cell can be a cell (e.g. a prokaryotic cell) that comprises a gene encoding a KDM4A-like enzyme. Non-limiting examples of prokaryotic cells that comprise a gene encoding a KDM4A-like enzyme are provided in Tables 1 and 2 herein. In some embodiments, the method further comprises the step of determining that the prokaryotic cell comprises a gene encoding a KDM4A-like enzyme. One of skill in the art is familiar with methods of determining if a cell comprises and/or expresses a particular gene and/or a gene with a particular domain or sequence, e.g. RT-PCT, hybridization, Western blotting, etc. Genomic and proteome information is also readily available in a number of databases.

As described herein, “KDM4A,” “Lysine-specific demethylase 4A,” or “JMJD2A” refers to a H3K9/36me3 lysine demethylase of the Jumonji domain 2 (JMJD2) family which converts specific trimethylated histone residues to the dimethylated form. KDM4A encodes a polypeptide having a JmjN domain, JmjC domain, two TUDOR domains, and two PHD-type zinc fingers. The sequence of KDM4A for a number of species is well known in the art, e.g., human KDM4A (e.g. NCBI Gene ID: 9682; (mRNA: SEQ ID NO: 1, NCBI Ref Seq: NM_014663)(polypeptide: SEQ ID NO: 2, NCBI Ref Seq:NP_055478). The sequences of KDM family members are known in the art, e.g. human KDM4B (NCBI Gene ID: 23030 (polypeptide, NCBI Ref Seq: NP_055830, SEQ ID NO: 6)(mRNA, NCBI Ref Seq: NM_015015, SEQ ID NO: 5), human KDM4C (NCBI Gene ID: 23081 (polypeptide, NCBI Ref Seq: NP_055876, SEQ ID NO: 8)(mRNA, NCBI Ref Seq: NM_015061, SEQ ID NO: 7), human KDM4D (NCBI Gene ID: 55693 (polypeptide, NCBI Ref Seq: NP_060509, SEQ ID NO: 10)(mRNA, NCBI Ref Seq: NM_018039, SEQ ID NO: 9), and human KDM4E (NCBI Gene ID: 390245 (polypeptide, NCBI Ref Seq: NP_001155102, SEQ ID NO: 12)(mRNA, NCBI Ref Seq: NM_001161630, SEQ ID NO: 11).

As used herein, “KDM4A-like enzyme” refers to an enzyme with a cupin β barrel domain. The cupin β barrel is a flattened beta-barrel structure with two sheets of five antiparallel beta strands that form the walls of a zinc-binding cleft. In KDM4A, the 3 barrel forms an enzymatic pocket that coordinates Fe(III) and alphaKG. The 3 barrel is located within the JmjC domain of KDM4A. A Cupin protein is a protein comprises at least one cupin β barrel structure.

The cupin β barrel is further described, and can be searched for in other proteins, in the Interpro database (see, e.g. IPR003347); Expasy Prosite (see, e.g. PDOC51183 and PRU00538); PDB (see, e.g. 1H2K); and SMART (see, e.g., SM00558). Non-limiting examples of KDM4A-like enzymes can include KDM4A; KDM5A; KDM6B; KDM4B; KDM4C; a member of the JmjC enzyme family (e.g., KDM2A, KDM2B, KDM3A, KDM3B, KDM4A, KDM4B, KDM4C, KDM5C, KDM6B, and KDM7); a Cupin protein; the proteins listed in Tables 1 and 2 and/or homologs thereof, and Uniprot Gene No FIC_02536. Further discussion of the cupin β barrel can also be found, e.g. in Clissold and Pontig et al. TRENDS in Biochemical Sciences 2001 26:7-9; which is incorporated by reference herein in its entirety.

As used herein, the term “inhibitor” refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, KDM4A, e.g. its ability to decrease the level and/or activity of KDM4A can be determined, e.g. by measuring the level of an expression product of KDM4A and/or the activity of KDM4A. Methods for measuring the level of a given mRNA and/or polypeptide are known to one of skill in the art, e.g. RTPCR with primers can be used to determine the level of RNA and Western blotting with an antibody (e.g. an anti-KDM4A antibody, e.g. Cat No. ab105953; Abcam; Cambridge, Mass.) can be used to determine the level of a polypeptide. The activity of, e.g. KDM4A can be determined using methods known in the art and described above herein. In some embodiments, the inhibitor of KDM4A can be an inhibitory nucleic acid or an aptamer.

Non-limiting examples of inhibitors of KDM4A-like enzymes can include an inhibitory nucleic acid; an aptamer; a miRNA; antibody reagent; an antibody; a small molecule; Suv39H1; HP1; increased oxygen levels; an inhibitor of a KDM4A-targeting KMT; an inhibitor of Tudor or PHD domain interaction; succinate; and JIB-04 and derivatives thereof. In some embodiments, the inhibitor can be an allosteric or enzymatic inhibitor, e.g., succinate. miRNAs can include, e.g. miR23a, miR23b, miR200a, miR200b, miR200c, and miR137a or variants thereof. In some embodiments, the miRNA can be selected from the group consisting of miR23a (e.g. NCBI Gene ID: 407010; SEQ ID NO: 21), miR23b (e.g. NCBI Gene ID: 407011; SEQ ID NO: 22), miR200a (e.g. NCBI Gene ID: 406983; SEQ ID NO: 23), miR200b (e.g. NCBI Gene ID: 406984; SEQ ID NO: 24), miR200c (e.g. NCBI Gene ID: 406985; SEQ ID NO: 25), miR137a (e.g. NCBI Gene ID: 406928; SEQ ID NO: 26) or variants thereof. In some embodiments, the miRNA can be selected from the group consisting of miR23a, miR23b, miR200b, miR200c, miR137a or variants thereof. In some embodiments, the KDM4A inhibitor can be the small molecule JIB-04 or derivatives thereof, 8-(1H-pyrazol-3-yl)pyrido[3,4-d]pyrimidin-4(3H)-ones or derivatives thereof, 3-((furan-2-ylmethyl)amino)pyridine-4-carboxylic acid or derivatives thereof, and 3-(((3-methylthiophen-2-yl)methyl)amino)pyridine-4-carboxylic acid or derivatives thereof (for further details, see, e.g. Wang et al. Nature Communciations 2013 4; Bavetsias et al. J. Med. Chem., 2016, 59 (4), pp 1388-1409; and Westaway et al. Med. Chem., 2016, 59 (4), pp 1357-1369; each of which is incorporated by reference herein in its entirety).

In some embodiments, the inhibitor of KDM4A can be a nucleic acid comprising the sequence of hsa-mir-23a-3p (miRBase Accession No. MIMAT0000078), hsa-mir-23b-3p (miRBase Accession No. MIMAT0000418) and/or hsa-mir-137 (miRBase Accession No. MI0000454). In some embodiments, the inhibitor of KDM4A can be a nucleic acid consistently essentially of the sequence of hsa-mir-23a-3p (miRBase Accession No. MIMAT0000078), hsa-mir-23b-3p (miRBase Accession No. MIMAT0000418) and/or hsa-mir-137 (miRBase Accession No. MI0000454). In some embodiments, the inhibitor of KDM4A can be a nucleic acid consisting of the sequence of hsa-mir-23a-3p (miRBase Accession No. MIMAT0000078), hsa-mir-23b-3p (miRBase Accession No. MIMAT0000418) and/or hsa-mir-137 (miRBase Accession No. MI0000454).

As used herein, “KDM4A-targeting KMT” refers to a lysine (K) specific histone methyltransferase (KMT) that targets at least one target shared by KDM4A, e.g., a target such that KDM4A is recruited to the appropriate location to facilitate copy gains and/or drug resistance. Non-limiting examples of KDM4A-targeting KMTs can include SETD1B (e.g., NCBI Gene ID: 23067); KMTs for H3K4 and H4K20methylation (e.g., MLL1-4 (e.g., NCBI Gene ID: 4297, 9757, 8085, and 58508), SETD1A,B (KMT2 family) (e.g., NCBI Gene ID: 9739 and 23067); KMT5 (e.g., NCBI Gene IDs: 387893, 51111, and 84787) and KMT3 (e.g., NCBI Gene IDs: 29072, 64324, 56950, 150572, and 64754) families (e.g., KMT3B (e.g., NCBI Gene ID: 64324)) or other enzymes that modify these methylation states. Such enzymes are further described in, e.g., Black, et al. Mol Cell 2012 48:491-507; which is incorporated by reference herein in its entirety.

As used herein, “an inhibitor of Tudor or PHD domain interaction” refers to an agent that inhibits the ability of Tudor and/or PHD domains to interact with target histones. Non-limiting examples of such inhibitors can include histone mimetics, small molecules, or a polypeptide comprising at least one PhD domain and one Tudor domain.

In some embodiments, a KDM4A inhibitor can inhibit KDM4A; KDM5; and/or KDM6. For example, JIB-04 can inhibit all three of KDM4A; KDM5; and KDM6.

In some embodiments, the inhibitor of KDM4A can be a nucleic acid comprising the sequence of hsa-mir-23a-3p, hsa-mir-23b-3p and/or hsa-mir-137.

Other enzymes that hydroxylate nucleic acids and/or histone or histone-like proteins are known in the art, see, e.g. Shi et al. Mol Cell. 2007 Jan. 12;25(1): 1-14; which is incorporated by reference herein in its entirety.

In one aspect, described herein is a method of treating an infection in a subject, the method comprising administering an inhibitor of a KDM4A-like enzyme or an inhibitor of an enzyme that hydroxylates nucleic acids and/or histones or histone-like proteins. In some embodiments, the inhibitor of a KDM4A-like enzyme or an inhibitor of an enzyme that hydroxylates nucleic acids and/or histones or histone-like proteins can prevent and/or reduce the emergence of drug resistance in the pathogen. In some embodiments, the inhibitor of a KDM4A-like enzyme or an inhibitor of an enzyme that hydroxylates nucleic acids and/or histones or histone-like proteins can prevent and/or reduce gain of receptors for cell entry (e.g. as used by bacterial and/or viral pathogens to infect a cell). In some embodiments, the inhibitor of a KDM4A-like enzyme or an inhibitor of an enzyme that hydroxylates nucleic acids and/or histones or histone-like proteins can prevent and/or reduce rereplication of viral and/or pathogen genomes in host cells, e.g. host cells with increased KDMs or KMTs. In some embodiments, the inhibitor of a KDM4A-like enzyme or an inhibitor of an enzyme that hydroxylates nucleic acids and/or histones or histone-like proteins can inhibit translation of the pathogen genes, e.g. by inhibiting KDMs and/or KMTs. In some embodiments, the method can further comprise administering a pathogen translation inhibitor. In some embodiments, the inhibitor of a KDM4A-like enzyme or an inhibitor of an enzyme that hydroxylates nucleic acids and/or histones or histone-like proteins can reduce and/or prevent mutation of the pathogen.

In one aspect, described herein is a method of treating an infection in a subject, the method comprising administering: a) an antibiotic and b) an inhibitor of a KDM4A-like enzyme or an inhibitor of an enzyme that hydroxylates nucleic acids and/or histones or histone-like proteins. As used herein, “antibiotic” refers to an agent that reduces or prevents microbial growth.

In some embodiments, the antibiotic is a DNA damage inducing agent. Non-limiting examples of DNA damage-inducing antibiotics can include quinolones (e.g. sparfloxacin, ciprofloxacin, and norfloxacin), beta-lactams (e.g. penams, cephalosporins, monobactams, and carbapenens) and aminoglycosides (e.g. streptomyscin, kamamycin, tobramycin, gentamicin, and neomycin). In some embodiments, the antibiotic can be an antibiotic used to treat an anaerobe infection. Non-limiting examples of antibiotics used to treat anaerobic infections can include clindamycin; metronidazole; carbapenems (eg, imipenem/cilastatin, meropenem, ertapenem), β-lactam/β-lactamase combinations (eg, piperacillin/tazobactam, ampicillin/sulbactam, amoxicillin/clavulanate, ticarcillin/clavulanate), cefoxitin; cefotetan; tigecycline, and moxifloxacin.

In some embodiments, the infection can be a fungal infection; a yeast infection; a eurkaryotic infection; a prokaryotic infection; or a bacterial infection. In some embodiments, the infection comprises an organism comprising a gene encoding a KDM4A-like enzyme. In some embodiments, the method can further comprise the step of determining that the infection comprises an organism comprising a gene encoding a KDM4A-like enzyme.

In one aspect, described herein is a method of reducing and/or preventing the development of drug resistance in a subject in need of treatment for cancer, the method comprising administering a) a chemotherapeutic agent and b) an inhibitor of a KDM4A-like enzyme or an inhibitor of an enzyme that hydroxylates nucleic acids and/or histones or histone-like proteins. In some embodiments, the chemotherapeutic agent is selected from the group consisting of: DNA-damaging agents (e.g. anthracyclines, nitrogen mustards, nitrosoureas, tetrazines, aziridines, cisplatins and derivatives, procarbazine, hexamethylmelamine, bleomycin, doxorubicin, and the like); S-phase chemotherapeutics; mTOR inhibitors; protein synthesis inhibitors; Braf inhibitors; PI3K inhibitors; Cdk inhibitors; Aurora B inhibitors; FLT3 inhibitors; PLK1/2/3 inhibitors; Eg5 inhibitors; β-tubulin inhibitors; BMP inhibitors; HDAC inhibitors; Akt inhibitors; IGF1R inhibitors; p53 inhibitors; hdm2 inhibitors; STAT3 inhibitors; VEGFR inhibitors; angiogenesis inhibitors; proteasomal inhibitors; ubiquitin-targeting drugs; and bortezomib.

In one aspect, described herein is a method of reducing and/or preventing the development of drug resistance in a subject in need of treatment with an angiogenesis inhibitor, the method comprising administering: a) the angiogeneisis inhibitor and b) an inhibitor of a KDM4A-like enzyme or an inhibitor of an enzyme that hydroxylates nucleic acids and/or histones or histone-like proteins. In one aspect, described herein is a method comprising administering: a) an angiogenesis inhibitor and b) an inhibitor of a KDM4A-like enzyme or an inhibitor of an enzyme that hydroxylates nucleic acids and/or histones or histone-like proteins to a subject in need of anti-angiogenic therapy. In some embodiments, the angiogenesis inhibitor is selected from the group consisting of: bevacizumab; itraconzaole; carboxyamidotriazole; TNP-470; CM101; IFN-α; IL-12; platelet factor-4; suramin; SU5416; thrombospondin; VEGFR antagonists; cartilage-derived angiogenesis inhibitory factor; matrix metalloproteinase inhibitors; angiostatin; endostatin; 2-methoxyestradiol; tecogala; tetrathiomolybdate; thalidomide; thrombospondin; prolactin; αVβ3 inhibitors; linomide; tasquinimod; ranibizumab; sorefenib; sunitinib; pazopanib; and everolimus. In some embodiments, a subject in need of anti-angiogenic therapy can be a subject having or diagnosed as having cancer. In some embodiments, a subject in need of anti-angiogenic therapy can be a subject having or diagnosed as having macular degeneration.

In one aspects, the methods described herein relate to reducing and/or preventing the development of drug resistance in a subject experiencing hypoxia, the method comprising administering an inhibitor of a KDM4A-like enzyme or an inhibitor of an enzyme that hydroxylates nucleic acids and/or histones or histone-like proteins to the subject. In some embodiments, the hypoxia occurs in at least one tissue. In some embodiments, the hypoxia occurs in a tumor or cancer cells. In some embodiments, the subject is a subject with cancer or in need of treatment for cancer.

In some embodiments, the methods described herein relate to treating a subject having or diagnosed as having cancer with a composition or treatment described herein. Subjects having cancer can be identified by a physician using current methods of diagnosing cancer. Symptoms and/or complications of cancer which characterize these conditions and aid in diagnosis are well known in the art and include but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer, or exposure to risk factors for cancer (e.g. tobacco products, radiation, etc.) can also aid in determining if a subject is likely to have cancer or in making a diagnosis of cancer.

In some embodiments, the methods described herein relate to treating a subject having or diagnosed as having an infection with a composition or treatment described herein. Subjects having an infection can be identified by a physician using current methods of diagnosing infections. Symptoms and/or complications of infections which characterize these conditions and aid in diagnosis are well known in the art and include but are not limited to, fever, microbial growth, impairment of infection tissues and/or organs etc. Tests that may aid in a diagnosis of, e.g. infection include, but are not limited to, microbial culture of samples. Exposure to risk factors for infections can also aid in determining if a subject is likely to have cancer or in making a diagnosis of infection.

The compositions and methods described herein can be administered to a subject having or diagnosed as having cancer and/or infections. In some embodiments, the methods described herein comprise administering an effective amount of compositions described herein to a subject in order to alleviate a symptom of a disease. As used herein, “alleviating a symptom” of a disease is ameliorating any condition or symptom associated with the disease. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount needed to alleviate at least one or more symptom of the disease or disorder, and relates to a sufficient amount of pharmacological composition to provide the desired effect. The term “therapeutically effective amount” therefore refers to an amount of an agent that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active agent which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for tumor growth, among others. The dosage can be determined by a physician and adjusted, as necessary, to suit observed effects of the treatment.

In some embodiments, the technology described herein relates to a pharmaceutical composition, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, as described herein.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of a composition as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B 1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. In some embodiments, a second agent and/or treatment can comprise dietary succinate supplementation. Non-limiting examples of a second agent and/or treatment can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

In addition, the methods of treatment can further include the use of radiation or radiation therapy. Further, the methods of treatment can further include the use of surgical treatments.

In certain embodiments, an effective dose of a composition as described herein can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. cancer by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the composition. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration, according to the methods described herein depend upon, for example, the form of the composition, its potency, and the extent to which symptoms, markers, or indicators of a condition described herein are desired to be reduced, for example the percentage reduction desired for tumor size or growth. The dosage should not be so large as to cause adverse side effects, such as toxicity in healthy tissue. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein (e.g. reduced growth of cancer cells) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. a reduction in tumor growth). It is well within the ability of one skilled in the art to monitor efficacy of administration and/or treatment by measuring any one of such parameters, or any combination of parameters. Efficacy can be assessed in animal models of a condition described herein, for example treatment of cancer. When using an experimental animal model, efficacy of treatment is evidenced when a statistically significant change in a marker is observed, e.g. tumor growth.

In vitro and animal model assays are provided herein which allow the assessment of a given dose of a composition. By way of non-limiting example, the effects of a dose can be assessed by contacting a tumor cell line grown in vitro with a composition described herein and/or treating it in accordance with the methods described herein. The efficacy of a given dosage combination can also be assessed in an animal model, e.g. a mouse model of any of the cancer described herein.

As described herein, the levels of KDM4A-like enyzmes can regulate cellular processes that contribute to the development of drug resistance. Accordingly, the propensity of a cell to develop drug resistance (e.g., the likelihood that the cell is undergoing processes that promote drug resistance or is likely to undergo such processes in the presence of a drug) can be determined according to the methods provided herein. In one aspect, described herein is a method of detecting a drug-resistance promoting state in a subject, the method comprising: detecting the presence of a copy-gained region in a sample of cell-free DNA obtained from the subject. In one aspect, described herein is a method of detecting a drug-resistance promoting state in a subject, the method comprising: detecting the presence of a copy-gained region in a sample of DNA obtained from the subject. In some embodiments, the copy-gained region can be detected by DNA FISH, e.g., slides, tissue, and cell DNA FISH.

As used herein, “copy-gained region” refers to a region of the genome that is subject to preferential copy number increase, copy number variation and/or gene amplification in cancer cells as opposed to healthy cells. In some embodiments, the copy-gained region comprises the 1q12h (hsat2), 1q12h/21 (e.g., ANK (eg., NCBI Gene ID No: 286)) CKS1B (e.g., NCBI Gene ID No: 1163), DH FR (e.g., NCBI Gene ID No: 1719), BCL9 (e.g., NCBI Gene ID No: 607), and/or Xp13.1 gene. In some embodiments, the copy-gained region is selected from the group consisting of: 1q12-1q25; 1q12h: 1q21.2; and Xq31.1. In some embodiments, the copy-gained region comprises the 1q21-23 locus.

In some embodiments, the method further comprises the step of treating the subject with an inhibitor of a KDM4A-like enzyme or an inhibitor of an enzyme that hydroxylates nucleic acids and/or histones or histone-like proteins.

Techniques for the detection of DNA, e.g. DNA comprising a copy-gained region is known by persons skilled in the art, and can include but not limited to, PCR procedures, quantitative PCR, Northern blot analysis, differential gene expression, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified.

In some embodiments, the level of DNA sequence in a sample can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the copy-gained regions and/or the genes contained therein described herein have been assigned NCBI accession numbers for different species such as human, mouse and rat. Accordingly, a skilled artisan can design an appropriate primer based on the known sequence for detecting and/or measuring the level of a copy-gained region.

Nucleic acid molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, the level of a copy-gained region in cell-free DNA can be compared to a reference sample or level. In some embodiments, the reference level can be the level in a healthy subject not diagnosed as having or not having cancer. In some embodiments, the reference level can be the level in a healthy, non-cancerous cell from the same subject.

The term “sample” or “test sample” as used herein denotes a sample taken or isolated from a biological organism, e.g., a tumor sample from a subject. Exemplary biological samples include, but are not limited to, a biofluid sample; serum; plasma; urine; saliva; a tumor sample; a tumor biopsy and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, a test sample can be a tumor cell test sample, e.g. the sample can comprise cancerous cells, cells from a tumor, and/or a tumor biopsy.

The test sample can be obtained by removing a sample from a subject, but can also be accomplished by using previously isolated samples (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent. Chemical and/or biological reagents can be employed to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of the presence of a cop-gained region as described herein.

In some embodiments of any of the aspects, the sample obtained from a subject can be a blood or serum sample. In some embodiments, the sample is a tissue sample, urine sample, or plasma sample.

In some embodiments, the methods, assays, and systems described herein can further comprise a step of obtaining a test sample from a subject. In some embodiments, the subject can be a human subject.

In some embodiments, measurement of the level of a target and/or detection of the level or presence of a target, e.g. of an expression product (nucleic acid or polypeptide of one of the genes described herein) or a mutation can comprise a transformation. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample, e.g., from whole blood to blood serum by differential centrifugation. A biological/chemical transformation can involve the action of at least one enzyme and/or a chemical reagent in a reaction. For example, a DNA sample can be digested into fragments by one or more restriction enzymes, or an exogenous molecule can be attached to a fragmented DNA sample with a ligase. In some embodiments, a DNA sample can undergo enzymatic replication, e.g., by polymerase chain reaction (PCR).

Transformation, measurement, determining of the precence of, and/or detection of a target molecule, e.g. a DNA sequence, a mRNA, or, a polypeptide can comprise contacting a sample obtained from a subject with a reagent (e.g. a detection reagent) which is specific for the target, e.g., a target-specific reagent. In some embodiments, the target-specific reagent is detectably labeled. In some embodiments, the target-specific reagent is capable of generating a detectable signal. In some embodiments, the target-specific reagent generates a detectable signal when the target molecule is present.

Methods to measure gene expression products are known to a skilled artisan. Such methods to measure gene expression products, e.g., protein level, include ELISA (enzyme linked immunosorbent assay), western blot, immunoprecipitation, and immunofluorescence using detection reagents such as an antibody or protein binding agents. Alternatively, a peptide can be detected in a subject by introducing into a subject a labeled anti-peptide antibody and other types of detection agent. For example, the antibody can be labeled with a detectable marker whose presence and location in the subject is detected by standard imaging techniques.

For example, antibodies for the various targets described herein are commercially available and can be used for the purposes of the invention to measure protein expression levels, e.g. anti-KDM4A (Cat. No. ab105953; Abcam, Cambridge Mass.). Alternatively, since the amino acid sequences for the targets described herein are known and publically available at the NCBI website, one of skill in the art can raise their own antibodies against these polypeptides of interest for the purpose of the invention.

The amino acid sequences of the polypeptides described herein have been assigned NCBI accession numbers for different species such as human, mouse and rat.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience a change of color, upon encountering the targeted molecules. In some instances, signal amplification can be integrated into the particular protocol, wherein a secondary antibody, that includes the marker stain or marker signal, follows the application of a primary specific antibody.

In some embodiments, the assay can be a Western blot analysis. Alternatively, proteins can be separated by two-dimensional gel electrophoresis systems. Two-dimensional gel electrophoresis is well known in the art and typically involves iso-electric focusing along a first dimension followed by SDS-PAGE electrophoresis along a second dimension. These methods also require a considerable amount of cellular material. The analysis of 2D SDS-PAGE gels can be performed by determining the intensity of protein spots on the gel, or can be performed using immune detection. In other embodiments, protein samples are analyzed by mass spectroscopy.

Immunological tests can be used with the methods and assays described herein and include, for example, competitive and non-competitive assay systems using techniques such as Western blots, radioimmunoassay (RIA), ELISA (enzyme linked immunosorbent assay), “sandwich” immunoassays, immunoprecipitation assays, immunodiffusion assays, agglutination assays, e.g. latex agglutination, complement-fixation assays, immunoradiometric assays, fluorescent immunoassays, e.g. FIA (fluorescence-linked immunoassay), chemiluminescence immunoassays (CLIA), electrochemiluminescence immunoassay (ECLIA, counting immunoassay (CIA), lateral flow tests or immunoassay (LFIA), magnetic immunoassay (MIA), and protein A immunoassays. Methods for performing such assays are known in the art, provided an appropriate antibody reagent is available. In some embodiments, the immunoassay can be a quantitative or a semi-quantitative immunoassay.

An immunoassay is a biochemical test that measures the concentration of a substance in a biological sample, typically a fluid sample such as urine, using the interaction of an antibody or antibodies to its antigen. The assay takes advantage of the highly specific binding of an antibody with its antigen. For the methods and assays described herein, specific binding of the target polypeptides with respective proteins or protein fragments, or an isolated peptide, or a fusion protein described herein occurs in the immunoassay to form a target protein/peptide complex. The complex is then detected by a variety of methods known in the art. An immunoassay also often involves the use of a detection antibody.

Enzyme-linked immunosorbent assay, also called ELISA, enzyme immunoassay or EIA, is a biochemical technique used mainly in immunology to detect the presence of an antibody or an antigen in a sample. The ELISA has been used as a diagnostic tool in medicine and plant pathology, as well as a quality control check in various industries.

In one embodiment, an ELISA involving at least one antibody with specificity for the particular desired antigen (e.g., any of the targets as described herein) can also be performed. A known amount of sample and/or antigen is immobilized on a solid support (usually a polystyrene micro titer plate). Immobilization can be either non-specific (e.g., by adsorption to the surface) or specific (e.g. where another antibody immobilized on the surface is used to capture antigen or a primary antibody). After the antigen is immobilized, the detection antibody is added, forming a complex with the antigen. The detection antibody can be covalently linked to an enzyme, or can itself be detected by a secondary antibody which is linked to an enzyme through bio-conjugation. Between each step the plate is typically washed with a mild detergent solution to remove any proteins or antibodies that are not specifically bound. After the final wash step the plate is developed by adding an enzymatic substrate to produce a visible signal, which indicates the quantity of antigen in the sample. Older ELISAs utilize chromogenic substrates, though newer assays employ fluorogenic substrates with much higher sensitivity.

In another embodiment, a competitive ELISA is used. Purified antibodies that are directed against a target polypeptide or fragment thereof are coated on the solid phase of multi-well plate, i.e., conjugated to a solid surface. A second batch of purified antibodies that are not conjugated on any solid support is also needed. These non-conjugated purified antibodies are labeled for detection purposes, for example, labeled with horseradish peroxidase to produce a detectable signal. A sample (e.g., a blood sample) from a subject is mixed with a known amount of desired antigen (e.g., a known volume or concentration of a sample comprising a target polypeptide) together with the horseradish peroxidase labeled antibodies and the mixture is then are added to coated wells to form competitive combination. After incubation, if the polypeptide level is high in the sample, a complex of labeled antibody reagent-antigen will form. This complex is free in solution and can be washed away. Washing the wells will remove the complex. Then the wells are incubated with TMB (3, 3′, 5, 5′-tetramethylbenzidene) color development substrate for localization of horseradish peroxidase-conjugated antibodies in the wells. There will be no color change or little color change if the target polypeptide level is high in the sample. If there is little or no target polypeptide present in the sample, a different complex in formed, the complex of solid support bound antibody reagents-target polypeptide. This complex is immobilized on the plate and is not washed away in the wash step. Subsequent incubation with TMB will produce significant color change. Such a competitive ELSA test is specific, sensitive, reproducible and easy to operate.

There are other different forms of ELISA, which are well known to those skilled in the art. The standard techniques known in the art for ELISA are described in “Methods in Immunodiagnosis”, 2nd Edition, Rose and Bigazzi, eds. John Wiley & Sons, 1980; and Oellerich, M. 1984, J. Clin. Chem. Clin. Biochem. 22:895-904. These references are hereby incorporated by reference in their entirety.

In one embodiment, the levels of a polypeptide in a sample can be detected by a lateral flow immunoassay test (LFIA), also known as the immunochromatographic assay, or strip test. LFIAs are a simple device intended to detect the presence (or absence) of antigen, e.g. a polypeptide, in a fluid sample. There are currently many LFIA tests used for medical diagnostics, either for home testing, point of care testing, or laboratory use. LFIA tests are a form of immunoassay in which the test sample flows along a solid substrate via capillary action. After the sample is applied to the test strip it encounters a colored reagent (generally comprising antibody specific for the test target antigen) bound to microparticles which mixes with the sample and transits the substrate encountering lines or zones which have been pretreated with another antibody or antigen. Depending upon the level of target polypeptides present in the sample the colored reagent can be captured and become bound at the test line or zone. LFIAs are essentially immunoassays adapted to operate along a single axis to suit the test strip format or a dipstick format. Strip tests are extremely versatile and can be easily modified by one skilled in the art for detecting an enormous range of antigens from fluid samples such as urine, blood, water, and/or homogenized tissue samples etc. Strip tests are also known as dip stick tests, the name bearing from the literal action of “dipping” the test strip into a fluid sample to be tested. LFIA strip tests are easy to use, require minimum training and can easily be included as components of point-of-care test (POCT) diagnostics to be use on site in the field. LFIA tests can be operated as either competitive or sandwich assays. Sandwich LFIAs are similar to sandwich ELISA. The sample first encounters colored particles which are labeled with antibodies raised to the target antigen. The test line will also contain antibodies to the same target, although it may bind to a different epitope on the antigen. The test line will show as a colored band in positive samples. In some embodiments, the lateral flow immunoassay can be a double antibody sandwich assay, a competitive assay, a quantitative assay or variations thereof. Competitive LFIAs are similar to competitive ELISA. The sample first encounters colored particles which are labeled with the target antigen or an analogue. The test line contains antibodies to the target/its analogue. Unlabelled antigen in the sample will block the binding sites on the antibodies preventing uptake of the colored particles. The test line will show as a colored band in negative samples. There are a number of variations on lateral flow technology. It is also possible to apply multiple capture zones to create a multiplex test.

The use of “dip sticks” or LFIA test strips and other solid supports have been described in the art in the context of an immunoassay for a number of antigen biomarkers. U.S. Pat. Nos. 4,943,522; 6,485,982; 6,187,598; 5,770,460; 5,622,871; 6,565,808, U.S. patent application Ser. No. 10/278,676; U.S. Ser. No. 09/579,673 and U.S. Ser. No. 10/717,082, which are incorporated herein by reference in their entirety, are non-limiting examples of such lateral flow test devices. Examples of patents that describe the use of “dip stick” technology to detect soluble antigens via immunochemical assays include, but are not limited to U.S. Pat. Nos. 4,444,880; 4,305,924; and 4,135,884; which are incorporated by reference herein in their entireties. The apparatuses and methods of these three patents broadly describe a first component fixed to a solid surface on a “dip stick” which is exposed to a solution containing a soluble antigen that binds to the component fixed upon the “dip stick,” prior to detection of the component-antigen complex upon the stick. It is within the skill of one in the art to modify the teachings of this “dip stick” technology for the detection of polypeptides using antibody reagents as described herein.

Other techniques can be used to detect the level of a polypeptide in a sample. One such technique is the dot blot, and adaptation of Western blotting (Towbin et at., Proc. Nat. Acad. Sci. 76:4350 (1979)). In a Western blot, the polypeptide or fragment thereof can be dissociated with detergents and heat, and separated on an SDS-PAGE gel before being transferred to a solid support, such as a nitrocellulose or PVDF membrane. The membrane is incubated with an antibody reagent specific for the target polypeptide or a fragment thereof. The membrane is then washed to remove unbound proteins and proteins with non-specific binding. Detectably labeled enzyme-linked secondary or detection antibodies can then be used to detect and assess the amount of polypeptide in the sample tested. The intensity of the signal from the detectable label corresponds to the amount of enzyme present, and therefore the amount of polypeptide. Levels can be quantified, for example by densitometry.

In some embodiments, the level of a target can be measured, by way of non-limiting example, by Western blot; immunoprecipitation; enzyme-linked immunosorbent assay (ELISA); radioimmunological assay (RIA); sandwich assay; fluorescence in situ hybridization (FISH); immunohistological staining; radioimmunometric assay; immunofluoresence assay; mass spectroscopy and/or immunoelectrophoresis assay.

In certain embodiments, the gene expression products as described herein can be instead determined by determining the level of messenger RNA (mRNA) expression of the genes described herein. Such molecules can be isolated, derived, or amplified from a biological sample, such as a blood sample. Techniques for the detection of mRNA expression is known by persons skilled in the art, and can include but not limited to, PCR procedures, RT-PCR, quantitative RT-PCR Northern blot analysis, differential gene expression, RNAse protection assay, microarray based analysis, next-generation sequencing; hybridization methods, etc.

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, the level of an mRNA can be measured by a quantitative sequencing technology, e.g. a quantitative next-generation sequence technology. Methods of sequencing a nucleic acid sequence are well known in the art. Briefly, a sample obtained from a subject can be contacted with one or more primers which specifically hybridize to a single-strand nucleic acid sequence flanking the target gene sequence and a complementary strand is synthesized. In some next-generation technologies, an adaptor (double or single-stranded) is ligated to nucleic acid molecules in the sample and synthesis proceeds from the adaptor or adaptor compatible primers. In some third-generation technologies, the sequence can be determined, e.g. by determining the location and pattern of the hybridization of probes, or measuring one or more characteristics of a single molecule as it passes through a sensor (e.g. the modulation of an electrical field as a nucleic acid molecule passes through a nanopore). Exemplary methods of sequencing include, but are not limited to, Sanger sequencing, dideoxy chain termination, high-throughput sequencing, next generation sequencing, 454 sequencing, SOLiD sequencing, polony sequencing, Illumina sequencing, Ion Torrent sequencing, sequencing by hybridization, nanopore sequencing, Helioscope sequencing, single molecule real time sequencing, RNAP sequencing, and the like. Methods and protocols for performing these sequencing methods are known in the art, see, e.g. “Next Generation Genome Sequencing” Ed. Michal Janitz, Wiley-VCH; “High-Throughput Next Generation Sequencing” Eds. Kwon and Ricke, Humanna Press, 2011; and Sambrook et al., Molecular Cloning: A Laboratory Manual (4 ed.), Cold Spring Harbor Laboratory Press, Cold Spring Harbor, N.Y., USA (2012); which are incorporated by reference herein in their entireties.

The nucleic acid sequences of the genes described herein have been assigned NCBI accession numbers for different species such as human, mouse and rat. For example, the human KDM4A mRNA is known. Accordingly, a skilled artisan can design an appropriate primer based on the known sequence for determining the mRNA level of the respective gene.

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; heat and alkaline lysis procedures can be useful for obtaining nucleic acid molecules from urine; and proteinase K extraction can be used to obtain nucleic acid from blood (Roiff, A et al. PCR: Clinical Diagnostics and Research, Springer (1994)).

In some embodiments, one or more of the reagents (e.g. an antibody reagent and/or nucleic acid probe) described herein can comprise a detectable label and/or comprise the ability to generate a detectable signal (e.g. by catalyzing reaction converting a compound to a detectable product). Detectable labels can comprise, for example, a light-absorbing dye, a fluorescent dye, or a radioactive label. Detectable labels, methods of detecting them, and methods of incorporating them into reagents (e.g. antibodies and nucleic acid probes) are well known in the art.

In some embodiments, detectable labels can include labels that can be detected by spectroscopic, photochemical, biochemical, immunochemical, electromagnetic, radiochemical, or chemical means, such as fluorescence, chemifluoresence, or chemiluminescence, or any other appropriate means. The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies). The detectable label can be linked by covalent or non-covalent means to the reagent. Alternatively, a detectable label can be linked such as by directly labeling a molecule that achieves binding to the reagent via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In other embodiments, the detection reagent is label with a fluorescent compound. When the fluorescently labeled reagent is exposed to light of the proper wavelength, its presence can then be detected due to fluorescence. In some embodiments, a detectable label can be a fluorescent dye molecule, or fluorophore including, but not limited to fluorescein, phycoerythrin, phycocyanin, o-phthaldehyde, fluorescamine, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxyfhiorescein (commonly known by the abbreviations FAM and F), 6-carboxy-2′,4′,7′,4,7-hexachlorofiuorescein (HEX), 6-carboxy-4′,5′-dichloro-2′,7′-dimethoxyfiuorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes. In some embodiments, a detectable label can be a radiolabel including, but not limited to ³H, ¹²⁵I, ³⁵S, ¹⁴C, ³²P, and ³³P. In some embodiments, a detectable label can be an enzyme including, but not limited to horseradish peroxidase and alkaline phosphatase. An enzymatic label can produce, for example, a chemiluminescent signal, a color signal, or a fluorescent signal. Enzymes contemplated for use to detectably label an antibody reagent include, but are not limited to, malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-VI-phosphate dehydrogenase, glucoamylase and acetylcholinesterase. In some embodiments, a detectable label is a chemiluminescent label, including, but not limited to lucigenin, luminol, luciferin, isoluminol, theromatic acridinium ester, imidazole, acridinium salt and oxalate ester. In some embodiments, a detectable label can be a spectral colorimetric label including, but not limited to colloidal gold or colored glass or plastic (e.g., polystyrene, polypropylene, and latex) beads.

In some embodiments, detection reagents can also be labeled with a detectable tag, such as c-Myc, HA, VSV-G, HSV, FLAG, V5, HIS, or biotin. Other detection systems can also be used, for example, a biotin-streptavidin system. In this system, the antibodies immunoreactive (i. e. specific for) with the biomarker of interest is biotinylated. Quantity of biotinylated antibody bound to the biomarker is determined using a streptavidin-peroxidase conjugate and a chromagenic substrate. Such streptavidin peroxidase detection kits are commercially available, e. g. from DAKO; Carpinteria, Calif. A reagent can also be detectably labeled using fluorescence emitting metals such as ¹⁵²Eu, or others of the lanthanide series. These metals can be attached to the reagent using such metal chelating groups as diethylenetriaminepentaacetic acid (DTPA) or ethylenediaminetetraacetic acid (EDTA).

A level which is less than a reference level can be a level which is less by at least about 10%, at least about 20%, at least about 50%, at least about 60%, at least about 80%, at least about 90%, or less than the reference level. In some embodiments, a level which is less than a reference level can be a level which is statistically significantly less than the reference level.

A level which is more than a reference level can be a level which is greater by at least about 10%, at least about 20%, at least about 50%, at least about 60%, at least about 80%, at least about 90%, at least about 100%, at least about 200%, at least about 300%, at least about 500% or more than the reference level. In some embodiments, a level which is more than a reference level can be a level which is statistically significantly greater than the reference level.

In some embodiments, the reference can be a level of the target molecule in a population of subjects who do not have or are not diagnosed as having, and/or do not exhibit signs or symptoms of a condition or state described herein. In some embodiments, the reference can also be a level of expression of the target molecule in a control sample, a pooled sample of control individuals or a numeric value or range of values based on the same. In some embodiments, the reference can be the level of a target molecule in a sample obtained from the same subject at an earlier point in time, e.g., the methods described herein can be used to determine if a subject's state or condition (e.g., likelihood of developing drug resistance) is changing over time.

In some embodiments, the level of expression products of no more than 200 other genes is determined. In some embodiments, the level of expression products of no more than 100 other genes is determined. In some embodiments, the level of expression products of no more than 20 other genes is determined. In some embodiments, the level of expression products of no more than 10 other genes is determined.

In some embodiments of the foregoing aspects, the expression level of a given gene can be normalized relative to the expression level of one or more reference genes or reference proteins.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer or infection. A subject can be male or female.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art.

A subject can be one who has been previously diagnosed with or identified as suffering from or having a condition in need of treatment (e.g. cancer or infection) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the term “antibiotic” refers to any compound known to one of ordinary skill in the art that will inhibit or reduce the growth of, or kill, one or more microorganisms, including bacterial species and fungal species. Many antibacterial compounds are relatively small molecules with a molecular weight of less than 2000 atomic mass units. The term “antibiotic” includes semi-synthetic modifications of various natural compounds, such as, for example, the beta-lactam antibiotics, which include penicillins (produced by fungi in the genus Penicillium), the cephalosporins, the monobactams, and the carbapenems. Accordingly, the term “antibiotic” includes, but is not limited to, aminoglycosides (e.g., gentamicin, streptomycin, kanamycin), β-lactams (e.g., penicillins, cephalosporins, monobactams, and carbapenems), vancomycins, bacitracins, macrolides (e.g., erythromycins), lincosamides (e.g., clindomycin), chloramphenicols, tetracyclines, amphotericins, cefazolins, clindamycins, mupirocins, sulfonamides and trimethoprim, rifampicins, metronidazoles, quinolones, novobiocins, polymyxins, gramicidins, or any salts or variants thereof. The antibiotic used in addition to the aminoglycoside antibiotic various embodiments of the therapeutic compositions and methods described herein will depend on the type of bacterial infection.

As used herein, the term “cancer” or “tumor” refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastases. Cancers which migrate from their original location and seed vital organs can eventually lead to the death of the subject through the functional deterioration of the affected organs.

As used herein “gene copy number” refers to the number of copies of a given gene that occur in the genome. As used herein, “gene amplification” refers to the presence of a greater than normal gene copy number within the cell. In some embodiments, the copies are located on the same chromosome. In some embodiments, the copies are located on more than one chromosome. In some embodiments, gene copy number can include partial copies of a gene, e.g. less than the full coding sequence.

The term “agent” refers generally to any entity which is normally not present or not present at the levels being administered to a cell, tissue or subject. An agent can be selected from a group including but not limited to: polynucleotides; polypeptides; small molecules; and antibodies or antigen-binding fragments thereof. A polynucleotide can be RNA or DNA, and can be single or double stranded, and can be selected from a group including, for example, nucleic acids and nucleic acid analogues that encode a polypeptide. A polypeptide can be, but is not limited to, a naturally-occurring polypeptide, a mutated polypeptide or a fragment thereof that retains the function of interest. Further examples of agents include, but are not limited to a nucleic acid aptamer, peptide-nucleic acid (PNA), locked nucleic acid (LNA), small organic or inorganic molecules; saccharide; oligosaccharides; polysaccharides; biological macromolecules, peptidomimetics; nucleic acid analogs and derivatives; extracts made from biological materials such as bacteria, plants, fungi, or mammalian cells or tissues and naturally occurring or synthetic compositions. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety selected, for example, from unsubstituted or substituted alkyl, aromatic, or heterocyclyl moieties including macrolides, leptomycins and related natural products or analogues thereof. Agents can be known to have a desired activity and/or property, or can be selected from a library of diverse compounds. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

As used herein the term “chemotherapeutic agent” refers to any chemical or biological agent with therapeutic usefulness in the treatment of diseases characterized by abnormal cell growth. Such diseases include tumors, neoplasms and cancer as well as diseases characterized by hyperplastic growth. These agents can function to inhibit a cellular activity upon which the cancer cell depends for continued proliferation. In some aspect of all the embodiments, a chemotherapeutic agent is a cell cycle inhibitor or a cell division inhibitor. Categories of chemotherapeutic agents that are useful in the methods of the invention include alkylating/alkaloid agents, antimetabolites, hormones or hormone analogs, and miscellaneous antineoplastic drugs. Most of these agents are directly or indirectly toxic to cancer cells. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In some embodiments, the chemotherapeutic agent can be a cytotoxic chemotherapeutic. The term “cytotoxic agent” as used herein refers to a substance that inhibits or prevents the function of cells and/or causes destruction of cells. The term is intended to include radioactive isotopes (e.g. At211, I131, I125, Y90, Re186, Re188, Sm153, Bi212, P32 and radioactive isotopes of Lu), chemotherapeutic agents, and toxins, such as small molecule toxins or enzymatically active toxins of bacterial, fungal, plant or animal origin, including fragments and/or variants thereof.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to KDM4A.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

Aptamers are short synthetic single-stranded oligonucleotides that specifically bind to various molecular targets such as small molecules, proteins, nucleic acids, and even cells and tissues. These small nucleic acid molecules can form secondary and tertiary structures capable of specifically binding proteins or other cellular targets, and are essentially a chemical equivalent of antibodies. Aptamers are highly specific, relatively small in size, and non-immunogenic. Aptamers are generally selected from a biopanning method known as SELEX (Systematic Evolution of Ligands by Exponential enrichment) (Ellington et al. Nature. 1990; 346(6287):818-822; Tuerk et al., Science. 1990; 249(4968):505-510; Ni et al., Curr Med Chem. 2011; 18(27):4206-14; which are incorporated by reference herein in their entireties). Methods of generating an apatmer for any given target are well known in the art. Preclinical studies using, e.g. aptamer-siRNA chimeras and aptamer targeted nanoparticle therapeutics have been very successful in mouse models of cancer and HIV (Ni et al., Curr Med Chem. 2011; 18(27):4206-14).

Inhibitors of the expression of a given gene can be an inhibitory nucleic acid. In some embodiments, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of KDM4A. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 564,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂—[known as a methylene (methylimino) or MMI backbone], —CH₂—O—N(CH₃)—CH₂—, —CH₂—N(CH₃)—N(CH₃)—CH₂— and —N(CH₃)—CH₂—CH₂—[wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the invention involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments, a nucleic acid encoding a polypeptide as described herein, or a nucleic acid comprising a miRNA sequence as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding encoding a polypeptide as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. infection or cancer. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of reducing and/or preventing the development of drug resistance in a cell, the method comprising contacting the cell with an inhibitor of a KDM4A-like enzyme or an inhibitor of an enzyme that hydroxylates nucleic acids and/or histones or histone-like proteins.
2. The method of paragraph 1, wherein the cell is a prokaryotic cell.
3. The method of paragraph 2, wherein the drug resistance is antibiotic resistance.
4. The method of paragraph 1, wherein the cell is a eukaryotic cell.
5. The method of paragraph 4, wherein the cell is selected from the group consisting of: a yeast cell and a mammalian cell.
6. The method of paragraph 3, wherein the cell is a cancer cell.
7. The method of paragraph 4, wherein the drug resistance is chemotherapeutic resistance.
8. The method of any of paragraphs 1-7, wherein the cell is contacted with an inhibitor of a KDM4A-like enzyme.
9. The method of any of paragraphs 1-8, wherein the KDM4A-like enzyme comprises a cupin β barrel domain.
10. The method of any of paragraphs 1-9, wherein the KDM4A-like enzyme is selected from the group consisting of:
   KDM4A; KDM5A; KDM6B; KDM4B; KDM4C; a Cupin protein; and the proteins listed in Tables 1 and 2 and/or homologs thereof.
11. The method of any of paragraphs 1-10, wherein the KDM4A-like enzyme is KDM4A.
12. The method of any of paragraphs 1-10, wherein the inhibitor of a KDM4A-like enzyme is selected from the group consisting of:
   an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; and JIB-04.
13. The method of any of paragraphs 1-11, wherein the cell is a cell determined to be experiencing hypoxic conditions.
14. The method of any of paragraphs 1-12, wherein the prokaryotic cell comprises a gene encoding a KDM4A-like enzyme.
15. The method of any of paragraphs 1-13, further comprising the step of determining that the prokaryotic cell comprises a gene encoding a KDM4A-like enzyme.
16. A method of treating an infection in a subject, the method comprising administering inhibitor of a KDM4A-like enzyme or an inhibitor of an enzyme that hydroxylates nucleic acids and/or histones or histone-like proteins.
17. A method of treating an infection in a subject, the method comprising administering:
   a) an antibiotic and
   b) an inhibitor of a KDM4A-like enzyme or an inhibitor of an enzyme that hydroxylates nucleic acids and/or histones or histone-like proteins.
18. The method of paragraph 16, wherein the antibiotic is a DNA damage inducing agent or an antibiotic used to treat an anaerobe infection.
19. The method of any of paragraphs 16-17, wherein the infection is selected from the group consisting of:
   a fungal infection; a yeast infection; a eurkaryotic infection; a prokaryotic infection; and a bacterial infection.
20. The method of any of paragraphs 16-18, wherein the infection comprises an organism comprising a gene encoding a KDM4A-like enzyme.
21. The method of any of paragraphs 16-19, further comprising the step of determining that the infection comprises an organism comprising a gene encoding a KDM4A-like enzyme.
22. A method of reducing and/or preventing the development of drug resistance in a subject in need of treatment for cancer, the method comprising administering
   a) a chemotherapeutic agent and
   b) an inhibitor of a KDM4A-like enzyme or an inhibitor of an enzyme that hydroxylates nucleic acids and/or histones or histone-like proteins.
23. The method of paragraph 21, wherein the chemotherapeutic agent is selected from the group consisting of:
   DNA-damaging agents; S-phase chemotherapeutics; mTOR inhibitors; protein synthesis inhibitors; Braf inhibitors; PI3K inhibitors; Cdk inhibitors; Aurora B inhibitors; FLT3 inhibitors; PLK1/2/3 inhibitors; Eg5 inhibitors; P3-tubulin inhibitors; BMP inhibitors; HDAC inhibitors; Akt inhibitors; IGF1R inhibitors; p53 inhibitors; hdm2 inhibitors; STAT3 inhibitors; VEGFR inhibitors; angiogenesis inhibitors; proteasomal inhibitors; ubiquitin-targeting drugs; and bortezomib.
24. A method of reducing and/or preventing the development of drug resistance in a subject in need of treatment with an angiogenesis inhibitor, the method comprising administering:
   a. the angiogeneisis inhibitor and
   b. an inhibitor of a KDM4A-like enzyme or an inhibitor of an enzyme that hydroxylates nucleic acids and/or histones or histone-like proteins.
25. A method comprising administering:
   a. an angiogenesis inhibitor and
   b. an inhibitor of a KDM4A-like enzyme or an inhibitor of an enzyme that hydroxylates nucleic acids and/or histones or histone-like proteins to a subject in need of anti-angiogenic therapy.
26. The method of any of paragraphs 23-24, wherein the angiogenesis inhibitor is selected from the group consisting of:
   bevacizumab; sorefenib; sunitinib; pazopanib; and everolimus
27. The method of any of paragraphs 16-25, wherein the subject is administered an inhibitor of a KDM4A-like enzyme.
28. The method of paragraph 26, wherein the KDM4A-like enzyme comprises a cupin β barrel domain.
29. The method of any of paragraphs 26-27, wherein the KDM4A-like enzyme is selected from the group consisting of:
   KDM4A; KDM5A; KDM6B; KDM4B; KDM4C; a Cupin protein; and the proteins listed in Tables 1 and 2 and/or homologs thereof.
30. The method of any of paragraphs 26-28, wherein the inhibitor of a KDM4A-like enzyme is selected from the group consisting of:
   an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; and JIB-04.
31. A method of detecting a drug-resistance promoting state in a subject, the method comprising: detecting the presence of a copy-gained region in a sample of cell-free DNA obtained from the subject.
32. The method of paragraph 30, wherein the copy-gained region comprises the CKS1B, DHFR, or BCL9 gene.
33. The method of any of paragraphs 30-31, wherein the copy-gained region is a region of the genome that is subject to copy number variation in cancer cells.
34. The method of any of paragraphs 30-32, wherein the copy-gained region is selected from the group consisting of:
   1q12-1q25;1q12h; 1q21.2; and Xq31.1;
35. The method of any of paragraphs 30-33, wherein the sample is a urine or plasma sample.
36. The method of paragraph 30-34, further comprising the step of treating the subject with an inhibitor of a KDM4A-like enzyme or an inhibitor of an enzyme that hydroxylates nucleic acids and/or histones or histone-like proteins.

Some embodiments of the technology described herein can be defined according to any of the following numbered paragraphs:
1. A method of reducing and/or preventing the development of drug resistance in a cell, the method comprising contacting the cell with an inhibitor of a KDM4A-like enzyme or an inhibitor of an enzyme that hydroxylates nucleic acids and/or histones or histone-like proteins.
2. The method of paragraph 1, wherein the cell is a prokaryotic cell.
3. The method of paragraph 2, wherein the drug resistance is antibiotic resistance.
4. The method of paragraph 1, wherein the cell is a eukaryotic cell.
5. The method of paragraph 4, wherein the cell is selected from the group consisting of: a yeast cell and a mammalian cell.
6. The method of paragraph 3, wherein the cell is a cancer cell.
7. The method of paragraph 4, wherein the drug resistance is chemotherapeutic resistance.
8. The method of any of paragraphs 1-7, wherein the cell is contacted with an inhibitor of a KDM4A-like enzyme.
9. The method of any of paragraphs 1-8, wherein the KDM4A-like enzyme comprises a cupin β barrel domain.
10. The method of any of paragraphs 1-9, wherein the KDM4A-like enzyme is selected from the group consisting of:
   KDM4A; KDM5A; KDM6B; KDM4B; KDM4C; a member of the JmjC enzyme family; a Cupin protein; and the proteins listed in Tables 1 and 2 and/or homologs thereof.
11. The method of any of paragraphs 1-10, wherein the KDM4A-like enzyme is KDM4A.
12. The method of any of paragraphs 1-11, wherein the inhibitor of a KDM4A-like enzyme is selected from the group consisting of:
   an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; JIB-04; a 8-(1H-pyrazol-3-yl)pyrido[3,4-d]pyrimidin-4(3H)-one; 3-((furan-2-ylmethyl)amino)pyridine-4-carboxylic acid; and 3-(((3-methylthiophen-2-yl)methyl)amino)pyridine-4-carboxylic acid.
13. The method of any of paragraphs 1-11, wherein the inhibitor of a KDM4A-like enzyme is a nucleic acid comprising the sequence of hsa-mir-23a-3p, hsa-mir-23b-3p and/or hsa-mir-137.
14. The method of any of paragraphs 1-13, wherein the cell is a cell determined to be experiencing hypoxic conditions.
15. The method of any of paragraphs 1-14, wherein the prokaryotic cell comprises a gene encoding a KDM4A-like enzyme.
16. The method of any of paragraphs 1-15, further comprising the step of determining that the prokaryotic cell comprises a gene encoding a KDM4A-like enzyme.
17. A method of treating an infection in a subject, the method comprising administering inhibitor of a KDM4A-like enzyme or an inhibitor of an enzyme that hydroxylates nucleic acids and/or histones or histone-like proteins.
18. A method of treating an infection in a subject, the method comprising administering:
   a) an antibiotic and
   b) an inhibitor of a KDM4A-like enzyme or an inhibitor of an enzyme that hydroxylates nucleic acids and/or histones or histone-like proteins.
19. The method of paragraph 18, wherein the antibiotic is a DNA damage inducing agent or an antibiotic used to treat an anaerobe infection.
20. The method of any of paragraphs 17-19, wherein the infection is selected from the group consisting of:
   a fungal infection; a yeast infection; a eurkaryotic infection; a prokaryotic infection; and a bacterial infection.
21. The method of any of paragraphs 17-20, wherein the infection comprises an organism comprising a gene encoding a KDM4A-like enzyme.
22. The method of any of paragraphs 17-21, further comprising the step of determining that the infection comprises an organism comprising a gene encoding a KDM4A-like enzyme.
23. A method of reducing and/or preventing the development of drug resistance in a subject in need of treatment for cancer, the method comprising administering
   a) a chemotherapeutic agent and
   b) an inhibitor of a KDM4A-like enzyme or an inhibitor of an enzyme that hydroxylates nucleic acids and/or histones or histone-like proteins.
24. The method of paragraph 23, wherein the chemotherapeutic agent is selected from the group consisting of:
   DNA-damaging agents; S-phase chemotherapeutics; mTOR inhibitors; protein synthesis inhibitors; Braf inhibitors; PI3K inhibitors; Cdk inhibitors; Aurora B inhibitors; FLT3 inhibitors; PLK1/2/3 inhibitors; Eg5 inhibitors; 3-tubulin inhibitors; BMP inhibitors; HDAC inhibitors; Akt inhibitors; IGF1R inhibitors; p53 inhibitors; hdm2 inhibitors; STAT3 inhibitors; VEGFR inhibitors; angiogenesis inhibitors; proteasomal inhibitors; ubiquitin-targeting drugs; and bortezomib.
25. A method of reducing and/or preventing the development of drug resistance in a subject experiencing hypoxia, the method comprising administering an inhibitor of a KDM4A-like enzyme or an inhibitor of an enzyme that hydroxylates nucleic acids and/or histones or histone-like proteins to the subject.
26. A method of reducing and/or preventing the development of drug resistance in a subject in need of treatment with an angiogenesis inhibitor, the method comprising administering:
   a. the angiogeneisis inhibitor and
   b. an inhibitor of a KDM4A-like enzyme or an inhibitor of an enzyme that hydroxylates nucleic acids and/or histones or histone-like proteins.
27. A method comprising administering:
   a. an angiogenesis inhibitor and
   b. an inhibitor of a KDM4A-like enzyme or an inhibitor of an enzyme that hydroxylates nucleic acids and/or histones or histone-like proteins to a subject in need of anti-angiogenic therapy.
28. The method of any of paragraphs 26-27, wherein the angiogenesis inhibitor is selected from the group consisting of:
   bevacizumab; sorefenib; sunitinib; pazopanib; and everolimus.
29. The method of any of paragraphs 17-28, wherein the subject is administered an inhibitor of a KDM4A-like enzyme.
30. The method of paragraph 29, wherein the KDM4A-like enzyme comprises a cupin β barrel domain.
31. The method of any of paragraphs 29-30, wherein the KDM4A-like enzyme is selected from the group consisting of:
   KDM4A; KDM5A; KDM6B; KDM4B; KDM4C; a member of the JmjC enzyme family;
   a Cupin protein; and the proteins listed in Tables 1 and 2 and/or homologs thereof.
32. The method of any of paragraphs 29-31, wherein the inhibitor of a KDM4A-like enzyme is selected from the group consisting of:
   an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; JIB-04; a 8-(1H-pyrazol-3-yl)pyrido[3,4-d]pyrimidin-4(3H)-one; 3-((furan-2-ylmethyl)amino)pyridine-4-carboxylic acid; and 3-(((3-methylthiophen-2-yl)methyl)amino)pyridine-4-carboxylic acid.
33. The method of any of paragraphs 29-32, wherein the inhibitor of a KDM4A-like enzyme is a nucleic acid comprising the sequence of hsa-mir-23a-3p, hsa-mir-23b-3p and/or hsa-mir-137.
34. A method of detecting a drug-resistance promoting state in a subject, the method comprising: detecting the presence of a copy-gained region in a sample of cell-free DNA obtained from the subject.
35. The method of paragraph 34, wherein the copy-gained region comprises the 1q12h (hsat2), 1q12h/21 (e.g., ANK) CKS1B, DHFR BCL9, or Xp13.1 gene,
36. The method of any of paragraphs 34-35, wherein the copy-gained region is a region of the genome that is subject to copy number variation in cancer cells.
37. The method of any of paragraphs 34-35, wherein the copy-gained region is selected from the group consisting of
   1q12-1q25;1q12h; 1q21.2; Xq31.1; and 1q21-23 locus.
38. A method of detecting a drug-resistance promoting state in a subject, the method comprising: detecting the presence of an increased level of hsa-mir-23a-3p, hsa-mir-23b-3p and/or hsa-mir-137 in s sample obtained from the subject.
39. The method of any of paragraphs 34-38, wherein the sample is a tissue sample, urine sample, or plasma sample.
40. The method of paragraph 34-39, further comprising the step of treating the subject with an inhibitor of a KDM4A-like enzyme or an inhibitor of an enzyme that hydroxylates nucleic acids and/or histones or histone-like proteins.
41. A therapeutically effective amount of an inhibitor of a KDM4A-like enzyme or an inhibitor of an enzyme that hydroxylates nucleic acids and/or histones or histone-like proteins for reducing and/or preventing the development of drug resistance in a cell.
42. The inhibitor of paragraph 41, wherein the cell is a prokaryotic cell.
43. The inhibitor of paragraph 42, wherein the drug resistance is antibiotic resistance.
44. The inhibitor of paragraph 41, wherein the cell is a eukaryotic cell.
45. The inhibitor of paragraph 42, wherein the cell is selected from the group consisting of: a yeast cell and a mammalian cell.
46. The inhibitor of paragraph 43, wherein the cell is a cancer cell.
47. The inhibitor of paragraph 44, wherein the drug resistance is chemotherapeutic resistance.
48. The inhibitor of any of paragraphs 41-47, wherein the KDM4A-like enzyme comprises a cupin β barrel domain.
49. The inhibitor of any of paragraphs 41-49, wherein the KDM4A-like enzyme is selected from the group consisting of:
   KDM4A; KDM5A; KDM6B; KDM4B; KDM4C; a member of the JmjC enzyme family;
   a Cupin protein; and the proteins listed in Tables 1 and 2 and/or homologs thereof.
50. The inhibitor of any of paragraphs 41-49, wherein the KDM4A-like enzyme is KDM4A.
51. The inhibitor of any of paragraphs 41-50, wherein the inhibitor of a KDM4A-like enzyme is selected from the group consisting of:
   an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; JIB-04; a 8-(1H-pyrazol-3-yl)pyrido[3,4-d]pyrimidin-4(3H)-one; 3-((furan-2-ylmethyl)amino)pyridine-4-carboxylic acid; and 3-(((3-methylthiophen-2-yl)methyl)amino)pyridine-4-carboxylic acid.
52. The inhibitor of any of paragraphs 41-50, wherein the inhibitor of a KDM4A-like enzyme is a nucleic acid comprising the sequence of hsa-mir-23a-3p, hsa-mir-23b-3p and/or hsa-mir-137.
53. The inhibitor of any of paragraphs 41-52, wherein the cell is a cell determined to be experiencing hypoxic conditions.
54. The inhibitor of any of paragraphs 41-53, wherein the prokaryotic cell comprises a gene encoding a KDM4A-like enzyme.
55. A therapeutically effective amount of an inhibitor of a KDM4A-like enzyme or an inhibitor of an enzyme that hydroxylates nucleic acids and/or histones or histone-like proteins for treating an infection in a subject.
56. A therapeutically effective amount of 1) an inhibitor of a KDM4A-like enzyme or an inhibitor of an enzyme that hydroxylates nucleic acids and/or histones or histone-like proteins and 2) an antibiotic for treating an infection in a subject.
57. The composition(s) of paragraph 56, wherein the antibiotic is a DNA damage inducing agent or an antibiotic used to treat an anaerobe infection.
58. The composition(s) of any of paragraphs 55-57, wherein the infection is selected from the group consisting of:
   a fungal infection; a yeast infection; a eurkaryotic infection; a prokaryotic infection; and a bacterial infection.
59. The composition(s) of any of paragraphs 55-58, wherein the infection comprises an organism comprising a gene encoding a KDM4A-like enzyme.
60. A therapeutically effective amount of 1) an inhibitor of a KDM4A-like enzyme or an inhibitor of an enzyme that hydroxylates nucleic acids and/or histones or histone-like proteins and 2) a chemotherapeutic agent for treating a cancer in a subject.
61. The composition(s) of paragraph 60, wherein the chemotherapeutic agent is selected from the group consisting of:
   DNA-damaging agents; S-phase chemotherapeutics; mTOR inhibitors;
   protein synthesis inhibitors; Braf inhibitors; PI3K inhibitors; Cdk inhibitors; Aurora B inhibitors; FLT3 inhibitors; PLK1/2/3 inhibitors; Eg5 inhibitors; P3-tubulin inhibitors; BMP inhibitors; HDAC inhibitors; Akt inhibitors; IGF1R inhibitors; p53 inhibitors; hdm2 inhibitors; STAT3 inhibitors; VEGFR inhibitors; angiogenesis inhibitors; proteasomal inhibitors; ubiquitin-targeting drugs; and bortezomib.
62. A therapeutically effective amount of an inhibitor of a KDM4A-like enzyme or an inhibitor of an enzyme that hydroxylates nucleic acids and/or histones or histone-like proteins for treating hypoxia in a subject.
63. A therapeutically effective amount of 1) an inhibitor of a KDM4A-like enzyme or an inhibitor of an enzyme that hydroxylates nucleic acids and/or histones or histone-like proteins and 2) an angiogenesis inhibitor for treating a subject in need of anti-angiogenic therapy.
64. The composition(s) of paragraph 63, wherein the angiogenesis inhibitor is selected from the group consisting of:
   bevacizumab; sorefenib; sunitinib; pazopanib; and everolimus.
65. The composition(s) of any of paragraphs 55-64, wherein the KDM4A-like enzyme comprises a cupin β barrel domain.
66. The composition(s) of any of paragraphs 55-65, wherein the KDM4A-like enzyme is selected from the group consisting of:
   KDM4A; KDM5A; KDM6B; KDM4B; KDM4C; a member of the JmjC enzyme family;
   a Cupin protein; and the proteins listed in Tables 1 and 2 and/or homologs thereof. 67. The composition(s) of any of paragraphs 55-66, wherein the inhibitor of a KDM4A-like enzyme is selected from the group consisting of:
   an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; JIB-04; a 8-(1H-pyrazol-3-yl)pyrido[3,4-d]pyrimidin-4(3H)-one; 3-((furan-2-ylmethyl)amino)pyridine-4-carboxylic acid; and 3-(((3-methylthiophen-2-yl)methyl)amino)pyridine-4-carboxylic acid.
68. The composition(s) of any of paragraphs 55-66, wherein the inhibitor of a KDM4A-like enzyme is a nucleic acid comprising the sequence of hsa-mir-23a-3p, hsa-mir-23b-3p and/or hsa-mir-137.

### EXAMPLES

### Example 1

Changes in copy number of chromosomes have been identified in lower organisms as well as in certain tissues in mammals. Copy number variations mostly have harmful consequences for the organism and underlie the development of diseases. Described herein are examples of copy alterations from developmental biology and discussion of its role as a source of adaptive response in normal tissues as well as in cancer. Since proper DNA replication and cell division is required for maintaining genome stability, some of the underlying mechanisms resulting in genome instability and rereplication and a new paradigm of generating transient site-specific copy gains (TSSGs) in the genome are discussed. Also discusses are how site-specific genome alterations can serve as a source of intra-tumoral heterogeneity and can influence therapeutic responses and patient outcome in cancer.

Introduction

Maintaining the appropriate genetic composition is a fundamental basis of life. Any alterations from the normal genome content can create imbalances in gene dosages¹ resulting in a variety of diseases². With recent major technological advances such as fluorescence in situ hybridization (FISH), spectral karyotyping (SKY), comparative genome hybridization (CGH), genome-scanning array technologies and single-cell sequencing approaches, changes in the structure and number of chromosomes including sub-chromosomal alterations has been identified. Based on the length of chromosomal changes, copy number alterations have been classified by different terms³ Copy number could result from the insertion of new sequences (1bp-1 kb in size), deletion of already existing sequences, due to a larger sequence change (1 kb-1 Mb; copy number variation) and changes with more than or equal to 1 Mb (referred to as microdeletions or microduplications). When a region within a specific chromosome is changed (sub-chromosomal changes), it is referred to as segmental aneuploidy. Changes resulting from specific chromosomal regions that are transiently amplified are referred to as transient site-specific copy gains (TSSGs). Majority of copy number changes are harmful and can cause diseases either alone or in combination with other genetic or environmental factors⁴. However, copy number changes are frequently observed during development and in lower as well as higher organisms as normal physiological processes.

Described herein are examples emphasizing the biological as well as pathological consequences of copy number variation especially in cancer. The recently identified role of a chromatin regulator in generating transient site-specific copy gains and its major implications as well as some of the critical questions in the field are discussed.

Copy Number Alterations During Development and Physiologic Processes.

Changes in chromosomal copy number and the associated gene amplifications/losses can be observed during normal development in both lower as well as higher eukaryotes. Their presence during normal biology indicates that copy number changes can have functional consequences under selective pressure. Examples from developmental biology and their functional implications during tissue homeostasis under selective conditions such as nutrient or metabolic stress, toxic challenges, injury and replicative stress are provided.

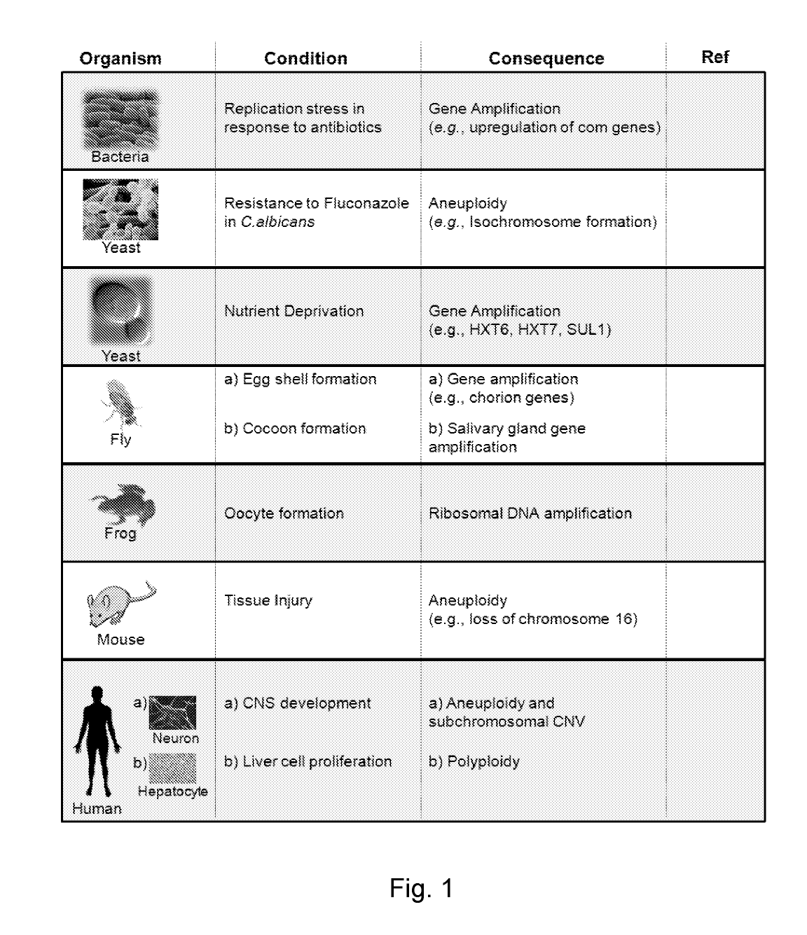

Gene amplification is a fundamental mechanism for meeting increasing demands for structural and growth requirements during tissue development and differentiation. There are several examples from developmental biology showing gene amplifications in response to developmental signals in lower eukaryotes (FIG. 1). Experiments during early 70s from electron microscopy studies showed that ribosomal genes are amplified for the production of large amounts of ribosomes required during early embryogenesis⁵. Ribosomal DNA (rDNA) amplifications were observed during oocyte formation in amphibians Xenopus leavis, insects such as water beetles⁶, molluscs⁷ and teleost fish, and in the macronuclear rDNA in Paramecium⁸ as well as Tetrahymena⁹. Besides ribosomal DNA amplifications, specific chromosomal regions in the salivary gland identified as “DNA puffs” are amplified and expressed to form structural proteins required for cocoon formation in the salivary glands in sciarid flies^{10,11}. This amplification of the DNA puff gene occurs in response to the hormone, ecdysone, required during larval development¹¹. Another example of gene amplification triggered by developmental signals can be observed during eggshell formation in Drosophila¹². Eggshells require amplification of chorion genes in the follicle cells of the ovary and these genes are expressed late in differentiation^{12,13}.

Examples of copy number alterations are also reported in various tissues in mammals under normal conditions. Using techniques such as spectral karyotyping (SKY), fluorescence in situ hybridization (FISH) and single cell sequencing approaches, various groups have reported both small as well as large-scale changes in chromosomal copy number in the tissues in mouse and humans, particularly in neurons, liver and skin fibroblasts (FIG. 1). Around 33% of neuroblasts in the embryonic mouse brain and 20% of neurons in the adult mouse cerebral cortex showed aneuploidy according to a study by Rehen et al.¹⁴. This reduction in aneuploidy in the adult brain was hypothesized due to a programmed cell death mechanism of neuroblasts during brain development¹⁵. Westra et al.¹⁶ also identified between 15-20% aneuploidy in the neural progenitor cells of both mouse as well as human cerebellum underscoring the notion that generation of copy number alterations is an essential process in the development of central nervous system. A recent study identified high levels of subchromosomal CNVs with deletion and duplication events in the frontal cortex neurons of human brain and multiple alterations within a small set of neurons suggesting that CNVs might be restricted to either individual cells or specific neural lineages¹⁷. Besides neurons, chromosomal changes have also been observed in skin as well as mammalian hepatocytes. Hepatocytes have polyploidy chromosomal content and study by Duncan and colleagues suggest that around 50% of normal adult diploid hepatocytes have changes in chromosomal numbers with either gains or losses such that genetically diverse sets of cells are present^{18,19}._ENREF_17 A study by Knouse et al. found that 2.7% of mouse keratinocytes are aneuploid and a much lower level of aneuploidy (under 5%) exist for both liver and human neurons. These differences in the reported levels of aneuploidies could result from the types of assays employed to follow copy alterations (i.e. FISH, SKY vs single cell sequencing). Regardless, determining the biological impact of these events in the brain, liver or skin is an important area to be explored in the future.

Varying degrees of aneuploidy in brain, liver or skin indicate that alterations in copy number can be a mechanism employed during tissue development. This genetic variation can help achieve diversity in cell populations during tissue development so that effective responses can be achieved. For example, copy variations can allow developing tissues to adapt to cellular and growth requirements during tissue expansion and organ development. Another advantage for the observed CNVs can be to help adapt to metabolic or toxic challenges as encountered especially by hepatocytes (discussed in the following section). These examples indicate that biological selection and use of CNV is part of normal biology.

Copy Number Changes as an Adaptive Mechanism.

Many studies in bacteria, yeast and mammals have shown that copy number changes can arise as a consequence of selection during adaptive experiments. This response can provide an “increased fitness” or survival advantage to the cells. Described herein is evidence for copy number variations emerging with stress such as bacterial response to antibiotics, C. albicans response to anti-fungal agents, budding yeast response to nutrient deprivation or replicative stress and human tissue response to selective conditions.

Bacterial. Acquisition of antibiotic resistance can occur through the uptake of foreign DNA harboring resistance genes through a mechanism called the bacterial competence pathway²⁰. A recent study by Slager et al. demonstrated that different species of bacteria could increase the copy number of genes involved in the competence pathway (com genes) in response to antibiotics causing replication stress²¹. These genes are located closer to the origin of replication (OriC) and amplification of genes occurs through multiple origin firing events at OriC, which increases their copy number as well as transcription rates. These data illustrate the impact selective pressure can have on regions and gene products.

Yeast. Gene rearrangements and copy number changes have been observed in Candida albicans when passaged through a murine host²². It has been hypothesized that these changes in ploidy could generate genetic and phenotypic diversity required for adaptation in the new host environment. However, in the case of anti-fungal drug resistance, CNV was associated with adaptive benefits. For example, fluconazole treatment in C. albicans results in the development of whole chromosome gains and aneuploidy²³. Selmecki et al. upon CGH analyses for the copy number in 70 azole-resistant and azole-sensitive strains found increased levels of aneuploidy in resistant strains (50%) compared to fluconazole-sensitive strains (7.14%)^{24,25}. Interestingly, trisomies of chromosome 5, including an isochromosome were associated with resistance. Isochromosomes are formed by the attachment of two left arms of chromosome 5 in an inverted repeat sequence around a single centromere. Gains of this isochromosome were associated with an increased expression of genes involved in drug resistance²⁵. Some of these genes encoded efflux pump proteins: an ABC and a multi-drug resistance transporter (MDR)²⁶. Other genes were ERG11²⁷ (a target of fluconazole) and TAC1 (a transcription factor that upregulates ABC transporter gene expression)²⁵. Consistent with gene amplification conferring selective advantage, budding yeast exposed to nutrient deprivation results in amplifications of genes that benefits the cell²⁸. For example, glucose limitation in cultures resulted in the amplification of genes encoding glucose transporters (HXT6 and HXT7), while sulphate-limitation resulted in the amplifications of SUL1, a gene that encodes for a high affinity sulphate transporter. Taken together, these examples in yeast illustrate how the genome can be selectively modulated for the survival or growth of cells.

Mammals. Diet induced selective pressure also impacts copy number in mammals. For example, copy number of the human salivary amylase gene AMY1, which aids in the hydrolysis of starch, is increased in certain populations that have a higher starch-content in their diets when compared to low-starch consuming populations²⁹. The increased copy number of AMY1 also correlated with increases in the protein levels of salivary amylase. Hence, copy number variations could be a source of diet-induced selection advantage. Increases or decreases in copy number of certain genes can also predispose an individual to diseases. Susceptibility of individuals to HIV/AIDS infection is increased in populations with a decreased copy number of the chemokine gene CCL3L1. This chemokine serves as a ligand for HIV co-receptor CCR5 and this inhibits viral entry by binding to CCR5. H1V resistant individuals show duplications of CCL3L1 locus (17q21.1) with increased CCL3L1 copies imparting resistance to HIV infections³⁰. These observations points to the benefits of germline variation on the population. Copy number variation can serve as a mechanism to adapt to tissue injury. The best example to illustrate this point is the work from Duncan et al. in a chronic liver injury model in mice. Fumaryl acetoacetate hydrolase (FAH) is an enzyme required in tyrosine catabolism that catalyzes the conversion of fumaryl acetoacetotate (FAA) into fumarate and acetoacetate. Deficiency of FAH causes hereditary tyrosinemia from the build up of FAA and toxic metabolites that results in liver failure. Loss of enzymes involved in tyrosine catabolism upstream of FAH (e.g., hydroxyphenyl pyruvate dioxygenase (HPD) which forms homogentisic acid, a precursor to FAA) is found to be protective for this disease. Duncan et al. demonstrated that loss of chromosome 16 with a complete functional loss of HGD (HGD resides in 16qB2) produces hepatocytes that are resistant to tyrosinemia in a chronic liver injury model in mice³¹. Importantly, these injury resistant hepatocyte cells (characterized by the loss of chromosome 16) are already present in the liver and these cells are selected for or undergo expansion when liver is exposed to injury. However, it is not clear whether cells with chromosomal gains/losses are also selected during tissue injury in humans. However, a recent discovery illustrates the ability of regions of the genome to be site-specifically selected under physiological signals such as hypoxia in humans as well as zebrafish cells. Hence, some of the above discussed examples of copy number changes from different species could be a “compensatory mechanism” employed by organisms/cells to increase the survival or fitness under selective environmental, nutritional or therapeutic pressures.

Copy Number Changes in Cancer.

Besides the role of copy number changes observed during development and in normal tissues as discussed in the previous section, copy number alterations are often thought of as a pathological event. This section will discuss pathologically associated copy alteration, with an underlying emphasis on the pathology being a consequence of a defective biological process driving copy selection. In this section, the phenotypic consequences of copy number changes in specific genes or chromosomal regions related to cancer are discussed. These changes can affect either whole chromosomes and/or specific chromosomal regions causing amplifications/deletions of smaller genomic fragments. For example, genome-wide analysis of copy number changes in cancer has identified that 25% of the genome is affected by whole chromosome alterations and 10% by short chromosomal changes (focal)³². The focally amplified regions had previously validated oncogenes (e.g., MYC, CCND1, EGFR, NKX2-1, KRAS), while focally deleted regions contained tumor suppressor genes (ETV6, TMPRSS2-ERG). These analyses also revealed that 10.5% of cancers have focal amplifications of 1q21.2 with MCL1 as a novel amplified target gene. Another gene identified from the focally amplified region of 20q11.21 was BCL2L1³². Both these genes are important for cell survival, hence their somatic amplifications in tumors could confer survival advantage. In fact, Beroukhim et al. demonstrate that increased expression of these genes protected tumor cells from chemotherapy.

Focal amplification in chromosome copy number harboring oncogenes can impact tumorigenesis and drug resistance. In multiple myeloma, disease progression is characterized partly by the focal amplifications of a proximal region of chromosome 1q. Several studies have identified a region of proximal 1q with a marked enrichment of genes showing an amplification/gain spanning a region of 10-15 Mb corresponding to a 1q12-23 amplicon in multiple myeloma (MM)_ENREF_43. This region contains a large number of genes with amplifications or deregulated expression involved in myeloma pathogenesis. Some of these genes include CKS1B, MUC1³³, MCL1³⁴, PDZK1³⁵, IL-6R³⁶, BCL9³⁷, and UBE2Q1³⁸, among others. The amplification of a drug resistant gene, CKS1B and the proximal 1q21 region has been reported in about 40% of newly diagnosed multiple myeloma cases and in 70% of patients with tumor relapse^{39,40}. Interestingly, the increases of gains observed in CKS1B are low (1-3 copies)^{41,42}. These focal amplifications of CKS1B are associated with poor prognosis and reduced response to cisplatin therapy⁴¹. Amplifications of PDZK1 gene within the 1q12-q22 region were observed in primary cases of MM and overexpression of this gene in cells conferred resistance to melphalan, vincristine and cisplatin induced cell deaths³⁵. Besides multiple myeloma, gene amplifications are associated with drug resistance in several other tumors. For example, ovarian cell lines that have amplifications of 1q12-21 chromosomal regions are found to be more resistant to cisplatin treatment^{43,44}. An 11-13-fold higher copy number of region 7q21.12 was detected in an acquired paclitaxel-resistant non-small cell lung cancer model (NCI-H460/PTX250) compared with the parental cell line NCI-H460 using microarray-based comparative genomic hybridization. Most of the genes within this region were also highly expressed including a multidrug transporter gene MDR1/ABCB1⁴⁵. These few examples highlight how distinct regions in the genome are focally selected. The fact that multiple cell types select for regions such as 1q12-21 raise the question that possibly there are mechanisms modulating selection of these regions.

Focal gains or losses of chromosomes can result in diversity within cells in a tumor population (intratumoral heterogeneity). Copy number heterogeneity within tumors has become apparent with genome wide sequencing analysis in melanoma⁴⁶_ENREF_69, medulloblastoma⁴⁷, bladder cancer⁴⁸. Sequencing of five bladder tumors from patients with transitional cell carcinoma of the urinary bladder showed genomic rearrangements and mutational heterogeneity within tumors⁴⁹ Whole exome sequencing of samples from eighteen patients with chronic lymphocytic leukemia (CLL) by Landau and colleagues revealed the emergence of sub-clones within selected population of cells treated with chemotherapy⁵⁰. These populations of cells might be more fit than the pre-treatment counterpart and could contribute to relapse after therapy. Thus, identifying the mutational landscape before and after chemotherapy can not only identify mechanisms of tumor relapse but also help to design effective therapeutic options for elimination of the dominant subclones arising after chemotherapeutic selection pressures thereby decreasing the likelihood of tumor relapse.

Chromatin, Rereplication and TSSG.

There are several mechanisms implicated in genome instability that would lead to changes in gene copy number and chromosome structure^{2,51}. Described herein is the role of chromatin in DNA replication and the recently identified mechanisms of transient site-specific copy gains.

The eukaryotic genome is organized into a highly ordered structure called chromatin. Chromatin is composed of nucleosomes that contain 147 bp of DNA wrapped around a histone octamer with 2 copies each of core histones H2A, H2B, H3 and H4. Histones are modified by a number of post-translational modifications, which influences a range of DNA-templated processes including transcription, replication and DNA repair⁵²⁻⁵⁵_ENREF_104.

Chromatin and chromosomal architecture play fundamental roles in DNA replication. Chromatin modifications such as acetylation and methylation of histones impact origin recognition complex (ORC) recruitment and define replication timing. For example, the H4 histone acetyltransferase Hbo1 (histone acetylase binding to ORC), which was initially identified as an interactor of ORC, Cdt1 and MCM2⁵⁶, is required for the pre-RC assembly in in Xenopus extracts⁵⁷. The recruitment of a catalytically inactive Hbo1 to a mammalian origin of replication hinders the loading of Mcm2-7 proteins⁵⁸. In the case of origin firing, a local increase in histone acetylation mediated by Gcn5 histone acetyl transferase at a late-activating origin promotes origin firing significantly earlier in S phase⁵⁹. Similarly, targeting HAT or HDAC activity to a beta-globin locus can also shift the time of replication from late to early and vice versa⁶⁰.

Besides histone acetylation, other histone modifications such as methylation can also influence DNA replication. Lysine methylation states define chromatin structures such as euchromatin and heterochromatin and impact pre-RC formation and replication timing⁶¹′6². For example, the levels of histone H4 Lys 20 mono-methylation (H4K20me1) are important for helicase loading and pre-RC formation⁶³. The lysine methyltransferase SET-domain containing protein 8 (Set8; also known as PR-Set 7 and KMT5A) monomethylates H4K20. Set 8 promotes recruitment of pre-RC machinery (by recruitment of ORC1, MCM2 and MCM5) to a specific genomic locus by increasing H4K20me1 at replication origins⁶⁴. After the onset of S phase, Set 8 is targeted for proteasomal degradation in a PCNA-dependent manner that contributes to the loss of H4K20me1 at origins and inhibition on licensing, preventing rereplication⁶⁴⁻⁶⁶. Therefore, Set8 levels are critical for maintaining genome stability, as the loss of Set8 function would result in S phase delay, chromosome decondensation, increased DNA damage, DNA content and rereplication^{67,68}.

Histone 3 lysine 9 methylation and heterochromatin formation and maintenance also have important roles in regulating replication. For example, the deletion of yeast H3K9me3 methyltransferase (CLR4) and SWI6 [yeast HP1 (heterochromatin binding protein 1) homolog] result in altered S phase progression and replication stress⁶⁹′7⁰. Mammalian HP1 proteins (HP1α/γ) also directly interact with components of the replication fork and are important for maintaining the late replication of pericentric heterochromatin during S phase⁷¹. Heterochromatin protein 1 (HP1) as well as others can also influence replication initiation by directly binding to ORC and targeting it to specific regions^{72,73}. It was shown in drosophila that HP1 had a dual role in affecting replication timing of heterochromatic regions⁷⁴. RNAi depletion of HP1 advanced the replication timing of centromeric repeat regions; while, replication timing of other regions in pericentromeric heterochromatin was delayed⁷⁴. These data provide a link between the chromatin microenvironment, methylation states and replication initiation.

In addition to the readers and writers of lysine methylation, the lysine demethylases are able to modulate replication timing and rereplication at selective regions of the genome, which impacts the generation of TSSGs within the human genome. For example, the JmjC-domain containing protein KDM4A (also called JMJD2A) demethylates trimethylated histone H3 lysine 9 and 36 (H3K9/36me3) to a dimethylated state (K3K9/36 me2)⁷⁵⁻⁷⁸. KDM4A overexpression promoted faster S-phase progression and altered replication timing at specific regions in the genome in a catalytic-dependent manner^{79,80}. The faster S phase and regulation of replication timing was conserved from C. elegans to human cells and was the result of dysregulating specific HP1 members in the genome (HPL-2 in C. elegans and HP1γ in human cells)⁸⁰. With regard to replication, this direct antagonism between the reader and the eraser established the basis for highly selective regulation within the genome especially replication timing control. Consistent with this intricate relationship between demethylation and reading heterochromatin, KDM4A protein expression is finely tuned throughout cell cycle by the Skp1-Cul1-F-box ubiquitin ligase complexes_ENREF_127⁸¹. These data illustrate another way in which the ubiquitin ligase complexes are able to modulate DNA replication through regulating chromatin modulators such as KDM4A.

Consistent with chromatin modulation being important in regulating replication fidelity, KDM4A was recently shown to promote TSSGs and rereplication within the human genome. KDM4A was shown to promote S phase-dependent TSSGs upon overexpression⁷⁹. The regions that underwent copy gains were dependent on the enzyme activity, the Tudor domains and cells being in S phase. These requirements strongly supported the need to rereplicated regions. Consistent with this notion, KDM4A purified with the majority of the replication machinery-licensing factors, polymerase, etc. The ability of KDM4A to generate TSSGs was antagonized by Suv39H1 and HP1γ, which emphasize the regional regulation and importance of certain modulators in regulating the copy number of specific regions. Furthermore, interference with the methylation of H3K9 or K36 also generated the TSSGs as observed with KDM4A overexpression. These gains were not driven by KDM4A levels but altered chromatin state. Taken together, these data identified the first enzyme capable of generating transient site-specific copy gains and established that chromatin states are involved in modulating the sensitivity to copy alterations at distinct regions in the genome.

The impact KDM4A had on cells in vitro was further verified in human tumors. In fact, analyses of tumors from the cancer genome atlas database (TCGA) allowed the identification of additional regions being regulated by KDM4A. KDM4A was shown to be amplified in ˜20% of the tumors, which correlated with KDM4A RNA levels⁷⁹. These analyses confirmed that KDM4A was in fact significantly correlated with copy gains of distinct regions such as 1q21 (a frequently amplified/gain region in cancer that associates with drug resistance and poor patient outcome—see above section in review). These data are consistent with a previous study that demonstrated that 1p34 and 1q21 were frequently amplified in tumors³². KDM4A resides on 1p34, and in turn, drives 1q21. These relationships were confirmed in cell culture models across diverse cell types. Taken together, these data illustrate that chromatin states are not just important in modulating DNA replication but enabling certain regions to undergo site-specific selection for gains. These data also support a model by which the cell uses these types of mechanisms to selectively gain regions for adaptive advantage. Consistent with TSSGs providing a selective advantage, Black et al. recently demonstrated that physiological stimulus such as hypoxia can also generate TSSGs in normal cells as well as human tumors. These copy gains were KDM4A dependent and were generated with every round of DNA replication. Importantly, hypoxia resulted in the amplification of a drug resistant oncogene CKS1B that was blocked when cells were reverted to normoxia or upon KDM4A inhibition. Hence, generation of transient copy gains and gene amplifications can be an adaptive cellular response of cells to external stresses or stimuli.

These data also reiterate that the point in cell cycle would determine whether copy gains are observed, which can contribute to the intra-tumor heterogeneity observed for copy number in tumors. In fact, genome-wide analyses look across cells in a population and not at a single cell level. Since the transient exposure was sufficient to promote gains of regions associated with drug resistance and poor outcome in patients, other input signals could be involved in allowing these regions to undergo TSSG. This notion is not unprecedented since chemotherapy has been shown to select for TSSGs such as DHFR and EGFR amplifications.

Roles for TSSG and Future Perspective

TSSGs could be a mechanism employed by tumor cells for selective acquisition of drug resistance by the amplification of specific genes. This phenomenon in cultured mammalian cells was first reported in 1978 as a mechanism for the acquisition of drug resistance to the drug methotrexate⁸² The drug methotrexate competitively inhibits the enzyme dihydrofolate reductase (DHFR), which catalyzes the conversion of dihydrofolate to active tetrahydrofolate, required for the de novo synthesis of thymidine. Cells developed resistance to methotrexate by overproduction of DHFR as a result of selective gene amplification⁸². These data highlight the possibility of mechanisms driving TSSGs that could be used by tumor cells for protection. Gene amplification in tumor cells forms two common structures: extrachromosomal double minutes (DM) and intra-chromosomal homogenously staining regions (HSRs). Storlazzi et al. investigated these structures of MYCN amplifications using 8 neuroblastoma and 2 small cell carcinoma cell lines⁸³. The study provided evidence of generation of HSRs from DMs by an episome model wherein DNA segments are excised from a chromosome, that are then circularized and amplified to form DM or HSRs. It is suggested that DMs are unstable and can be eliminated after drug treatment⁸⁴′8⁵, however HSRs are more stable⁸⁶. A number of oncogenes have been identified on extrachromosomal DNA, enabling the tumor cells to respond rapidly to drug treatment^{87,88}. The reversion of malignant phenotype and cellular differentiation by the elimination of DMs has been shown extensively in a variety of tumors and cancer cell lines⁸⁹⁻⁹¹. This shows that changes in copy number can be an effective strategy for quick adaptation to selective pressures in tumor cells.

Transient changes in copy number can be another mechanism for generating intratumoral heterogeneity in cells, which can contribute to cancer drug resistance. A recent study by Nathanson et al. demonstrated that oncogenes maintained on extrachromosomal DNA are gained/lost in response to drug treatment⁹². Glioblastoma patients harbor a constitutively active oncogenic variant of epidermal growth factor receptor (EGFR-vIII) formed by the in-frame deletion of exon2-7 in EGFR gene and is present primarily on extrachromosomal DNA called double minute chromosomes. The presence of EGFR-vIII makes tumor cells more sensitive to EGFR tyrosine kinase inhibitors (TKIs). In their experiments, the continued treatment with an EGFR TKI (e.g., Erlotinib) resulted in a loss of EGFR-vIII on extrachromosomal DNA, thus conferring resistance to the TKI. When the drug was withdrawn for a short period of time, there was an increase in EGFR-vIII on extrachromosomal DNA and the cells were sensitized again to erlotinib treatment. The authors suggest that instead of a continuous therapeutic regimen, withdrawal of drugs during therapy might be a more effective mechanism to restore the sensitivity of tumor cells to drugs.

Another important area of investigation is the understanding of how the rereplicated regions are generated transiently or EGFR-vIII on extrachromosomal DNA in the above case is lost with targeted therapies. An active surveillance mechanism involving degradation or clearance of these extra DNA fragments could be a possibility.

### REFERENCES

1 Hughes, T. R. et al. Widespread aneuploidy revealed by DNA microarray expression profiling. Nat Genet 25, 333-337, doi:10.1038/77116 (2000).
2 Gordon, D. J., Resio, B. & Pellman, D. Causes and consequences of aneuploidy in cancer. Nat Rev Genet 13, 189-203, doi:10.1038/nrg3123 (2012).
3 Tang, Y. C. & Amon, A. Gene copy-number alterations: a cost-benefit analysis. Cell 152, 394-405, doi:10.1016/j.cell.2012.11.043 (2013).
4 Feuk, L., Carson, A. R. & Scherer, S. W. Structural variation in the human genome. Nat Rev Genet 7, 85-97, doi:10.1038/nrg1767 (2006).
Buongiorno-Nardelli, M., Amaldi, F. & Lava-Sanchez, P. A. Electron microscope analysis of amplifying ribosomal DNA from Xenopus laevis. Exp Cell Res 98, 95-103 (1976).
6 Gall, J. G. & Rochaix, J. D. The amplified ribosomal DNA of dytiscid beetles. Proc Natl Acad Sci USA 71, 1819-1823 (1974).
7 Brown, D. D. & Dawid, I. B. Specific gene amplification in oocytes. Oocyte nuclei contain extrachromosomal replicas of the genes for ribosomal RNA. Science 160, 272-280 (1968).
8 Findly, R. C. & Gall, J. G. Free ribosomal RNA genes in Paramecium are tandemly repeated. Proc Natl Acad Sci USA 75, 3312-3316 (1978).
9 Engberg, J. The ribosomal RNA genes of Tetrahymena: structure and function. Eur J Cell Biol 36, 133-151 (1985).
Lara, F. J., Stocker, A. J. & Amabis, J. M. DNA sequence amplification in sciarid flies: results and perspectives. Braz J Med Biol Res 24, 233-248 (1991).
11 Candido-Silva, J. A. et al. Amplification and expression of a salivary gland DNA puff gene in the prothoracic gland of Bradysia hygida (Diptera: Sciaridae). J Insect Physiol 74C, 30-37, doi:10.1016/j.jinsphys.2015.01.014 (2015).
12 Orr-Weaver, T. L. Drosophila chorion genes: cracking the eggshell's secrets. Bioessays 13, 97-105, doi:10.1002/bies.950130302 (1991).
13 Claycomb, J. M., Benasutti, M., Bosco, G., Fenger, D. D. & Orr-Weaver, T. L. Gene amplification as a developmental strategy: isolation of two developmental amplicons in Drosophila. Dev Cell 6, 145-155 (2004).
14 Rehen, S. K. et al. Chromosomal variation in neurons of the developing and adult mammalian nervous system. Proc Natl Acad Sci USA 98, 13361-13366, doi:10.1073/pnas.231487398 (2001).
Blaschke, A. J., Staley, K. & Chun, J. Widespread programmed cell death in proliferative and postmitotic regions of the fetal cerebral cortex. Development 122, 1165-1174 (1996).
16 Westra, J. W. et al. Aneuploid mosaicism in the developing and adult cerebellar cortex. J Comp Neurol 507, 1944-1951, doi:10.1002/cne.21648 (2008).
17 McConnell, M. J. et al. Mosaic copy number variation in human neurons. Science 342, 632-637, doi:10.1126/science.1243472 (2013).
18 Duncan, A. W. et al. The ploidy conveyor of mature hepatocytes as a source of genetic variation. Nature 467, 707-710, doi:10.1038/nature09414 (2010).
19 Duncan, A. W. et al. Frequent aneuploidy among normal human hepatocytes. Gastroenterology 142, 25-28, doi:10.1053/j.gastro.2011.10.029 (2012).
Guiral, S., Mitchell, T. J., Martin, B. & Claverys, J. P. Competence-programmed predation of noncompetent cells in the human pathogen Streptococcus pneumoniae: genetic requirements. Proc Natl Acad Sci USA 102, 8710-8715, doi:10.1073/pnas.0500879102 (2005).
21 Slager, J., Kjos, M., Attaiech, L. & Veening, J. W. Antibiotic-induced replication stress triggers bacterial competence by increasing gene dosage near the origin. Cell 157, 395-406, doi:10.1016/j.cell.2014.01.068 (2014).
22 Forche, A., Magee, P. T., Selmecki, A., Berman, J. & May, G. Evolution in Candida albicans populations during a single passage through a mouse host. Genetics 182, 799-811, doi:10.1534/genetics.109.103325 (2009).
23 Morrow, C. A. & Fraser, J. A. Ploidy variation as an adaptive mechanism in human pathogenic fungi. Semin Cell Dev Biol 24, 339-346, doi:10.1016/j.semcdb.2013.01.008 (2013).
24 Selmecki, A., Forche, A. & Berman, J. Aneuploidy and isochromosome formation in drug-resistant Candida albicans. Science 313, 367-370, doi: 10.1126/science. 1128242 (2006).
Selmecki, A., Gerami-Nejad, M., Paulson, C., Forche, A. & Berman, J. An isochromosome confers drug resistance in vivo by amplification of two genes, ERG11 and TAC1. Mol Microbiol 68, 624-641, doi: 10.1111/j. 1365-2958.2008.06176.x (2008).
26 Coste, A. T., Karababa, M., Ischer, F., Bille, J. & Sanglard, D. TAC1, transcriptional activator of CDR genes, is a new transcription factor involved in the regulation of Candida albicans ABC transporters CDR1 and CDR2. Eukaryot Cell 3, 1639-1652, doi:10.1128/EC.3.6.1639-1652.2004 (2004).
27 White, T. C. Increased mRNA levels of ERG16, CDR, and MDR1 correlate with increases in azole resistance in Candida albicans isolates from a patient infected with human immunodeficiency virus. Antimicrob Agents Chemother 41, 1482-1487 (1997).
28 Gresham, D. et al. The repertoire and dynamics of evolutionary adaptations to controlled nutrient-limited environments in yeast. PLoS Genet 4, e1000303, doi:10.1371/journal.pgen.1000303 (2008).
29 Perry, G. H. et al. Diet and the evolution of human amylase gene copy number variation. Nat Genet 39, 1256-1260, doi:10.1038/ng2123 (2007).
Gonzalez, E. et al. The influence of CCL3L1 gene-containing segmental duplications on HIV-1/AIDS susceptibility. Science 307, 1434-1440, doi:10.1126/science.1101160 (2005).
31 Duncan, A. W. et al. Aneuploidy as a mechanism for stress-induced liver adaptation. J Clin Invest 122, 3307-3315, doi:10.1172/JCI64026 (2012).
32 Beroukhim, R. et al. The landscape of somatic copy-number alteration across human cancers. Nature 463, 899-905, doi: 10.1038/nature08822 (2010).
33 Yin, L., Kosugi, M. & Kufe, D. Inhibition of the MUC1-C oncoprotein induces multiple myeloma cell death by down-regulating TIGAR expression and depleting NADPH. Blood 119, 810-816, doi:10.1182/blood-2011-07-369686 (2012).
34 Fan, F. et al. Targeting Mcl-1 for multiple myeloma (MM) therapy: drug-induced generation of Mcl-1 fragment Mcl-1(128-350) triggers MM cell death via c-Jun upregulation. Cancer Lett 343, 286-294, doi: 10. 1016/j.canlet.2013.09.042 (2014).
Inoue, J. et al. Overexpression of PDZK1 within the 1q12-q22 amplicon is likely to be associated with drug-resistance phenotype in multiple myeloma. Am J Pathol 165, 71-81, doi: 10.1016/S0002-9440(10)63276-2 (2004).
36 Hideshima, T., Mitsiades, C., Tonon, G., Richardson, P. G. & Anderson, K. C. Understanding multiple myeloma pathogenesis in the bone marrow to identify new therapeutic targets. Nat Rev Cancer 7, 585-598, doi:10.1038/nrc2189 (2007).
37 Mani, M. et al. BCL9 promotes tumor progression by conferring enhanced proliferative, metastatic, and angiogenic properties to cancer cells. Cancer Res 69, 7577-7586, doi: 10.1158/0008-5472.CAN-09-0773 (2009).
38 Fabris, S. et al. Transcriptional features of multiple myeloma patients with chromosome 1q gain. Leukemia 21, 1113-1116, doi:10.1038/sj.leu.2404616 (2007).
39 Hanamura, I. et al. Frequent gain of chromosome band 1q21 in plasma-cell dyscrasias detected by fluorescence in situ hybridization: incidence increases from MGUS to relapsed myeloma and is related to prognosis and disease progression following tandem stem-cell transplantation. Blood 108, 1724-1732, doi: 10.1182/blood-2006-03-009910 (2006).
40 Chang, H., Yeung, J., Xu, W., Ning, Y. & Patterson, B. Significant increase of CKS1B amplification from monoclonal gammopathy of undetermined significance to multiple myeloma and plasma cell leukaemia as demonstrated by interphase fluorescence in situ hybridisation. Br J Haematol 134, 613-615, doi:10.1111/j.1365-2141.2006.06237.x (2006).
41 Shaughnessy, J. Amplification and overexpression of CKS1B at chromosome band 1q21 is associated with reduced levels of p27Kip1 and an aggressive clinical course in multiple myeloma. Hematology 10 Suppl 1, 117-126, doi: 10.1080/10245330512331390140 (2005).
42 Fonseca, R. et al. Prognostic value of chromosome 1q21 gain by fluorescent in situ hybridization and increase CKS1B expression in myeloma. Leukemia 20, 2034-2040, doi: 10.1038/sj.leu.2404403 (2006).
43 Kudoh, K. et al. Gains of 1q21-q22 and 13q12-q14 are potential indicators for resistance to cisplatin-based chemotherapy in ovarian cancer patients. Clin Cancer Res 5, 2526-2531 (1999).
44 Takano, M. et al. Analyses by comparative genomic hybridization of genes relating with cisplatin-resistance in ovarian cancer. Hum Cell 14, 267-271 (2001).
45 Yabuki, N. et al. Gene amplification and expression in lung cancer cells with acquired paclitaxel resistance. Cancer Genet Cytogenet 173, 1-9, doi: 10.1016/j.cancergencyto.2006.07.020 (2007).
46 Hirsch, D. et al. Chromothripsis and focal copy number alterations determine poor outcome in malignant melanoma. Cancer Res 73, 1454-1460, doi:10.1158/0008-5472.CAN-12-0928 (2013).
47 Rausch, T. et al. Genome sequencing of pediatric medulloblastoma links catastrophic DNA rearrangements with TP53 mutations. Cell 148, 59-71, doi: 10.1016/j.cell.2011.12.013 (2012).
48 Hurst, C. D., Platt, F. M., Taylor, C. F. & Knowles, M. A. Novel tumor subgroups of urothelial carcinoma of the bladder defined by integrated genomic analysis. Clin Cancer Res 18, 5865-5877, doi: 10.1158/1078-0432.CCR-12-1807 (2012).
49 Morrison, C. D. et al. Whole-genome sequencing identifies genomic heterogeneity at a nucleotide and chromosomal level in bladder cancer. Proc Natl Acad Sci US A 111, E672-681, doi:10.1073/pnas.1313580111 (2014).
50 Landau, D. A. et al. Evolution and impact of subclonal mutations in chronic lymphocytic leukemia. Cell 152, 714-726, doi:10.1016/j.cell.2013.01.019 (2013).
51 Hastings, P. J., Lupski, J. R., Rosenberg, S. M. & Ira, G. Mechanisms of change in gene copy number. Nat Rev Genet 10, 551-564, doi:10.1038/nrg2593 (2009).
52 Black, J. C., Van Rechem, C. & Whetstine, J. R. Histone lysine methylation dynamics: establishment, regulation, and biological impact. Mol Cell 48, 491-507, doi:10.1016/j.molcel.2012.11.006 (2012).
53 Gardner, K. E., Allis, C. D. & Strahl, B. D. Operating on chromatin, a colorful language where context matters. J Mol Biol 409, 36-46, doi:10.1016/j.jmb.2011.01.040 (2011).
54 MacAlpine, D. M. & Almouzni, G. Chromatin and DNA replication. Cold Spring Harb Perspect Biol 5, a010207, doi: 10.1101/cshperspect.a010207 (2013).
55 Rhind, N. & Gilbert, D. M. DNA Replication Timing. Cold Spring Harb Perspect Med 3, 1-26 (2013).
56 Iizuka, M. & Stillman, B. Histone acetyltransferase HBO1 interacts with the ORC1 subunit of the human initiator protein. J Biol Chem 274, 23027-23034 (1999).
57 Iizuka, M., Matsui, T., Takisawa, H. & Smith, M. M. Regulation of replication licensing by acetyltransferase Hbo1. Mol Cell Biol 26, 1098-1108, doi:10.1128/MCB.26.3.1098-1108.2006 (2006).
58 Miotto, B. & Struhl, K. HBO1 histone acetylase activity is essential for DNA replication licensing and inhibited by Geminin. Mol Cell 37, 57-66, doi:10.1016/j.molcel.2009.12.012 (2010).
59 Vogelauer, M., Rubbi, L., Lucas, I., Brewer, B. J. & Grunstein, M. Histone acetylation regulates the time of replication origin firing. Mol Cell 10, 1223-1233 (2002).
60 Goren, A., Tabib, A., Hecht, M. & Cedar, H. DNA replication timing of the human beta-globin domain is controlled by histone modification at the origin. Genes Dev 22, 1319-1324, doi: 10.1101/gad.468308 (2008).
61 Romanoski, C. E., Glass, C. K., Stunnenberg, H. G., Wilson, L. & Almouzni, G. Epigenomics: Roadmap for regulation. Nature 518, 314-316, doi: 10.1038/518314a (2015).
62 Black, J. C. & Whetstine, J. R. Tipping the lysine methylation balance in disease. Biopolymers 99, 127-135, doi:10.1002/bip.22136 (2013).
63 Tardat, M., Murr, R., Herceg, Z., Sardet, C. & Julien, E. PR-Set7-dependent lysine methylation ensures genome replication and stability through S phase. J Cell Biol 179, 1413-1426, doi: 10.1083/jcb.200706179 (2007).
64 Tardat, M. et al. The histone H4 Lys 20 methyltransferase PR-Set7 regulates replication origins in mammalian cells. Nat Cell Biol 12, 1086-1093, doi: 10.1038/ncb2113 (2010).
65 Jorgensen, S. et al. SET8 is degraded via PCNA-coupled CRL4(CDT2) ubiquitylation in S phase and after UV irradiation. J Cell Biol 192, 43-54, doi:10.1083/jcb.201009076 (2011).
66 Centore, R. C. et al. CRL4(Cdt2)-mediated destruction of the histone methyltransferase Set8 prevents premature chromatin compaction in S phase. Mol Cell 40, 22-33, doi:10.1016/j.molcel.2010.09.015 (2010).
67 Jorgensen, S. et al. The histone methyltransferase SET8 is required for S-phase progression. J Cell Biol 179, 1337-1345, doi:10.1083/jcb.200706150 (2007).
68 Karachentsev, D., Sarma, K., Reinberg, D. & Steward, R. PR-Set7-dependent methylation of histone H4 Lys 20 functions in repression of gene expression and is essential for mitosis. Genes Dev 19, 431-435, doi:10.1101/gad.1263005 (2005).
69 Kim, J. H. & Workman, J. L. Histone acetylation in heterochromatin assembly. Genes Dev 24, 738-740, doi:10.1101/gad.1922110 (2010).
70 Biswas, D. et al. A role for Chd1 and Set2 in negatively regulating DNA replication in Saccharomyces cerevisiae. Genetics 178, 649-659, doi: 10.1534/genetics. 107.084202 (2008).
71 Quivy, J. P., Gerard, A., Cook, A. J., Roche, D. & Almouzni, G. The HP1-p150/CAF-1 interaction is required for pericentric heterochromatin replication and S-phase progression in mouse cells. Nat Struct Mol Biol 15, 972-979 (2008).
72 Pak, D. T. et al. Association of the origin recognition complex with heterochromatin and HP1 in higher eukaryotes. Cell 91, 311-323 (1997).
73 Thomae, A. W. et al. Interaction between HMGA1a and the origin recognition complex creates site-specific replication origins. Proc Natl Acad Sci USA 105, 1692-1697, doi: 10.1073/pnas.0707260105 (2008).
74 Schwaiger, M., Kohler, H., Oakeley, E. J., Stadler, M. B. & Schubeler, D. Heterochromatin protein 1 (HP1) modulates replication timing of the Drosophila genome. Genome Res 20, 771-780, doi:10.1101/gr.101790.109 (2010).
75 Chen, Z. et al. Structural insights into histone demethylation by JMJD2 family members. Cell 125, 691-702, doi:10.1016/j.cell.2006.04.024 (2006).
76 Whetstine, J. R. et al. Reversal of histone lysine trimethylation by the JMJD2 family of histone demethylases. Cell 125, 467-481, doi: 10.1016/j.cell.2006.03.028 (2006).
77 Cloos, P. A. et al. The putative oncogene GASC1 demethylates tri- and dimethylated lysine 9 on histone H3. Nature 442, 307-311, doi:10.1038/nature04837 (2006).
78 Klose, R. J. et al. The transcriptional repressor JHDM3A demethylates trimethyl histone H3 lysine 9 and lysine 36. Nature 442, 312-316, doi: 10.1038/nature04853 (2006).
79 Black, J. C. et al. KDM4A lysine demethylase induces site-specific copy gain and rereplication of regions amplified in tumors. Cell 154, 541-555, doi:10.1016/j.cell.2013.06.051 (2013).
80 Black, J. C. et al. Conserved antagonism between JMJD2A/KDM4A and HP1gamma during cell cycle progression. Mol Cell 40, 736-748, doi: 10.1016/j.molcel.2010.11.008 (2010).
81 Van Rechem, C. et al. The SKP1-Cul1-F-box and leucine-rich repeat protein 4 (SCF-FbxL4) ubiquitin ligase regulates lysine demethylase 4A (KDM4A)/Jumonji domain-containing 2A (JMJD2A) protein. J Biol Chem 286, 30462-30470, doi:10.1074/jbc.M111.273508 (2011).
82 Schimke, R. T. Methotrexate resistance and gene amplification. Mechanisms and implications. Cancer 57, 1912-1917 (1986).
83 Storlazzi, C. T. et al. Gene amplification as double minutes or homogeneously staining regions in solid tumors: origin and structure. Genome Res 20, 1198-1206, doi:10.1101/gr.106252.110 (2010).
84 Ambros, I. M. et al. Neuroblastoma cells can actively eliminate supernumerary MYCN gene copies by micronucleus formation—sign of tumour cell revertance? Eur J Cancer 33, 2043-2049 (1997).
85 Narath, R. et al. Induction of senescence in MYCN amplified neuroblastoma cell lines by hydroxyurea. Genes Chromosomes Cancer 46, 130-142, doi:10.1002/gcc.20393 (2007).
86 Balaban-Malenbaum, G. & Gilbert, F. Double minute chromosomes and the homogeneously staining regions in chromosomes of a human neuroblastoma cell line. Science 198, 739-741 (1977).
87 Benner, S. E., Wahl, G. M. & Von Hoff, D. D. Double minute chromosomes and homogeneously staining regions in tumors taken directly from patients versus in human tumor cell lines. Anticancer Drugs 2, 11-25 (1991).
88 Shimizu, N. Extrachromosomal double minutes and chromosomal homogeneously staining regions as probes for chromosome research. Cytogenet Genome Res 124, 312-326, doi:10.1159/000218135 (2009).
89 Von Hoff, D. D. et al. Elimination of extrachromosomally amplified MYC genes from human tumor cells reduces their tumorigenicity. Proc Natl Acad Sci USA 89, 8165-8169 (1992).
90 Eckhardt, S. G. et al. Induction of differentiation in HL60 cells by the reduction of extrachromosomally amplified c-myc. Proc Natl Acad Sci USA 91, 6674-6678 (1994).
91 Nielsen, J. L. et al. Evidence of gene amplification in the form of double minute chromosomes is frequently observed in lung cancer. Cancer Genet Cytogenet 65, 120-124 (1993).
92 Nathanson, D. A. et al. Targeted therapy resistance mediated by dynamic regulation of extrachromosomal mutant EGFR DNA. Science 343, 72-76, doi:10.1126/science.1241328 (2014).

### Example 2

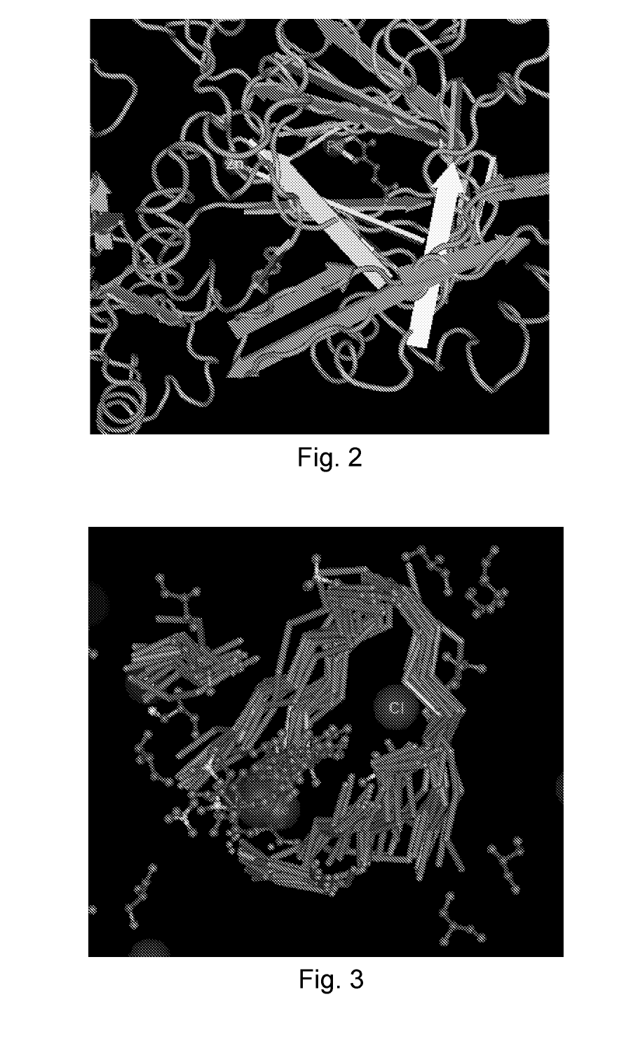

The three-dimensional structure of JMJD2A (NP_055478.2), particularly 2GP5 (3), and its peculiar beta-sheet coiling pattern is described herein (FIG. 2). As described herein, this I “barrel” structure is evolutionarily conserved and over 250 known crystallized structures exist with this sort of pattern. A list of all non-animal VAST hits (2) is provided in Table 1.

TABLE 1
Non-animal proteins comprising the beta-sheet or “barrel” coiling pattern of JMJD2A.

| Structure | | | Alignment | | Exemplary NCBI |
|---|---|---|---|---|---|
| ID (PDB) | Organism | Function | Length | Ligands Present | Gene ID |
| 1VRB | Bacillus subtilis | putative | 141 | 4 irons | 937640 |
| 2CSG | Salmonella enterica | putative | 140 | Isocitric acid, | 1252360 |
| | | | | succinic acid, citric | |
| | | | | acid, iron | |
| 2Q4A | Arabidopis thaliana | | 117 | 4 sulfuric acids, 2 | 821690 |
| | | | | irons | |
| 1OS7 | Escherichia coli | dioxygenase | 105 | 3 taurines, 4 alpha- | 945021 |
| | | | | ketoglutaric acids, | |
| | | | | 4 irons | |
| 3DKQ | Shewanalla baltica | Putative | 102 | 3 nickels, 6 | |
| | | | | imidazoles, 6 | |
| | | | | glycerols | |
| 1OIJ | Pseudomonas putida | dioxygenase | 100 | 4 Sodiums, 4 alpha- | |
| | | | | ketoglutaric acids | |
| 1S4C | Haemophilus influenzae | unknown | 100 | 4 copper, acetic | 951142 |
| | | | | acid | |
| 2OG6 | Streptomyces coelicolor | Asparagines | 99 | Iron, 2 Chloride | 1098670 |
| | | oxygenase | | ions | |
| 1SQ4 | Pseudomonas aeruginosa | putative | 97 | 4 thiocyanic acids | 878039 |
| 2R6S | Escherichia coli | unknown | 97 | Iron, 3 sulfuric | 948076 |
| | | | | acids, N,N-bis(2- | |
| | | | | hydroxyethyl)glycine, | |
| | | | | 21 glycerols | |
| 3EAT | Pseudomonas aeruginosa | Tyrosine | 95 | Sodium ion | 878457 |
| | | derivative | | | |
| | | synthesis | | | |
| 1J58 | Bacillus subtilis | Oxalate | 93 | 2 manganese, | 938620 |
| | | decarboxylase | | magnesium ion, | |
| | | | | formic acid | |
| 1O4T | Thermotoga maritima | Putative | 93 | 2 oxalic acids, 2 | 898196 |
| | | | | manganese | |
| 1DZR | Salmonella enterica | epimerase | 92 | 4 sulfuric acids, 5 | 1253615 |
| | | | | glycerols | |
| 2IXK | Pseudomonas aeruginosa | epimerase | 92 | 2 dTDP-4-oxo-L- | 879991 |
| | | | | rhamnose | |
| 1QWR | Bacillus subtilis | isomerase | 91 | 3 sulfuric acids, 2 | 936815 |
| | | | | zincs, acetic acid, | |
| | | | | formic acid | |
| 1JR7 | Escherichia coli | Oxidoreductase | 90 | Ferrous ion | 948076 |
| 1DS1 | Streptomyces clavuligerus | Clavaminate | 89 | alpha-ketoglutaric | |
| | | Synthase | | acid, 2 | |
| | | | | CID445135s, 4 | |
| | | | | sulfuric acids, iron | |
| 2P17 | Geobacillus kaustophilus | unknown | 89 | Iron | 3184214 |
| 2B9U | Sulfolobus tokodaii | epimerase | 88 | none | 1460029 |
| 1E5R | Streptomyces | hydroxylase | 87 | none | |
| 1TQ5 | Escherichia coli | putative | 88 | 6 cadmiums | 947945 |
| 3ES4 | Agrobacterium tumefacians | unknown | 87 | Chloride ion, 11 | 1135060 |
| | | | | ethylene glycols | |
| 1NX4 | Pectobacterium carotovorum | Carbapenem | 85 | 3 alpha- | |
| | | Synthase | | ketoglutaric acids, | |
| | | | | 3 irons | |
| 1VJ2 | Thermotoga maritima | cupin | 85 | CID449048, 2 | 898018 |
| | | | | manganese | |
| 1SEF | Enterococcus faecalis | cupin | 85 | none | 1201846 |
| 2PFW | Shewanella frigidimarina | cupin | 85 | 5 ethylene glycols | |
| 1Y9Q | Vibrio cholerae | transcriptional | 84 | methionine, zinc | 2613472 |
| | | regulator | | | |
| 2D40 | Escherichia coli | dioxygenase | 84 | 4 irons | 916734 |
| 1SFN | Deinococcus radiodurans | unknown | 83 | none | 1798812 |
| 1V7O | Thermus thermophilus | antibiotic | 82 | Sodium ion | 1444180 |
| | | synthesis | | | |
| 2BNN | Streptomyces wedmorensis | epoxidase | 82 | 2 Zinc, 2 | |
| | | | | fosfomycin | |
| 2RG4 | Oceanicola granulosus | unknown | 82 | 2 Irons | |
| 3BCW | Bordetella bronchiseptica | unknown | 81 | 8 ethylene glycols, | 2662434 |
| | | cupin | | acetic acid, | |
| | | | | diethylene glycol | |
| 1LKN | Thermotoga maritima | putative cupin | 80 | none | 898653 |
| 1ZX5 | Archeaglobus fulgidus | putative | 71 | Beta-L- | 1483245 |
| | | isomerase | | fructofuranose, 2 | |
| | | | | ethylene glycols, 6 | |
| | | | | acetic acids, glycerol | |
| 3FJS | Ralstonia eutropha | unknown | 72 | none | 3613500 |
| | | cupin | | | |
| 2B8M | Methanocaldococcus jannaschii | phosphoserine | 80 | 3 sulfuric acids, | 1451641 |
| | | aminotransferase | | chloride ion, 3 | |
| | | | | ethylene glycols | |
| 3EO6 | Clostridium difficile | unknown | 80 | 2 magnesium ions, | |
| | | | | tromethamine | |
| 2Q1Z | Rhodobacter sphaeroides | transcriptional | 79 | 4 zinc | 3720372 |
| | | stress | | | |
| | | response | | | |
| 2OZJ | Desulfitobacterium hafniense | cupin | 78 | Glycerol | 7260920 |
| 1YLL | Pseudomonas aeruginosa | unknown | 77 | none | 883033 |
| 3BU7 | Rugeria pomeroyi | dioxygenase | 76 | 4 irons | 3196910 |
| 3DL3 | Vibrio fischeri | tellurite | 72 | none | 3278998 |
| | | resistance | | | |
| 2Q30 | Desulfovibrio desulfuricans | unknown | 66 | Sulfuric acid, 7 | 3757314 |
| | | | | ethylene glycols | |

It was then found that only those structures containing iron as a ligand proved useful for alignment. The other ligands tended to either shift or change the size of the barrel's pocket. A more detailed list of only the non-animal hits containing iron as a ligand (2) is provided in Table 2.

TABLE 2
Non-animal proteins comprising the beta-sheet or “barrel” coiling pattern of JMJD2A and an iron ligand.

| | | | Alignment | | Exemplary NCBI |
|---|---|---|---|---|---|
| Structure ID | Organism | Function | Length | Ligands Present | Gene ID |
| 1VRB | Bacillus subtilis | Putative- | 141 | 4 irons | 937640 |
| | | Asparaginyl | | | |
| | | Hydroxylase | | | |
| 2CSG | Salmonella enterica | Putative- | 140 | Isocitric acid, succinic | 1252360 |
| | | Oxidoreductase | | acid, citric acid, iron | |
| 2Q4A | Arabidopis thaliana | Oxidoreductase | 117 | 4 sulfuric acids, 2 | 821690 |
| | | | | irons | |
| 1OS7 | Escherichia coli | dioxygenase | 105 | 3 taurines, 4 alpha- | 945021 |
| | | | | ketoglutaric acids, 4 | |
| | | | | irons | |
| 2OG6 | Streptomyces coelicolor | Asparagines | 99 | Iron, 2 Chloride ions | 1098670 |
| | | oxygenase | | | |
| 2R6S | Escherichia coli | unknown | 97 | Iron, 3 sulfuric acids, | 948076 |
| | | | | N,N-bis(2- | |
| | | | | hydroxyethyl)glycine, | |
| | | | | 21 glycerols | |
| 1DS1 | Streptomyces clavuligerus | Clavaminate | 89 | alpha-ketoglutaric | |
| | | Synthase | | acid, 2 CID445135s, 4 | |
| | | | | sulfuric acids, iron | |
| 2P17 | Geobacillus kaustophilus | Pirin-related | 89 | Iron | 3184214 |
| 1NX4 | Pectobacterium carotovorum | Carbapenem | 85 | 3 alpha-ketoglutaric | |
| | | Synthase | | acids, 3 irons | |
| 2D40 | Escherichia coli | dioxygenase | 84 | 4 irons | 916734 |
| 2RG4 | Oceanicola granulosus | unknown | 82 | 2 Irons | |
| 3BU7 | Rugeria pomeroyi | dioxygenase | 76 | 4 irons | 3196910 |

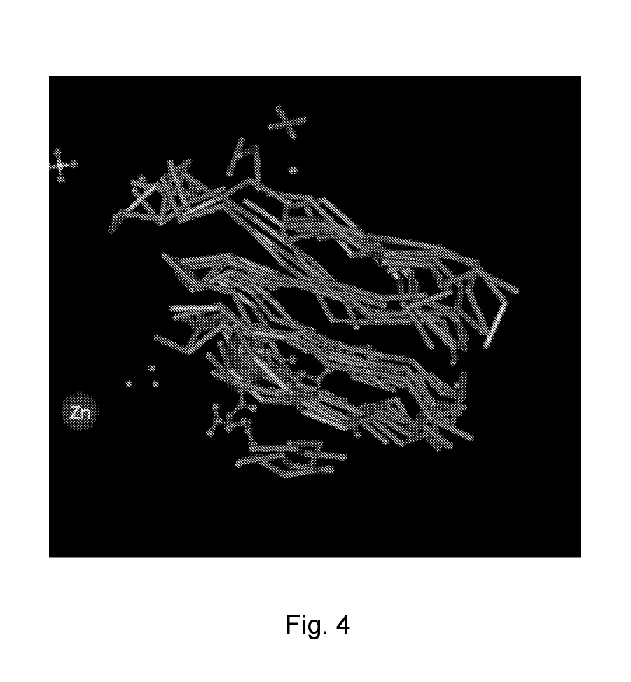

With these 13 hits, a structural alignment was first attempted. The resulting aligned structure was obtained (2) (FIG. 3). This demonstrates that the barrel is fairly well conserved in the iron-containing structures. More of the barrel was conserved when just using the top three hits (2) (FIG. 4).

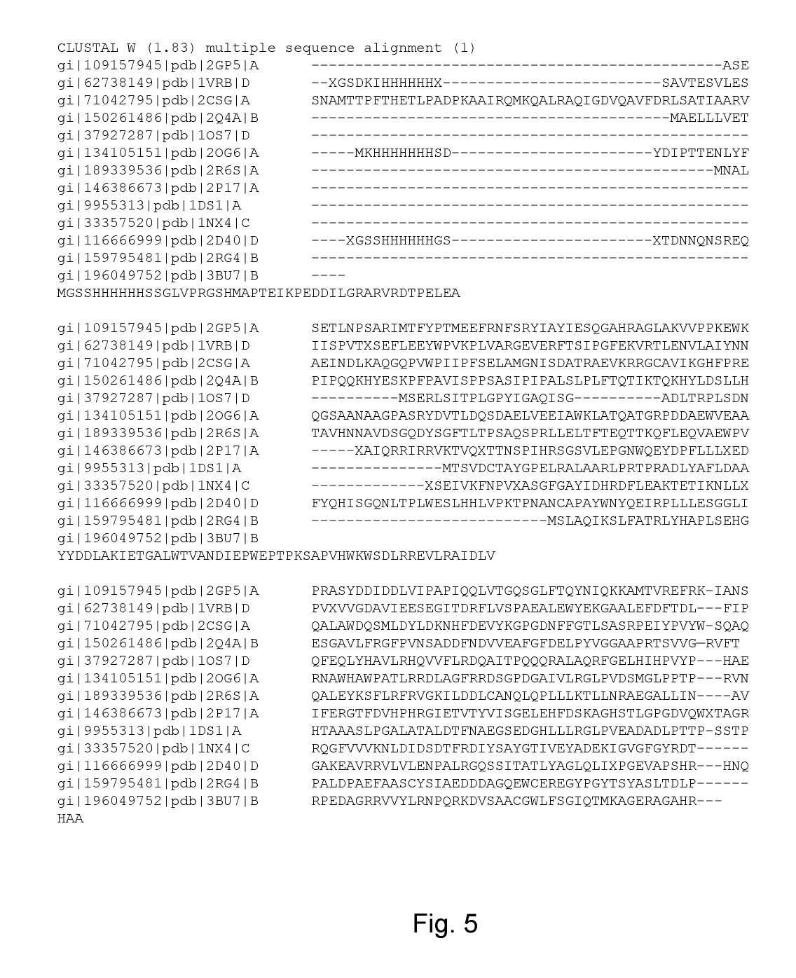

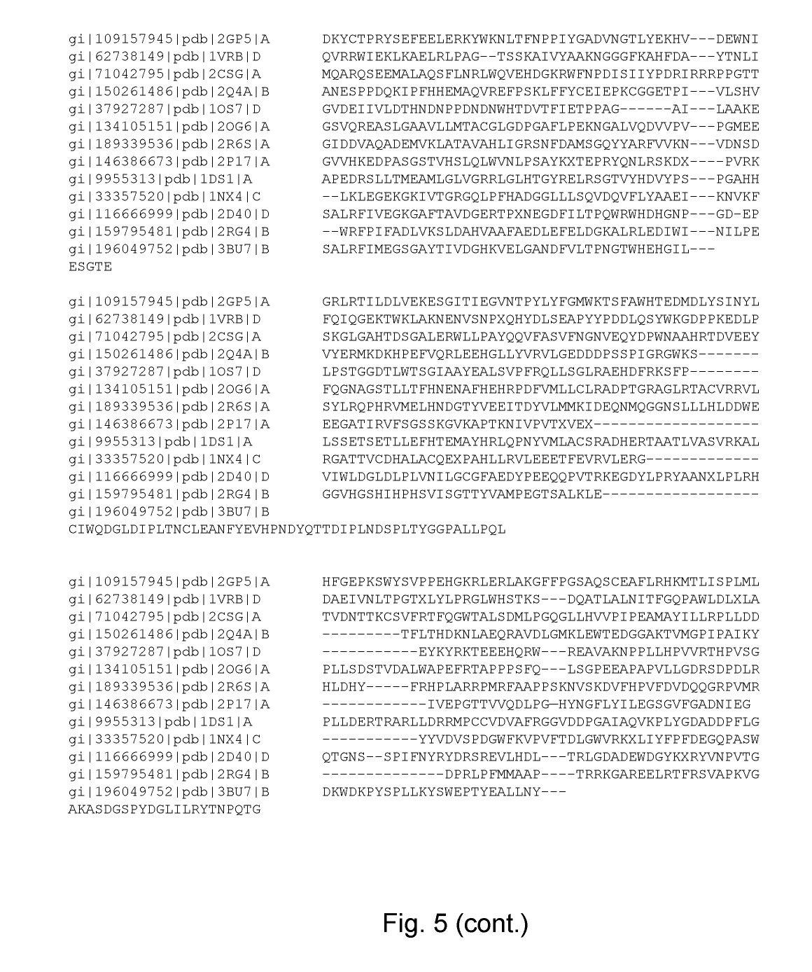

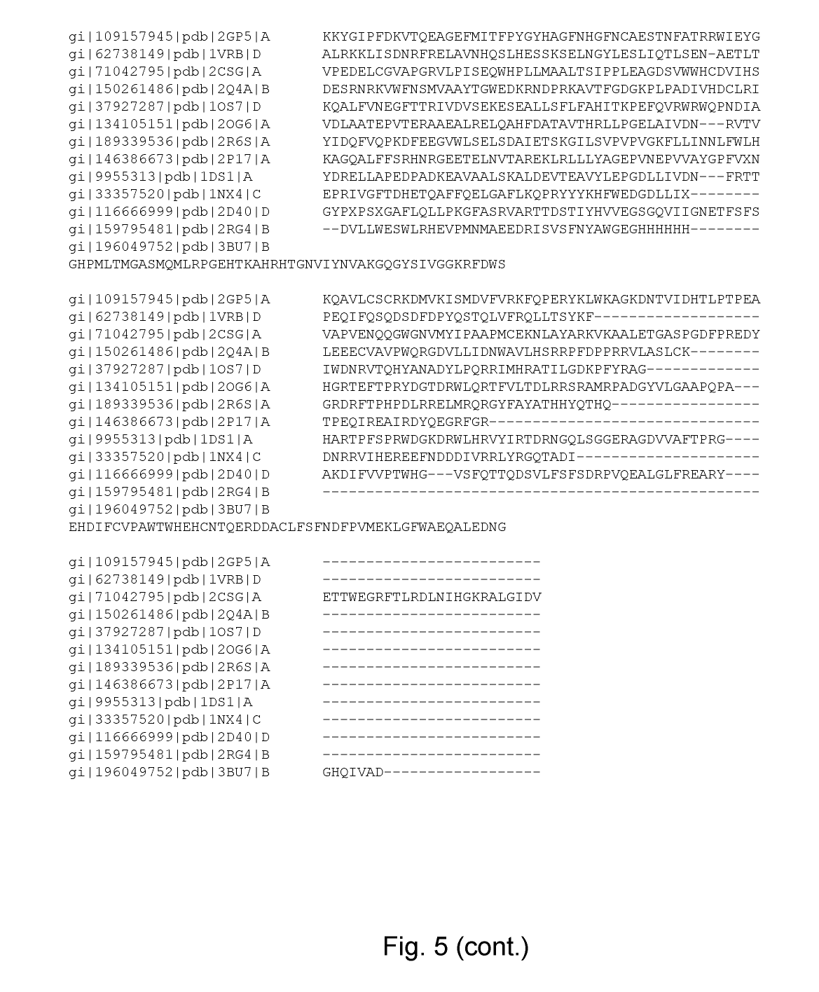

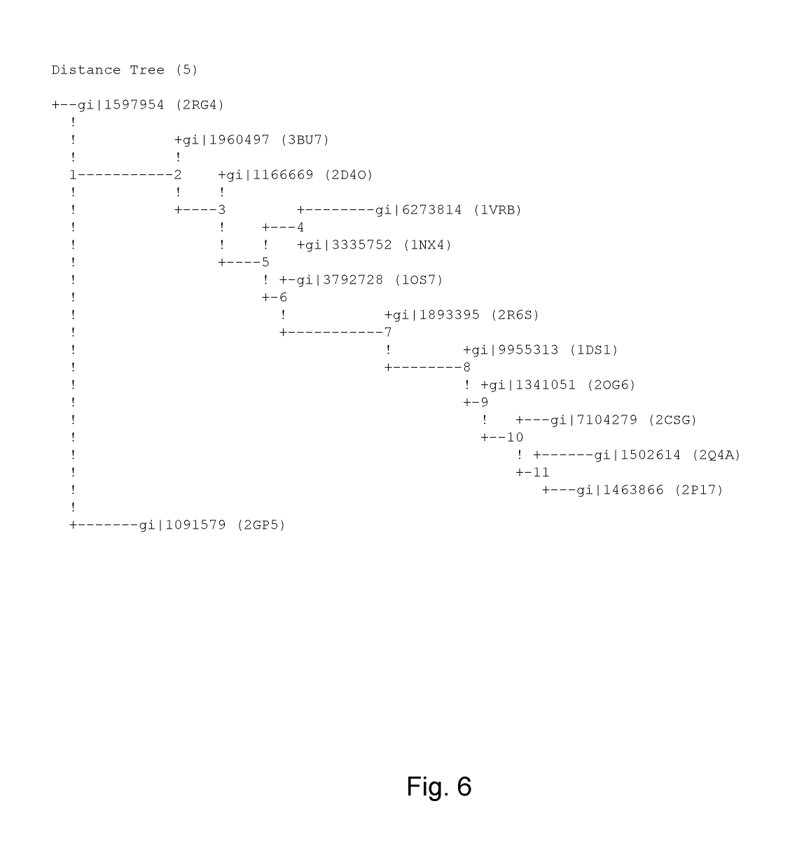

A sequence alignment was conducted for the iron-containing structure proteins to see if the similarity to JMJD2A was purely through 3D structure or could be traced through primary amino acid sequence as well (FIGS. 5 and 6) The distance tree (FIG. 6) demonstrates that human JMJD2A is an out-group to all of the other proteins. The other proteins included in FIG. 6 come from bacterial sources and would share some significant phylogeny. It also demonstrated that the conserved structural domains could not be identified through regular sequence alignments.

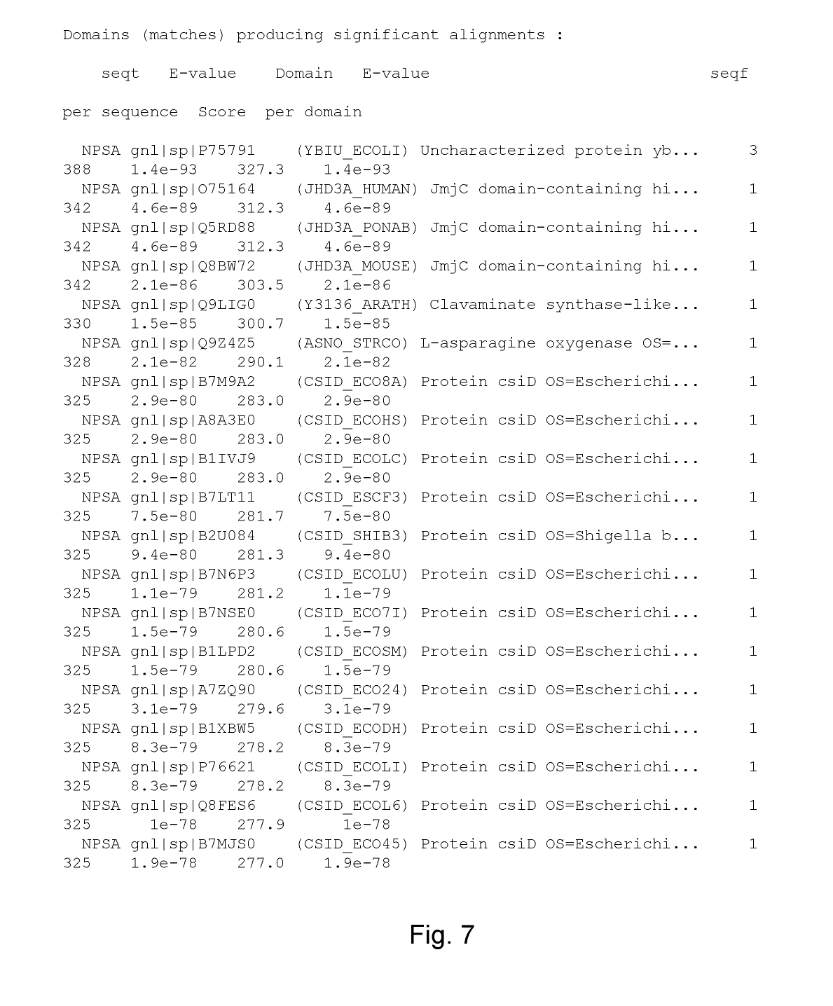

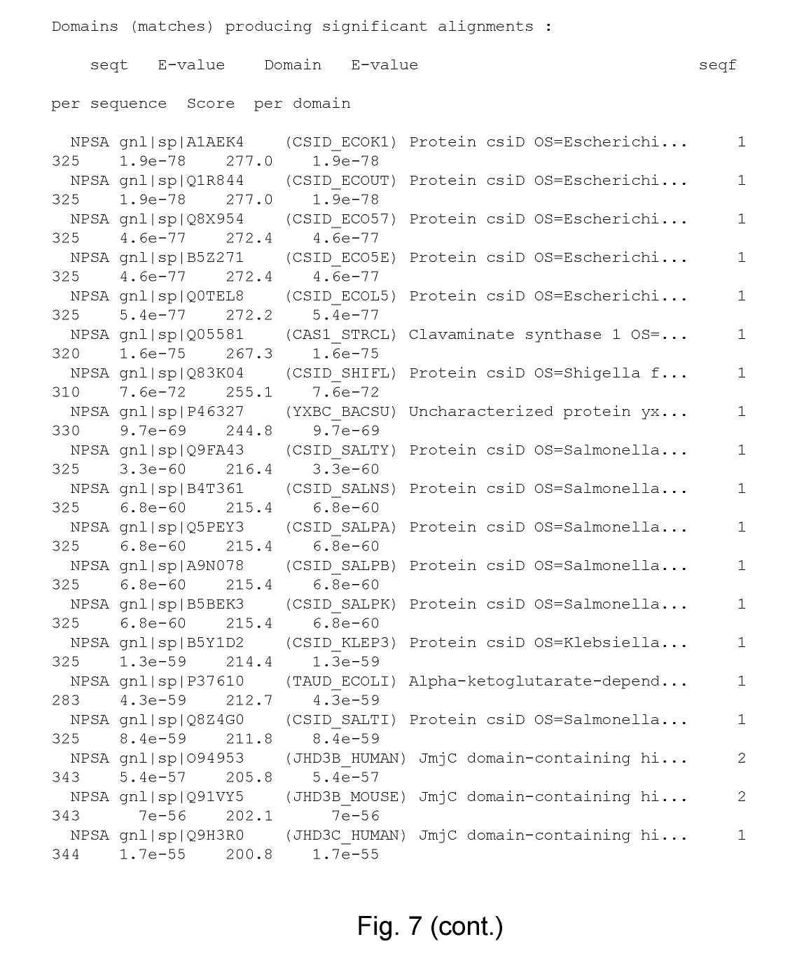

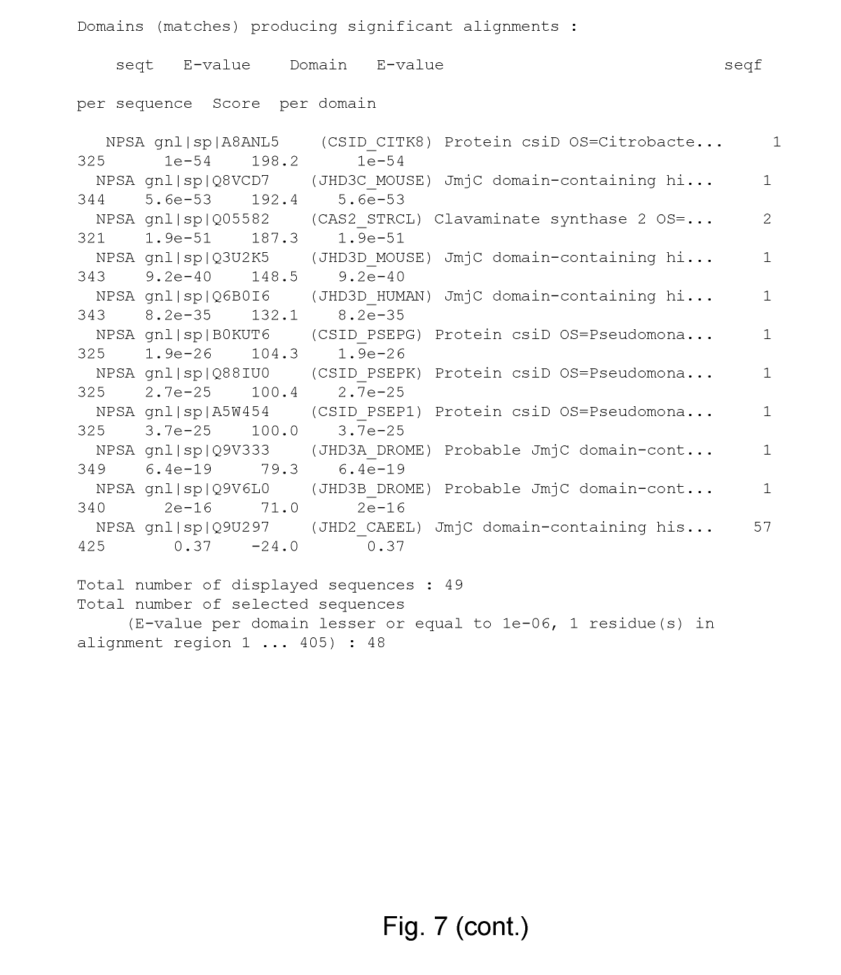

An HMM profile search for the structurally aligned regions of the iron containing structures (4) was performed (FIG. 7). The results of the search demonstrate that many domains from different proteins share these similar patterns (the redundant hits were excluded). 46 of the 49 sequences found have excellent e-values below 10e-20 aiding our search for relevant profiles.

The barrel structure of human JMJD2A does show conservation outside of the animal kingdom. The proteins with similar structure come from many different bacterial and archea species and serve a vast array of functions. However, the similarity between JMJD2A exists on a 3-dimensional basis. The conserved domains found through the profile search and structural alignment demonstrate that this peculiar beta-sheet barrel is highly conserved in at least the iron-containing bacterial and archea proteins.

### REFERENCES

1. ClustalW at www.ch.embnet.org/software/ClustalW.html.
2. Gibrat J F, Madej T, Bryant S H, “Surprising similarities in structure comparison”, Curr Opin Struct Biol. 1996 June; 6(3):377-85. Obtained at www.ncbi.nlm.nih.gov/Structure/vast/vastsrv.cgi?sdid=167337.
3. www.ncbi.nlm.nih.gov/Structure/mmdb/mmdbsrv.cgi?Dopt=s&uid=39411. Deposition provided by Chen Z, Zang J, Whetstine J, Hong X, Davrazou F, Kutateladze T G, Simpson M, Dai S, Hagman J, Shi Y, Zhang G, 2006/4/16.
4. NPS@: Network Protein Sequence Analysis TIBS 2000 March Vol. 25, No 3 [291]:147-150. Obtained at npsa-pbil.ibcp.fr/cgi-bin/profile_hmmsearch.pl.
5. PHYLIP at mobyle.pasteur.fr/cgi-bin/portal.py?form=neighbor.

### Example 3: Hypoxia Drives Transient Site-Specific Copy Gain and Drug Resistant Gene Expression

Copy number heterogeneity is a prominent feature within tumors. The molecular bases for this heterogeneity remain poorly characterized. It is demonstrated herein that hypoxia induces transient, site-specific copy gains (TSSGs) in primary, non-transformed and transformed human cells. Hypoxia-driven copy gains are not dependent on HIF1α or HIF2α; however, they are dependent upon the KDM4A histone demethylase and are blocked by inhibition of KDM4A with a small molecule or the natural metabolite succinate. Furthermore, this response is conserved at a syntenic region in zebrafish cells. Regions with site-specific copy-gain are also enriched for amplifications in hypoxic primary tumors. These tumors exhibited amplification and overexpression of the drug resistance gene CKS1B, which were recapitulated in hypoxic breast cancer cells. These results demonstrate that hypoxia provides a biological stimulus to create transient site-specific copy alterations that can result in heterogeneity within tumors and cell populations. These findings have major implications in our understanding of copy number heterogeneity and the emergence of drug resistance genes in cancer.

Introduction

Cancer is often characterized by copy gains or losses of chromosome arms, whole chromosomes, and/or amplifications/deletions of smaller genomic fragments (Hook et al. 2007; Stratton et al. 2009; Beroukhim et al. 2010). While it has long been understood that tumors within the same pathological subtype have different mutational and copy number profiles (Burrell et al. 2013), it has recently become apparent that intra-tumoral heterogeneity likely plays an important role in tumor development, metastatic potential and acquired drug resistance (Gerlinger et al. 2012; Burrell et al. 2013; Junttila and de Sauvage 2013; Nathanson et al. 2014). Traditionally, somatic copy number alterations (SCNA) and copy number variations (CNV) have been thought of as heritable genetic events in cancer cells that emerge through an adaptive advantage; however, recent work suggests that at least some copy gains may be transient and could arise given the correct genetic, therapeutic or environmental conditions (Black et al. 2013; Nathanson et al. 2014). For example, analysis of epidermal growth factor receptor (EGFR) mutations and amplifications in glioblastoma patients revealed a transient extrachromosomal amplification of a specific EGFR isoform (Nathanson et al. 2014). In addition, amplification and overexpression of the H3K9/36 tri-demethylase KDM4A/JMJD2A caused rereplication and transient site-specific copy gains (TSSGs). Furthermore, impairing H3K9 or H3K36 methylation with lysine to methionine substitutions (K9M or K36M) resulted in site-specific gains (Black et al. 2013; Lewis et al. 2013).

These initial observations highlighted a pathological state that could promote copy gains. However, a major question remained: “are there physiological signals or cues that cells encounter, that in turn, cause copy gains within defined regions of the genome?” It was reasoned that tumor cells encounter various stresses that could promote copy gains, which could ultimately contribute to the copy number heterogeneity observed in tumors. Regions of SCNA often observed in tumors may be prone to transient amplification (i.e., 1q12-1q21) and contribute to their observed copy gains in tumors. This same notion could also explain why CNV of specific regions (e.g., 1q21) emerge in other diseases such as autism and schizophrenia (Stefansson et al. 2008; Levinson et al. 2011).

Therefore, site-specific copy gains were systemically screened after cells were treated with a panel of cellular stresses that occur during development and tumorigenesis. Surprisingly, only one condition, hypoxia, promotes site-specific copy gain of regions frequently observed in tumors. Hypoxia-dependent copy gain occurs at tumor-relevant oxygen levels (1% O₂) in diverse cancer cell lines and in primary T cells. Hypoxia-dependent site-specific copy gains are transient, require S phase and undergo rereplication. It is demonstrated herein that copy gains were not dependent on HIF1α or HIF2a; however, the a-ketoglutarate-dependent lysine demethylase KDM4A was required for the copy gains. Upon hypoxic exposure, KDM4A was stabilized through reduced association with the SCF ubiquitin ligase complex, increased association with chromatin, and retained enzyme activity. Finally, pretreatment of cells with succinate (a naturally occurring metabolite that inactivates a-ketoglutarate-dependent enzymes) or a lysine demethylase (KDM) chemical inhibitor block hypoxia-induced gains. These observations highlight the dynamics associated with copy gain and indicate that enzyme levels, S phase status, cellular stresses and metabolic state can contribute to the copy number heterogeneity observed in human tumors.

Consistent with hypoxia-induced copy gains being a biological response, it is demonstrated that copy gain following hypoxia is conserved at a syntenic region in zebrafish cells, while a non-syntenic region was not gained. In addition, primary breast and lung tumors with a defined hypoxic gene signature are enriched for focal copy number changes in the same regions generated in human and zebrafish cell culture. Most importantly, the present analyses of hypoxic breast and lung tumors identified increased copy number and expression of a drug resistance oncogene CKS1B (Shaughnessy 2005). It is further demonstrated in breast cancer cells that CKS1B exhibited site-specific copy gain and had increased expression upon hypoxic exposure. These results indicate that hypoxia can promote site-specific copy gain and increased expression of drug resistance genes such as CKS1B. These data uncover a mechanism that can account for both copy number and expression heterogeneity observed in solid tumors and establish a molecular basis for drug resistance gene selection (Patel et al. 2014).

Results

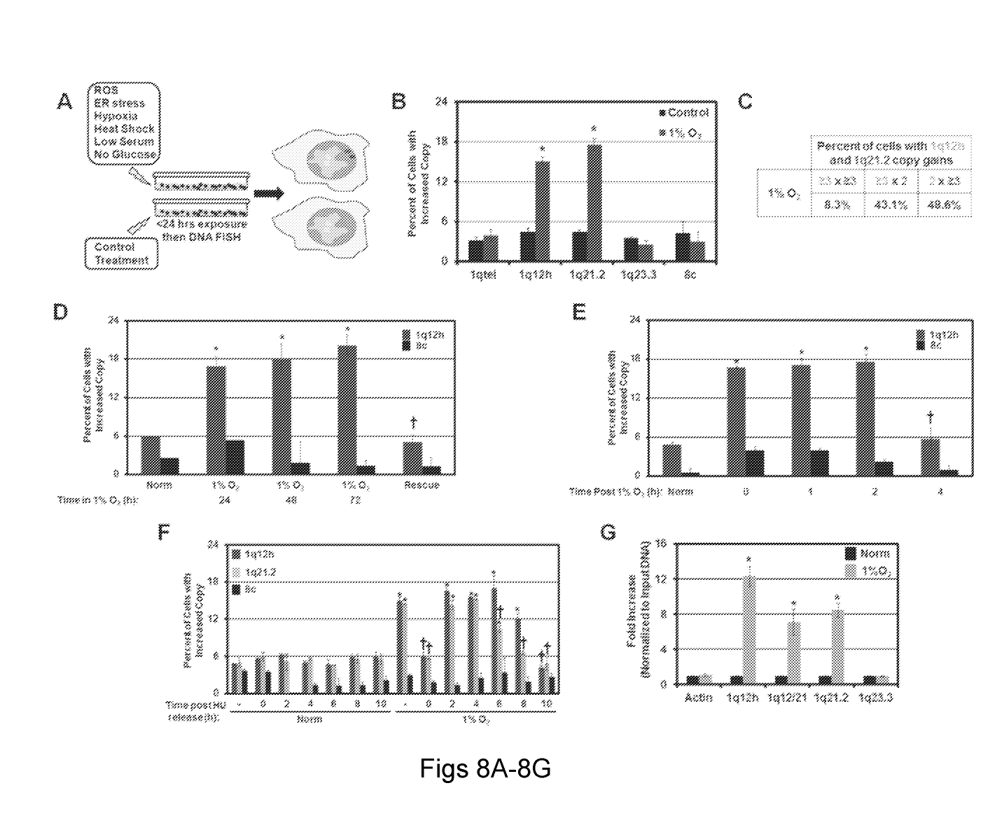

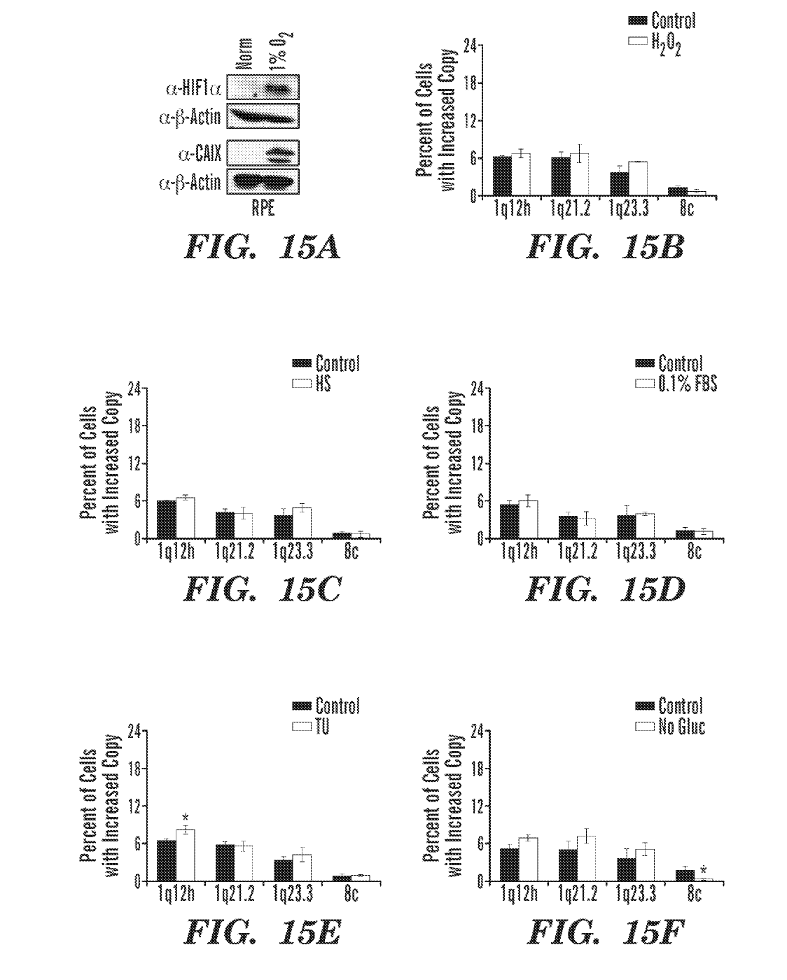

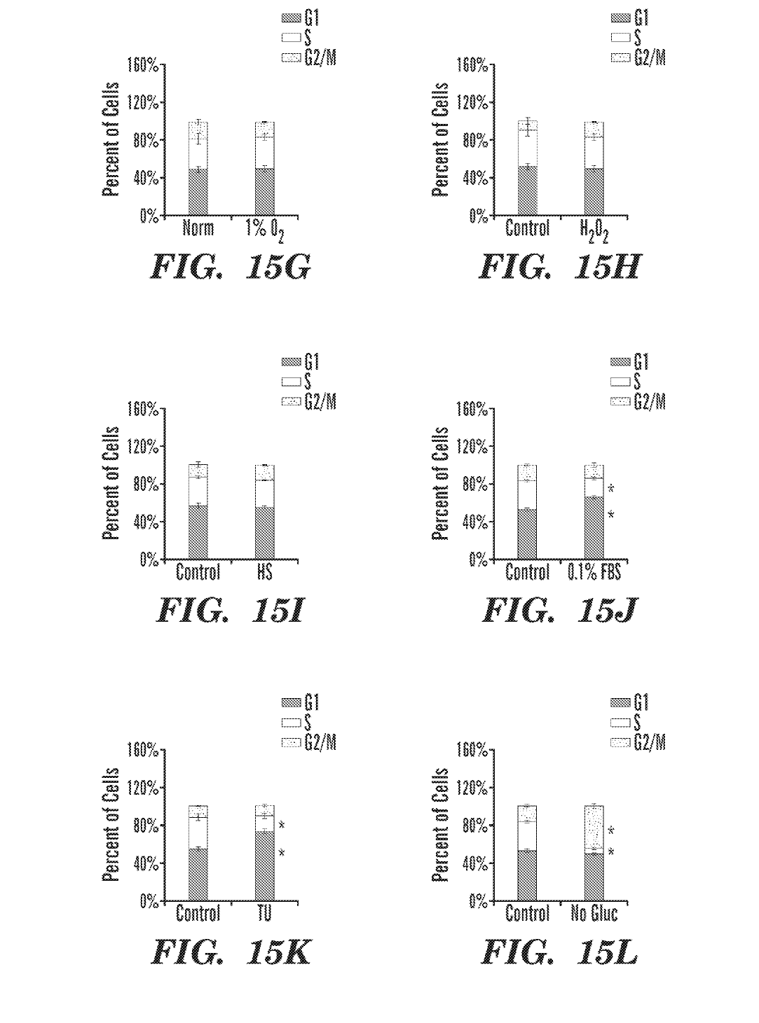

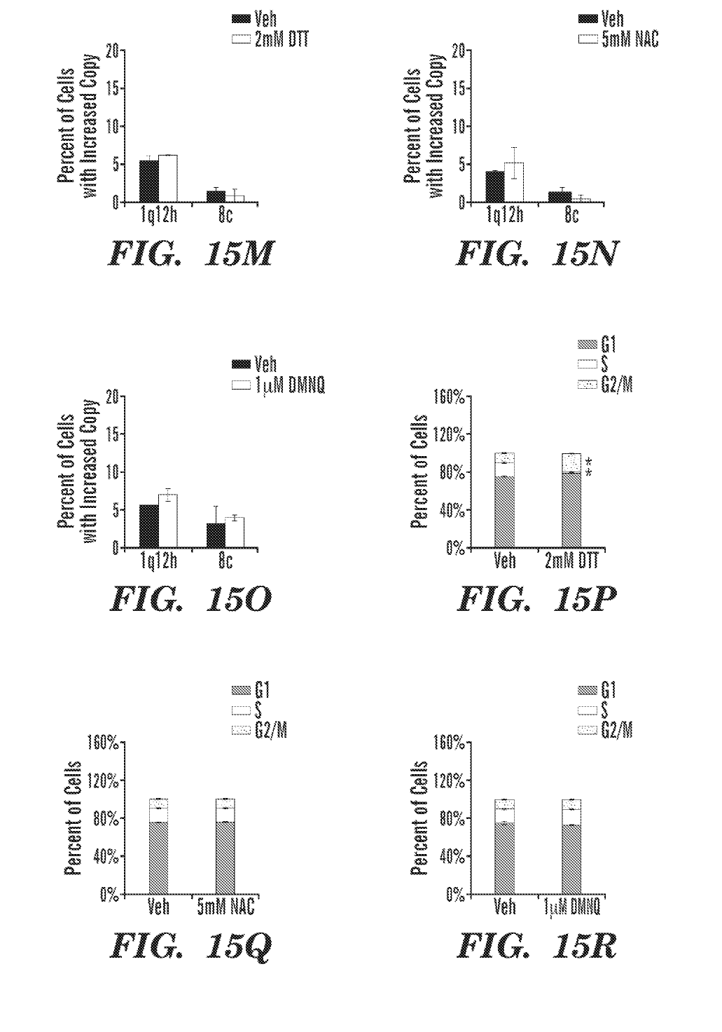

Hypoxia promotes site-specific copy gain. It was reasoned that tumor cells experience various stresses that promote copy gains, which could ultimately contribute to the copy number heterogeneity observed in tumors. Therefore, the impact that environmental conditions observed during development and tumorigenesis have on regions frequently gained in tumors and that are susceptible to transient site-specific copy gains (TSSGs; i.e., 1q12-1q21; (Beroukhim et al. 2010; Black et al. 2013; Tang and Amon 2013)) was monitored. Specifically, copy gain was screened in the nearly diploid, immortalized, but non-transformed RPE cell line (Jiang et al. 1999; Black et al. 2013) that was exposed to: reactive oxygen species (ROS, H₂O₂), ER stress (Tunicamycin, TU), temperature stress (heat shock, 43° C.), metabolic stress (low serum. 0.1% FBS; no glucose), and hypoxia (1% O₂) (FIG. 8A, FIG. 15A-15L). Cells were exposed to the indicated stresses (see Experimental Procedures) and assayed for site-specific copy gain by fluorescent in situ hybridization (FISH) and cell cycle profiles after 24 hours (FIG. 8B, FIG. 15B-15L). Using this approach, only hypoxia generated site-specific gains (FIG. 8B); while, other stresses were not drastically different from control conditions (FIG. 15D-15L). For example, 1q12h and 1q21.2 copy gains were induced in as little as 24 hours of hypoxic exposure; however, no change was observed for other chromosomal regions (e.g., 1q23.3) (FIG. 8B). Since hypoxic exposure alters the redox state of the cell (Solaini et al. 2010), it was examined whether other redox modulators impacted copy gain. Cells exposed to other reducing (DTT and N-Acetyl Cysteine) or oxidizing (DMNQ) agents did not induce site-specific copy gain, indicating that the observed gains are specific to hypoxia (FIG. 15M-15R). Spectral karyotyping analysis of hypoxic cells did not show widespread genome instability (data not shown), which was consistent with the normal cell cycle profiles observed in hypoxia (FIG. 15G). Furthermore, analysis of 1q12h and 1q21.2 in the same cells revealed that the gains in hypoxic conditions were predominantly mutually exclusive (FIG. 8C), which further underscored the site-specific nature of the gains. These results indicate that hypoxia promotes site-specific copy gain.

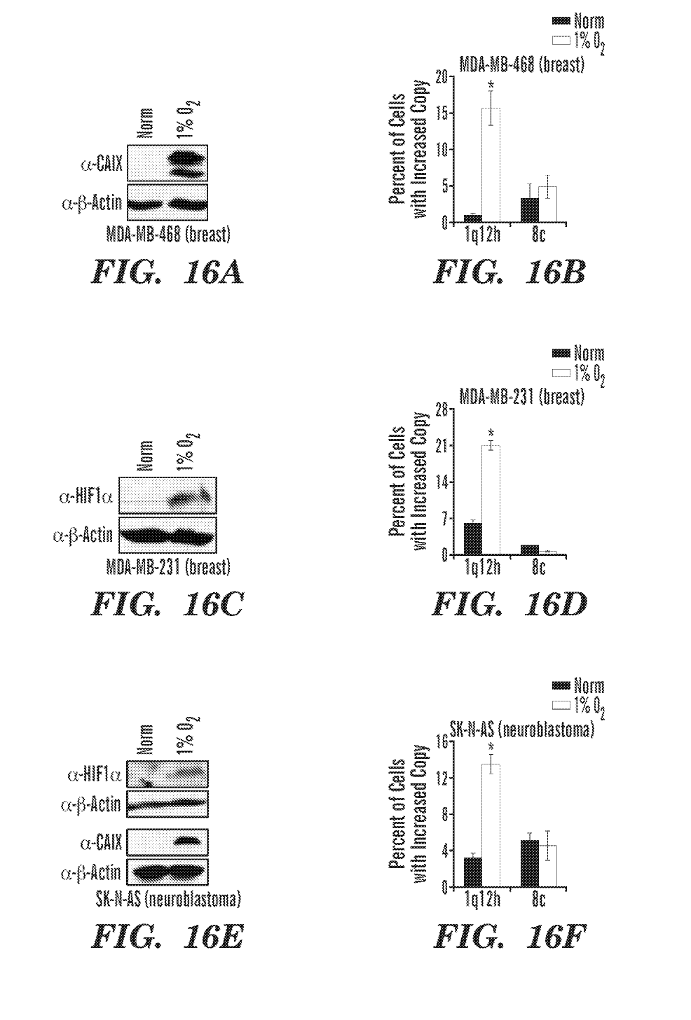

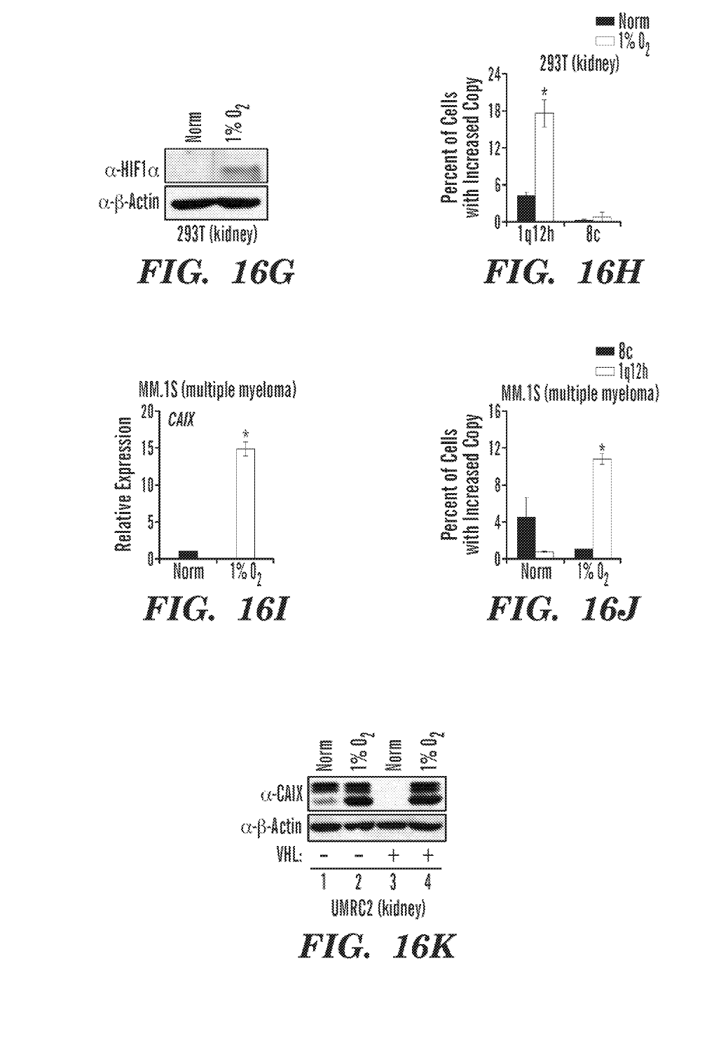

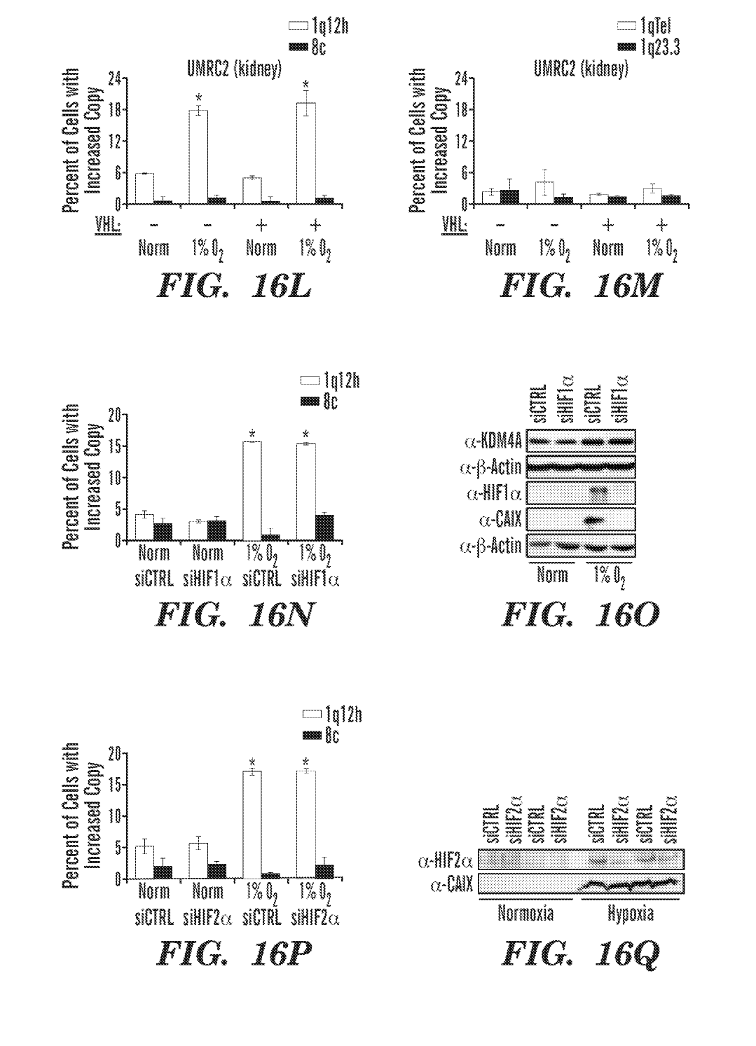

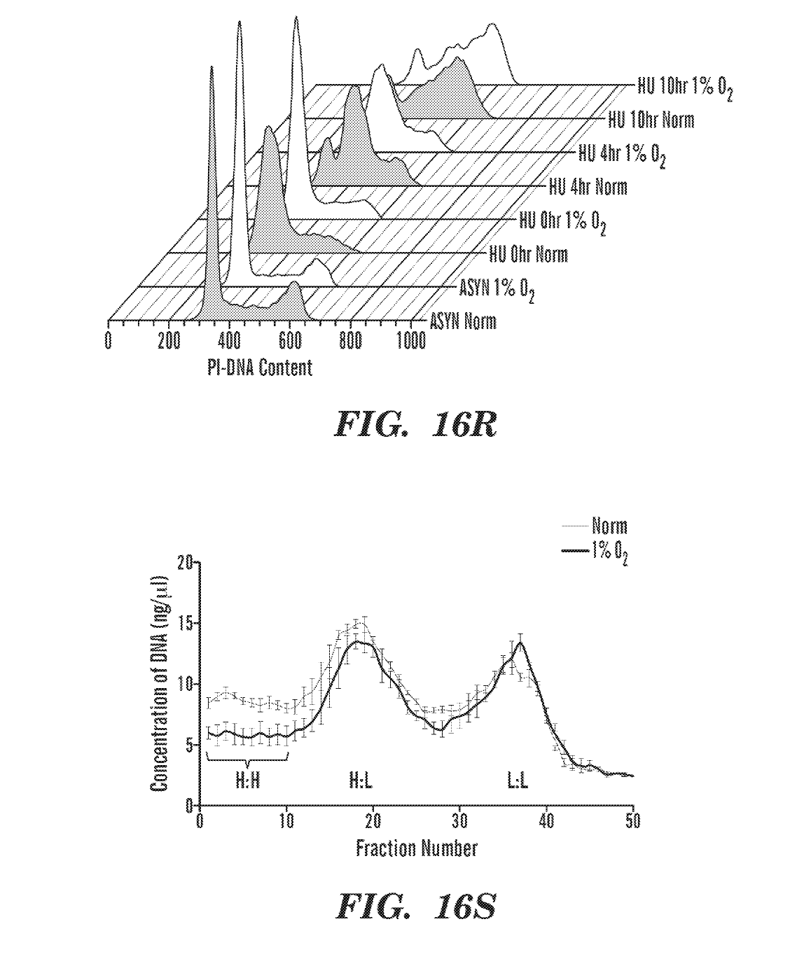

To address whether copy gain was a prevalent response to hypoxia, a diverse panel of cancer cell lines was analyzed, including: breast cancer (MDA-MB 4658, MDA-MB 231), neuroblastoma (SK-N-AS), multiple myeloma (MM.1S) and kidney cell lines (HEK293T and UMRC2) for copy gain of 1q12h by FISH following growth in hypoxia (FIG. 16A-16M). In each cell line, copy gain of 1q12h was observed under hypoxic conditions, but no change in chromosome 8 centromere (Sc). Furthermore, HIF1α or HIF2α depletion by two independent siRNAs did not prevent copy gain in hypoxic RPE cells despite blocking induction of the hypoxia-inducible target gene CAIX (FIG. 16N-16Q). Consistent with these observations, UMRC2 cells, which lack VHL and have a functionally stable HIF1α and HIF2α (Gameiro et al. 2013) resulting in hypoxia gene program activation in normoxic conditions, do not generate copy gain without hypoxia (FIG. 16K-16M). Therefore, HIF1α and HIF2α stabilization was not sufficient to promote copy gain. Together, these data strongly indicate that hypoxia-dependent copy gains are a common response that does not require the HIF1/2a pathway.

Hypoxia-Induced Copy Gains Require Proliferation.

Oxygen levels change during development and tumorigenesis (Vaupel 2004; Dunwoodie 2009); therefore, it was assessed whether site-specific copy gains are reversible upon return to normal oxygen levels (FIG. 8D). FISH analysis for 1q12h copy gain revealed an increased percentage of cells with copy gain at 24, 48, and 72 hours of growth in hypoxia; however, upon return to normoxia, the number of cells with extra. copies of 1q12h returned to baseline (FIG. 8D). In fact, copy gain of 1q12h persists for the first two hours following release from hypoxia but is lost by four hours after return to normoxia (FIG. 5E). These data indicate that hypoxia-dependent copy gains are dynamic and reversible.

To demonstrate that hypoxia-dependent copy gains require proliferation, cells were arrested using hydroxyurea (HU) under hypoxic conditions (FIG. 16R). Cells arrested at G1/S in hypoxia did not exhibit copy gains (FIG. 8F). However, upon release from the arrest, hypoxic cells rapidly accumulated copy gain of both 1q12h and 1q21.2. Intriguingly, these gains were lost prior to the end of S phase, with loss of 1q21.2 copy gains occurring slightly faster than 1q12h loss, indicating that individual regions exhibit site-specific copy gain with different kinetics. Furthermore, it was demonstrated that these regions were rereplicated by performing quantitative PCR on DNA purified from the heavy:heavy (H:H) fraction from a CsCl density gradient (FIG. 8G, FIG. 16S). These results demonstrate that hypoxia-induced copy gains occur during S phase and are reversible.

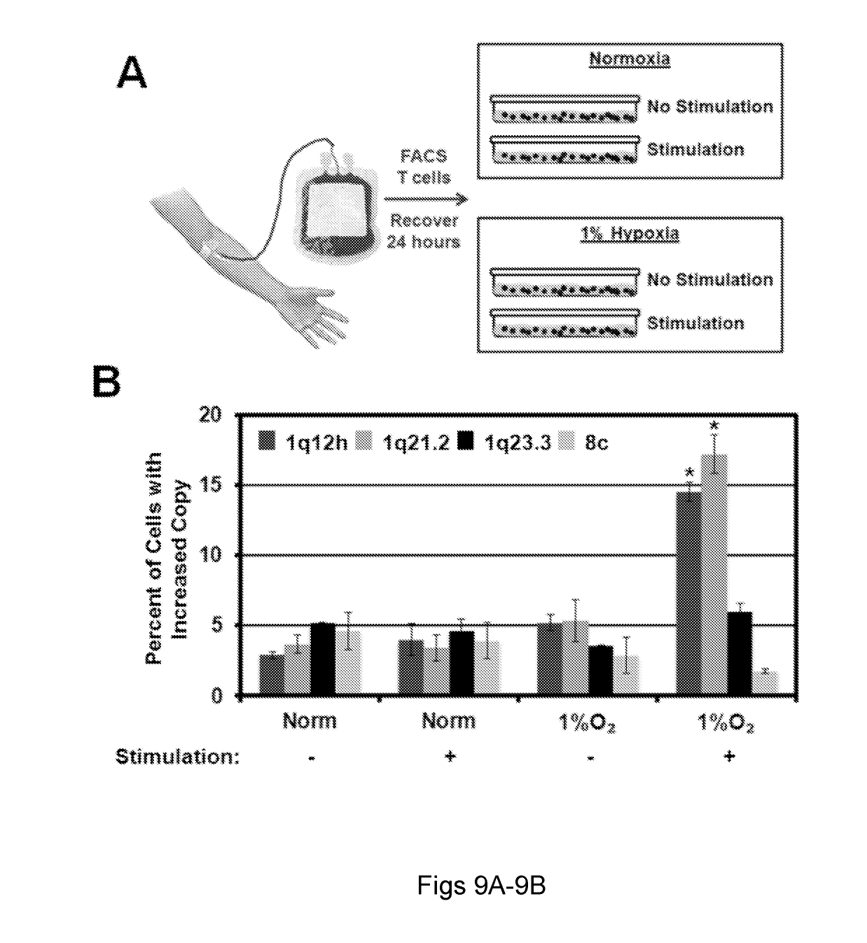

The next major question was whether a hypoxic signal could drive site-specific copy alterations in non-immortalized or non-cancer cells. To address this question, CD4+ T cells were isolated by fluorescence-activated cell sorting (FACS) from buffy coat and peripheral blood of healthy individuals (FIG. 9A). Following isolation, T cells were allowed to recover in normoxia (i.e., 21% O₂, which is “normnoxia” for cell culture similar to the 13.2% O₂ observed in arterial blood not associated with hemoglobin (Carreau et al. 2011)) for 24 hours in the presence of 1L2 with or without stimulation with anti-human CD3 and CD28 antibodies. Following recovery, T cells were maintained in normoxia or transferred to hypoxia for an additional 24 hours and analyzed by FISH for site-specific copy gain. Only stimulated T cells grown in hypoxia for 24 hours exhibited copy gain of 1q12h and 1q21.2, but not gain of 1q23.3 or 8c (FIG. 9B). These results demonstrate that primary cells subjected to hypoxic conditions promote site-specific copy gain in a proliferation-dependent manner,

KDM4A Stabilization Promotes Hypoxia-Induced Copy Gain.

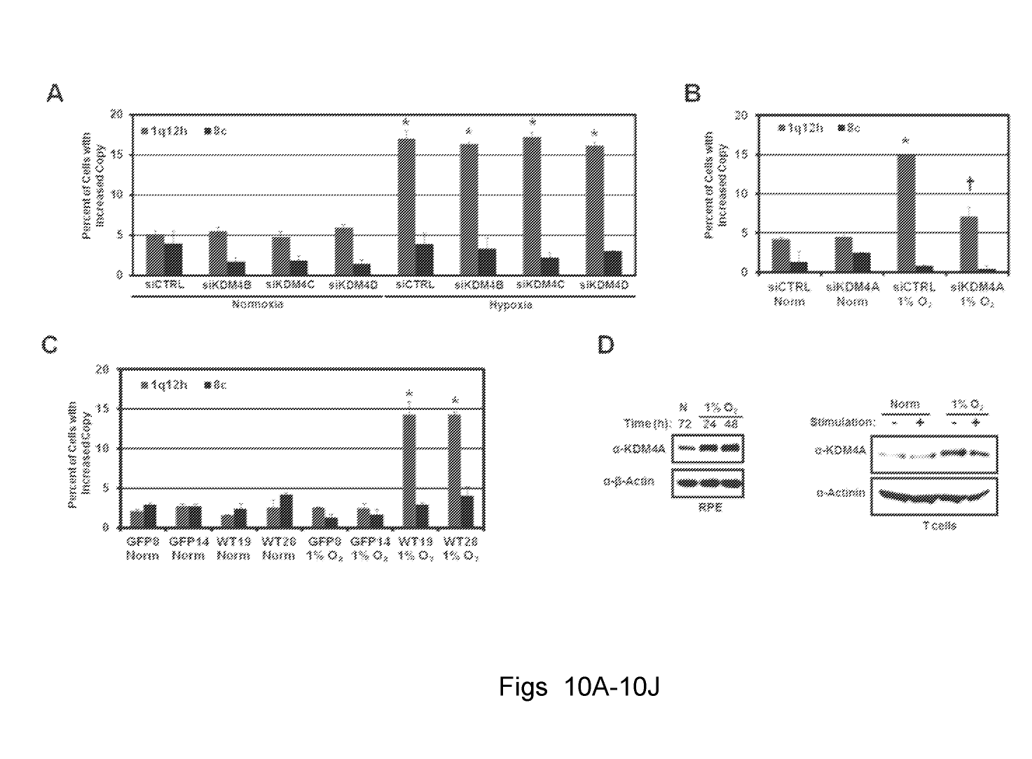

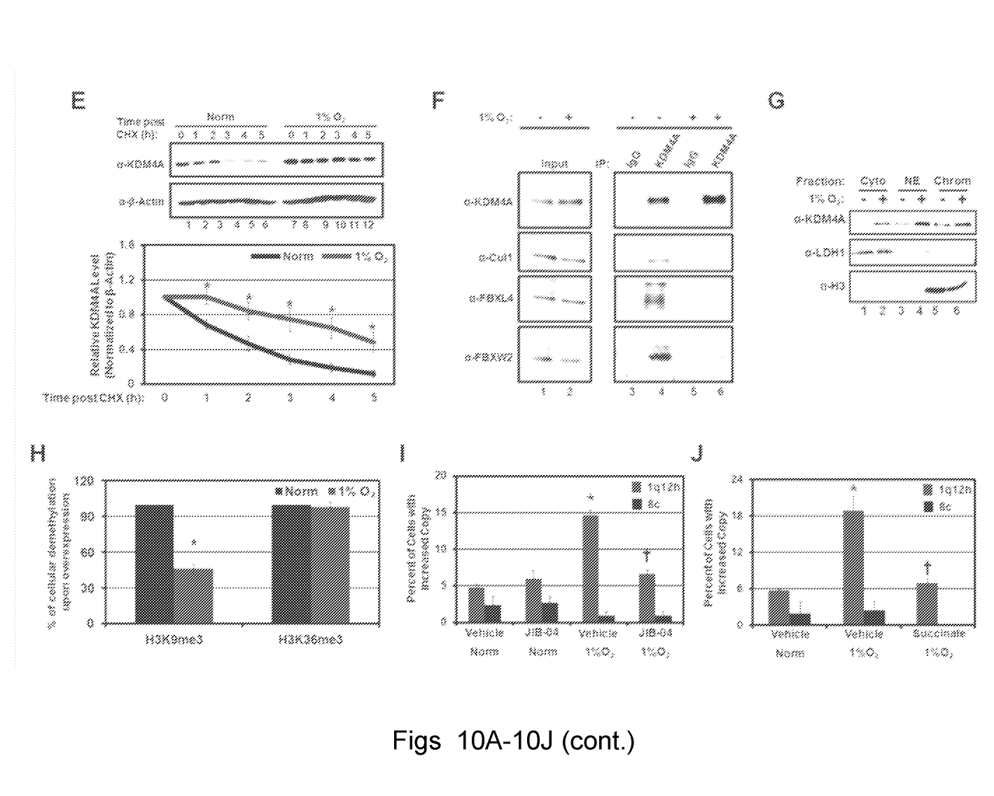

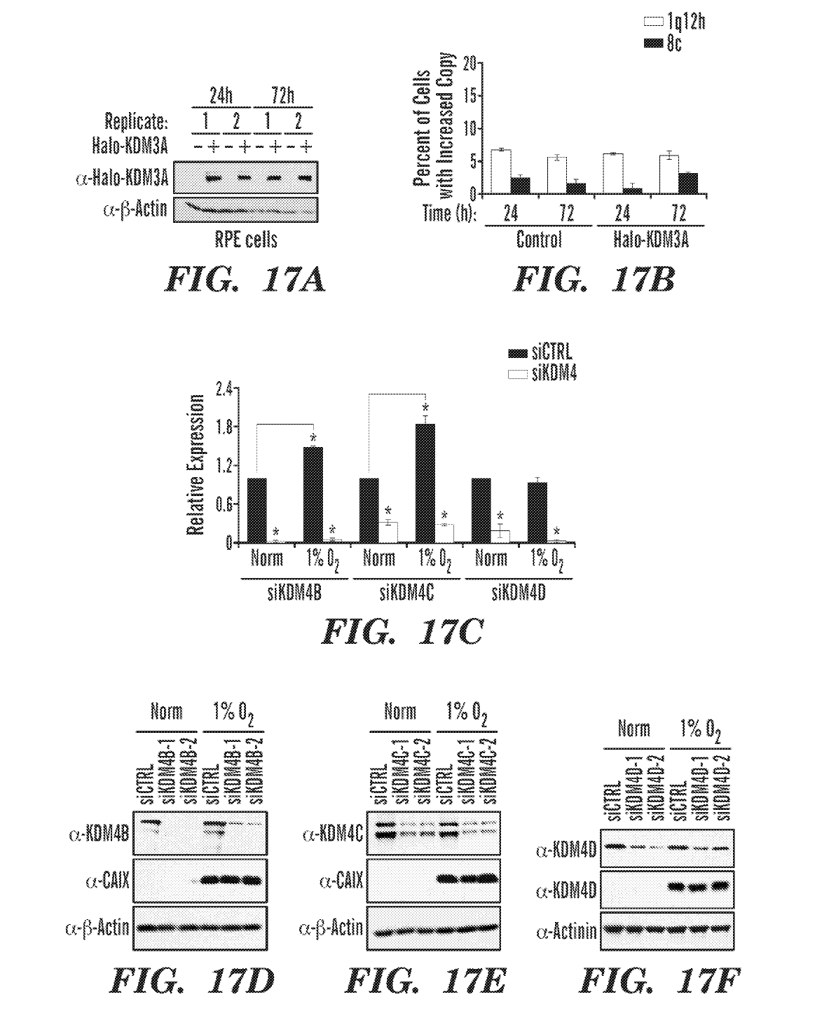

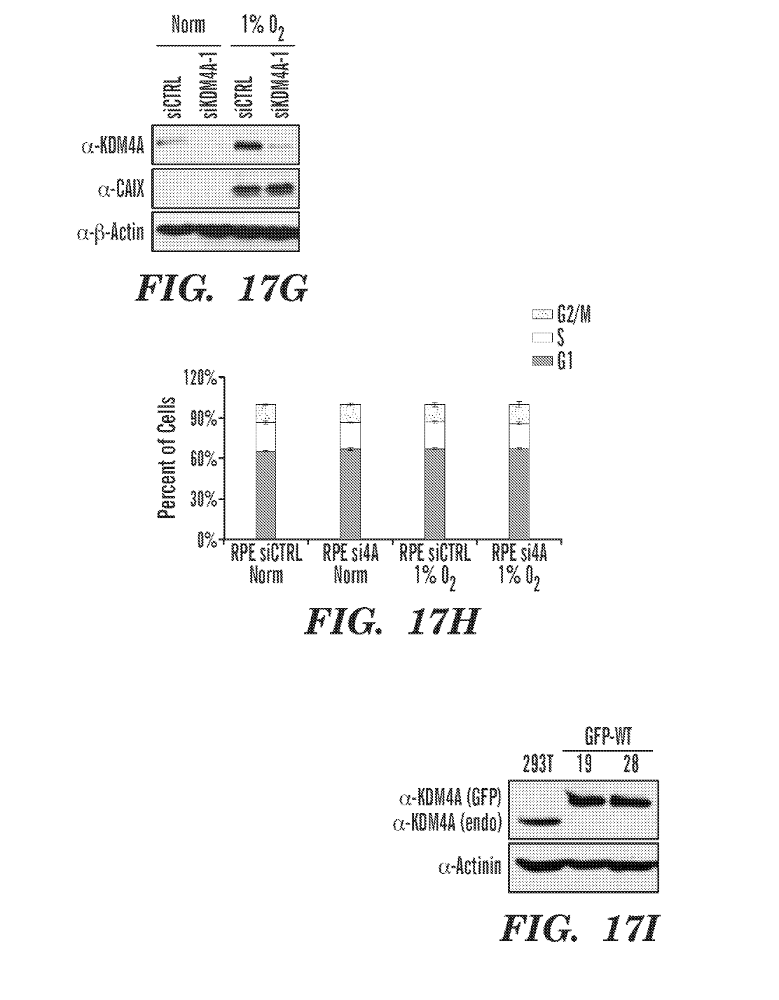

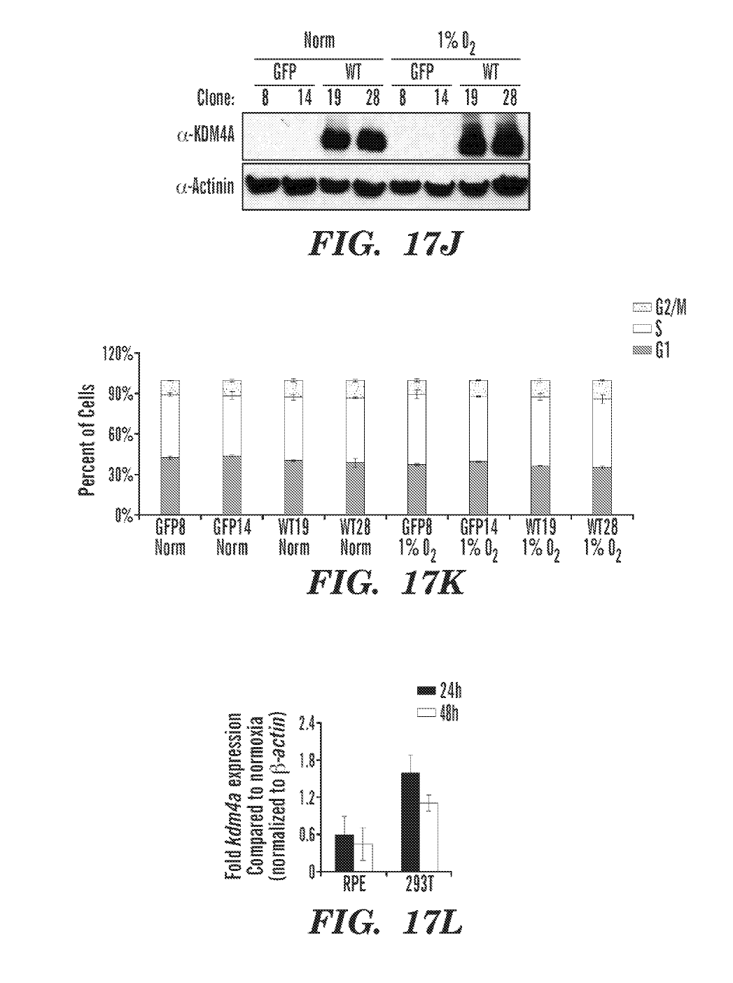

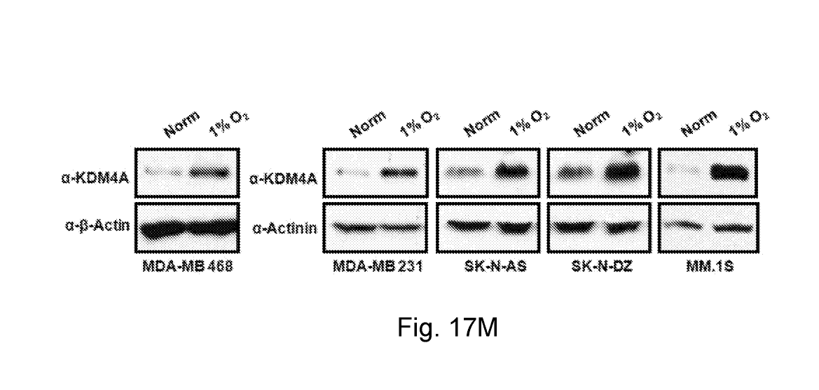

Since depletion of either H3K9me3 or H3K36me3 was sufficient to promote site-specific copy gain (Black et al. 2013), it was reasoned histone demethylases may mediate hypoxia-induced copy gain. JmjC-containing demethylases use molecular oxygen as a cofactor for demethylation, and thus hypoxia has been proposed to inactivate the JmjC-containing demethylases. However, previous reports have shown that certain JmjC-containing lysine demethylases that target H3K9 methylation are transcriptionally upregulated (KDM4B and KDM4C), or retain their activity (KDM3A) upon hypoxic exposure (Krieg et al. 2010; Lee et al. 2013). It was tested whether KDM3A overexpression or siRNA depletion of KDM4 enzymes with independent siRNAs during hypoxia was responsible for site-specific gain. KDM3A overexpression was not sufficient to promote site-specific copy gain (FIG. 17A,17B), In addition, depletion of KDM4B-D with two independent siRNAs did not block hypoxia-induced copy gain, despite increased KDM4B/C expression in hypoxia (FIG. 10A, FIG. 17C-17F). However, depletion of KDM4A blocked the hypoxia-dependent copy gain (FIG. 10B, FIG. 17G), without altering cell cycle distribution (FIG. 17H).

To demonstrate a genetic requirement for KDM4A, KDM4A knockout 293T cells were generated using CRISPR/Cas9. Either G7 P or GFP-KDM4A (WT) were reintroduced and single cell clones generated. GFP-KDM4A clones that had expression levels similar to those of endogenous KDM4A in parental 293T cells were selected (FIG. 17I), Importantly, the restored GFP-KDM4A was induced under hypoxic conditions (FIG. 17J). Two independent GFP clones (lacking endogenous KDM4A) were unable to generate site-specific copy gain in hypoxia, while both GFP-KDM4A rescue clones were able to generate site-specific copy gains (FIG. 10C), without altering cell cycle distribution (FIG. 17K). These results demonstrate that KDM4A is necessary for the generation of site-specific copy gain in response to hypoxia.

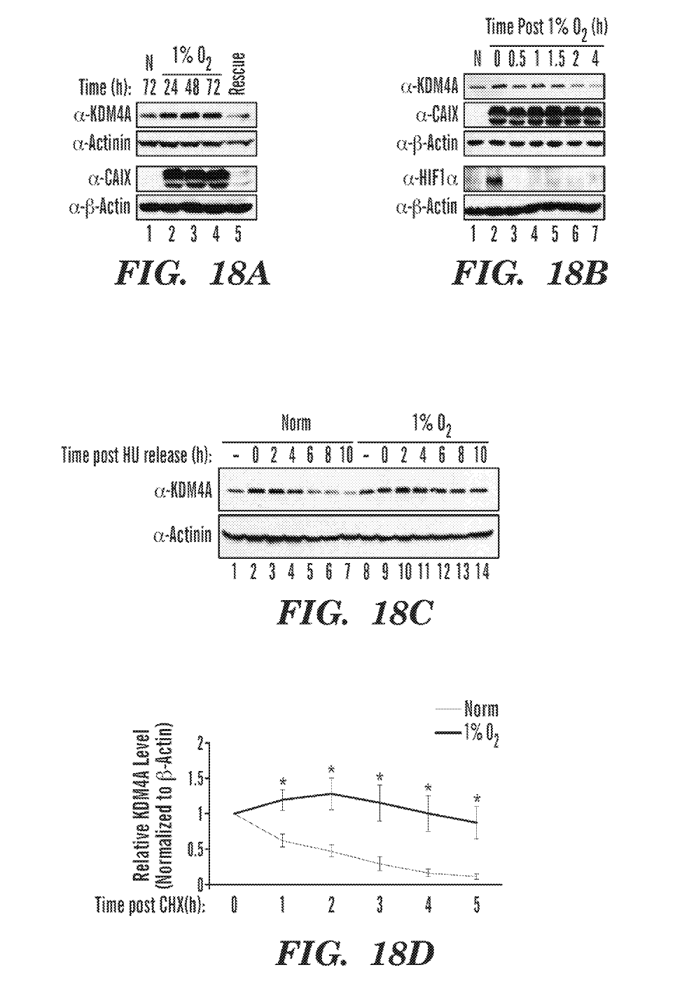

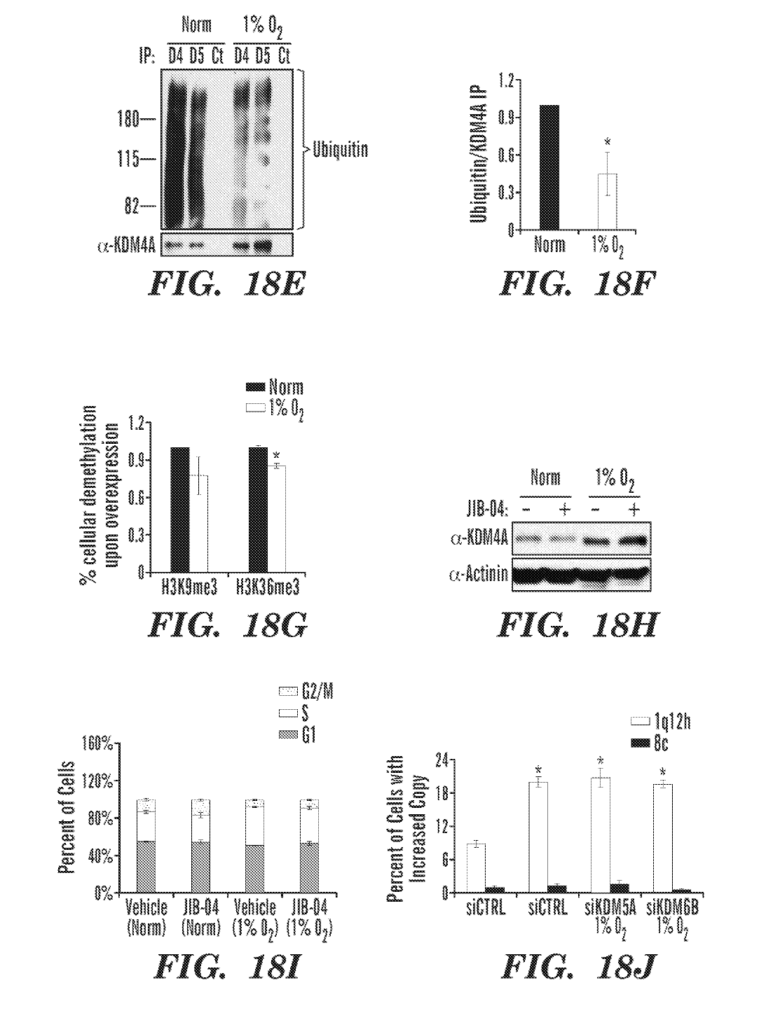

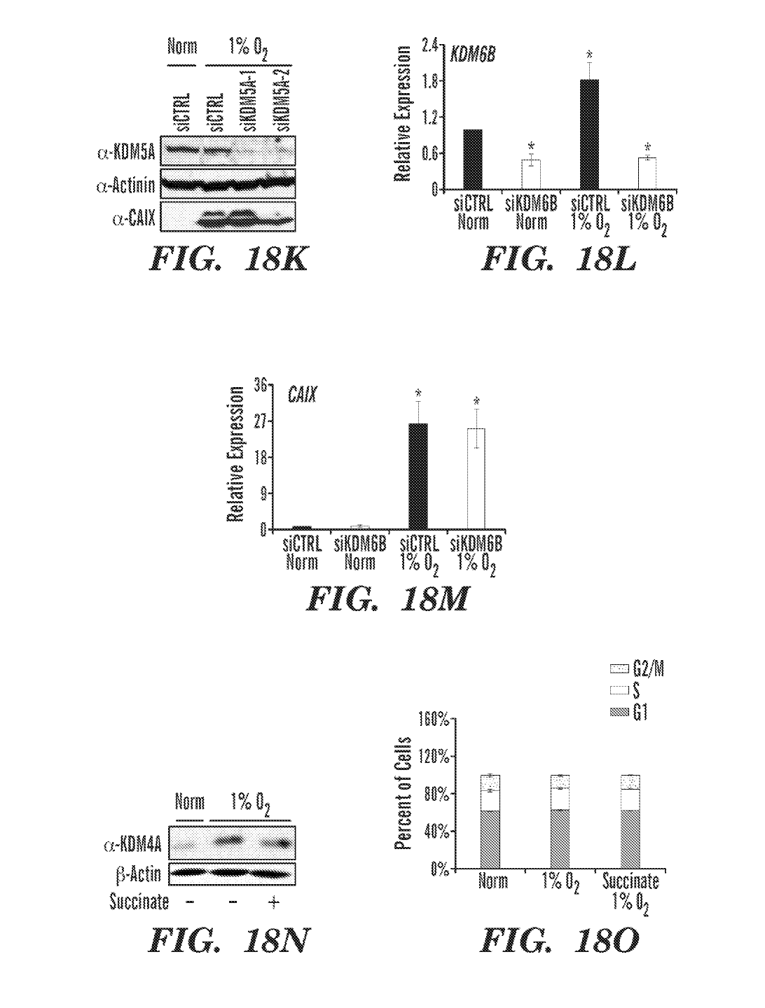

In agreement with previous reports, increased KDM4A transcript was not observed upon hypoxic exposure (FIG. 17L) (Beyer et al. 2008). However, KDM4A protein levels were increased with as little as 24 hours of exposure to hypoxia in all cell lines tested (FIG. 10D, left panel; FIG. 17M) as well as in the primary CD4+ T cells treated with hypoxia (FIG. 10D, right panel), In fact, KDM4A protein levels were regulated in the same temporal fashion as the copy gains upon hypoxic exposure and return to normoxia (FIG. 18A-C). Furthermore, hypoxia resulted in KDM4A protein stabilization (e.g., increased half life from 1 hr 49 min to 4 hrs 56 min; FIG. 10E and FIG. 18D). KDM4A proteins levels are regulated by the SKP1-Cul1-F-box (SCF) containing ubiquitin ligase complex (Tan et al. 2011; Van Rechem et al. 2011). KDM4A interacts with the SCF-ubiquitin ligase complex and is ubiquitinated and degraded in a cell cycle-dependent manner. Therefore, it was reasoned that this complex may influence KDM4A ubiquitination and protein stability during hypoxia exposure. Consistent with previous results and the increased half-life of KDM4A in hypoxia, KDM4A had a reduced association with the SCF complex and less ubiquitination under hypoxic conditions (FIG. 10F, FIG. 18E,18F) (Van Rechem et al. 2011).

KDM4A overexpression results in increased chromatin association throughout the genome and is associated with rereplication of specific regions (Van Rechem et al. 2011; Black et al. 2013). In agreement with these observations, hypoxia resulted in stabilized KDM4A that also increased in the chromatin fraction (FIG. 10). To determine whether KDM4A remained active under hypoxic conditions, demethylation was assessed using standard immunofluorescence assays (Whetstine et al. 2006). Importantly, KDM4A retained enzymatic activity under hypoxic conditions. Twenty-four hour exposure to hypoxic conditions resulted in a reduction but not a loss in H3K9me3 activity, while not affecting H3K36me3 demethylation (FIG. 10H). KDM4A remained active, with modest reduction in demethylase activity, even after 48 hours in hypoxic conditions (FIG. 18G). These results demonstrate that KDM4A was stabilized, enriched on the chromatin and able to retain enzymatic activity under hypoxic conditions.

Small Molecule Inhibition of Hypoxia-Induced Copy Gains.

Based on these observations, it was hypothesized that KDM4A inhibition could serve as a tool to modulate the copy number alterations observed in hypoxia. To test this hypothesis, cells were pre-treated with an inhibitor of JmjC demethylases, JIB-04 (Wang et al. 2013; Van Rechem et al. 2015). JIB-04 is not a selective inhibitor of KDM4A but inhibits the KDM4 family as well as KDM5A and KDM6B (Wang et al. 2013). JIB-04 did not substantially alter KDM4A protein levels or cell cycle profiles in hypoxia (FIG. 18I-H,18I). However, treatment with JIB-04 significantly reduced hypoxia-dependent copy gain of 1q12h (FIG. 10I). Since JIB-04 also targets KDM5A and KDM6B, these KDMs were depleted with siRNAs under hypoxic conditions. Depletion of KDM5A or KDM6B was insufficient to rescue hypoxic induction of site-specific copy gain (FIG. 18J-18M). Since depletion of KDM4B-D, KDM5A or KDM6B failed to rescue site-specific copy gain in hypoxia, JIB-04 is likely suppressing site-specific gain through KDM4A inhibition.

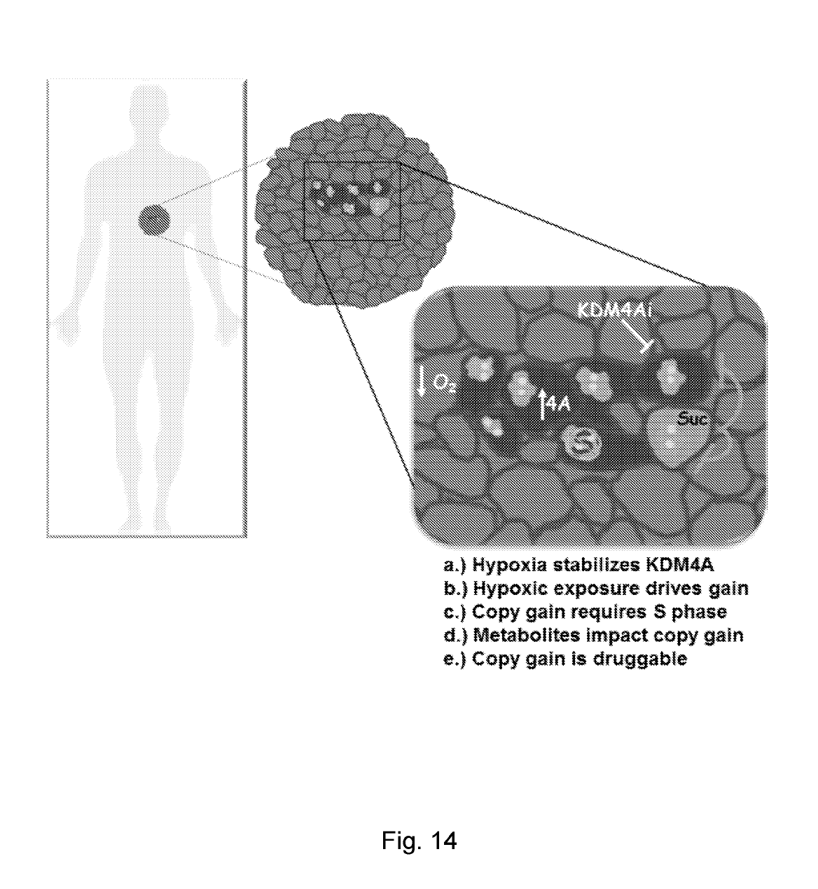

Since all JmjC-containing proteins can be inhibited by the natural metabolite succinate (Smith et al. 2007: Black et al. 2012), RPE cells were treated with succinate prior to growth in hypoxia. Succinate treatment did not alter KDM4A stabilization or cell cycle progression (FIG. 18N,18O) but was sufficient to abrogate hypoxia-dependent copy gain of 1q12h (FIG. 10J). These results establish that hypoxia-dependent copy gains are a biological response that can be pharmacologically regulated and emphasize the impact that metabolic state can have on copy number. In addition, these data illustrate how a metabolic change can counteract hypoxia-induced gains, which provides another basis for copy number heterogeneity within tumors (see FIG. 14₁).

Hypoxia-Induced Copy Gains are Conserved.

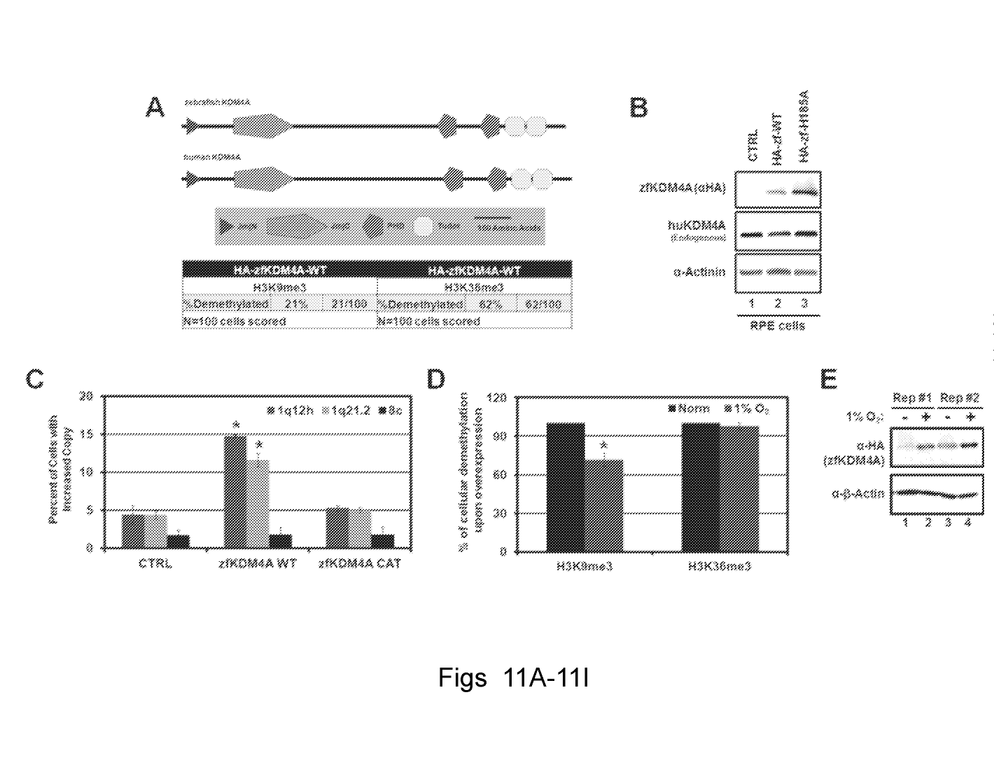

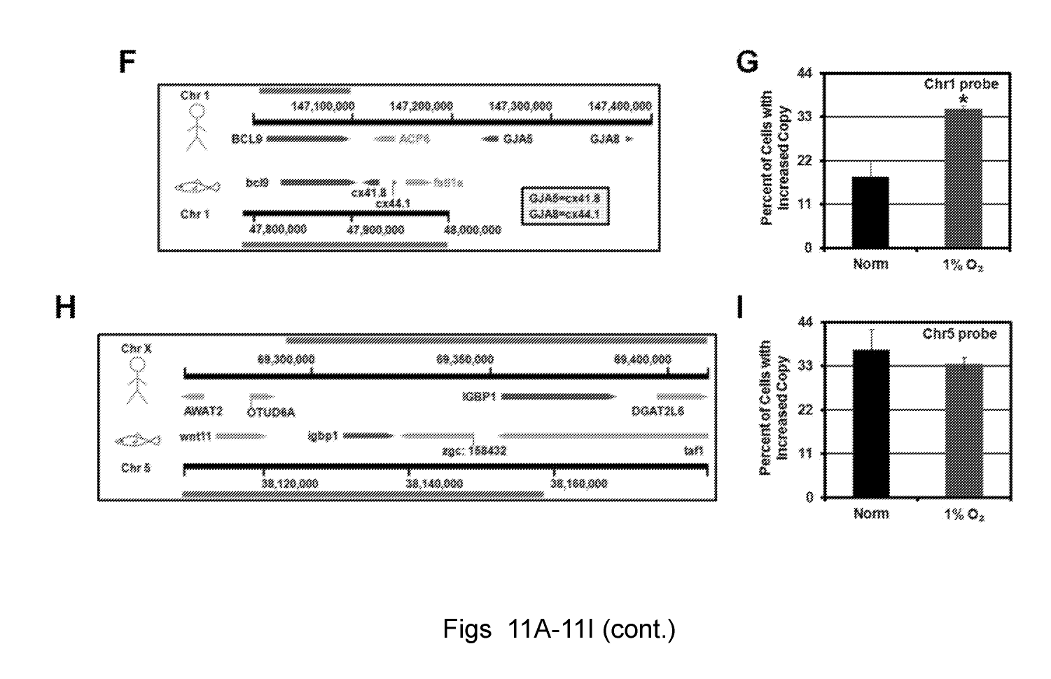

Based on the findings in primary and cancer cells, it was hypothesized that hypoxia-induced KDM4A stabilization and copy gains were an evolutionarily conserved response. In order to test this possibility, zebrafish KDM4A was examined. Wild type zebrafish KDM4A (zfKDM4A-WT). which has a similar architecture to human KDM4A (huKDM4A; FIG. 11A), was able to demethylate both H3K9me3 and H3K36me3 (FIG. 11A). In addition, overexpression of the catalytically active zfKDM4A (zfKMD4A-WT) in human cells was sufficient to promote copy gain of regions regulated by human KDM4A (huKDM4A) (FIG. 11B,11C). Similar to huKDM4A, zfKDM4A retained catalytic activity in hypoxia, albeit with reduced activity on H3K9me3, and was stabilized under hypoxic treatment (FIG. 11D,11E).

This prompted the evaluation of whether hypoxia promoted copy gain in zebrafish cells. Zebrafish are cultured in water saturated with atmospheric oxygen levels (21%) and zebrafish cell lines are considered to be hypoxic at or below 3% O₂ (Jopling et al. 2012). Using the zebrafish cell line AB.9 (Paw and Zon 1999), the ability of hypoxia to promote copy gain of a region syntenic to the human BCL9 gene on 1q21.2 (FIG. 11F) was assessed. This syntenic region was gained in AB.9 cells upon hypoxia exposure (FIG. 11G). However, a second homologous, but non-syntenic region to the human IGBP1 gene on zebrafish chromosome 5 (FIG. 11H; region covered by the Xq13.1 probe in human cells, green bar top of schematic) was not copy gained in response to growth in hypoxia (FIG. 11I). These data demonstrate that copy gain is a conserved response to hypoxia.

Hypoxic Tumors are Enriched for Hypoxia-Induced Copy Gains.

Since primary cells, cultured cancer lines and zebrafish cells promote site-specific gain in response to hypoxia, it was hypothesized that hypoxic conditions within primary tumors may contribute to SCNA observed in tumors (Beroukhim et al. 2010). By analyzing tumors, physiological hypoxia that is occurring within the tumor is controlled for. This analysis will circumvent the issue of standard cell culture conditions (21% O₂; normoxia in vitro) and establish if the relationship observed in culture is occurring in tumors. Ultimately, this analysis will permit in vivo validation, and in turn, allow testing of newly identified regions in cell culture models.

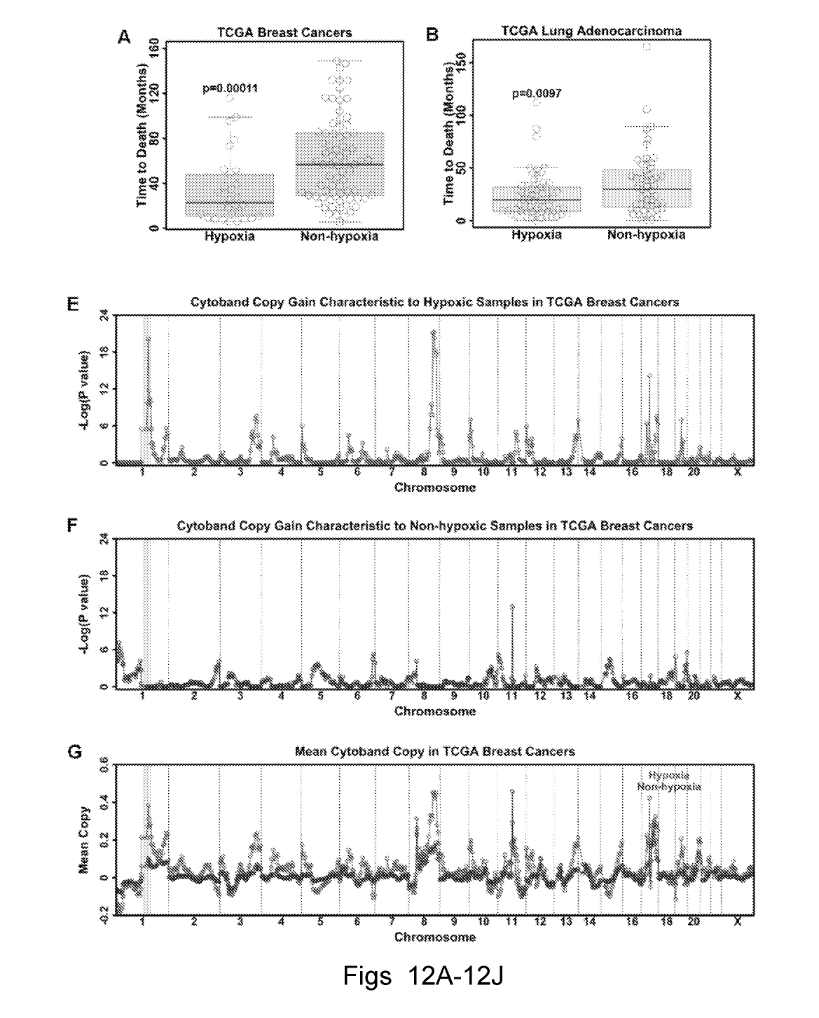

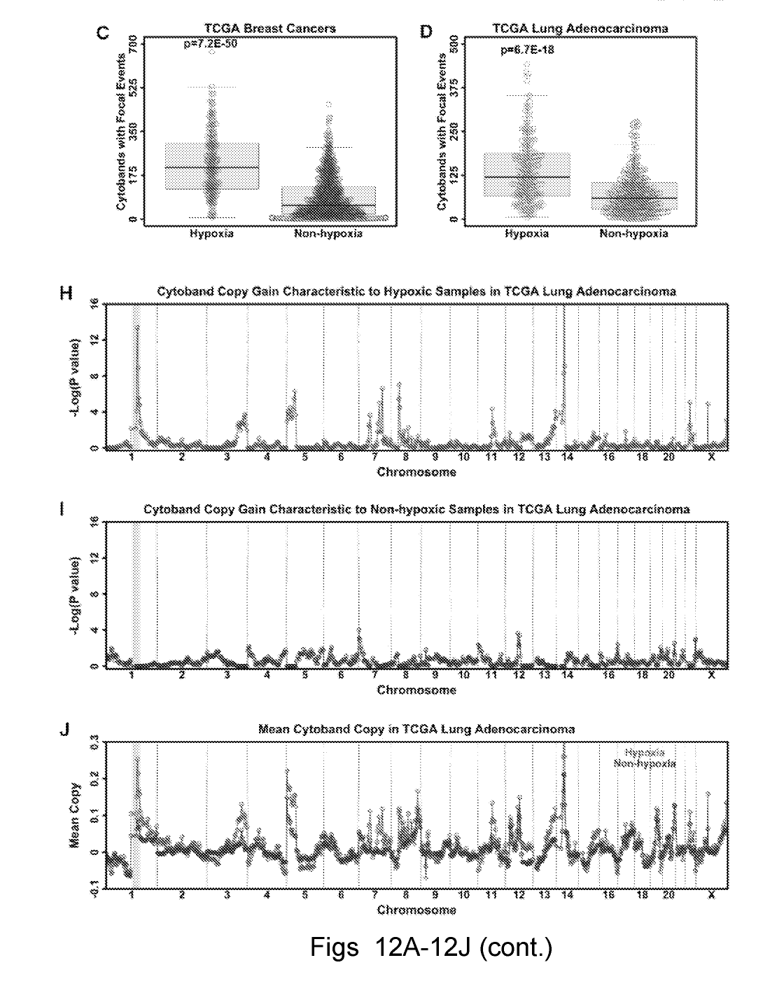

To address the hypothesis, primary breast (BRCA) and lung (LUAD) tumors from the TCGA were analyzed for SCNA in hypoxic compared to non-hypoxic tumors. To identify hypoxic tumors, the hypoxia gene signature derived by Winter and colleagues (Winter et al. 2007) was utilized to perform an unbiased consensus hierarchical clustering of BRCA and LUAD (Network 2012) (data not shown) (See data processing in Materials and Methods). As validation of this gene set and clustering approach, 65 out of 88 basal BRCA samples reside in the hypoxic cluster. Basal breast cancer has been previously demonstrated to be more hypoxic than other molecular subtypes of breast cancer, which supports the computational analyses (Perou 2010). Furthermore, previous reports have demonstrated that hypoxia is a negative prognostic marker in multiple tumor types (Hockel et al. 1996; Eschmann et al. 2005; Wang et al. 2014). The present analyses further substantiated these observations since hypoxic BRCA and LUAD samples had a significantly higher risk (faster time to death) in both BRCA (FIG. 12A) and LUAD (FIG. 12B).

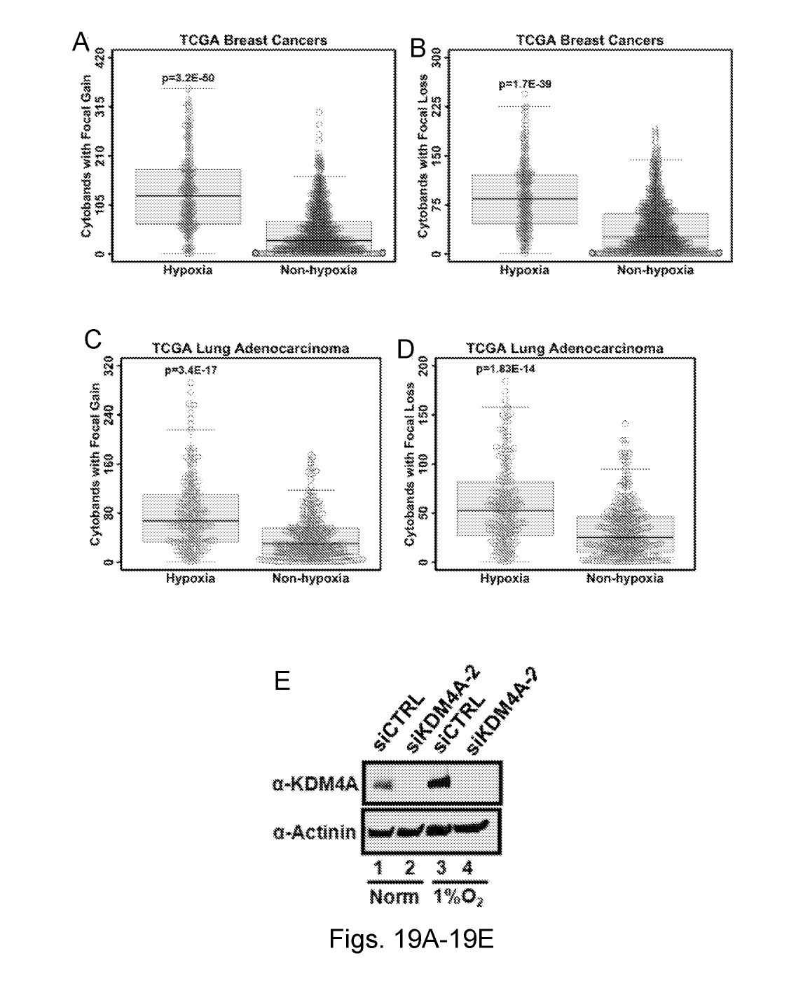

It was next asked whether specific cytogenetic bands exhibit focal amplifications in hypoxic BRCA and LUAD samples. In fact, BRCA and LUAD samples also had increased number of focal copy number events in hypoxic samples (FIG. 12C,12D, FIG. 19A-19E). A strong enrichment of copy gain of 1p11.2 through 1q23.3 (blue shaded region) was observed in hypoxic BRCA (FIG. 12E) and LUAD (FIG. 12H) that was not present in non-hypoxic samples (FIG. 12F-12J). Taken together, these data highlight that hypoxic conditions are associated with worse outcome and focal SCNA in tumors and that regions with hypoxia-dependent copy gain in cell culture are also focally gained in hypoxic primary tumors in two different cancer types. These data further emphasize the relationship between hypoxia and driving site-specific copy gain in vitro and in vivo.

Hypoxia Induces Copy Gain and Expression of a Drug Resistant Oncogene.

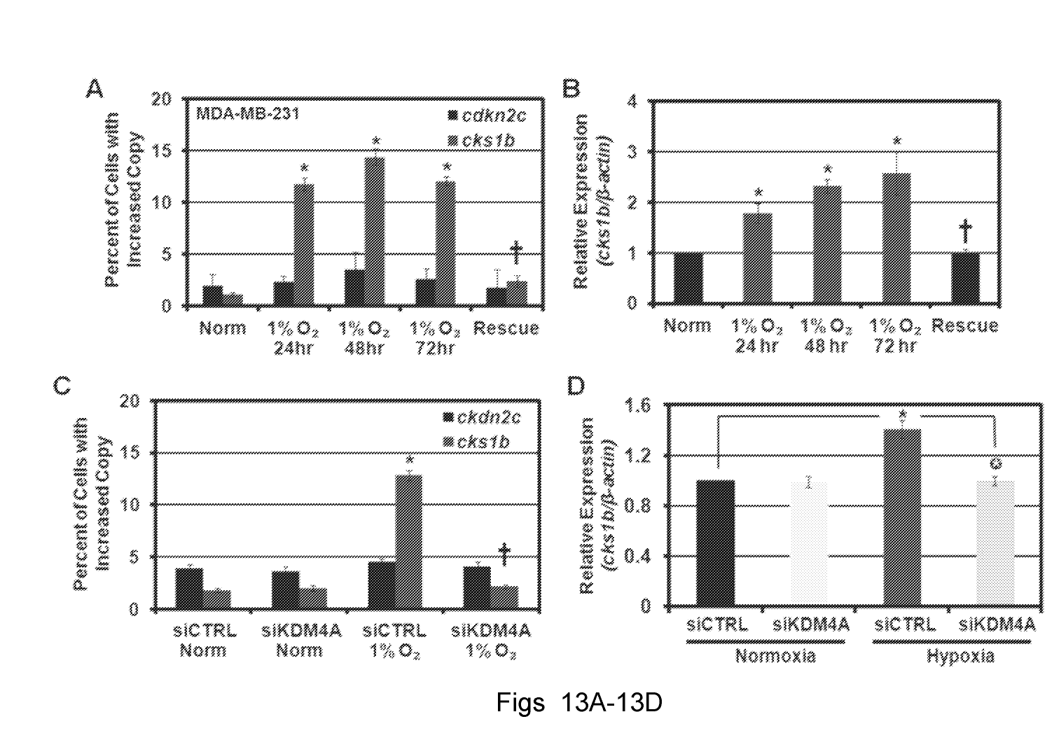

To date, a function for transient site-specific copy gains has yet to be assigned. Therefore, it was asked whether hypoxic exposure served as a mechanism to promote gene amplification, and in turn, increase gene expression. Analysis of both BRCA and LUAD identified seven genes that were amplified and had altered expression in both tumor types (data not shown). Of particular interest was the drug resistance oncogene CKS1B, which has low-level copy gains (1-3 copies) in several cancers (Shaughnessy 2005). This level of gain corresponds to comparable increased expression in tumors, which is associated with drug resistance and worse outcome in patients (Wang et al. 2009; Shi et al. 2010; Martin-Ezquerra et al. 2011; Khattar and Thottassery 2013). Since this target emerged from our in silico analyses and has major implications in tumor drug response and patient outcome, it was determined whether CKS1B was copy-gained. Using the breast cancer line MDA-MB-231, copy gain was observed for CKS1B upon hypoxic exposure, which was reversed upon return to nonnoxia (FIG. 13A). The gain of CKS1B also correlated with an increase in transcription of CKS1B, which was rescued upon returning the cells to normnoxia (FIG. 13B). It was further demonstrated that KDM4A depletion was sufficient to block both the copy gain and transcriptional increase observed for CKS1B under hypoxic conditions (FIG. 13C,13D; FIG. 19E). Taken together, these results indicate that hypoxia can promote site-specific copy gain and increased expression of drug resistance genes such as CKS1B. These data uncover a mechanism that can account for both copy number and expression heterogeneity observed in solid tumors (Patel et al. 2014).

Discussion

Described herein is a cellular mechanism of transient site-specific genomic copy gains (TSSGs) in response to hypoxic stress; this mechanism does not require genetic manipulation or drug treatment. Cells exposed to tumor-relevant hypoxia (1% O₂) (Rofstad 2000), but not other physiological stresses, exhibited copy gain in as little as 24 hours. Hypoxia promoted site-specific gains not only in transformed cancer cells, but also in primary human T cells. The generation of site-specific copy gains was conserved across species, as a syntenic region in zebrafish cells was also gained when exposed to hypoxia. Analysis of primary human tumors from TCGA demonstrated that breast and lung tumors that exhibit a hypoxic gene signature were associated with copy gains in the regions generated in human and zebrafish cell culture. Most importantly, it was demonstrated that hypoxic tumors predicted amplification and expression for the drug resistant oncogene CKS1B, which was confirmed in a human breast cancer cell line treated with hypoxia. These copy gains were the result of KDM4A stabilization, which was reversible upon normoxia exposure. It was further demonstrated that hypoxia-dependent copy gains are druggable, as pretreatment of cells with succinate or a KDM chemical inhibitor blocked hypoxia-induced copy gains. Taken together, this work uncovered a conserved response to hypoxia from zebrafish to man that generates site-specific copy gains. These results also highlight how hypoxia can contribute to tumor heterogeneity and indicate that KDM4A inhibitors can be utilized as co-therapeutics to suppress copy gains.

This study provides a mechanistic view of how tumors could acquire intra-tumoral heterogeneity and how variations in copy number could arise during tumor development. This work also indicates that intra-tumoral heterogeneity could include not only stable, heritable SCNA from different subclones, but also include transient heterogeneity arising from environmental factors, changes in cell cycle, metabolism or altered chromatin state. Furthermore, the present results underscore how non-genetic alterations in the tumor microenvironment, including the availability of oxygen or metabolites (i.e., succinate), can contribute to or limit intra-tumoral heterogeneity (Junttila and de Sauvage 2013) (FIG. 14). These findings highlight the conserved impact that stress, metabolic state and proliferative capacity can have on intra-tumor copy number variation, which has been documented across cancers (Gerlinger et al. 2012; Burrell et al. 2013; Junttila and de Sauvage 2013; Nathanson et al. 2014). It is not yet clear if this environmental control of copy gain can be tumorigenic under specific circumstances. Without wishing to be bound by theory, mechanism described may not be a transforming event, but an adaptive response to stress, which could also serve to modify oncogenic potential. The results indicate that cells control amplification of specific regions of their genome in response to different stimuli to facilitate stress response, survival and adaptation to new or challenging environmental conditions.

Several KDMs have now been shown to be transcriptionally upregulated under hypoxic conditions, including KDM4B, KDM4C and KDM6B (Krieg et al. 2010; Lee et al. 2013; Guo et al. 2015). Similar results have been observed in RPE cells in response to hypoxia. However, it is demonstrated herein that KDM4A regulation under hypoxic conditions is distinct from these other JmjC KDMs as it is regulated primarily at the protein level and not at the transcriptional level. It is also demonstrated that KDM4A remains active, albeit with reduced activity under hypoxic conditions. The fact that H3K9me3 is more affected after 24 hours of hypoxia raises the possibility that hypoxia could also affect substrate specificity. This could be accomplished through posttranslational modification of KDM4A or by altering association with a cofactor that may regulate activity. These same posttranslational modifications could also be important for altering association of KDM4A with the SCF complex under hypoxic conditions. Identifying what modifications or alterations allow dissociation of KDM4A from the SCF complex will be important and could identify additional pathways that if misregulated in cancer could promote TSSG.

It is also demonstrated herein that hypoxia induces copy gain of a syntenic region to human 1q21.2 in zebrafish cells. Importantly, this reveals that copy gains of related chromosomal domains are conserved across species in response to hypoxia. It is interesting to note that the surrounding gene position and chromosome architecture is conserved between human 1q21.2 and zebrafish BCL9, indicating a conserved syntenic structure. In contrast, a zebrafish region homologous to human Xq13.1 IGBP1 locus, which was amplified in response to hypoxia in human cells, was not amplified in zebrafish cells. This region did not have a conserved genic or chromosomal architecture and was thus non-syntenic. This indicates that syntenic regions or chromosome domains can influence the ability for regions to undergo site-specific copy gains.

The fact that a conserved region amplifies in response to hypoxia in zebrafish and human cells implies that the gained regions may have a function in response to hypoxia.

Previous reports have highlighted the selection of regions during cancer progression and development. For example, the DHFR gene is amplified upon methotrexate chemotherapy (Alt et al. 1978). In a similar fashion, EGFR amplification is lost upon chemotherapy but extrachromosomal amplification reappears upon drug removal (Nathanson et al. 2014). In addition, several developmentally regulated gene-specific amplifications have been documented, including: egg shell gene amplification in Drosophila follicle cells, the amplification of genes important for saliva proteins in Sciara, and rRNA gene amplification in Tetrahymena (Tower 2004). However, the molecular basis for these phenomena has not yet been determined. In the case of cancer, it has been thought to be random selection, while during development there are thoughts of a specialized process. The appearance of these specific loci coupled to the findings described herein indicates that specific regulatory factors are involved in the amplification of distinct regions within genome. Understanding how cells specify these regions and regulate amplification provides fundamental insights into both developmental and pathological processes.

Described herein is the conserved role of hypoxia on site-specific copy gains and demonstrations that this process has a molecular basis. KDM4A is identified herein as a key enzymatic regulator of this response.

Materials and Methods

Cell Culture and Transfections.

HEK293T (called 293T throughout), hTERT-RPE-1 (called RPE throughout), MDA-MB 231, MDA-MB 468, and UMRC2 cells were maintained in DMEM with 10% fetal bovine serum, 1% penicillin/streptomycin, and L-glutamine. SK-N-AS cells were maintained in DMEM/F12 (GIBCO) with 10% fetal bovine serum, 1% penicillin/streptomycin, and L-glutamine. MM. 1S cells were maintained in suspension in RPMI with 10% fetal bovine serum, 1% penicillin/streptomycin, and L-glutamine. Zebrafish AB.9 cells (Paw and Zon 1999) were purchased from ATCC and maintained in DMEM with 20% fetal bovine serum, 1% penicillin/streptomycin, and L-glutamine at 28° C. Transient transfection experiments were performed using Roche X-tremeGENE 9™ or Lipofectamine 3000™ transfection reagent in OPTI-MEM I media (Gibco) for four hours or overnight. No selection was used in transient transfection experiments. siRNA transfections were carried out using Roche X-tremeGENE 9™ siRNA reagent or Lipofectamine 3000™ in OPTI-MEM I™ for four hours or overnight. Each siRNA experiment represents the average of at least two different siRNAs for each target gene.

Hypoxic Conditions.

Cells were plated onto culture dishes and allowed to adhere for 20-24 hours in normoxia (5% CO₂, 21% O₂, and 74% N₂). For hypoxic treatment, cells were maintained in a HERA™ Cell 150 incubator (Thermo Scientific) flushed with 5% CO₂, 1% O₂, and balanced with N₂ for the duration of the experiment. Incubator calibrations and verifications were carried out by Bianchi Associates Calibrations/Verifications.

Drug Treatments and Synchronization.

Cells were treated with the following chemical and metabolic stresses for 24 hours at doses used in the literature: 2 μg/ml Tunicamycin (TU, Abcam), 60 μM H₂O₂(Thermo Fisher Scientific), reduced-serum DMEM (0.1% FBS), Glucose-free DMEM (No Gluc, GIBCO), 2 mM DTT (Sigma), 5 mM N-acetyl Cysteine (NAC, Sigma), and 1 μM 2,3-Dimethoxy-1,4-naphthoquinone (DMNQ, Sigma). For heat shock (HS) treatment, cells were incubated at 43° C. for 30 minutes and returned to 37° C. for 24 hours prior to collection.

For G1/S synchronization, cells were treated with 2 mM hydroxyurea (HU, Sigma) for 20 hours. To release, cells were washed twice with culture medium pre-conditioned in normoxia or hypoxia, and supplied with fresh pre-conditioned media. For JIB-04 treatment, normoxic cells were pre-treated with 62.5 nM JIB-04 (Xcessbio) for 24 hours, and then treated again with JIB-04 and either transferred to 1% O₂ or maintained in normoxia for an additional 24 hours. Succinate (Sigma, S9637) was administered at a final concentration of 2 mM and cells were either maintained in normoxia for 72 hours or maintained in normoxia for 48 hours prior to being transferred to 1% O₂ for 24 hours.

Fluorescent In Situ Hybridization (FISH),

FISH was performed as described in (Manning et al. 2010; Black et al. 2013). For RPE cells, copy gain was scored as any cell with 3 or more distinct foci. Approximately 100 cells for each replicate were scored for all experiments. All FISH experiments include at least 2 biological replicates. For each experiment at least one replicate includes FACS and western blot from the same cells used for FISH. For knockdown experiments, at least two different siRNA were used for each target. Results are presented as the average from both of the independent siRNAs.

Western Blots.

Western blots were performed as in (Black et al. 2010).

Expression Plasmids and siRNAs.

pCS2-3HA-huKDM4A and pCS2-3HA-zfKDM4A WT and catalytic mutants were prepared by gateway transfer into pCS2-3HA. All clones were sequence verified. Silencer Select siRNAs were purchased from Life Technologies, as follows: KDM4A (s18636, s18637, s18635), KDM4B (s22867, s229325), KDM4C (s22989, s225929), KDM4D (s31266, s31267), KDM5A (s11834, S11836), KDM6B (s23109, s23110), HIF1α (s6539, s6541), HIF2a (s4698, s4700). Results for FISH with each siRNA (at least 2 independent siRNA per target) were averaged together in all knockdown experiments presented.

RNA Extraction and Quantitative PCR.

Cells for RNA isolation were collected by scraping or trypsinization and washed twice with PBS. Cells were resuspended in Tri-Reagent (Roche) and stored at −80° C. until use. RNA was isolated using the miRNAeasy™ Plus kit with on-column DNAse digestion (Qiagen) following the manufacturer's instructions and quantified using a Nanodrop 1000D™. Single strand cDNA was prepared using the Transcriptor First Strand cDNA Synthesis Kit (Roche) with oligo dT primers. Expression levels were analyzed by quantitative real time PCR in a Lightcycler 480™ with FastStart Universal SYBR Green™ Master (Roche) following the manufacturers protocols. All samples were normalized by comparison to β-actin transcript and hypoxia induction was verified with primers for CAIX. For CKS1B transcript analysis, we observed transcript induction in hypoxia in all samples from untreated MDA-MB-231 cells (FIG. 13A). However, transfection of MDA-MB-231 cells reduced the induction level of CKS1B (we considered >1.15-fold induced, FIG. 13D) and resulted in induction in 16 of 24 replicates, siKDM4A depletion resulted in reduced CKS1B transcript in 15 of 16 induced replicates. Replicates included three different KDM4A siRNA. The data represent an average of all replicates that exhibited induction of CKS1B in hypoxia (16 of 24). CKS1B was amplified (FISH) in all replicates and not amplified upon KDM4A depletion.

Catalytic Activity of huKDM4A and zfKDM4A in Hypoxia.

Assays for demethylase activity were performed using immunofluorescence as described in (Whetstine et al. 2006). Briefly, The indicated HA-tagged KDM4A constructs were transfected into RPE cells grown on coverslips in 6-well dishes using X-tremeGENE 9™ (Roche) or Lipofectamine 3000™ (Life Technologies) DNA transfection reagent. Following 24 or 48 hours in hypoxia, H3K36me3 and H3K9me3 were assayed by examining transfected cells (positive for HA staining; HA.11 Covance) following fixation (Whetstine et al. 2006; Black et al. 2013). Approximately fifty highly transfected cells in each of two biological replicates were scored for each condition. Data presented for normoxia is an average of the two replicates. For hypoxia, data are presented as the percent of activity of the same construct under normoxic conditions for each of two biological replicates, which were averaged together.

Human CD4+ T Cell Purification and In Vitro Culture,

CD4+ T cells were isolated from peripheral blood of healthy donors or buffy coats (Sanguine Biosciences) by flow cytometry.

Half-Life Determination.

Protein turnover was assessed as outlined in (Van Rechem et al. 2011). Briefly, cells maintained in normoxia and hypoxia were treated with 400 μM Cycloheximide (Sigma) for the indicated time, after which lysates were prepared and analyzed by western blot.

Immunoprecipitation.

Immunoprecipitations were carried out as in (Van Rechem et al. 2011) on cells grown in normoxia or hypoxia for 24 hours. KDM4A was immunoprecipitated from whole-cell lysates using KDM4A-P006, KDM4A-P014, and KDM4A rabbit polyclonal antibody (Black et al. 2010; Van Rechem et al. 2015). For ubiquitination determination, KDM4A IPs were washed under denaturing conditions as in (Van Rechem et al. 2011). Ubiquitination of KDM4A was quantitated using ImageJ™ and normalized to the amount of KDM4A IP'd.

Cesium Chloride Gradient Centrifugation.

CsCl density gradient centrifugation was performed as in (Black et al. 2013).

Flow Cytometry and Cell Cycle Analysis.

Asynchronously growing, or G1/S arrested cells were prepared and fixed as in (Black et al. 2010). Cells were stained with 10 μM EdU for 1 hour prior to collection. Cell cycle was analyzed by PI staining or EdU incorporation using Click-IT EdU™ Flow Cytometry Assay Kit (Life Technologies). Flow cytometry of CD4+ T cells and cell cycle distribution were analyzed using a BD FACS ARIA II™.

Cell Fractionation

Cytoplasmic, nuclear and chromatin fractions were prepared from RPE cells as described in (Van Rechem et al. 2015).

Generation of KDM4A Knockout 293T Cells Using CRISPR/Cas9.

KDM4A knockout 293T cells as previously described (Fu et al. 2014). Complete methods can be found in the supplemental material. Genetic rescue lines were generated by reintroducing GFP or GFP-KDM4A. KDM4A deficient cell lines expressing either GFP or GFP-KDM4A were generated using retroviral infections of pMSCV-GFP or pMSCV-GFP-KDM4A as described in (Black et al. 2013). Expression of GFP or GFP-KDM4A, was confirmed by western blot and no detectable endogenous KDM4A was observed. As clones were derived from 293T cells, clonal variability for chromosome numbers was observed (i.e. chromosome 1). The independent clones presented had the vast majority of cells with same number of copies of chromosome 1 (four per cell) and chromosome 8 (2 per cell). As such, 5 copies of 1q12h was considered a gain and 3 copies of 8c a gain in these populations. However, it was not verified that the clones had similar numbers of all other chromosomes.

### REFERENCES

Alt F W, Kellems R E, Bertino J R, Schimke R T. 1978. Selective multiplication of dihydrofolate reductase genes in methotrexate-resistant variants of cultured murine cells. J Biol Chem 253: 1357-1370.
Beroukhim R, Mermel C H, Porter D, Wei G, Raychaudhuri S, Donovan J, Barretina J, Boehm J S, Dobson J, Urashima M et al. 2010. The landscape of somatic copy-number alteration across human cancers. Nature 463: 899-905.
Beyer S, Kristensen M M, Jensen K S, Johansen J V, Staller P. 2008. The histone demethylases JMJDIA and JMJD2B are transcriptional targets of hypoxia-inducible factor HIF. J Biol Chem 283: 36542-36552.
Black J C, Allen A, Van Rechem C, Forbes E, Longworth M, Tschop K, Rinehart C, Quiton J, Walsh R, Smallwood A et al. 2010. Conserved antagonism between JMJD2A/KDM4A and HPlgamma during cell cycle progression. Mol Cell 40: 736-748.
Black J C, Manning A L, Van Rechem C, Kim J, Ladd B, Cho J, Pineda C M, Murphy N, Daniels D L, Montagna C et al. 2013. KDM4A lysine demethylase induces site-specific copy gain and rereplication of regions amplified in tumors. Cell 154: 541-555.
Black J C, Van Rechem C, Whetstine J R. 2012. Histone lysine methylation dynamics: establishment, regulation, and biological impact. Mol Cell 48: 491-507.
Burrell R A, McGranahan N, Bartek J, Swanton C. 2013. The causes and consequences of genetic heterogeneity in cancer evolution. Nature 501: 338-345.
Carreau A, El Hafny-Rahbi B, Matejuk A, Grillon C, Kieda C. 2011. Why is the partial oxygen pressure of human tissues a crucial parameter? Small molecules and hypoxia. J Cell Mol Med 15: 1239-1253.
Dunwoodie S L. 2009. The role of hypoxia in development of the Mammalian embryo. Dev Cell 17: 755-773.
Eschmann S M, Paulsen F, Reimold M, Dittmann H, Welz S, Reischl G, Machulla H J, Bares R. 2005. Prognostic impact of hypoxia imaging with 18F-misonidazole PET in non-small cell lung cancer and head and neck cancer before radiotherapy. J Nucl Med 46: 253-260.
Fu Y, Reyon D, Joung J K. 2014. Targeted genome editing in human cells using CRISPR/Cas nucleases and truncated guide RNAs. Methods Enzymol 546: 21-45.
Gameiro P A, Yang J, Metelo A M, Perez-Carro R, Baker R, Wang Z, Arreola A, Rathmell W K, Olumi A, Lopez-Larrubia P et al. 2013. In vivo HIF-mediated reductive carboxylation is regulated by citrate levels and sensitizes VHL-deficient cells to glutamine deprivation. Cell Metab 17: 372-385.
Gerlinger M, Rowan A J, Horswell S, Larkin J, Endesfelder D, Gronroos E, Martinez P, Matthews N, Stewart A, Tarpey P et al. 2012. Intratumor heterogeneity and branched evolution revealed by multiregion sequencing. N Engl J Med 366: 883-892.
Guo X, Tian Z, Wang X, Pan S, Huang W, Shen Y, Gui Y, Duan X, Cai Z. 2015. Regulation of histone demethylase KDM6B by hypoxia-inducible factor-2alpha. Acta Biochim Biophys Sin (Shanghai) 47: 106-113.
Hockel M, Schlenger K, Aral B, Mitze M, Schaffer U, Vaupel P. 1996. Association between tumor hypoxia and malignant progression in advanced cancer of the uterine cervix. Cancer Res 56: 4509-4515.
Hook S S, Lin J J, Dutta A. 2007. Mechanisms to control rereplication and implications for cancer. Curr Opin Cell Biol 19: 663-671.
Jiang X R, Jimenez G, Chang E, Frolkis M, Kusler B, Sage M, Beeche M, Bodnar A G, Wahl G M, Tlsty T D et al. 1999. Telomerase expression in human somatic cells does not induce changes associated with a transformed phenotype. Nat Genet 21: 111-114.
Jopling C, Sune G, Faucherre A, Fabregat C, Izpisua Belmonte J C. 2012. Hypoxia induces myocardial regeneration in zebrafish. Circulation 126: 3017-3027.
Junttila M R, de Sauvage F J. 2013. Influence of tumour micro-environment heterogeneity on therapeutic response. Nature 501: 346-354.
Khattar V, Thottassery J V. 2013. Cks1: Structure, Emerging Roles and Implications in Multiple Cancers. J Cancer Ther 4: 1341-1354.
Krieg A J, Rankin E B, Chan D, Razorenova O, Fernandez S, Giaccia A J. 2010. Regulation of the histone demethylase JMJDIA by hypoxia-inducible factor 1 alpha enhances hypoxic gene expression and tumor growth. Mol Cell Biol 30: 344-353.
Lee H Y, Yang E G, Park H. 2013. Hypoxia enhances the expression of prostate-specific antigen by modifying the quantity and catalytic activity of Jumonji C domain-containing histone demethylases. Carcinogenesis 34: 2706-2715.
Levinson D F, Duan J, Oh S, Wang K, Sanders A R, Shi J, Zhang N, Mowry B J, Olincy A, Amin F et al. 2011. Copy number variants in schizophrenia: confirmation of five previous findings and new evidence for 3q29 microdeletions and VIPR2 duplications. Am J Psychiatry 168: 302-316.
Lewis P W, Muller M M, Koletsky M S, Cordero F, Lin S, Banaszynski L A, Garcia B A, Muir T W, Becher O J, Allis C D. 2013. Inhibition of PRC2 Activity by a Gain-of-Function H3 Mutation Found in Pediatric Glioblastoma. Science.
Manning A L, Longworth M S, Dyson N J. 2010. Loss of pRB causes centromere dysfunction and chromosomal instability. Genes Dev 24: 1364-1376.
Martin-Ezquerra G, Salgado R, Toll A, Baro T, Mojal S, Yebenes M, Garcia-Muret M P, Sole F, Quitllet F A, Espinet B et al. 2011. CDC28 protein kinase regulatory subunit 1B (CKS1B) expression and genetic status analysis in oral squamous cell carcinoma. Histol Histopathol 26: 71-77.
Nathanson D A, Gini B, Mottahedeh J, Visnyei K, Koga T, Gomez G, Eskin A, Hwang K, Wang J, Masui K et al. 2014. Targeted therapy resistance mediated by dynamic regulation of extrachromosomal mutant EGFR DNA. Science 343: 72-76.
Network CGA. 2012. Comprehensive molecular portraits of human breast tumours. Nature 490: 61-70.
Patel A P, Tirosh I, Trombetta J J, Shalek A K, Gillespie S M, Wakimoto H, Cahill D P, Nahed B V, Curry W T, Martuza R L et al. 2014. Single-cell RNA-seq highlights intratumoral heterogeneity in primary glioblastoma. Science 344: 1396-1401.
Paw B H, Zon L I. 1999. Primary fibroblast cell culture. Methods Cell Biol 59: 39-43.
Perou C M. 2010. Molecular stratification of triple-negative breast cancers. Oncologist 15 Suppl 5: 39-48.
Rofstad E K. 2000. Microenvironment-induced cancer metastasis. Int J Radiat Biol 76: 589-605.
Shaughnessy J. 2005. Amplification and overexpression of CKS1B at chromosome band 1q21 is associated with reduced levels of p27Kip1 and an aggressive clinical course in multiple myeloma. Hematology 10 Suppl 1: 117-126.
Shi L, Wang S, Zangari M, Xu H, Cao T M, Xu C, Wu Y, Xiao F, Liu Y, Yang Y et al. 2010. Over-expression of CKS1B activates both MEK/ERK and JAK/STAT3 signaling pathways and promotes myeloma cell drug-resistance. Oncotarget 1: 22-33.
Smith E H, Janknecht R, Maher L J, 3rd. 2007. Succinate inhibition of alpha-ketoglutarate-dependent enzymes in a yeast model of paraganglioma. Hum Mol Genet 16: 3136-3148.
Solaini G, Baracca A, Lenaz G, Sgarbi G. 2010. Hypoxia and mitochondrial oxidative metabolism. Biochim Biophys Acta 1797: 1171-1177.
Stefansson H, Rujescu D, Cichon S, Pietilainen O P, Ingason A, Steinberg S, Fossdal R, Sigurdsson E, Sigmundsson T, Buizer-Voskamp J E et al. 2008. Large recurrent microdeletions associated with schizophrenia. Nature 455: 232-236.
Stratton M R, Campbell P J, Futreal P A. 2009. The cancer genome. Nature 458: 719-724.
Tan M K, Lim H J, Harper J W. 2011. SCF(FBXO22) regulates histone H3 lysine 9 and 36 methylation levels by targeting histone demethylase KDM4A for ubiquitin-mediated proteasomal degradation. Mol Cell Biol 31: 3687-3699.
Tang Y C, Amon A. 2013. Gene copy-number alterations: a cost-benefit analysis. Cell 152: 394-405.
Tower J. 2004. Developmental gene amplification and origin regulation. Annu Rev Genet 38: 273-304.
Van Rechem C, Black J C, Abbas T, Allen A, Rinehart C A, Yuan G C, Dutta A, Whetstine J R. 2011. The SKP1-Cul1-F-box and leucine-rich repeat protein 4 (SCF-FbxL4) ubiquitin ligase regulates lysine demethylase 4A (KDM4A)/Jumonji domain-containing 2A (JMJD2A) protein. J Biol Chem 286: 30462-30470.
Van Rechem C, Black J C, Boukhali M, Aryee M J, Graslund S, Haas W, Benes C H, Whetstine J R. 2015. Lysine Demethylase KDM4A Associates with Translation Machinery and Regulates Protein Synthesis. Cancer Discov.
Vaupel P. 2004. The role of hypoxia-induced factors in tumor progression. Oncologist 9 Suppl 5: 10-17.
Wang L, Chang J, Varghese D, Dellinger M, Kumar S, Best A M, Ruiz J, Bruick R, Pena-Llopis S, Xu J et al. 2013. A small molecule modulates Jumonji histone demethylase activity and selectively inhibits cancer growth. Nat Commun 4: 2035.
Wang W, He Y F, Sun Q K, Wang Y, Han X H, Peng D F, Yao Y W, Ji C S, Hu B. 2014. Hypoxia-inducible factor 1alpha in breast cancer prognosis. Clin Chim Acta 428: 32-37.
Wang X C, Tian J, Tian L L, Wu H L, Meng A M, Ma T H, Xiao J, Xiao X L, Li C H. 2009. Role of Cks1 amplification and overexpression in breast cancer. Biochem Biophys Res Commun 379: 1107-1113.
Whetstine J R, Nottke A, Lan F, Huarte M, Smolikov S, Chen Z, Spooner E, Li E, Zhang G, Colaiacovo M et al. 2006. Reversal of histone lysine trimethylation by the JMJD2 family of histone demethylases. Cell 125: 467-481.
Winter S C, Buffa F M, Silva P, Miller C, Valentine H R, Turley H, Shah K A, Cox G J, Corbridge R J, Homer J J et al. 2007. Relation of a hypoxia metagene derived from head and neck cancer to prognosis of multiple cancers. Cancer Res 67: 3441-3449.

Materials and Methods

Cell Culture and Transfections.

HEK293T (called 293T throughout), hTERT-RPE-1 (called RPE throughout), MDA-MB 231, MDA-MB 468, and UMRC2 cells were maintained in DMEM with 10% fetal bovine serum, 1% penicillin/streptomycin, and L-glutamine. SK-N-AS cells were maintained in DMEM/F12 (GIBCO) with 10% fetal bovine serum, 1% penicillin/streptomycin, and L-glutamine. MM. 1S cells were maintained in suspension in RPMI with 10% fetal bovine serum, 1% penicillin/streptomycin, and L-glutamine. Zebrafish AB.9 cells (Paw and Zon 1999) were purchased from ATCC and maintained in DMEM with 20% fetal bovine serum, 1% penicillin/streptomycin, and L-glutamine at 28° C. Transient transfection experiments were performed using Roche X-tremeGENE 9™ or Lipofectamine 3000™ transfection reagent in OPTI-MEM I media (Gibco) for four hours or overnight. No selection was used in transient transfection experiments. siRNA transfections were carried out using Roche X-tremeGENE 9™ siRNA reagent or Lipofectamine 3000™ in OPTI-MEM I for four hours or overnight. Each siRNA experiment represents the average of at least two different siRNAs for each target gene.

Hypoxic Conditions.

Cells were plated onto culture dishes and allowed to adhere for 20-24 hours in normoxia (5% CO₂, 21% O₂, and 74% N₂). For hypoxic treatment, cells were maintained in a HERA™ Cell 150 incubator (Thermo Scientific) flushed with 5% CO₂, 1% O₂, and balanced with N₂ for the duration of the experiment. Incubator calibrations and verifications were carried out by Bianchi Associates Calibrations/Verifications.

Drug Treatments and Synchronization.

Cells were treated with the following chemical and metabolic stresses for 24 hours at doses used in the literature: 2 μg/ml Tunicamycin (TU, Abcam), 60 μM H₂O₂(Thermo Fisher Scientific), reduced-serum DMEM (0.1% FBS), Glucose-free DMEM (No Glue, GIBCO), 2 mM DTT (Sigma), 5 mM N-acetylcysteine (NAC, Sigma), and 1 μM 2,3-Dimethoxy-1,4-naphthoquinone (DMNQ, Sigma). For heat shock (HS) treatment, cells were incubated at 43° C. for 30 minutes and returned to 37° C. for 24 hours prior to collection.

For G1/S synchronization, cells were treated with 2 mM hydroxyurea (HU, Sigma) for 20 hours. To release, cells were washed twice with culture medium pre-conditioned in normoxia or hypoxia, and supplied with fresh pre-conditioned media. For JIB-04 treatment, normoxic cells were pre-treated with 62.5 nM JIB-04 (Xcessbio) for 24 hours, and then treated again with JIB-04 and either transferred to 1% O₂ or maintained in normoxia for an additional 24 hours. Succinate (Sigma, S9637) was administered at a final concentration of 2 mM and cells were either maintained in normoxia for 72 hours or maintained in normoxia for 48 hours prior to being transferred to 1% O₂ for 24 hours.

Fluorescent In Situ Hybridization (FISH).

FISH was performed as described in (Manning et al. 2010; Black et al. 2013). Probes for 1q12h, 1q telomere, chromosome 8 centromere (alpha satellite), and X centromere (alpha satellite) were purchased from Rainbow Scientific. Probes for Zebrafish BCL9 (CH73-15J19) and Zebrafish IGBP1 (CH73-223D24) were purchased as BAC clones from Children's Hospital Oakland Research Institute (CHORI BacPac) clone repository. Probes for 1q21.2 (BCL9) and 1q23.3 were purchased from Agilent (SureFISH). BACS were prepared utilizing PureLink HiPure™ Plasmid Filter Maxiprep kit (Life Technologies) using the recommended modified wash buffer. Probes were nick translated (Abbot Molecular Kit) in the presence of fluorescently labeled dTTP (Enzo Life Science). Images of multiple planes of fields of nuclei were acquired on an Olympus IX81™ Spinning Disk Microscope and analyzed using Slidebook 5.0™ software. We used a conservative scoring metric for copy gain. Any foci that were touching were scored as a single copy to prevent increased numbers due to normally replicated foci. For RPE cells, copy gain was scored as any cell with 3 or more distinct foci. For 293T cells, copy gain was scored for any cell with 5 or more distinct foci. For UMRC2 cells, copy gain was scored for any cell with 6 or more foci. For SK-N-AS cells, copy gain was scored for any cell with 5 or more foci. For MDA-MB-468 cells, copy gain was scored for any cell with 5 or more foci. For MDA-MB-231 cells, copy gain was scored for any cell with 7 or more foci. Approximately 100 cells for each replicate were scored for all experiments. All FISH experiments include at least 2 biological replicates. For each experiment, at least one replicate includes FACS and western blot from the same cells used for FISH. For knockdown experiments, at least two different siRNA were used for each target. Results are presented as the average from both of the independent siRNAs.

Antibodies.

Antibodies used were: KDM4A (Neuro mAB, 75-189), KDM4B (Santa Cruz, sc-67192), KDM4C (Abcam, ab85454), KDM4D (Abcam, ab93694), KDM5A (Abcam, ab70892), β-actin (Millipore), RFP (Abcam, ab62341), Halo (Promega), Actinin (Santa Cruz, sc-17829), HA 12CA5 (Roche), HIF1α (Santa Cruz, sc-10790), HIF2a (Cell Signaling, Clone D9E3), CAIX (Abcam, ab108351), LDH1 (Santa Cruz, sc-133123), Histone H3 (Abcam, ab1791), HA.11 (Covance), KDM4A-P006 FAB (SGC), FBXL4 (Santa Cruz, sc-54489), FBXW2 (abcam ab83467), Cul1 (Santa Cruz, sc-17775), Ubiquitin (Santa Cruz, sc-8017).

Western Blots. Western blots were performed as in (Black et al. 2010). Briefly, adherent cells were either scraped directly into PBS, or washed with PBS, trypsinized and collected by centrifuging at 2,000 RPM for 5 minutes. For preparation of whole-cell lysates, cell pellets were washed once in ice-cold PBS and resuspended in RIPA lysis buffer [50 mM Tris pH 7.4, 150 mM NaCl, 0.25% Sodium Deoxycholate, 1% NP40, 1 mM EDTA, 10% Glycerol] supplemented with cOmplete protease inhibitor and PhosSTOP phosphatase inhibitor cocktails (Roche). Cells were lysed on ice for 15 minutes and immediately frozen at −80° C. for 10 minutes. Lysates were subsequently sonicated at 70% amplitude for 15 minutes in a QSonica Q700 sonicator and cleared of cell debris by centrifuging at 12,000 RPM for 15 minutes, before being analyzed by western blotting. For HIF1α and HIF2a expression, adherent cells were washed twice with ice-cold PBS and scraped directly in warmed 1× Laemmli buffer. Samples were sonicated at 70% amplitude for 15 minutes in a QSonica Q700™ and boiled at 95° C. for 10 minutes immediately prior to western blotting.

Expression Plasmids and siRNAs.

pCS2-3HA-huKDM4A and pCS2-3HA-zfKDM4A WT and catalytic mutants were prepared by gateway transfer into pCS2-3HA. All clones were sequence verified. Silencer Select siRNAs were purchased from Life Technologies, as follows: KDM4A (s18636, s18637, s18635), KDM4B (s22867, s229325), KDM4C (s22989, s225929), KDM4D (s31266, s31267), KDM5A (s11834, S11836), KDM6B (s23109, s23110), HIF1α (s6539, s6541), HIF2a (s4698, s4700). Results for FISH with each siRNA (at least 2 independent siRNA per target) were averaged together in all knockdown experiments presented.

RNA Extraction and Quantitative PCR.

Cells for RNA isolation were collected by scraping or trypsinization and washed twice with PBS. Cells were resuspended in Tri-Reagent (Roche) and stored at −80° C. until use. RNA was isolated using the miRNAeasy™ Plus kit with on-column DNAse digestion (Qiagen) following the manufacturer's instructions and quantified using a Nanodrop 1000D. Single strand cDNA was prepared using the Transcriptor First Strand™ cDNA Synthesis Kit (Roche) with oligo dT primers. Expression levels were analyzed by quantitative real time PCR in a Lightcycler 480™ with FastStart Universal SYBR Green™ Master (Roche) following the manufacturers protocols. All samples were normalized by comparison to β-actin transcript and hypoxia induction was verified with primers for CAIX. For CKS1B transcript analysis, we observed transcript induction in hypoxia in all samples from untreated MDA-MB-231 cells (FIG. 13A). However, transfection of MDA-MB-231 cells reduced the induction level of CKS1B (we considered >1.15-fold induced, FIG. 13D) and resulted in induction in 16 of 24 replicates, siKDM4A depletion resulted in reduced CKS1B transcript in 15 of 16 induced replicates. Replicates included three different KDM4A siRNA. The data represent an average of all replicates that exhibited induction of CKS1B in hypoxia (16 of 24). CKS1B was amplified (FISH) in all replicates and not amplified upon KDM4A depletion. Primers available upon request.

Catalytic Activity of huKDM4A and zfKDM4A in Hypoxia.

Assays for Demethylase activity were performed using immunofluorescence as described in (Whetstine et al. 2006). Briefly, The indicated HA-tagged KDM4A constructs were transfected into RPE cells grown on coverslips in 6-well dishes using X-tremeGENE 9™ (Roche) or Lipofectamine 3000 (Life Technologies) DNA transfection reagent. Following 24 or 48 hours in hypoxia, H3K36me3 and H3K9me3 were assayed by examining transfected cells (positive for HA staining; HA.11 Covance) following fixation (Whetstine et al. 2006; Black et al. 2013). Approximately fifty highly transfected cells in each of two biological replicates were scored for each condition. Data presented for normoxia is an average of the two replicates. For hypoxia, data are presented as the percent of activity of the same construct under normoxic conditions for each of two biological replicates, which were averaged together.

Human CD4+ T Cell Purification and In Vitro Culture.

Buffy coats (Sanguine Biosciences) or peripheral blood of healthy controls was diluted 1:2 in room-temperature PBS lacking Ca²⁺/Mg²⁺. Mononuclear cells were isolated by Ficoll-Paque Plus™ (GE Healthcare) density-gradient centrifugation following the manufacturer's protocol. PBMCs were resuspended at a density of 20×10⁶ cells/mL and reacted with Fc receptor blocking solution (Human TruStain FcX, Biolegend), followed by surface staining with APC anti-human CD4 antibody (Clone OKT4, Biolegend) for 45 minutes on ice. Antibody-stained cells were resuspended in HBSS (GIBCO) supplemented with 10 mM glucose and sorted by flow cytometry. Sorted cells (including CD4+ T cells) were collected in 5 mL tubes containing 1 mL collection medium (DMEM supplemented with 30% FBS) and reanalyzed by flow cytometry to ensure >99% purity in defined gates. Sorted cells were allowed to recover in RPMI medium (GIBCO) supplemented with 10% FBS for 2 hours. For resting CD4+ T cell culture, cells were seeded onto 60 mm dishes and maintained in complete medium supplemented with 10 ng/mL recombinant human interleukin-2 (rhIL-2, R&D Systems). For stimulated CD4+ T cell culture, 60 mm dishes were pre-coated with a cocktail containing 5 μg/mL anti-human CD3 (Clone HIT3a, Biolegend) and 3 μg/mL anti-human CD28 (Clone CD28.2, Biolegend) for 1 hour, after which cells were seeded onto the coated dish. Stimulated CD4+ T cells were maintained in complete medium supplemented with 10 ng/mL rhIL-2, and anti-CD3/CD28 antibodies. Resting and stimulated CD4+ T cells were allowed to recover for 24 hours in normoxia (21% O₂), followed by an additional 24 hours in normoxia or in hypoxia (1% O₂) prior to being collected.

Half-Life Determination.

Protein turnover was assessed as outlined in (Van Rechem et al. 2011). Briefly, cells maintained in normoxia and hypoxia were treated with 400 μM Cycloheximide (Sigma) for the indicated time, after which lysates were prepared and analyzed by western blot.

Immunoprecipitation.

Immunoprecipitations were carried out as in (Van Rechem et al. 2011) on cells grown in normoxia or hypoxia for 24 hours. KDM4A was immunoprecipitated from whole-cell lysates using KDM4A-P006, KDM4A-P014, and KDM4A rabbit polyclonal antibody (Black et al. 2010; Van Rechem et al. 2015). For ubiquitination determination, KDM4A IPs were washed under denaturing conditions as in (Van Rechem et al. 2011). Ubiquitination of KDM4A was quantitated using ImageJ and normalized to the amount of KDM4A IP'd.

Cesium Chloride Gradient Centrifugation.

CsCl density gradient centrifugation was performed as in (Black et al. 2013). Briefly, RPE cells were grown in normoxia or 1% O₂ for 24 hours prior to addition of BrdU. Cells were labeled with BrdU for 12 hours and 45 minutes. Each rereplicated fraction was diluted to 15 ng/ul stock and 7.5 ng of rereplicated DNA pool was analyzed by qPCR on a Roche LC480 using FastStart Universal SYBR Green™ Master Mix (Roche) following the manufacturer's instructions. 7.5 ng of input DNA was analyzed by qPCR at the same time. Each sample was normalized to its own input prior to determination of fold-change in rereplication.

Flow Cytometry and Cell Cycle Analysis.

Asynchronously growing, or G1/S arrested cells were prepared and fixed as in (Black et al. 2010). Cells were stained with 10 μM EdU for 1 hour prior to collection. Cell cycle was analyzed by PI staining or EdU incorporation using Click-IT EdU™ Flow Cytometry Assay Kit (Life Technologies). Flow cytometry of CD4+ T cells and cell cycle distribution were analyzed using a BD FACS ARIA II™.

Cell Fractionation.

Cytoplasmic, nuclear and chromatin fractions were prepared from RPE cells. Cell pellets were washed twice in ice cold PBS and resuspended in ice cold Buffer A (10 mM HEPES pH 7.9, 10 mM KCl, 0.1M EDTA, 0.5M EGTA) and incubated on ice for 15 minutes. Swollen cells were lysed by addition of NP-40 to 0.8% with 10 seconds of vortexing. Lysed cells were centrifuged and the supernatant kept as cytoplasm. The nuclear pellet was resuspended in Buffer C (10 mM HEPES pH 7.9, 400 mM NaCl, 1 mM EDTA, 5 mM EGTA), dounced to resuspend the nuclei and incubated at 4′C for 30 minutes with rotation. Extracts were centrifuged and the supernatant kept as nuclear extract. Chromatin pellets were resuspended in N-Buffer (20 mM Trish pH 7.5, 100 mM KCl, 2 mM MgCl2, 1 mM CaCl2, 0.3M Sucrose, 0.1% Triton X-100, 3U per ml micrococcal nuclease). Samples were sonicated for 10 minutes at 70% amplitude in a Q700™ cup horn (QSonica) and then incubated at room temperature for 15 minutes for MNase digestion. Reactions were stopped by addition of 5 mM EGTA and centrifuged to clear. Supernatant was kept as chromatin extract.

Spectral Karyotyping.

Two biological replicates of cells grown in normoxia or hypoxia for 24 hours were analyzed by SKY.

Generation of KDM4A Knockout 293T Cells Using CRISPR/Cas9.

A KDM4A-targeting CRISPR guide RNA (gRNA) was designed using the ZiFiT Targeter web server as previously described (Fu et al. 2014). This guide sequence targeted (CTTTACTCAGTACAACATAC) at position 243-262 in KDM4A cDNA. The gRNA was cloned into the BsmBI-digested expression plasmid pMLM3636 as described (Fu et al. 2014).

For generation of KDM4A knockout CRISPR cell lines, 293T cells were seeded onto 24-well dishes and transfected with the Cas9 nuclease (pJDS246) and gRNA using Lipofectamine 3000™ (Fu et al. 2013). Forty-eight hours post-transfection, cells were collected and plated as single cells in 96-well dishes. Twenty-eight days post-seeding, genomic DNA and whole cell lysates were collected and clones exhibiting mutations in KDM4A were identified using T7E1 assays and western blotting (Fu et al. 2014). Homozygous deletion of KDM4A in the selected cell line was further validated by sequencing of genomic loci.

We generated genetic rescue lines by reintroducing GFP or GFP-KDM4A. KDM4A deficient cell lines expressing either GFP or GFP-KDM4A were generated using retroviral infections of pMSCV-GFP or pMSCV-GFP-KDM4A as described in (Black et al. 2013). GFP-positive cells were isolated by cell sorting on a FACS ARIA II™. Following recovery, GFP and GFP-KDM4A cells were replated as single cells. Independently derived, single-cell clonal lines were established. Expression of GFP or GFP-KDM4A, was confirmed by western blot and no detectable endogenous KDM4A was observed. As clones were derived from 293T cells, clonal variability for chromosome numbers was observed (i.e. chromosome 1). The independent clones presented had the vast majority of cells with same number of copies of chromosome 1 (four per cell) and chromosome 8 (2 per cell). As such, we considered 5 copies of 1q12h a gain and 3 copies of 8c a gain in these populations. However, we did not verify that the clones had similar numbers of all other chromosomes.

Data Processing for TCGA Breast Cancer and Lung Adenocarcinoma,

All genomic data of mutation, copy number, and mRNA expression for TCGA Breast Cancer (BRCA) and Lung Adenocarcinoma (LUAD) were downloaded from Broad GDAC (Genome Data Analysis Center) Firehose analysis run named “15 Jan. 2014” (doi: 10.7909/C1H41PXV).

Copy Number Data: The segmented copy number data for 1007 BRCA samples and 493 LUAD samples was processed by GISITC2.0™ (Mermel et al. 2011) to annotate the somatic copy number alterations (SCNAs) for 24,174 genes. Copy-number data were dissociated to arm-level and focal copy-number alterations as described in the GISTIC2.0™ paper (Mermel et al. 2011). In addition to the copy number annotation for each gene, the mean focal copy number for 807 cytobands including X chromosome were calculated for each sample by taking the average of the focal SCNA values across all genes within a cytoband. The contribution of arm-level SCNAs to the mean cytoband focal copy was eliminated by only considering GISTIC annotated focal copy numbers spanning a much smaller region than a chromosome arm.

RNA-seq Data: The mRNA expression levels for 18264 genes in 1019 BRCA samples and 488 LUAD samples were annotated by the log₂-normalized RSEM (RNASeq by Expectation Maximization (Li and Dewey 2011)) values. RSEM values for 956 BRCA samples and 486 LUAD samples having copy number data were median-centered (by subtracting the median expression across tumor samples), yielding log₂ (Fold Changes) and utilized in the downstream analysis.

Somatic Mutation Data: The MAF (Mutation Annotation Format) file for 976 BRCA samples and 229 LUAD samples contained 73,729 and 92,133 somatic mutations, respectively.

BRCA subtype information: The subtype information for 504 BRCA samples based on PAM50 gene set was extracted from the supplemental data (BRCA.547.PAM50. SigClust. Subtypes.txt) of TCGA BRCA paper (Network 2012).

Hypoxia Signature Gene Set.

The hypoxia metagene (Winter et al. 2007), was downloaded from MSigDB (Subramanian et al. 2005) and used as a hypoxia signature gene set in a downstream analysis. The efficacy of this gene set was demonstrated as a significant prognostic factor for overall survivals in both HNSC and BRCA data set. The final hypoxia signature gene set (data not shown) was comprised of 92 up-regulated (HS-up) and 52 down-regulated (HS-down) genes including well-known hypoxia biomarkers such as HIF1A, CA9, and VEGFA.

Identifying Hypoxia Samples Using Consensus Hierarchical Clustering.

Consensus hierarchical clustering was used to identify a cluster of samples that showed the most concordant expression pattern to the previously-defined hypoxia signature gene set (Winter et al. 2007). Using the mRNA expression data, we first computed the Spearman correlation coefficients between pairs of samples using the median-centered log₂-normalized RSEM values. We applied the consensus hierarchical clustering R package ConsensusClusterPlus™ (Wilkerson and Hayes 2010), with 1-Spearman correlation as a distance metric, and run over 1000 iterations of the “average linkage” method and 80% resampling rate. We varied the number of clusters from K=2 to 8. We determined the hypoxia cluster by examining the stability of the chosen cluster throughout K and the concordance of mRNA expression levels in each cluster to the known expression patterns of hypoxia up or down signatures. This process finally resulted in the choice of K=3 in BRCA and K=4 in LUAD (data not shown). Details described below:

(1) TCGA Breast Cancers. The cluster membership of samples across K, where the most hypoxia-related cluster at any given K (chosen based on the mean expression levels of the hypoxia-up genes) was highlighted. At K=2 almost 60% samples belonged to the hypoxia cluster (black). Half of these samples were separated from the large black cluster and formed their own cluster (green) at K=3. The samples in the black cluster (35%) at K=3 had the most concordant expression pattern to both the up and down genes in the signature (“hypoxia-signature concordant cluster”), while the hypoxia-signature neutral cluster cluster (42%) had an overall down-regulations regardless of hypoxia signatures (“hypoxia-signature neutral cluster”). On the other hand, the magenta cluster at K=3 (23%) had an opposite expression pattern to the known hypoxia signature (“hypoxia-signature discordant cluster”), which is also observed. We also observed that most samples in the black cluster at K=3 consistently remained in the hypoxic cluster up to K=8, indicating the strong stability of this cluster throughout K. Interestingly, the Basal (65 out of 88) and Her2 (31 out of 55) breast cancer subtypes were significantly enriched in the hypoxia cluster, while most Luminal A/B (322 out of 341) and eight Normal-like samples were in the non-hypoxia cluster.

(2) TCGA Lung Adenocarcinoma. Both clustering results at K=2 and 3 had a very similar stratification of samples except for two outlier samples in K=3. Crossing from K=3 to 4 a small number of samples with a much weaker hypoxia-up signature were separated from the black hypoxia cluster, forming the hypoxia-signature neutral cluster at K=4. The majority of samples (42%) remained in the hypoxic cluster had the most concordant expression pattern to both up and down signatures (“hypoxia-signature concordant cluster”), while in the hypoxia-signature neutral cluster (11%) all hypoxia signature genes were down-regulated, hence called the “hypoxia-signature neutral cluster”. On the contrary, the hypoxia-signature discordant cluster at K=4 (46%) had a largely discordant expression pattern with respect to the hypoxia signature (called the “hypoxia-signature discordant cluster”). Ignoring four outlier samples at K=4 the stratification of samples into hypoxia-concordant, neutral, and discordant groups is analogous to the partitioning of BRCA at K=3. Most samples in the black cluster at K=4 remained in the hypoxic cluster up to K=8, demonstrating the strong stability of the chosen hypoxia cluster.

Time to Death Versus Hypoxia.

A short follow-up time for survival (median survival time—21.1 months in BRCA and 12.4 months in LUAD) and a high fraction of censored samples (820 out of 920 samples in BRCA and 313 out of 430 samples in LUAD) is a significant challenge in evaluating the association of hypoxia samples with clinical outcome. Instead, we examined the association with the number of deceased patients, illuminating a significant higher risk in the hypoxic samples in both BRCA (FIG. 12A, p<0.00011) and LUAD (FIG. 12B, p<0.0097) by the one-tailed Wilcoxon rank-sum test.

Chromosomal Instability Vs Hypoxia.

In order to examine whether the hypoxia samples had a significant enrichment of the chromosomal instability, the distributions of the number of cytobands harboring focal gains (mean cytoband focal copy >0) and/or losses (mean cytoband focal copy <0) per sample were compared between hypoxia and non-hypoxia samples for BRCA (FIG. 12C, FIG. 19A,19B) and for LUAD (FIG. 12D, FIG. 19C,19D).

Detecting Chromosomal Regions Significantly Associated to Hypoxia.

Since the amplification of KDM4A can induce a site-specific copy gain at 1q21 (Black et al. 2013), samples with a focal copy gain of the 1p34.1 cytoband (where KDM4A resides) were excluded from downstream analysis (76 samples in BRCA and 46 samples in LUAD). Detecting chromosomal regions (i.e., cytobands) significantly associated with hypoxic samples was performed by the statistical test based on the normal approximation for the null distribution of mean cytoband copy difference between hypoxia and non-hypoxia samples. The null distribution was approximated by a normal density function with the population mean difference, m1−m0, and the variances of S1/n1+S0/n0. Here ml and m0 are sample means, S1 and S0 are sample variances, and n1 and n0 are the number of samples in the hypoxia and the non-hypoxia group. The p-values for mean cytoband copy gains in hypoxia samples were computed by computing the probability of more extreme differences than the observed copy difference in the null distribution across 807 cytobands.

### REFERENCES

Black J C, Allen A, Van Rechem C, Forbes E, Longworth M, Tschop K, Rinehart C, Quiton J, Walsh R, Smallwood A et al. 2010. Conserved antagonism between JMJD2A/KDM4A and HPlgamma during cell cycle progression. Mol Cell 40: 736-748.
Black J C, Manning A L, Van Rechem C, Kim J, Ladd B, Cho J, Pineda C M, Murphy N, Daniels D L, Montagna C et al. 2013. KDM4A lysine demethylase induces site-specific copy gain and rereplication of regions amplified in tumors. Cell 154: 541-555.
Fu Y, Foden J A, Khayter C, Maeder M L, Reyon D, Joung J K, Sander J D. 2013. High-frequency off-target mutagenesis induced by CRISPR-Cas nucleases in human cells. Nat Biotechnol 31: 822-826.
Fu Y, Reyon D, Joung J K. 2014. Targeted genome editing in human cells using CRISPR/Cas nucleases and truncated guide RNAs. Methods Enzymol 546: 21-45.
Li B, Dewey C N. 2011. RSEM: accurate transcript quantification from RNA-Seq data with or without a reference genome. BMC Bioinformatics 12: 323.
Manning A L, Longworth M S, Dyson N J. 2010. Loss of pRB causes centromere dysfunction and chromosomal instability. Genes Dev 24: 1364-1376.
Mermel C H, Schumacher S E, Hill B, Meyerson M L, Beroukhim R, Getz G. 2011. GISTIC2.0 facilitates sensitive and confident localization of the targets of focal somatic copy-number alteration in human cancers. Genome Biol 12: R41.
Network TCGA. 2012. Comprehensive molecular portraits of human breast tumours. Nature 490: 61-70.
Paw B H, Zon L I. 1999. Primary fibroblast cell culture. Methods Cell Biol 59: 39-43.
Subramanian A, Tamayo P, Mootha V K, Mukherjee S, Ebert B L, Gillette M A, Paulovich A, Pomeroy S L, Golub T R, Lander E S et al. 2005. Gene set enrichment analysis: a knowledge-based approach for interpreting genome-wide expression profiles. Proc Natl Acad Sci USA 102: 15545-15550.
Van Rechem C, Black J C, Abbas T, Allen A, Rinehart C A, Yuan G C, Dutta A, Whetstine J R. 2011. The SKP1-Cul1-F-box and leucine-rich repeat protein 4 (SCF-FbxL4) ubiquitin ligase regulates lysine demethylase 4A (KDM4A)/Jumonji domain-containing 2A (JMJD2A) protein. J Biol Chem 286: 30462-30470.
Van Rechem C, Black J C, Boukhali M, Aryee M J, Graslund S, Haas W, Benes C H, Whetstine J R. 2015. Lysine Demethylase KDM4A Associates with Translation Machinery and Regulates Protein Synthesis. Cancer Discov.
Whetstine J R, Nottke A, Lan F, Huarte M, Smolikov S, Chen Z, Spooner E, Li E, Zhang G, Colaiacovo M et al. 2006. Reversal of histone lysine trimethylation by the JMJD2 family of histone demethylases. Cell 125: 467-481.
Wilkerson M D, Hayes D N. 2010. ConsensusClusterPlus: a class discovery tool with confidence assessments and item tracking. Bioinformatics 26: 1572-1573.
Winter S C, Buffa F M, Silva P, Miller C, Valentine H R, Turley H, Shah K A, Cox G J, Corbridge R J, Homer J J et al. 2007. Relation of a hypoxia metagene derived from head and neck cancer to prognosis of multiple cancers. Cancer Res 67: 3441-3449.

### Example 4

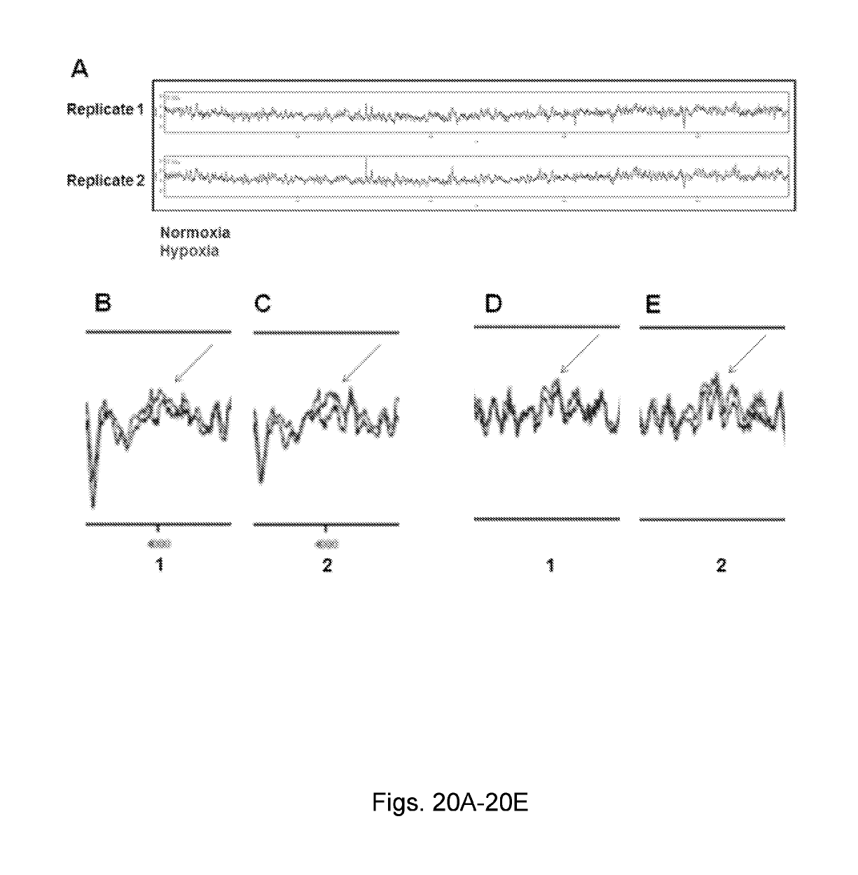

E. coli (Top10) were subjected to hypoxia (1%) and normoxia and genomic DNA was isolated and sequenced (FIGS. 20A-20E). The data demonstrates that altered DNA levels are occurring with hypoxic stress as observed with the KDM4-related regions in mammalian cells.

### Example 5: Regulation of Transient Site-Specific Copy Gain by MicroRNA

Intra-tumor copy number heterogeneity is commonly observed in cancer, however the molecular mechanisms that contribute to heterogeneity remain poorly understood. Upregulation of the histone demethylase KDM4A promotes transient site-specific copy gain (TSSG) in cells; therefore, uncovering how KDM4A levels are controlled is important to understanding the regulation of copy number heterogeneity. It is demonstrated herein that KDM4A is regulated by hsa-mir-23a-3p, hsa-mir-23b-3p and hsa-mir-137. Altering expression of these miRNAs regulates KDM4A-dependent TSSG. miRNA inhibition promoted copy gains and increased expression of the drug resistant oncogene CKS1B, which was further substantiated in primary breast tumors. Consistent with increased CKS1B expression, miRNA inhibition reduced breast cancer cell sensitivity to cisplatin. Our data identify these miRNAs as regulators of TSSG and copy gains of a drug resistance gene.

Genomic instability is a hallmark of cancer and contributes to drug resistance (1). Both adult and pediatric cancers have recurrent gains and losses of chromosomal regions, but little is known regarding the molecular mechanisms causing either transient or permanent copy number changes at specific sites within the genome. Such copy number gains, when contributing to increased expression of oncogenes, have been shown to impact cellular behavior and/or correlate with poor outcome and reduced chemotherapeutic response (2-5). For instance, tumors with worse outcome and reduced response to therapeutics often harbor chromosome 1q12-25 cytogenetic gains; however, the genes that contribute to this phenotype may vary depending on tumor type even though the same cytogenetic region is gained (2-10).

Overexpression of the lysine demethylase KDM4A/JMJD2A and the modulation of epigenetic states (i.e., histone 3 lysine 9 and 36 methylation) results in transient site-specific copy gains (TSSGs) through rereplication in the human genome (11-14). TSSGs are copy gain/amplification events that are reversible, occur during cell cycle, but are not permanently integrated into the genome (11-14). Moreover, TSSG is not just a cancer specific event, but can be regulated by physiologic stimuli. For example, hypoxia also promotes TSSGs through stabilization of KDM4A protein levels (11,13).

KDM4A protein levels are regulated, during cell cycle and in hypoxic exposure, by the SKP1-Cul1-F-box ubiquitin ligase complex and at least three F-box proteins (11,15-18). However, it is likely that other mechanisms exist to modulate KDM4A protein levels, which will play an important role in regulating TSSG in 1q12-21. Possible candidates for regulating KDM4A are microRNAs (miRNAs). MicroRNAs are short (19-22 nucleotides) non-coding RNAs, which in complex with the RNA-induced silencing complex (RISC) target the 3′-untranslated region (3′-UTR) through binding to specific complementary seed sequences (19). Transcripts targeted by RISC/miRNA are then translationally repressed or degraded (19).

It is demonstrated herein that KDM4A is regulated by hsa-mir-23a/b-3p (hereafter hsa-mir-23a/b) and hsa-mir-137. Addition of miRNA mimics to cells resulted in decreased KDM4A protein expression, while inhibition of the endogenous miRNA resulted in increased KDM4A protein levels. Addition of the KDM4A 3′-UTR to luciferase rendered it responsive to these miRNA, which was blocked by mutation of the hsa-mir-23a/b and hsa-mir-137 seed sequences. Interestingly, upregulation of KDM4A through depletion of these miRNA promotes TSSG of 1q12-21. Reciprocally, treatment with hsa-mir-23a/b or hsa-mir-137 mimics was sufficient to abrogate KDM4A-dependent TSSGs in response to hypoxia.

Consistent with these observations, miRNA inhibitors were used in MDA-MB-231 breast cancer cells to promote gain of 1q12-21 as well as the amplification and increased expression of CKS1B, which is a drug resistant oncogene (4,20-23). Furthermore, analysis of primary breast tumors (BRCA) in The Cancer Genome Atlas (TCGA) revealed that deletion of hsa-mir-23a correlates with increased copy number of 1q12-21 in primary tumors and associates with copy gain and increased expression of the drug resistant oncogene CKS1B. Consistent with these observations, miRNA inhibitors reduced breast cancer cell response to cisplatin. The present results implicate miRNA regulation as a modulator of TSSGs and indicate that miRNA therapy could be used to reduce KDM4A-driven copy number heterogeneity and potentially affect drug resistance.

Experimental Procedures

Cell Culture and Transfections—

hTERT-RPE-1 (called RPE throughout manuscript) and MDA-MB-231, cells were maintained in DMEM with 10% fetal bovine serum, 1% penicillin/streptomycin, and L-glutamine. SK-N-AS cells were maintained in DMEM/F12 (Gibco) with 10% fetal bovine serum, 1% penicillin/streptomycin, and L-glutamine. H2591 cells were maintained in RPMI (Gibco) with 10% fetal bovine serum, 1% penicillin/streptomycin, and L-glutamine. Transient transfection experiments with miRNA mimics or inhibitors were performed using Roche X-tremeGENE™ siRNA reagent in OPTI-MEM I media overnight (approximately 12 hours). Media was changed to DMEM (or DMEM/F12 or RPMI as appropriate) following the overnight incubation and cells were collected at 72 hours following transfection. Each miRNA experiment represents the average of at least two different transfections for each miRNA mimic or inhibitor. Transient transfection experiments with KDM4A siRNA were co-transfected with the miRNA using Roche X-tremeGENE™ siRNA reagent in OPTI-MEM I media overnight. Silencer Select™ siRNA for KDM4A was purchased from Life Technologies (s18636).

Hypoxic Conditions—

Cells were plated onto culture dishes and allowed to adhere for 20-24 hours in normoxia (5% CO₂, 21% O₂, and 74% N₂). For hypoxic treatment, cells were maintained in a HERA Cell 150 incubator (Thermo Scientific) flushed with 5% CO₂, 1% O₂, and balanced with N₂ for the duration of the experiment. Incubator calibrations and verifications were carried out by Bianchi Associates Calibrations/Verifications.

Fluorescent In Situ Hybridization (FISH)—

FISH was performed as described in (12). Probes for 1q12h, chromosome 8 centromere (alpha satellite) and CKS1B were purchased from Rainbow Scientific through Oxford Gene Technologies. Probes for 1q21.2 (BCL9) and 1q23.3 were purchased from Agilent (SureFISH™). Images of multiple planes of fields of nuclei were acquired on an Olympus IX81™ Spinning Disk Microscope using a 40× objective and analyzed using Slidebook 5.0™ software. We used a conservative scoring metric for copy gain. Any foci that were touching were scored as a single copy to prevent increased numbers due to normally replicated foci. For RPE cells, copy gain was scored as any cell with 3 or more distinct foci. For MDA-MB-231 cells, copy gain was scored for any cell with 7 or more foci for 1q12h and CKS1B and 5 or more for 8c or CDKN2C. For SK-N-AS cells copy gain was scored for any cell with 4 or more foci for 1q12h 3 or more for 8c. For H2591 cells copy gain was scored for any cell with 5 or more foci for 1q12h 4 or more for 8c. Approximately 100 cells for each replicate were scored for all experiments. All FISH experiments include at least 2 biological replicates.

Antibodies—

Antibodies used were: KDM4A (Neuro mAB, 75-189), β-actin (Millipore), Actinin (Santa Cruz, sc-17829), CAIX (Abcam, ab108351).

Western Blots—

Western blots were performed as in (15). Samples for western analysis were from the same collections used for FISH, FACS and RNA analyses. Quantitation was performed using ImageJ™ gel analysis with area under the curve. KDM4A levels were normalized to Actin or Actinin levels (as indicated in each figure) and then a ratio to the appropriate control sample was calculated. Data are thus presented as a fold change relative to the control.

Expression Plasmids and Luciferase Assays—

The WT and MT KDM4A 3′-UTR were cloned into pMIR as the 3′-UTR to luciferase. The pMIR-3′-UTR constructs and β-galactosidase construct for normalization were co-transfected with the indicated miRNA mimics for 48 hours using Roche xTremeGene™ siRNA transfection reagent (Roche) in OPTI-mem I media (Life Technologies). Cells were collected by scraping and lysates were prepared following the Dual-Ligh™t system instructions (Life Technologies). The dual luciferase and Beta-galactosidase assays were performed using the Dual-Light™ system following the manufacturer's instructions (Life Technologies). Measurements for two biological replicates were taken in triplicate and averaged.

miRNA Mimics and Inhibitors—

The miRNA mimics and inhibitors were purchased from Life Technologies. The mimics used were MirVana pre-miRNA23a (MC10644), MirVana pre-miRNA23b (MC10711), MirVana pre-miRNA137 (MC10513), MirVana pre-miRNA200b (MC10492), and MirVana pre-miRNA200c (MC11714) and MirVana Control (4464058). The inhibitors used were MiRVana anti-miRNA23a (MH10644), MiRVana anti-miRNA23b (MH10711), MiRVana anti-miRNA137 (MH10513), MiRVana anti-miRNA200b (MH10492), MiRVana anti-miRNA200c (MH11714), and MirVana Control (4464076).

Cisplatin Sensitivity by MTT Assay—

5000 MDA-MB-231 cells were plated overnight in each well of a 96 well plate. Cells were transfected with miRNA inhibitors using Roche X-tremeGENE™ siRNA reagent in OPTI-MEM I media overnight (approximately 12 hours). Media was changed to DMEM following the overnight incubation and cells were allowed to recover for eight hours. Cisplatin (abcam ab 141398) was resuspended in 0.9% NaCl right before use. Cisplatin was added following the eight hour recovery to a final concentration of 300 μM. Cells were processed using Cell Proliferation Kit I MTT (Roche) 48 hours after addition of cisplatin following the manufacturer's instructions. Each experiment consisted of four technical replicate wells that were averaged together and then taken as a ratio to the no cisplatin sample. The data presented are the average of eight biological replicates.

RNA Extraction and Quantitative PCR—

RNA extraction, cDNA synthesis and quantitative PCR were conducted as in (11). Expression levels were analyzed by quantitative real time PCR in a Lightcycler 480 with FastStart Universal SYBR™ Green Master (Roche) following the manufacturer's protocols. All samples were normalized by comparison to 3-actin transcript levels.

TCGA Data Set and Copy Number Determination—

The copy number and mRNA expression for TCGA Breast Cancer (BRCA) were download from Broad GDAC (Genome Data Analysis Center) Firehose analysis run of 2014_07_15 (doi:10.7908/C1TQ60P0). 1,030 common samples from two data platforms were used in this analysis. The somatic copy number alterations (SCNAs) for 23,246 genes and 928 microRNAs were annotated by GISTIC2.0 (24-26). The copy number change in each gene/miRNA is defined as possessing deep deletion (−2), shallow deletions (−1), neutral copy number (0), low gain (+1), and high gain (+2) in each sample using sample-specific thresholds. High gains are segments with copy number that exceed the maximum median chromosomal arm copy number for that sample by at least 0.1; low gains are segments with copy numbers from 2.1 to the high gain threshold; neutral segments have copy numbers between 1.9 and 2.1; shallow losses have copy numbers between 1.9 and the deep deletion threshold; and deep deletion have copy numbers that are below the minimum median chromosomal arm copy number for that sample by at least 0.1.

Determination of Cytoband Copy Number and Correlation with microRNA Loss—

In addition to the copy number annotation for each gene, the mean focal copy number for 807 cytobands including the X chromosome were annotated in each sample by taking an average of focal copy numbers of every genes within the same cytoband. Arm-level SCNA contributions to the mean focal copy number in each cytoband were removed by only considering GISTIC annotated focal copy numbers that are smaller than a chromosome arm or entire chromosome. Detecting chromosomal regions significantly co-amplified with microRNA copy loss or deletion was performed by approximating a null distribution of mean cytoband copy differences by a normal function

$$N ( {μ}_{12} - {μ}_{0} , \frac{{σ}_{0}^{2}}{{n}_{0}} + \frac{{σ}_{12}^{2}}{{n}_{12}} )$$

where μ₀ and μ₁₂ are samples means across all cytobands, σ₀² and σ₁₂² are mean sample-specific variances with each group, and n₀ and n₁₂ are the number of samples in microRNA copy-neutral (GISTIC annotation=0) and microRNA copy-loss (GISTIC annotation=−1 or −2) groups, respectively. This test is based on comparing the means of the two sets while permuting values within each of the samples (and using a Gaussian approximation). The p-values across 807 cytobands were annotated by computing the probability of more extreme differences than the corresponding cytoband copy difference in the null distribution. The QQ plot of those p-values is used to show that many genes follow the null hypothesis and their associated p-values behave appropriately.

Results

KDM4A is Regulated by miRNAs—

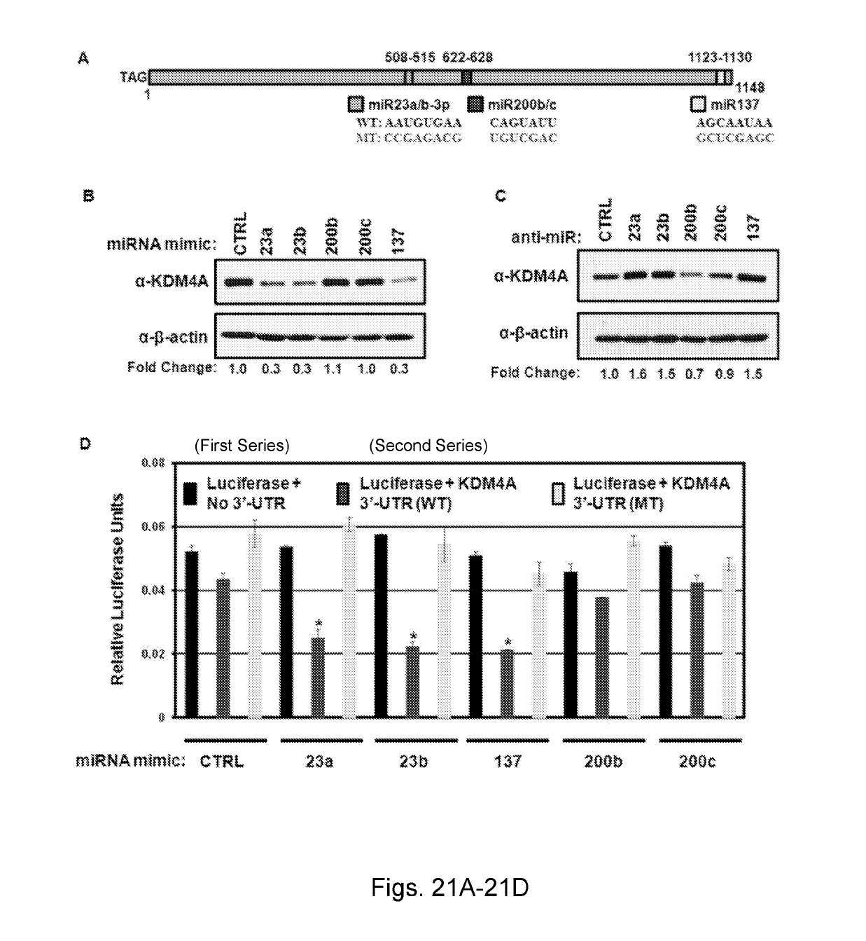

KDM4A is an important regulator of TSSGs (11-14). Uncovering how KDM4A protein levels are regulated is crucial to understanding how TSSGs can be regulated. KDM4A levels are largely regulated post-transcriptionally (11,12,15-18), suggesting that miRNAs may be ideal candidates to contribute to this regulation. To address this hypothesis, we analyzed the TARGETSCAN database for miRNAs that could target KDM4A. TARGETSCAN6.2 identified three conserved miRNA seed sequences in the KDM4A 3′-UTR (FIG. 21A) for hsa-mir-23a/b-3p (hereafter hsa-mir-23a/b), hsa-mir-137, and hsa-mir200b/c (27,28). To determine whether these miRNA could indeed regulate KDM4A, we treated the immortalized but non-transformed human retinal pigment epithelial cell line (RPE cells) with miRNA mimics (FIG. 21B). KDM4A protein levels were downregulated when cells were exposed to hsa-mir-23a/b and hsa-mir-137, but had minimal change when exposed to increased hsa-mir200b/c. Moreover, KDM4A protein levels increased when cells were treated with inhibitors of hsa-mir-23a/b and hsa-mir-137, but not with hsa-mir200b/c (FIG. 21C). These results are consistent with hsa-mir-23a/b and hsa-mir-137 regulating KDM4A in human cells.

In order to determine if the KDM4A 3′-UTR was the direct target of hsa-mir-23a/b and hsa-mir-137, we cloned the KDM4A 3′-UTR downstream of the luciferase cDNA. The miRNA seed sequences were left intact (WT UTR), or carried a series of point mutations removing the seed sequences for hsa-mir-23a/b, hsa-mir-137 and hsa-mir 200b/c (MT UTR; FIG. 21A). These constructs were then introduced into RPE cells in conjunction with mimics to hsa-mir-23a/b, hsa-mir-137, hsa-mir200b/c or a control miRNA mimic. Cells treated with hsa-mir-23a/b or hsa-mir-137 miRNA reduced luciferase expressions when luciferase was fused with the WT 3′-UTR, but not when attached to the mutated 3′-UTR (MT; FIG. 21D). Overexpression of hsa-mir200b/c did not induce significant change in luciferase expression. Taken together, these data demonstrate that KDM4A is a direct target for regulation by hsa-mir-23a/b and hsa-mir-137 in RPE cells.

Regulation of TSSGs by miRNA—

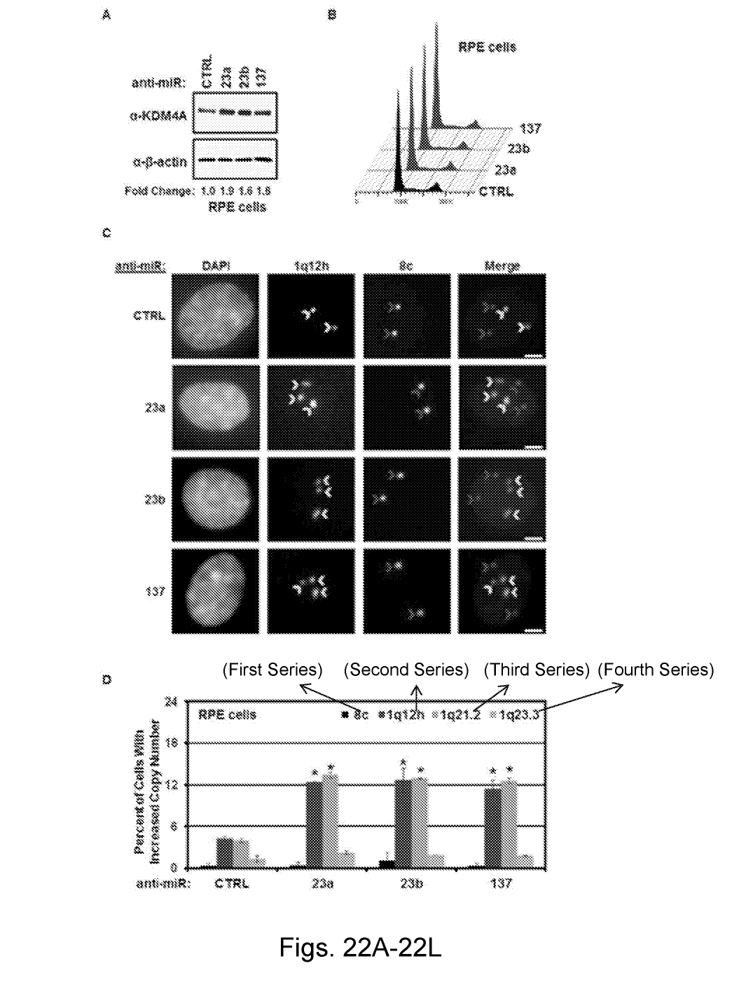

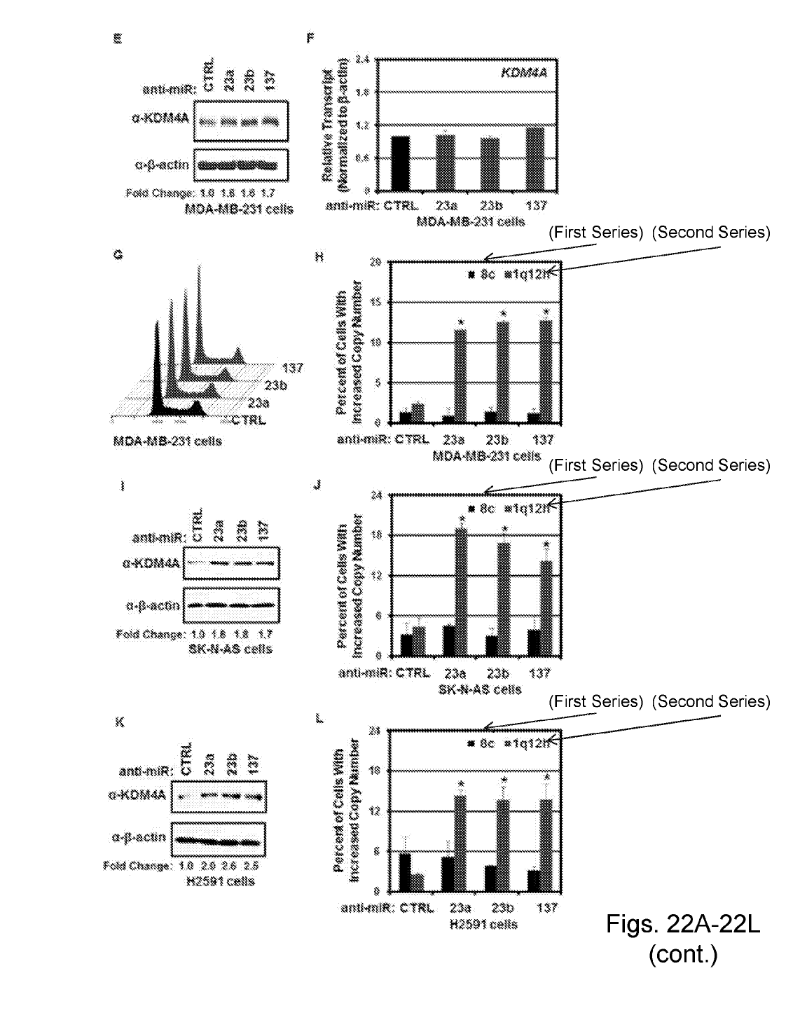

Increased expression of KDM4A is sufficient to promote TSSGs (11,12). TSSGs are characterized by cells with at least one additional copy of specific genomic loci that occur during S phase (11-14). The ability of miRNAs to regulate KDM4A protein levels suggested that decreasing hsa-mir-23a/b or hsa-mir-137 expression would be sufficient to increase KDM4A levels and thus promote TSSGs. Therefore, we introduced hsa-mir-23a/b or hsa-mir-137 inhibitors (anti-miRs) into RPE cells for 72 hours and assessed copy number by fluorescent in situ hybridization (DNA-FISH). The anti-miRs were sufficient to induce increased expression of KDM4A (FIG. 22A) without altering cell cycle distribution (FIG. 22B). We then determined the percentage of cells in the population that had at least one additional copy of the regions known to undergo TSSGs (i.e. 1q12h and 1q21.2) as well as control regions (i.e. 1q23.3 and chromosome 8 centromere) by DNA-FISH. Inhibition of hsa-mir-23a/b or hsa-mir-137 was sufficient to promote copy gain of 1q12h and 1q21.2, but did not alter the copy number of 1q23.3 or chromosome 8 centromere (FIG. 22C-22D). We confirmed these findings in MDA-MB-231 breast cancer cells. As in RPE cells, introduction of the anti-miRs resulted in increased KDM4A protein levels (FIG. 22E) without altering steady-state KDM4A transcript levels or cell cycle (FIG. 22F-22G). MicroRNA inhibition increased copy number of 1q12h (FIG. 22H), but not the chromosome 8 centromere. Treatment with microRNA inhibitors in neruoblastoma (SK-N-AS cells; FIGS. 22I-22J) and lung cancer cells (H2591; FIG. 22K-22L) also increased KDM4A protein levels and promoted copy gain of 1q12h but not chromosome 8 centromere.

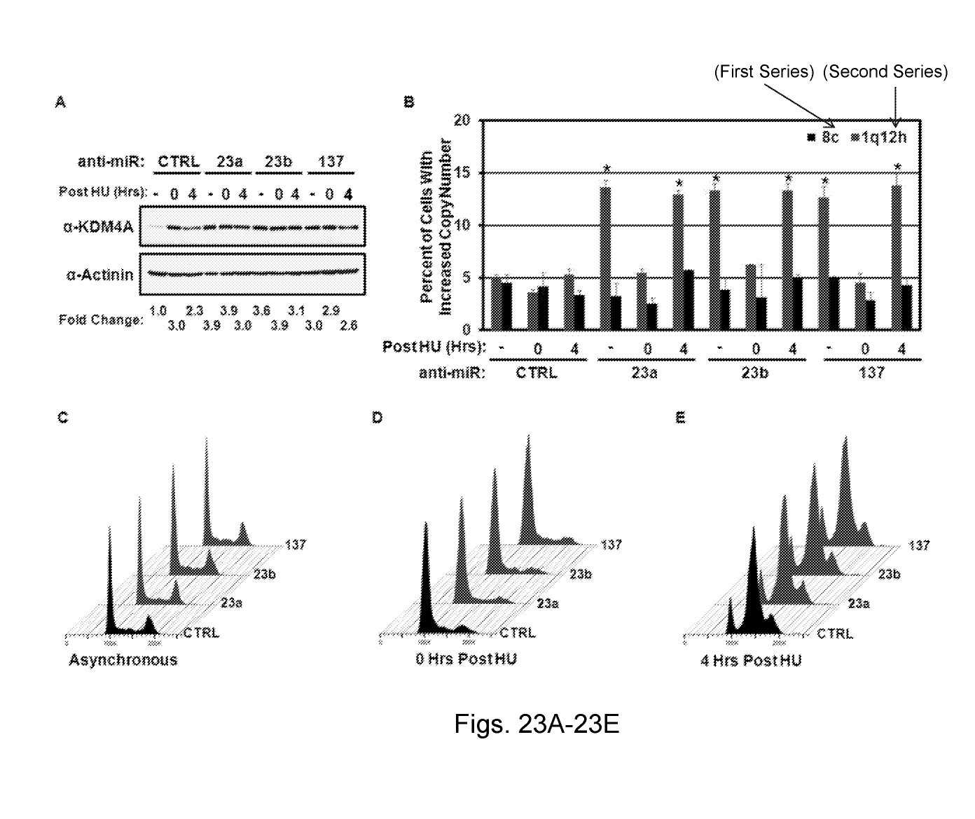

TSSGs are characterized by their transient appearance during S phase (11-14). Therefore, we tested whether the observed copy gains were S phase-dependent and by definition TSSGs. RPE cells were transfected with hsa-mir-23a/b or hsa-mir-137 anti-miRs prior to arrest with hydroxyurea (HU) for 20 hrs or arrested and released from HU for four hours. TSSG at 1q12h was assessed by DNA FISH (FIG. 23A-23E). Early S arrest with HU blocked the ability of the miRNA inhibitors to induce copy gain (FIG. 23B). However, once cells were released into S phase, hsa-mir-23a/b or hsa-mir-137 anti-miRs promoted copy gain (FIG. 23B). These results demonstrate that inhibition of miRNAs promotes TSSG.

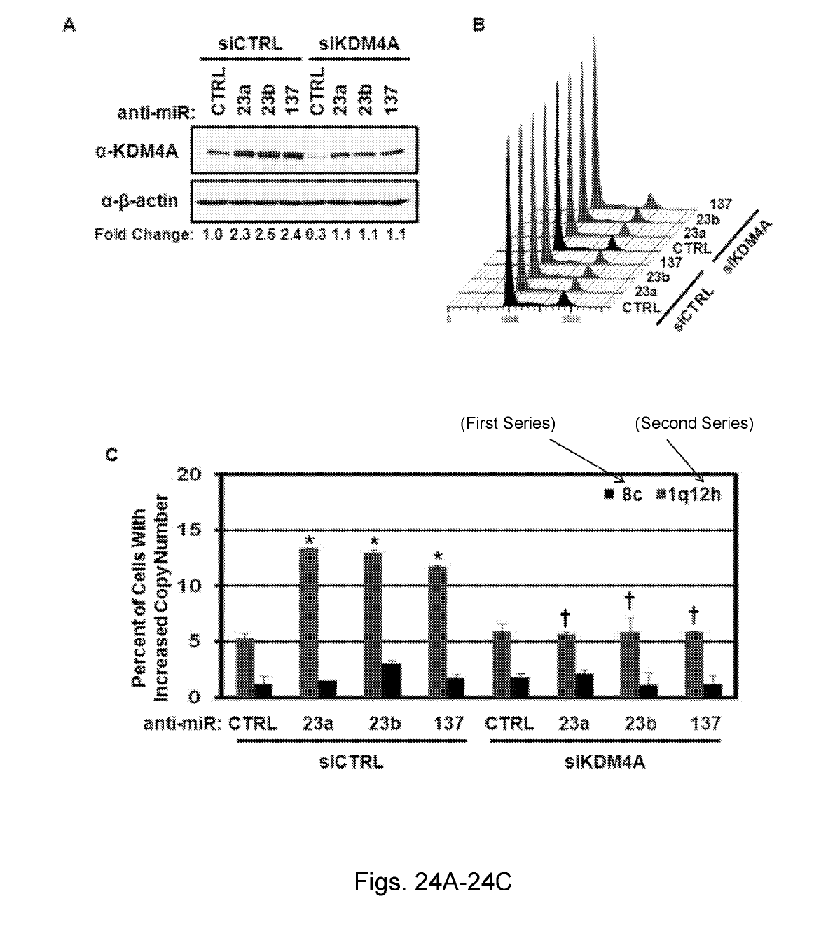

To determine whether the hsa-mir-23a/b or hsa-mir-137 anti-miRs caused TSSG through KDM4A, we co-depleted KDM4A using siRNAs with the KDM4A-targeting anti-miRs. Depletion of KDM4A by siRNA reduced KDM4A levels in the miRNA inhibitor treated cells (FIG. 24A). Cell cycle distribution was unaffected (FIG. 24B), while the reduction in KDM4A levels prevented induction of TSSG by the miRNA inhibitors (FIG. 24C). These results demonstrate that inhibition of hsa-mir-23a/b or hsa-mir-137 promotes TSSG in a KDM4A-dependent manner.

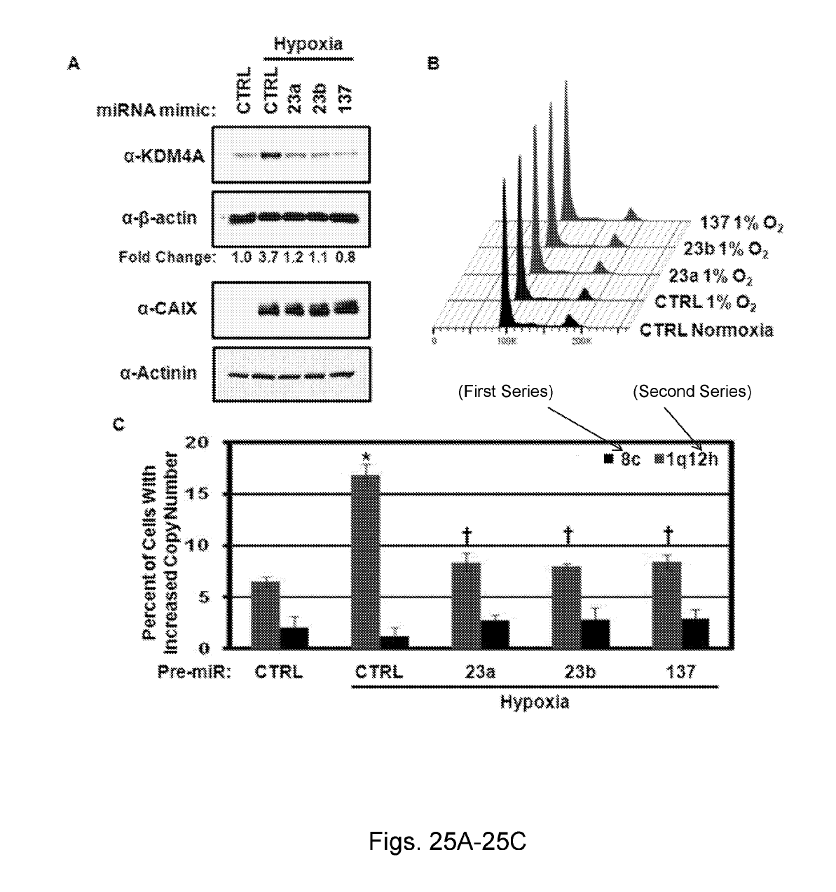

Hypoxia can induce TSSGs by stabilizing KDM4A protein levels (11). Since KDM4A protein levels respond to miRNAs, it was hypothesized that miRNA mimics would deplete KDM4A during hypoxia and prevent hypoxia-induced TSSGs. To test this hypothesis, we transfected RPE cells with miRNA mimics to hsa-mir-23a/b or hsa-mir-137 for 48 hours prior to moving the cells to hypoxia for 24 hours. Introduction of the miRNA mimics was sufficient to blunt the increased expression of KDM4A in hypoxia (FIG. 25A), but did not alter the cell cycle distribution of the treated cells (FIG. 25B). Consistent with the reduction in KDM4A levels, miRNA mimics were sufficient to abrogate hypoxia-dependent TSSG in RPE cells (FIG. 25C). Our results indicate that increasing hsa-mir-23a/b or hsa-mir-137 levels is effective in reducing hypoxia-induced or KDM4A-dependent TSSG.

Loss of hsa-mir-23a Associates with Increased CKS1B Expression in Primary Breast Tumors—

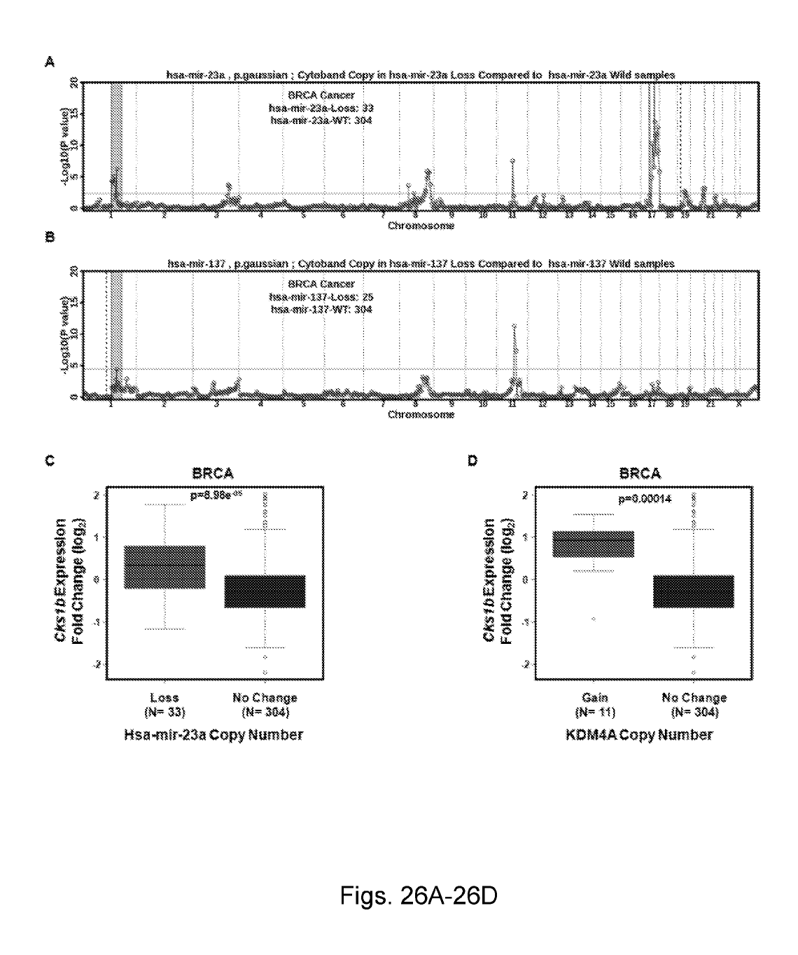

We further substantiated our in vitro findings by analyzing primary breast tumors (BRCA) in The Cancer Genome Atlas (TCGA). Specifically, we evaluated tumors that presented a loss of each miRNA alone and did not present another miRNA loss or KDM4A amplification or KDM4A loss. Using these criteria, we observed a significant gain for the 1p11.2-1q22 region in tumors presenting a loss for hsa-mir-23a (FIG. 26A). Tumors with loss of the other miRNAs showed a modest significance for this region (hsa-mir-137; FIG. 26B) or no significance (hsa-mir-23b; data not shown). Breast tumors and breast cancer cells are able to generate copy gain and increased expression of the drug resistant oncogene CKS1B (located at 1q21.3 inside the amplified region of tumors with has-mir-23a loss) upon hypoxic exposure (11). We asked if BRCA tumors with hsa-mir-23a loss had increased CKS1B expression. We observed that loss of hsa-mir-23a had increased CKS1B that was comparable to the increase in expression observed for samples with KDM4A amplification (FIG. 26C-26D). Loss of hsa-mir-23b or hsa-mir-137 resulted in a comparable trend for increased CKS1B expression, but was not significant (data not shown).

MicroRNAs Regulate Copy Number and Expression of the Drug Resistant Oncogene CKS1B—

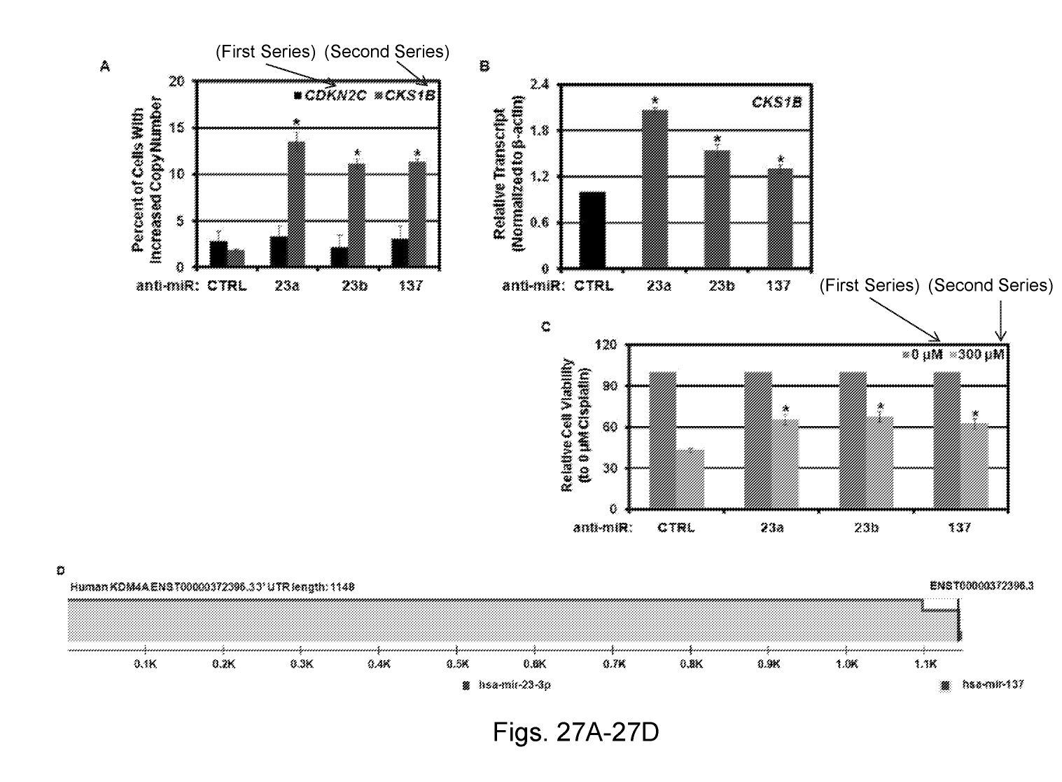

Copy gain and increased expression of CKS1B is associated with poor patient outcome and drug resistant cancer (4,20-23). Understanding mechanisms that can increase CKS1B levels has important clinical implications. Since CKS1B copy gain and increased expression in hypoxia were KDM4A-dependent, we hypothesized that hsa-mir-23a/b and hsa-mir-137 would promote gain and increased expression for CKS1B. In order to directly test this hypothesis, we transfected breast cancer cells (MDA-MB-231) with miRNA inhibitors for hsa-mir-23a, hsa-mir-23b or hsa-mir-137 and assessed copy gain and gene expression. All miRNA inhibitors resulted in increased KDM4A protein levels (FIG. 22D), while not altering the cell cycle profile (FIG. 22F). CKS1B was gained when cells were treated with hsa-mir-23a/b or hsa-mir-137 anti-miRs and had increased mRNA levels (FIG. 27A-27B), which was consistent with our recent findings in hypoxia (11). Since increased copy number and expression of CKS1B has been linked to cisplatin resistance (20,23,29,30), we hypothesized that the anti-miR induction would promote resistance to cisplatin in breast cancer cells. Indeed, prior treatment with inhibitors to hsa-mir-23a/b or hsa-mir-137 resulted in decreased sensitivity to cisplatin (FIG. 27C). Taken together, our data demonstrate that miRNAs modulate CKS1B gains and expression in vitro and show an association in vivo, which provides another mechanism for increased levels of this oncogene that contribute to resistance to cisplatin and other drugs in cancer.

Discussion

The results presented herein demonstrate that miRNAs can impact copy number heterogeneity through the regulation of TSSGs by directly regulating a chromatin modifying enzyme. Taken together, these findings illustrate the impact that miRNAs have on transient genome stability through chromatin modulation, which opens a new perspective on how non-coding RNAs can be involved in modulating tumor heterogeneity and promoting phenotypes such as drug resistance.

Our data indicates that extrinsic and intrinsic factors can modulate the composition of miRNAs within single cells or a population of cells to control the frequency of TSSGs. Alternatively, cells can alter the 3′-UTR length of key TSSG modulators, and in turn, increase heterogeneity that may impact phenotypes such as drug resistance. It remains possible that altered 3′-UTR length could be important in regulating KDM4A protein levels within tumors since amplification, altered stability and miRNAs are instrumental in regulating KDM4A levels (12,15,17,18). Consistent with this idea, TARGETSCAN7.0 indicates that the KDM4A 3′-UTR can use an alternative polyadenylation site that would eliminate the hsa-mir-137 site from the 3′-UTR (FIG. 27D) (31). Loss of the miRNA site from the 3′-UTR could result in increased KDM4A, and in turn, promote TSSGs. Alternatively, cells could select for differential 3′-UTR usage, which is frequently observed in cancer cells (32,33). Differential use of 3′-UTRs without miRNA binding sites could also increase KDM4A levels and promote TSSG and copy number heterogeneity.

MicroRNAs are often misregulated in cancer and hsa-mir-23a/b and hsa-mir-137 are no exception (34-41). For example, reduced expression of hsa-mir-137 and hsa-mir-23b has been implicated in cisplatin resistance in solid malignancies (34,37). Consistent with these previous observations, we observed copy gain and upregulation of CKS1B, which is a cell cycle regulator that has been linked to and promotes cisplatin and other drug resistance in myeloma, breast cancer and non-small-cell lung cancer (20,23,29,30). Therefore, tumors carrying the loss of hsa-mir-23a/b and hsa-mir-137 or the mis-regulation of miRNAs could mediate changes in cisplatin response by regulating KDM4A protein levels, promoting transient site-specific copy gains and heterogeneous overexpression of CKS1B. For these reasons, it may be beneficial to consider using hsa-mir-23a/b and hsa-mir-137 mimics or KDM4A anti-sense RNA strategies to reduce KDM4A protein levels in tumors that have lost these miRNAs or gained KDM4A. In fact, miRNA mimics and inhibitors are gaining traction in their use as therapies for metabolic disease and cancer (42,43). As new regulators of TSSGs are identified, it will be important to evaluate how they are regulated and consider miRNAs as a potential way to modulate their activity.

The present data underscores how changes in miRNA abundance can influence how tumors acquire intra-tumoral copy-number heterogeneity. The copy number changes we describe in cell culture models are transient. We observed correlations for these gains in primary tumors. Most of the miRNA loss events we observed were in the GISTIC (−1) category reflecting low level loss (as opposed, for example, to homozygous deletions) often caused by whole-chromosome or chromosome arm loss. This suggests miRNA loss events are not providing strong fitness advantage to the cells, but perhaps promote heterogeneity and plasticity that may serve as the basis of future selection. The observed intra-tumoral heterogeneity is likely the result of both permanent and transient heterogeneity. Uncovering how inappropriately amplified regions are lost will help identify pathways that may be mis-regulated in cancer leading to the accumulation and perhaps also inheritance of specific genomic regions. Without wishing to be bound by theory, we hypothesize that other defects in cancer cells could then promote incorporation of the TSSGs, and in turn, potentially have a permanent contribution to drug resistance.

This study highlights the important link between chromatin modulation, miRNA levels, heterogeneous and transient site-specific copy-number gains and potential phenotypes such as drug resistance. These findings also reiterate the importance in mapping the pathways and enzymes that are contributing to TSSGs and how the transient overexpression of genes may have a lasting effect on the cancer, even if present only transiently in a subset of the cells (such as potentiating drug resistance).

### REFERENCES

1. Hanahan, D., and Weinberg, R. A. (2011) Hallmarks of cancer: the next generation. Cell 144, 646-674
2. Dimova, I., Orsetti, B., Theillet, C., Dimitrov, R., and Toncheva, D. (2009) Copy Number Changes in 1q21.3 and 1q23.3 have Different Clinical Relevance in Ovarian Tumors. Balkan Journal of Medical Genetics 12, 29-37
3. Diskin, S. J., Hou, C., Glessner, J. T., Attiyeh, E. F., Laudenslager, M., Bosse, K., Cole, K., Mosse, Y. P., Wood, A., Lynch, J. E., Pecor, K., Diamond, M., Winter, C., Wang, K., Kim, C., Geiger, E. A., McGrady, P. W., Blakemore, A. I., London, W. B., Shaikh, T. H., Bradfield, J., Grant, S. F., Li, H., Devoto, M., Rappaport, E. R., Hakonarson, H., and Maris, J. M. (2009) Copy number variation at 1q21.1 associated with neuroblastoma. Nature 459, 987-991
4. Fonseca, R., Van Wier, S. A., Chng, W. J., Ketterling, R., Lacy, M. Q., Dispenzieri, A., Bergsagel, P. L., Rajkumar, S. V., Greipp, P. R., Litzow, M. R., Price-Troska, T., Henderson, K. J., Ahmann, G. J., and Gertz, M. A. (2006) Prognostic value of chromosome 1q21 gain by fluorescent in situ hybridization and increase CKS1B expression in myeloma. Leukemia 20, 2034-2040
5. Inoue, J., Otsuki, T., Hirasawa, A., Imoto, I., Matsuo, Y., Shimizu, S., Taniwaki, M., and Inazawa, J. (2004) Overexpression of PDZK1 within the 1q12-q22 amplicon is likely to be associated with drug-resistance phenotype in multiple myeloma. Am J Pathol 165, 71-81
6. Giulino-Roth, L., Wang, K., MacDonald, T. Y., Mathew, S., Tam, Y., Cronin, M. T., Palmer, G., Lucena-Silva, N., Pedrosa, F., Pedrosa, M., Teruya-Feldstein, J., Bhagat, G., Alobeid, B., Leoncini, L., Bellan, C., Rogena, E., Pinkney, K. A., Rubin, M. A., Ribeiro, R. C., Yelensky, R., Tam, W., Stephens, P. J., and Cesarman, E. (2012) Targeted genomic sequencing of pediatric Burkitt lymphoma identifies recurrent alterations in antiapoptotic and chromatin-remodeling genes. Blood 120, 5181-5184
7. Goeze, A., Schluns, K., Wolf, G., Thasler, Z., Petersen, S., and Petersen, I. (2002) Chromosomal imbalances of primary and metastatic lung adenocarcinomas. J Pathol 196, 8-16
8. Lestini, B. J., Goldsmith, K. C., Fluchel, M. N., Liu, X., Chen, N. L., Goyal, B., Pawel, B. R., and Hogarty, M. D. (2009) Mcl1 downregulation sensitizes neuroblastoma to cytotoxic chemotherapy and small molecule Bcl2-family antagonists. Cancer Biol Ther 8, 1587-1595
9. Vrana, J. A., Bieszczad, C. K., Cleaveland, E. S., Ma, Y., Park, J. P., Mohandas, T. K., and Craig, R. W. (2002) An MCL1-overexpressing Burkitt lymphoma subline exhibits enhanced survival on exposure to serum deprivation, topoisomerase inhibitors, or staurosporine but remains sensitive to 1-beta-D-arabinofuranosylcytosine. Cancer Res 62, 892-900
10. Weir, B. A., Woo, M. S., Getz, G., Perner, S., Ding, L., Beroukhim, R., Lin, W. M., Province, M. A., Kraja, A., Johnson, L. A., Shah, K., Sato, M., Thomas, R. K., Barletta, J. A., Borecki, I. B., Broderick, S., Chang, A. C., Chiang, D. Y., Chirieac, L. R., Cho, J., Fujii, Y., Gazdar, A. F., Giordano, T., Greulich, H., Hanna, M., Johnson, B. E., Kris, M. G., Lash, A., Lin, L., Lindeman, N., Mardis, E. R., McPherson, J. D., Minna, J. D., Morgan, M. B., Nadel, M., Orringer, M. B., Osborne, J. R., Ozenberger, B., Ramos, A. H., Robinson, J., Roth, J. A., Rusch, V., Sasaki, H., Shepherd, F., Sougnez, C., Spitz, M. R., Tsao, M. S., Twomey, D., Verhaak, R. G., Weinstock, G. M., Wheeler, D. A., Winckler, W., Yoshizawa, A., Yu, S., Zakowski, M. F., Zhang, Q., Beer, D. G., Wistuba, II, Watson, M. A., Garraway, L. A., Ladanyi, M., Travis, W. D., Pao, W., Rubin, M. A., Gabriel, S. B., Gibbs, R. A., Varmus, H. E., Wilson, R. K., Lander, E. S., and Meyerson, M. (2007) Characterizing the cancer genome in lung adenocarcinoma. Nature 450, 893-898
11. Black, J. C., Atabakhsh, E., Kim, J., Biette, K. M., Van Rechem, C., Ladd, B., Burrowes, P. D., Donado, C., Mattoo, H., Kleinstiver, B. P., Song, B., Andriani, G., Joung, J. K., Iliopoulos, O., Montagna, C., Pillai, S., Getz, G., and Whetstine, J. R. (2015) Hypoxia drives transient site-specific copy gain and drug-resistant gene expression. Genes Dev 29, 1018-1031
12. Black, J. C., Manning, A. L., Van Rechem, C., Kim, J., Ladd, B., Cho, J., Pineda, C. M., Murphy, N., Daniels, D. L., Montagna, C., Lewis, P. W., Glass, K., Allis, C. D., Dyson, N. J., Getz, G., and Whetstine, J. R. (2013) KDM4A lysine demethylase induces site-specific copy gain and rereplication of regions amplified in tumors. Cell 154, 541-555
13. Black, J. C., and Whetstine, J. R. (2015) Too little O2 Too much gain. Cell Cycle 14, 2869-2870
14. Mishra, S., and Whetstine, J. R. (2016) Different Facets of Copy Number Changes: Permanent, Transient, and Adaptive. Mol Cell Biol
15. Black, J. C., Allen, A., Van Rechem, C., Forbes, E., Longworth, M., Tschop, K., Rinehart, C., Quiton, J., Walsh, R., Smallwood, A., Dyson, N. J., and Whetstine, J. R. (2010) Conserved antagonism between JMJD2A/KDM4A and HPlgamma during cell cycle progression. Mol Cell 40, 736-748
16. Tan, M. K., Lim, H. J., and Harper, J. W. (2011) SCF(FBXO22) regulates histone H3 lysine 9 and 36 methylation levels by targeting histone demethylase KDM4A for ubiquitin-mediated proteasomal degradation. Mol Cell Biol 31, 3687-3699
17. Van Rechem, C., Black, J. C., Abbas, T., Allen, A., Rinehart, C. A., Yuan, G. C., Dutta, A., and Whetstine, J. R. (2011) The SKP1-Cul1-F-box and leucine-rich repeat protein 4 (SCF-FbxL4) ubiquitin ligase regulates lysine demethylase 4A (KDM4A)/Jumonji domain-containing 2A (JMJD2A) protein. J Biol Chem 286, 30462-30470
18. Van Rechem, C., Black, J. C., Greninger, P., Zhao, Y., Donado, C., Burrowes, P. D., Ladd, B., Christiani, D. C., Benes, C. H., and Whetstine, J. R. (2015) A Coding Single Nucleotide Polymorphism in Lysine Demethylase KDM4A Associates with Increased Sensitivity to mTOR Inhibitors. Cancer Discov
19. Ha, M., and Kim, V. N. (2014) Regulation of microRNA biogenesis. Nat Rev Mol Cell Biol 15, 509-524
20. Khattar, V., and Thottassery, J. V. (2013) Cks1: Structure, Emerging Roles and Implications in Multiple Cancers. J Cancer Ther 4, 1341-1354
21. Martin-Ezquerra, G., Salgado, R., Toll, A., Baro, T., Mojal, S., Yebenes, M., Garcia-Muret, M. P., Sole, F., Quitllet, F. A., Espinet, B., and Pujol, R. M. (2011) CDC28 protein kinase regulatory subunit 1B (CKS1B) expression and genetic status analysis in oral squamous cell carcinoma. Histol Histopathol 26, 71-77
22. Shaughnessy, J. (2005) Amplification and overexpression of CKS1B at chromosome band 1q21 is associated with reduced levels of p27Kip1 and an aggressive clinical course in multiple myeloma. Hematology 10 Suppl 1, 117-126
23. Shi, L., Wang, S., Zangari, M., Xu, H., Cao, T. M., Xu, C., Wu, Y., Xiao, F., Liu, Y., Yang, Y., Salama, M., Li, G., Tricot, G., and Zhan, F. (2010) Over-expression of CKS1B activates both MEK/ERK and JAK/STAT3 signaling pathways and promotes myeloma cell drug-resistance. Oncotarget 1, 22-33
24. Beroukhim, R., Getz, G., Nghiemphu, L., Barretina, J., Hsueh, T., Linhart, D., Vivanco, I., Lee, J. C., Huang, J. H., Alexander, S., Du, J., Kau, T., Thomas, R. K., Shah, K., Soto, H., Perner, S., Prensner, J., Debiasi, R. M., Demichelis, F., Hatton, C., Rubin, M. A., Garraway, L. A., Nelson, S. F., Liau, L., Mischel, P. S., Cloughesy, T. F., Meyerson, M., Golub, T. A., Lander, E. S., Mellinghoff, I. K., and Sellers, W. R. (2007) Assessing the significance of chromosomal aberrations in cancer: methodology and application to glioma. Proc Natl Acad Sci USA 104, 20007-20012
25. Beroukhim, R., Mermel, C. H., Porter, D., Wei, G., Raychaudhuri, S., Donovan, J., Barretina, J., Boehm, J. S., Dobson, J., Urashima, M., Mc Henry, K. T., Pinchback, R. M., Ligon, A. H., Cho, Y. J., Haery, L., Greulich, H., Reich, M., Winckler, W., Lawrence, M. S., Weir, B. A., Tanaka, K. E., Chiang, D. Y., Bass, A. J., Loo, A., Hoffman, C., Prensner, J., Liefeld, T., Gao, Q., Yecies, D., Signoretti, S., Maher, E., Kaye, F. J., Sasaki, H., Tepper, J. E., Fletcher, J. A., Tabernero, J., Baselga, J., Tsao, M. S., Demichelis, F., Rubin, M. A., Janne, P. A., Daly, M. J., Nucera, C., Levine, R. L., Ebert, B. L., Gabriel, S., Rustgi, A. K., Antonescu, C. R., Ladanyi, M., Letai, A., Garraway, L. A., Loda, M., Beer, D. G., True, L. D., Okamoto, A., Pomeroy, S. L., Singer, S., Golub, T. R., Lander, E. S., Getz, G., Sellers, W. R., and Meyerson, M. (2010) The landscape of somatic copy-number alteration across human cancers. Nature 463, 899-905
26. Network, C. G. A. R. (2008) Comprehensive genomic characterization defines human glioblastoma genes and core pathways. Nature 455, 1061-1068
27. Garcia, D. M., Baek, D., Shin, C., Bell, G. W., Grimson, A., and Bartel, D. P. (2011) Weak seed-pairing stability and high target-site abundance decrease the proficiency of 1sy-6 and other microRNAs. Nat Struct Mol Biol 18, 1139-1146
28. Lewis, B. P., Burge, C. B., and Bartel, D. P. (2005) Conserved seed pairing, often flanked by adenosines, indicates that thousands of human genes are microRNA targets. Cell 120, 15-20
29. Fujita, Y., Yagishita, S., Hagiwara, K., Yoshioka, Y., Kosaka, N., Takeshita, F., Fujiwara, T., Tsuta, K., Nokihara, H., Tamura, T., Asamura, H., Kawaishi, M., Kuwano, K., and Ochiya, T. (2015) The clinical relevance of the miR-197/CKS1B/STAT3-mediated PD-L1 network in chemoresistant non-small-cell lung cancer. Mol Ther 23, 717-727
30. Wang, X. C., Tian, J., Tian, L. L., Wu, H. L., Meng, A. M., Ma, T. H., Xiao, J., Xiao, X. L., and Li, C. H. (2009) Role of Cks1 amplification and overexpression in breast cancer. Biochem Biophys Res Commun 379, 1107-1113
31. Agarwal, V., Bell, G. W., Nam, J. W., and Bartel, D. P. (2015) Predicting effective microRNA target sites in mammalian mRNAs. Elife 4
32. Lembo, A., Di Cunto, F., and Provero, P. (2012) Shortening of 3′UTRs correlates with poor prognosis in breast and lung cancer. PLoS One 7, e31129
33. Mayr, C., and Bartel, D. P. (2009) Widespread shortening of 3′UTRs by alternative cleavage and polyadenylation activates oncogenes in cancer cells. Cell 138, 673-684
34. An, Y., Zhang, Z., Shang, Y., Jiang, X., Dong, J., Yu, P., Nie, Y., and Zhao, Q. (2015) miR-23b-3p regulates the chemoresistance of gastric cancer cells by targeting ATG12 and HMGB2. Cell Death Dis 6, e1766
35. Gao, P., Tchernyshyov, I., Chang, T. C., Lee, Y. S., Kita, K., Ochi, T., Zeller, K. I., De Marzo, A. M., Van Eyk, J. E., Mendell, J. T., and Dang, C. V. (2009) c-Myc suppression of miR-23a/b enhances mitochondrial glutaminase expression and glutamine metabolism. Nature 458, 762-765
36. Guo, J., Xia, B., Meng, F., and Lou, G. (2013) miR-137 suppresses cell growth in ovarian cancer by targeting AEG-1. Biochem Biophys Res Commun 441, 357-363
37. Li, P., Ma, L., Zhang, Y., Ji, F., and Jin, F. (2014) MicroRNA-137 down-regulates KIT and inhibits small cell lung cancer cell proliferation. Biomed Pharmacother 68, 7-12
38. Li, X., Liu, X., Xu, W., Zhou, P., Gao, P., Jiang, S., Lobie, P. E., and Zhu, T. (2013) c-MYC-regulated miR-23a/24-2/27a cluster promotes mammary carcinoma cell invasion and hepatic metastasis by targeting Sprouty2. J Biol Chem 288, 18121-18133
39. Smith, A. R., Marquez, R. T., Tsao, W. C., Pathak, S., Roy, A., Ping, J., Wilkerson, B., Lan, L., Meng, W., Neufeld, K. L., Sun, X. F., and Xu, L. (2015) Tumor suppressive microRNA-137 negatively regulates Musashi-1 and colorectal cancer progression. Oncotarget 6, 12558-12573
40. Zaman, M. S., Thamminana, S., Shahryari, V., Chiyomaru, T., Deng, G., Saini, S., Majid, S., Fukuhara, S., Chang, I., Arora, S., Hirata, H., Ueno, K., Singh, K., Tanaka, Y., and Dahiya, R. (2012) Inhibition of PTEN gene expression by oncogenic miR-23b-3p in renal cancer. PLoS One 7, e50203
41. Zhu, X., Li, Y., Shen, H., Li, H., Long, L., Hui, L., and Xu, W. (2013) miR-137 inhibits the proliferation of lung cancer cells by targeting Cdc42 and Cdk6. FEBS Lett 587, 73-81
42. Di Leva, G., Garofalo, M., and Croce, C. M. (2014) MicroRNAs in cancer. Annu Rev Pathol 9, 287-314
43. Kasinski, A. L., and Slack, F. J. (2011) Epigenetics and genetics. MicroRNAs en route to the clinic: progress in validating and targeting microRNAs for cancer therapy. Nat Rev Cancer 11, 849-864

1. A method of reducing and/or preventing the development of drug resistance in a cell, the method comprising contacting the cell with an inhibitor of a KDM4A-like enzyme or an inhibitor of an enzyme that hydroxylates nucleic acids and/or histones or histone-like proteins.

2. The method of claim 1, wherein the cell is a prokaryotic cell.

3. The method of claim 2, wherein the drug resistance is antibiotic resistance.

4. The method of claim 1, wherein the cell is a eukaryotic cell.

5. The method of claim 4, wherein the cell is selected from the group consisting of:
a yeast cell and a mammalian cell.

6. The method of claim 3, wherein the cell is a cancer cell.

7. The method of claim 4, wherein the drug resistance is chemotherapeutic resistance.

8. (canceled)

9. The method of claim 1, wherein the KDM4A-like enzyme comprises a cupin β barrel domain.

10. The method of claim 1, wherein the KDM4A-like enzyme is selected from the group consisting of:
KDM4A; KDM5A; KDM6B; KDM4B; KDM4C; a member of the JmjC enzyme family; a Cupin protein; and the proteins listed in Tables 1 and 2 and/or homologs thereof.

11. The method of claim 1, wherein the KDM4A-like enzyme is KDM4A.

12. The method of claim 1, wherein the inhibitor of a KDM4A-like enzyme is selected from the group consisting of:
an inhibitory nucleic acid; an aptamer; a miRNA; Suv39H1; HP1; increased oxygen levels; an inhibitor of a KDM4A-targeting KMT; an inhibitor of Tudor or PHD domain interaction; succinate; JIB-04; a 8-(1H-pyrazol-3-yl)pyrido[3,4-d]pyrimidin-4(3H)-one; 3-((furan-2-ylmethyl)amino)pyridine-4-carboxylic acid; and 3-(((3-methylthiophen-2-yl)methyl)amino)pyridine-4-carboxylic acid.

13. The method of claim 1, wherein the inhibitor of a KDM4A-like enzyme is a nucleic acid comprising the sequence of hsa-mir-23 a-3p, hsa-mir-23b-3p and/or hsa-mir-137.

14. The method of claim 1, wherein the cell is a cell determined to be experiencing hypoxic conditions.

15. The method of claim 2, wherein the prokaryotic cell comprises a gene encoding a KDM4A-like enzyme.

16.-65. (canceled)

66. A method of reducing and/or preventing the development of drug resistance in a subject, the method comprising administering:
a) i) a chemotherapeutic agent and ii) an inhibitor of a KDM4A-like enzyme or an inhibitor of an enzyme that hydroxylates nucleic acids and/or histones or histone-like proteins to a subject in need of treatment for cancer; or
b) an inhibitor of a KDM4A-like enzyme or an inhibitor of an enzyme that hydroxylates nucleic acids and/or histones or histone-like proteins to a subject in need of treatment for hypoxia; or
c) i) an angiogenesis inhibitor and ii) an inhibitor of a KDM4A-like enzyme or an inhibitor of an enzyme that hydroxylates nucleic acids and/or histones or histone-like proteins to a subject in need of treatment with an angiogenesis inhibitor;
d) an inhibitor of a KDM4A-like enzyme or an inhibitor of an enzyme that hydroxylates nucleic acids and/or histones or histone-like proteins to a subject in need of treatment for an infection; or
e) i) an antibiotic and ii) an inhibitor of a KDM4A-like enzyme or an inhibitor of an enzyme that hydroxylates nucleic acids and/or histones or histone-like proteins to a subject in need of treatment for an infection.

67. The method of claim 66, wherein the chemotherapeutic agent is selected from the group consisting of:
DNA-damaging agents; S-phase chemotherapeutics; mTOR inhibitors; protein synthesis inhibitors; Braf inhibitors; PI3K inhibitors; Cdk inhibitors; Aurora B inhibitors; FLT3 inhibitors; PLK1/2/3 inhibitors; Eg5 inhibitors; 3-tubulin inhibitors; BMP inhibitors; HDAC inhibitors; Akt inhibitors; IGF1R inhibitors; p53 inhibitors; hdm2 inhibitors; STAT3 inhibitors; VEGFR inhibitors; angiogenesis inhibitors; proteasomal inhibitors; ubiquitin-targeting drugs; and bortezomib.

68. The method of claim 66, wherein the angiogenesis inhibitor is selected from the group consisting of:
bevacizumab; sorefenib; sunitinib; pazopanib; and everolimus.


SEQUENCE LISTING (40 sequences)

SEQ ID NO: 1 - DNA, 4526 bp, Homo sapiens
acggctgcgcagatgccgactttagaggaggcggagtttcggccttcgcctgctggaaaagcagtaggatcggccagtggcgacagcaggagctgagcctaagccctggcggggctttgggctgtagattcctgtctgactaaagggacctcaaaaaggagggaaaatggcttctgagtctgaaactctgaatcccagtgctaggataatgaccttttatccaactatggaagagttccgaaacttcagtagatacattgcctacattgaatcccaaggagctcatcgggcagggctagccaaggttgttcctccaaaagagtggaagccacgagcatcctatgatgacattgatgatttggtcattcctgcccccattcaacagctggtgacggggcagtctggcctctttactcagtacaacatacagaagaaagccatgactgttcgagagttccgcaagatagccaatagcgataagtactgtaccccacgctatagtgagtttgaagagctcgagcggaaatactggaaaaatcttacattcaatcctccaatctatggtgcagatgtgaatggtaccctctatgaaaagcatgttgatgagtggaatattggccggctgagaacaatcctggacttggtggaaaaggagagtgggatcaccattgagggtgtgaacaccccatacctgtactttggcatgtggaagacatcctttgcttggcacactgaagacatggacctctacagcatcaactacctgcactttggagaaccaaagtcctggtactctgttccacctgagcatggaaagcggttggaacgcctcgccaaaggctttttcccaggaagtgctcaaagctgtgaggcatttctccgccacaagatgaccctgatttccccgttaatgctgaagaaatatggaattccctttgacaaggtgactcaagaggctggagagtttatgatcactttcccttatggttaccatgccggctttaaccatggttttaactgtgcggagtctaccaattttgctacccgtcggtggattgagtacggcaagcaagctgtgctgtgctcctgtagaaaggacatggtgaagatctccatggatgtgtttgtgagaaagttccagccagaaaggtacaaactttggaaagctgggaaggacaacacagttattgaccatactctgcccacgccagaagcagctgagtttcttaaggagagtgaactgcctccaagagctggcaacgaggaggagtgcccagaggaggacatggaaggggtggaggatggagaggaaggagacctgaagacaagcctggccaagcaccgaatagggacaaagaggcaccgagtttgtcttgaaataccacaggaggtgagtcagagtgagctcttccccaaggaggatctgagttctgagcagtatgagatgacggagtgcccggcagccctcgcccctgtgaggcccacccatagctctgtgcggcaagttgaggatggtcttaccttcccagattattctgactccactgaagtcaaatttgaagagcttaaaaatgtcaaactagaagaggaggatgaggaggaagaacaagcagcagctgccttggatctttctgtgaatcctgcgtctgtagggggacgccttgtcttctcaggctccaaaaagaaatcatcttctagcctgggctctggctcttcacgggattctatctcttctgattcagaaactagtgagcctctctcctgccgagcccaagggcaaacgggagttctcactgtgcacagttatgccaaaggggatggcagggtcactgtgggagagccatgcacgaggaagaaaggaagcgccgctagaagtttcagtgagcgggagctggcagaggttgcagatgaatacatgttttccctagaagagaataagaagtccaagggacgccgtcagcctttaagcaagctcccccgccatcacccacttgtgctgcaggagtgtgtcagtgatgatgagacatctgaacagctgacccctgaggaagaggctgaggagacagaggcctgggccaagcctctgagccaactgtggcagaaccgacctccaaactttgaggctgagaaggaattcaatgagaccatggcccaacaggcccctcactgcgctgtctgtatgatcttccagacttatcatcaggttgaatttggaggctttaatcagaactgtggaaatgcttcagatttagccccccagaagcagaggaccaagccattgattccagaaatgtgcttcacttcgactggctgcagcacggacatcaacctttctactccttatcttgaggaggatggcaccagcatactcgtttcctgcaagaagtgcagcgtccgggtccatgccagttgctatggggtcccccctgcaaaggcttctgaagactggatgtgttctcggtgttcagccaatgccctagaggaggactgctgtttatgctcattacgaggaggggccctgcagagagcaaatgatgacaggtgggtccacgtttcatgtgctgtggcaattctggaagcaaggtttgtcaacattgcagaaagaagtccggtggatgtgagcaaaatccccctgccccgcttcaaactgaaatgtatcttctgtaagaagcggaggaaaagaactgctggctgctgtgtgcagtgttctcacggccgctgcccaactgccttccatgtgagctgcgcccaggctgccggtgtgatgatgcagcctgacgactggccttttgtggtcttcattacctgctttcggcacaagattcctaatttggagcgtgccaagggggccttgcaaagcatcactgcaggccagaaagtcattagcaagcataagaacgggcgcttctaccagtgtgaagtggtcaggctcaccaccgagaccttctatgaagtcaactttgatgatggctccttcagcgacaatctttatcctgaggacatagtgagccaggactgtctccagtttggtcctcctgctgaaggggaagtggtccaagtgagatggacagacggccaagtctatggagccaagtttgtggcctcccaccctatccaaatgtaccaggtggagtttgaggatggctcacaacttgtggttaagagagatgatgtatacacactggatgaagagcttcccaagagagtcaaatctagactgtcagtagcctcagacatgcgcttcaatgagattttcacagagaaagaggttaagcaagaaaagaaacggcaacgagttatcaactcaagataccgggaagattatattgagcctgcactataccgggccatcatggagtaggtgcttccagggtccaagggattctcagccatccaggcaagagcactctgggttccacagcacagcagacatggaacgctgaagtctctgaaagtgaagttgtaaaaagaaaaggaatgaaataaccgacccatcatcttctcacccaccctcattgcattccgctgtagtgaaaggacgagccatttctgggcacgtggcagcagtcgctgatctcccagctgaggggctgagcactggaatgctgtggctgcactggccccagtccatagaggggtcaactatgctggctggactggctgccttgttcctggcctaggacttagcttcataactatcacctgcaccgactaggctgaggtgctggtacttgccccaacccctacttttgtatttatatgtgtgtgtgtgtgtgcgtgcgtgcgtgcgtgcgtgtatgtttggtctggaccagcttctgccagcccctggcctttactttcttccttgcctatgcagggcaaacaaaatgtgaaattctgccctcagctgagctgagtaagggctcctgggggttggctggagatgggtgtggcatctgtccaggcctggaaccgtctcaagacagtgctggcaaagctgcagtattgagatgctaaggagctgatgccacctctttgtcttcccctaaaggagaacatggggataacatgggtgtgtgcccacaacactctaggtgcagagcccctgtggcaaagtattacagggtgtgggtggggattaccctgaatcggggattttaatgatggaagcaggcagagcctggtgggtgattctgtcaacagaaaattgcaatcatgcaggggctgggagggttaggatgaaaaaactggggccattggaggcccactgtaggtgggagggagctgattttggggtggggggtgggactagagggcaatactgaaggggttaaacaggtttttgctcctcaagaatttgtttgcctgggcccaggattggagggcttcacaccaataccctgtgtatacaagaatcagatttataatacttccccttttttgttacgtatgaacactataaaccaaattattttgaaaactggtgcatcaccttgtccttagcaataaaatgtgttgagcagaggaaaaaaaaaaaaaaaaaa

SEQ ID NO: 2 - protein, 1064 aa, Homo sapiens
MASESETLNPSARIMTFYPTMEEFRNFSRYIAYIESQGAHRAGLAKVVPPKEWKPRASYDDIDDLVIPAPIQQLVTGQSGLFTQYNIQKKAMTVREFRKIANSDKYCTPRYSEFEELERKYWKNLTFNPPIYGADVNGTLYEKHVDEWNIGRLRTILDLVEKESGITIEGVNTPYLYFGMWKTSFAWHTEDMDLYSINYLHFGEPKSWYSVPPEHGKRLERLAKGFFPGSAQSCEAFLRHKMTLISPLMLKKYGIPFDKVTQEAGEFMITFPYGYHAGFNHGFNCAESTNFATRRWIEYGKQAVLCSCRKDMVKISMDVFVRKFQPERYKLWKAGKDNTVIDHTLPTPEAAEFLKESELPPRAGNEEECPEEDMEGVEDGEEGDLKTSLAKHRIGTKRHRVCLEIPQEVSQSELFPKEDLSSEQYEMTECPAALAPVRPTHSSVRQVEDGLTFPDYSDSTEVKFEELKNVKLEEEDEEEEQAAAALDLSVNPASVGGRLVFSGSKKKSSSSLGSGSSRDSISSDSETSEPLSCRAQGQTGVLTVHSYAKGDGRVTVGEPCTRKKGSAARSFSERELAEVADEYMFSLEENKKSKGRRQPLSKLPRHHPLVLQECVSDDETSEQLTPEEEAEETEAWAKPLSQLWQNRPPNFEAEKEFNETMAQQAPHCAVCMIFQTYHQVEFGGFNQNCGNASDLAPQKQRTKPLIPEMCFTSTGCSTDINLSTPYLEEDGTSILVSCKKCSVRVHASCYGVPPAKASEDWMCSRCSANALEEDCCLCSLRGGALQRANDDRWVHVSCAVAILEARFVNIAERSPVDVSKIPLPRFKLKCIFCKKRRKRTAGCCVQCSHGRCPTAFHVSCAQAAGVMMQPDDWPFVVFITCFRHKIPNLERAKGALQSITAGQKVISKHKNGRFYQCEVVRLTTETFYEVNFDDGSFSDNLYPEDIVSQDCLQFGPPAEGEVVQVRWTDGQVYGAKFVASHPIQMYQVEFEDGSQLVVKRDDVYTLDEELPKRVKSRLSVASDMRFNEIFTEKEVKQEKKRQRVINSRYREDYIEPALYRAIME

SEQ ID NO: 3-4 - skipped (2 entries)

SEQ ID NO: 5 - DNA, 5675 bp, Homo sapiens
agggctcggtcgccagcaaccgagcggggcccggcccgagcggggcctgggggtgcgacgccgagggcgggggagagcgcgccgctgctcccggaccgggccgcgcacgccgcctcaggaaccatcactgttgctggaggcacctgacaaatcctagcgaatttttggagcatctccacccaggaacctcgccatccagaagtgtgcttcccgcacagctgcagccatggggtctgaggaccacggcgcccagaaccccagctgtaaaatcatgacgtttcgcccaaccatggaagaatttaaagacttcaacaaatacgtggcctacatagagtcgcagggagcccaccgggcgggcctggccaagatcatccccccgaaggagtggaagccgcggcagacgtatgatgacatcgacgacgtggtgatcccggcgcccatccagcaggtggtgacgggccagtcgggcctcttcacgcagtacaatatccagaagaaggccatgacagtgggcgagtaccgccgcctggccaacagcgagaagtactgtaccccgcggcaccaggactttgatgaccttgaacgcaaatactggaagaacctcacctttgtctccccgatctacggggctgacatcagcggctctttgtatgatgacgacgtggcccagtggaacatcgggagcctccggaccatcctggacatggtggagcgcgagtgcggcaccatcatcgagggcgtgaacacgccctacctgtacttcggcatgtggaagaccaccttcgcctggcacaccgaggacatggacctgtacagcatcaactacctgcactttggggagcctaagtcctggtacgccatcccaccagagcacggcaagcgcctggagcggctggccatcggcttcttccccgggagctcgcagggctgcgacgccttcctgcggcataagatgaccctcatctcgcccatcatcctgaagaagtacgggatccccttcagccggatcacgcaggaggccggggaattcatgatcacatttccctacggctaccacgccggcttcaatcacgggttcaactgcgcagaatctaccaacttcgccaccctgcggtggattgactacggcaaagtggccactcagtgcacgtgccggaaggacatggtcaagatctccatggacgtgttcgtgcgcatcctgcagcccgagcgctacgagctgtggaagcagggcaaggacctcacggtgctggaccacacgcggcccacggcgctcaccagccccgagctgagctcctggagtgcatcccgggcctcgctgaaggccaagctcctccgcaggtctcaccggaaacggagccagcccaagaagccgaagcccgaagaccccaagttccctggggagggtacggctggggcagcgctcctagaggaggctgggggcagcgtgaaggaggaggctgggccggaggttgaccccgaggaggaggaggaggagccgcagccactgccacacggccgggaggccgagggcgcagaagaggacgggaggggcaagctgcggccaaccaaggccaagagcgagcggaagaagaagagcttcggcctgctgcccccacagctgccgcccccgcctgctcacttcccctcagaggaggcgctgtggctgccatccccactggagcccccggtgctgggcccaggccctgcagccatggaggagagccccctgccggcaccccttaatgtcgtgccccctgaggtgcccagtgaggagctagaggccaagcctcggcccatcatccccatgctgtacgtggtgccgcggccgggcaaggcagccttcaaccaggagcacgtgtcctgccagcaggcctttgagcactttgcccagaagggtccgacctggaaggaaccagtttcccccatggagctgacggggccagaggacggtgcagccagcagtggggcaggtcgcatggagaccaaagcccgggccggagaggggcaggcaccgtccacattttccaaattgaagatggagatcaagaagagccggcgccatcccctgggccggccgcccacccggtccccactgtcggtggtgaagcaggaggcctcaagtgacgaggaggcatcccctttctccggggaggaagatgtgagtgacccggacgccttgaggccgctgctgtctctgcagtggaagaacagggcggccagcttccaggccgagaggaagttcaacgcagcggctgcgcgcacggagccctactgcgccatctgcacgctcttctacccctactgccaggccctacagactgagaaggaggcacccatagcctccctcggagagggctgcccggccacattaccctccaaaagccgtcagaagacccgaccgctcatccctgagatgtgcttcacctctggcggtgagaacacggagccgctgcctgccaactcctacatcggcgacgacgggaccagccccctgatcgcctgcggcaagtgctgcctgcaggtccatgccagttgctatggcatccgtcccgagctggtcaatgaaggctggacgtgttcccggtgcgcggcccacgcctggactgcggagtgctgcctgtgcaacctgcgaggaggtgcgctgcagatgaccaccgataggaggtggatccacgtgatctgtgccatcgcagtccccgaggcgcgcttcctgaacgtgattgagcgccaccctgtggacatcagcgccatccccgagcagcggtggaagctgaaatgcgtgtactgccggaagcggatgaagaaggtgtcaggtgcctgtatccagtgctcctacgagcactgctccacgtccttccacgtgacctgcgcccacgccgcaggcgtgctcatggagccggacgactggccctatgtggtctccatcacctgcctcaagcacaagtcggggggtcacgctgtccaactcctgagggccgtgtccctaggccaggtggtcatcaccaagaaccgcaacgggctgtactaccgctgtcgcgtcatcggtgccgcctcgcagacctgctacgaagtgaacttcgacgatggctcctacagcgacaacctgtaccctgagagcatcacgagtagggactgtgtccagctgggacccccttccgagggggagctggtggagctccggtggactgacggcaacctctacaaggccaagttcatctcctccgtcaccagccacatctaccaggtggagtttgaggacgggtcccagctgacggtgaagcgtggggacatcttcaccctggaggaggagctgcccaagagggtccgctctcggctgtcactgagcacgggggcaccgcaggagcccgccttctcgggggaggaggccaaggccgccaagcgcccgcgtgtgggcaccccgcttgccacggaggactccgggcggagccaggactacgtggccttcgtggagagcctcctgcaggtgcagggccggcccggagcccccttctaggacagctggccgctcaggcgaccctcagcccggcggggaggccatggcatgccccgggcgttcgcttgctgtgaattcctgtcctcgtgtccccgacccccgagaggccacctccaagccgcgggtgccccctagggcgacaggagccagcgggacgccgcacgcggccccagactcagggagcagggccaggcgggctcgggggccggccaggggagcaccccactcaactactcagaattttaaaccatgtaagctctcttcttctcgaaaaggtgctactgcaatgccctactgagcaacctttgagattgtcacttctgtacataaaccacctttgtgaggctctttctataaatacatattgtttaaaaaaaagcaagaaaaaaaggaaaacaaaggaaaatatccccaaagttgttttctagatttgtggctttaagaaaaacaaaacaaaacaaacacattgtttttctcagaaccaggattctctgagaggtcagagcatctcgctgtttttttgttgttgttttaaaatattatgatttggctacagaccaggcagggaaagagacccggtaattggagggtgagcctcggggggggggcaggacgccccggtttcggcacagcccggtcactcacggcctcgctctcgcctcaccccggctcctgggctttgatggtctggtgccagtgcctgtgcccactctgtgcctgctgggaggaggcccaggctctctggtggccgcccctgtgcacctggccaggggaagcccgggggtctggggcctccctccgtctgcgcccacctttgcagaataaactctctcctggggtttgtctatctttgtttctctcacctgagagaaacgcaggtgttccagaggcttccttgcagacaaagcacccctgcacctcctatggctcaggatgagggaggcccccaggcccttctggttggtagtgagtgtggacagcttcccagctcttcgggtacaaccctgagcaggtcgggggacacagggccgaggcaggccttcggggcccctttcgcctgcttccgggcagggacgaggcctggtgtcctcgctccacccacccacgctgctgtcacctgaggggaatctgcttcttaggagtgggttgagctgatagagaaaaaacggccttcagcccaggctgggaagcgccttctccaggtgcctctccctcaccagctctgcacccctctggggagccttccccaccttagctgtctcctgccccagggagggatggaggagataatttgcttatattaaaaacaaaaaatggctgaggcaggagtttgggaccagcctgggctatatagcaagaccccatcactacaaattttttacaaattagctaggtgtggtggtgcgcacctgtggtcccagctactcgggaggctgtggtgggaggattgcttgagtccaggaggttgaggctgcagtcagctcagattgcaccactgcactccagcctgggcaacagagcgagaccctgtctccaaaaaaaaaaaaaagcaatgtttatattataaaagagtgtcctaacagtccccgggctagagaggactaaggaaaacagagagagtgttacgcaggagcaagcctttcatttccttggtgggggaggggggcggttgccctggagagggccggggtcggggaggttggggggtgtcagccaaaacgtggaggtgtccctctgcacgcagccctcgcccggcgtggcgctgacactgtattcttatgttgtttgaaaatgctatttatattgtaaagaagcgggcgggtgcccctgctgcccttgtcccttgggggtcacacccatcccctggtgggctcctgggcggcctgcgcagatgggccacagaagggcaggccggagctgcacactctccccacgaaggtatctctgtgtcttactctgtgcaaagacgcggcaaaacccagtgccctggtttttccccacccgagatgaaggatacgctgtattttttgcctaatgtccctgcctctaggttcataatgaattaaaggttcatgaacgctgcgaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 1096 aa, Homo sapiens
MGSEDHGAQNPSCKIMTFRPTMEEFKDFNKYVAYIESQGAHRAGLAKIIPPKEWKPRQTYDDIDDVVIPAPIQQVVTGQSGLFTQYNIQKKAMTVGEYRRLANSEKYCTPRHQDFDDLERKYWKNLTFVSPIYGADISGSLYDDDVAQWNIGSLRTILDMVERECGTIIEGVNTPYLYFGMWKTTFAWHTEDMDLYSINYLHFGEPKSWYAIPPEHGKRLERLAIGFFPGSSQGCDAFLRHKMTLISPIILKKYGIPFSRITQEAGEFMITFPYGYHAGFNHGFNCAESTNFATLRWIDYGKVATQCTCRKDMVKISMDVFVRILQPERYELWKQGKDLTVLDHTRPTALTSPELSSWSASRASLKAKLLRRSHRKRSQPKKPKPEDPKFPGEGTAGAALLEEAGGSVKEEAGPEVDPEEEEEEPQPLPHGREAEGAEEDGRGKLRPTKAKSERKKKSFGLLPPQLPPPPAHFPSEEALWLPSPLEPPVLGPGPAAMEESPLPAPLNVVPPEVPSEELEAKPRPIIPMLYVVPRPGKAAFNQEHVSCQQAFEHFAQKGPTWKEPVSPMELTGPEDGAASSGAGRMETKARAGEGQAPSTFSKLKMEIKKSRRHPLGRPPTRSPLSVVKQEASSDEEASPFSGEEDVSDPDALRPLLSLQWKNRAASFQAERKFNAAAARTEPYCAICTLFYPYCQALQTEKEAPIASLGEGCPATLPSKSRQKTRPLIPEMCFTSGGENTEPLPANSYIGDDGTSPLIACGKCCLQVHASCYGIRPELVNEGWTCSRCAAHAWTAECCLCNLRGGALQMTTDRRWIHVICAIAVPEARFLNVIERHPVDISAIPEQRWKLKCVYCRKRMKKVSGACIQCSYEHCSTSFHVTCAHAAGVLMEPDDWPYVVSITCLKHKSGGHAVQLLRAVSLGQVVITKNRNGLYYRCRVIGAASQTCYEVNFDDGSYSDNLYPESITSRDCVQLGPPSEGELVELRWTDGNLYKAKFISSVTSHIYQVEFEDGSQLTVKRGDIFTLEEELPKRVRSRLSLSTGAPQEPAFSGEEAKAAKRPRVGTPLATEDSGRSQDYVAFVESLLQVQGRPGAPF

SEQ ID NO: 7 - DNA, 4687 bp, Homo sapiens
gccataggtgcgcgtcggcgcccaggaggacgtgtggcgcgtggactacatcaggtccagccctgcgggaccccagccagcgcttccgggcaaggttctgtgcacctgttttctccttctacgcgagtatctttcccctccggaaagaatgggatatgcctgtgtccaaaggacaagaagatgcgcgccagcaagcctaagttaaccacagcgcggaagttgagcccaaagcaagagcgtgccgggcacctttaagctgtttgtaagcccacgtgactcaccaagtgcgggccccagcggtcacgtgacggcgcgcgcgccctcgcgcagggagagccggcggtgcgcgcgccttcgccgctgcctcccacccaccccctcgacgggagggtgaggcgcggcgcagtgatcgggcggccggggtcctgtgcgcgtgcgcagcgaacagctgtcacctagtgcggaacaagtctcccaaatttcccaaatctccctgggccggaggccactgtcttctcttcctcctccaccgagtcgtgctctcgccccaacccgcgcgccagacactgccctaaccatcatggaggtggccgaggtggaaagtcctctgaaccccagctgtaagataatgaccttcagaccctccatggaggagttccgggagttcaacaaataccttgcatacatggagtctaaaggagcccatcgtgcgggtcttgcaaaggtgattcctcctaaggagtggaagccaagacagtgctatgatgacattgataatttgctcattccagcaccaattcagcagatggtcacagggcagtcaggactgttcactcagtacaacatccagaaaaaagcgatgactgtgaaggagttcaggcagctggccaacagtggcaaatattgtactccaagatacttggattacgaagatttggagcgcaagtactggaagaacttaacttttgtggcacctatctatggtgcagatattaatgggagcatatatgatgagggtgtggatgaatggaacatagctcgcctcaatacagtcttggatgtggttgaagaagagtgtggcatttctattgagggtgtaaataccccatatctctattttggcatgtggaagaccacgtttgcatggcacaccgaagacatggacctctatagcattaattatctccactttggagagcccaagtcttggtatgctatacctccggagcatggaaaacgacttgaaagactagctcaaggttttttcccaagcagctcccaagggtgtgatgcatttcttcgccacaagatgacattgatttctccatcagtattgaagaaatatggtattccctttgacaagataacccaggaggctggagaattcatgatcactttcccatatggctaccatgctggttttaatcatggtttcaactgtgcagaatctacaaattttgctactgtcagatggattgactatggaaaagttgccaaattgtgcacttgcaggaaagacatggtgaagatttcaatggatatctttgtgaggaaatttcagccagacagatatcagctttggaaacaaggaaaggatatatacaccattgatcacacgaagcctactccagcatccacccctgaagtaaaagcatggctgcagaggaggaggaaagtaagaaaagcatcccgaagcttccagtgtgctaggtctacctctaaaaggcctaaggctgatgaggaagaggaagtgtcagatgaagtcgatggggcagaggtccctaaccccgactcagtcacagatgacctcaaggtcagtgaaaagtcagaagcagcagtgaagctgaggaacacagaagcatcttcagaagaagagtcatctgctagcaggatgcaggtggagcagaatttatcagatcatatcaaactctcaggaaacagctgcttaagtacatctgtaacagaagacataaaaactgaggatgacaaagcttatgcatatagaagtgtaccttctatatccagtgaggctgatgattccattccattgtctagtggctatgagaagcccgagaaatcagacccatccgagctttcatggccaaagtcacctgagtcatgctcatcagtggcagagagtaatggtgtgttaacagagggagaagagagtgatgtggagagccatgggaatggccttgaacctggggaaatcccagcggtccccagtggagagagaaatagcttcaaagtccccagtatagcagagggagagaacaaaacctctaagagttggcgccatccacttagcaggcctccagcaagatctccgatgactcttgtgaagcagcaggcgccaagtgatgaagaattgcctgaggttctgtccattgaggaggaagtggaagaaacagagtcttgggcgaaacctctcatccacctttggcagacgaagtcccctaacttcgcagctgagcaagagtataatgcaacagtggccaggatgaagccacactgtgccatctgcactctgctcatgccgtaccacaagccagatagcagcaatgaagaaaatgatgctagatgggagacaaaattagatgaagtcgttacatcggagggaaagactaagcccctcataccagagatgtgttttatttatagtgaagaaaatatagaatattctccacccaatgccttccttgaagaggatggaacaagtctccttatttcctgtgcaaagtgctgcgtacgggttcatgcaagttgttatggtattccttctcatgagatctgtgatggatggctgtgtgcccggtgcaaaagaaatgcgtggacagcagaatgctgtctctgcaatttgagaggaggtgctcttaagcaaacgaagaacaataagtgggcccatgtcatgtgcgccgttgcggtcccagaagttcgattcactaatgtcccagaaaggacacaaatagatgtaggcagaatacctttacagaggttaaaattgaaatgcatcttctgcagacaccgggttaagagggtctctggagcctgcatccagtgttcctacggtcgctgcccggcctccttccatgtcacttgtgcccatgctgctggggtactgatggagcctgatgactggccttatgtggtgaacattacatgctttcgacataaggtcaaccccaacgtgaagtccaaggcttgcgagaaggtcatttccgtgggtcaaacggtcatcacgaagcatcggaacacccggtattacagttgcagagtgatggctgtgacatcgcagaccttctatgaggtcatgtttgatgatggctcctttagcagagacacatttcctgaggatatcgtgagccgagactgtctgaagctgggcccacctgctgagggagaagtcgtccaagtcaagtggcccgatggcaaactctatggagcaaaatattttggatcaaatattgcccacatgtaccaggttgagtttgaagatggatcccagatagcaatgaagagagaggacatctacactttagatgaagagttacccaagagagtgaaagctcgattttccacagcctctgacatgcgatttgaagacacgttttatggagcagacattatccaaggggagagaaagagacaaagagtgctgagctccaggtttaagaatgaatatgtggccgaccctgtataccgcacttttttgaagagctctttccagaagaagtgccagaagagacagtagtctgcatacatcgctgcaggccacagagcagcttgggttggaagagagaagatgaagggacatccttggggctgtgccgtgagttttgctggcataggtgacagggtgtgtctctgacagtggtaaatcgggtttccagagtttggtcaccaaaaatacaaaatacacccaatgaattggacgcagcaatctgaaatcatctctagtcttgctttcacttgtgagcagttgtcttctatgatcccaaagaagttttctaagtgaaaggaaatactagtgaatcacccacaaggaaaagccactgccacagaggaggcgggtccccttgtgcggcttagggccctgtcaggaaacacacggggacctctctctctagctccagcaggtggcacctcggtacccagcgggtagggcgataatttatatattttccacagtcagggaaggactctcacttatttgtttcaaattgcagtttttataaaacatttttaaaacacaaatggcatgtatgctaatgagatttacccgtgtgctatctgtatttcccttgtacagaacttttacatttttgaatattcctattacttttgattgtgtctgatgggaactgagttgttggcctttgtgaaatgaaatttttggctcttgagaaagaattcttatgaattgttatgcgaattttatatatttaaagagggagatctggggctgttatttttaaacactttttttcataatacatattccgagtagatatttataaaatatatgtttctttcattatgtgtttgtaaaattagagtttaaataaatatgctttgatgcatagttttgaactaatgtaacatgatttttcttttttaaaacagcctgaaaatgtactagtgtttaaaaataaagatttccattttctccaaaaaaaaaaaaaaaaaa

SEQ ID NO: 8 - protein, 1056 aa, Homo sapiens
MEVAEVESPLNPSCKIMTFRPSMEEFREFNKYLAYMESKGAHRAGLAKVIPPKEWKPRQCYDDIDNLLIPAPIQQMVTGQSGLFTQYNIQKKAMTVKEFRQLANSGKYCTPRYLDYEDLERKYWKNLTFVAPIYGADINGSIYDEGVDEWNIARLNTVLDVVEEECGISIEGVNTPYLYFGMWKTTFAWHTEDMDLYSINYLHFGEPKSWYAIPPEHGKRLERLAQGFFPSSSQGCDAFLRHKMTLISPSVLKKYGIPFDKITQEAGEFMITFPYGYHAGFNHGFNCAESTNFATVRWIDYGKVAKLCTCRKDMVKISMDIFVRKFQPDRYQLWKQGKDIYTIDHTKPTPASTPEVKAWLQRRRKVRKASRSFQCARSTSKRPKADEEEEVSDEVDGAEVPNPDSVTDDLKVSEKSEAAVKLRNTEASSEEESSASRMQVEQNLSDHIKLSGNSCLSTSVTEDIKTEDDKAYAYRSVPSISSEADDSIPLSSGYEKPEKSDPSELSWPKSPESCSSVAESNGVLTEGEESDVESHGNGLEPGEIPAVPSGERNSFKVPSIAEGENKTSKSWRHPLSRPPARSPMTLVKQQAPSDEELPEVLSIEEEVEETESWAKPLIHLWQTKSPNFAAEQEYNATVARMKPHCAICTLLMPYHKPDSSNEENDARWETKLDEVVTSEGKTKPLIPEMCFIYSEENIEYSPPNAFLEEDGTSLLISCAKCCVRVHASCYGIPSHEICDGWLCARCKRNAWTAECCLCNLRGGALKQTKNNKWAHVMCAVAVPEVRFTNVPERTQIDVGRIPLQRLKLKCIFCRHRVKRVSGACIQCSYGRCPASFHVTCAHAAGVLMEPDDWPYVVNITCFRHKVNPNVKSKACEKVISVGQTVITKHRNTRYYSCRVMAVTSQTFYEVMFDDGSFSRDTFPEDIVSRDCLKLGPPAEGEVVQVKWPDGKLYGAKYFGSNIAHMYQVEFEDGSQIAMKREDIYTLDEELPKRVKARFSTASDMRFEDTFYGADIIQGERKRQRVLSSRFKNEYVADPVYRTFLKSSFQKKCQKRQ

SEQ ID NO: 9 - DNA, 2988 bp, Homo sapiens
aaggggcggggccgaagcggcccagggggcgggcgtttgaaatcagtgccttagagtagaccctaaacctcattttataccttcaagaaccaattacttaatgtctcttccgtcttttccgtccccgaccccctcccagactccttcattccggtactgcgtggacggaaagccccgggtagccgacaccacgtccccggctagcgggagagagcgtggaaaaggattacaccaaactgtttaaatccaacgactcctgcttccatcctttctcctgagctagaaccaacaaacctagagagttgggcttcggaaaaactagtgttttcatttaattggatatgaagaaagaacaaatatgtacggggcaaccacgatctttacaaagaacataagttccaggaaagcaggaaccttgtctctcttgttcactgggtgtatcctctgcatatagaacagtgcctggcacataataggtgctgaattttgttctaaacactgaggacattctctgctacatttgggtcgtacccccaggtctgagtaattcaatagacttaagaagacagagcccagcagcaaccgaaacataacagagttgcaggatcagctaacgtcaatgcctgggcaaagctgctgcccagagtggaatctcactagtgaataaacaagcccaagaaagattatcatctcatttgcaaaaaaaaaagtacgctggtagatcctgctacctcatagataacaccagtcaaatttttttttaaagtagcattttcctacattgtcaactatctagaacatacctaaaaactaagagtttactgcttattaaatggaaactatgaagtctaaggccaactgtgcccagaatccaaattgtaacataatgatatttcatccaaccaaagaagagtttaatgattttgataaatatattgcttacatggaatcccaaggtgcacacagagctggcttggctaagataattccacccaaagaatggaaagccagagagacctatgataatatcagtgaaatcttaatagccactcccctccagcaggtggcctctgggcgggcaggggtgtttactcaataccataaaaaaaagaaagccatgactgtgggggagtatcgccatttggcaaacagtaaaaaatatcagactccaccacaccagaatttcgaagatttggagcgaaaatactggaagaaccgcatctataattcaccgatttatggtgctgacatcagtggctccttgtttgatgaaaacactaaacaatggaatcttgggcacctgggaacaattcaggacctgctggaaaaggaatgtggggttgtcatagaaggcgtcaatacaccctacttgtactttggcatgtggaaaaccacgtttgcttggcatacagaggacatggacctttacagcatcaactacctgcaccttggggagcccaaaacttggtatgtggtgcccccagaacatggccagcgcctggaacgcctggccagggagctcttcccaggcagttcccggggttgtggggccttcctgcggcacaaggtggccctcatctcgcctacagttctcaaggaaaatgggattcccttcaatcgcataactcaggaggctggagagttcatggtgacctttccctatggctaccatgctggcttcaaccatggtttcaactgcgcagaggccatcaattttgccactccgcgatggattgattatggcaaaatggcctcccagtgtagctgtggggaggcaagggtgaccttttccatggatgccttcgtgcgcatcctgcaacctgaacgctatgacctgtggaaacgtgggcaagaccgggcagttgtggaccacatggagcccagggtaccagccagccaagagctgagcacccagaaggaagtccagttacccaggagagcagcgctgggcctgagacaactcccttcccactgggcccggcattccccttggcctatggctgcccgcagtgggacacggtgccacacccttgtgtgctcttcactcccacgccgatctgcagttagtggcactgctacgcagccccgggctgctgctgtccacagctctaagaagcccagctcaactccatcatccacccctggtccatctgcacagattatccacccgtcaaatggcagacgtggtcgtggtcgccctcctcagaaactgagagctcaggagctgaccctccagactccagccaagaggcccctcttggcgggcacaacatgcacagcttcgggcccagaacctgagcccctacctgaggatggggctttgatggacaagcctgtaccactgagcccagggctccagcatcctgtcaaggcttctgggtgcagctgggcccctgtgccctaagtccacgggctgtctttatatcccactgccctgctgtgtgacagtttgatgaaactggttacatttacatcccaaaactttggttgagtttgcaggactctaggcatgcatgaaagagcccccctggtgatgcccttggatgctgccaagtccatggtagttttcaattttgccatacttttgttcttcctaccggaccctggaatgtctttggatattgctaaaatctatttctgcagctgaggttttatccactggacacatttgtgtgtgagaactaggtcttgttgaggttagcgtaacctggtatatgcaactaccatcctctgggccaactgtggaagctgctgcacttgtgaagaatcctgagctttgattcctcttcagtctacgcatttctctcttcccctccctcaccccctttttcttataaaactaggttctttatacagataaggtcagtagagttccagaataaaagatatgacttttctgagttatttatgtacttaaaatatgttgtcacagtatttgttcccaaatatattaaaggtaaccaaaatgttaaaaaaaaaaaaaaaaaa

SEQ ID NO: 10 - protein, 523 aa, Homo sapiens
METMKSKANCAQNPNCNIMIFHPTKEEFNDFDKYIAYMESQGAHRAGLAKIIPPKEWKARETYDNISEILIATPLQQVASGRAGVFTQYHKKKKAMTVGEYRHLANSKKYQTPPHQNFEDLERKYWKNRIYNSPIYGADISGSLFDENTKQWNLGHLGTIQDLLEKECGVVIEGVNTPYLYFGMWKTTFAWHTEDMDLYSINYLHLGEPKTWYVVPPEHGQRLERLARELFPGSSRGCGAFLRHKVALISPTVLKENGIPFNRITQEAGEFMVTFPYGYHAGFNHGFNCAEAINFATPRWIDYGKMASQCSCGEARVTFSMDAFVRILQPERYDLWKRGQDRAVVDHMEPRVPASQELSTQKEVQLPRRAALGLRQLPSHWARHSPWPMAARSGTRCHTLVCSSLPRRSAVSGTATQPRAAAVHSSKKPSSTPSSTPGPSAQIIHPSNGRRGRGRPPQKLRAQELTLQTPAKRPLLAGTTCTASGPEPEPLPEDGALMDKPVPLSPGLQHPVKASGCSWAPVP

SEQ ID NO: 11 - DNA, 2339 bp, Homo sapiens
ccaagcctgagaatcaggagaagcctcacagtgacacccccaactgaggaaactcacagagctgggacatactctacttcttcagaaaaaagtatactgactagagtggagtccccctggggagtcagaaagcctgtgaaagatctcacttgttcaaaagtccaagtgtgaattactgtctcacagataaaccaaagtattttgaaaaacaaaggggagaaaagaaattactccccagaactctcaggcatctagaggacacccaagaacgtgggagtcagctgcttcttgtgtgcagccatgaagtctgtgcactccagtccccagaacacgagtcataccatcatgacgttttacccaaccatggaagaatttgcagatttcaacacatatgttgcttacatggagtcccaaggcgcacatcaagctggccttgccaaggtaattccacccaaggaatggaaagccagacagatgtatgatgatatcgaagacatcttaatagccactcccctccagcaggtgacctctgggcagggaggtgtgtttactcaataccataaaaagaagaaagccatgagggtggggcagtatcgccgcttggcaaacagtaaaaaatatcagactccgccacaccagaattttgcagatttggagcaacgatactggaagagccaccccggtaatccaccaatttatggtgctgatatcagcggctccttatttgaagaaagcactaaacaatggaacctaggacacctgggaacaattctggacctgttggagcaggaatgtggggttgtcatcgagggtgtcaacacaccctacctgtactttggcatgtggaagaccacgtttgcctggcacacagaggacatggacctttacagcatcaactacctgcactttggggagcccaaaacttggtacgtggtgcccccagaacatggtcagcacctggaacgcctggccagggagctcttcccagacatttctcggggctgtgaggccttcctgcggcacaaagtggccctcatctcgcctacagttctcaaggaaaatgggattcccttcaattgcatgactcaggaggctggggagttcatggtgacctttccctatggctaccatgctggcttcaatcacggcttcaactgcgcagaagccattaattttgccactccacgatggattgattatggcaaaatggcctctcagtgtagctgtggggagtcgacagtgaccttttccatggacccctttgtgcgcattgtgcaacccgagagttatgagctctggaaacacaggcaagacttggccattgtggaacacacagagcccagggttgcagaaagccaagagctgagcaactggagagatgatatagtacttagaagagctgctctgggcctgaggcttctcccaaacctcacagcccagtgtcccacacagcctgtgtcctcagggcactgttacaacccaaaaggctgtggcactgatgctgtgcctggatccgcattccaaagctctgcatatcatacccagacccagtcacttaccctggggatgtcagccagggttcttctcccttccactggaagctggggttctggtcgtggtcgtggtcgtggtcaaggtcaaggtcgaggttgcagtcgtggtcgtggtcatggttgttgtactcgagaactggggactgaggagccaactgttcagcctgcatccaagaggcgccttttaatgggtacaaggagtagagctcaaggccacaggcctcagctcccgcttgccaatgatttgatgacaaatctgtccctttgagtggtggccttcagcatcttgccaaggcttctggctgctgctgtgtccctgatcttcaactcctggggcccccactggatcgtgatgaaaccatgcaccctggcctgtgcctgctatccctcaacagcactactagtaatctccctgatgttgtctgcatgactcctcccaatgtcattgtgcctttgattaagttttccagggacactggtggggactggaactgattaagttcaccagggacacttgcctggtgaacatgggcaaggctgtagcaatggaccacttttacggctctagggttctgactccaactaagttttccagaatctcctgggctcctgactcatctgctgggtctaaagacactgagtttagggatattttcctccaatacatgatcaatcctctggatccacggctatggaatatggtgacaaatgtcagtgtctctcttattccaaccccaggatcagagaagattctttacctgcagtaactgacacatttccaaggcc

SEQ ID NO: 12 - protein, 506 aa, Homo sapiens
MKSVHSSPQNTSHTIMTFYPTMEEFADFNTYVAYMESQGAHQAGLAKVIPPKEWKARQMYDDIEDILIATPLQQVTSGQGGVFTQYHKKKKAMRVGQYRRLANSKKYQTPPHQNFADLEQRYWKSHPGNPPIYGADISGSLFEESTKQWNLGHLGTILDLLEQECGVVIEGVNTPYLYFGMWKTTFAWHTEDMDLYSINYLHFGEPKTWYVVPPEHGQHLERLARELFPDISRGCEAFLRHKVALISPTVLKENGIPFNCMTQEAGEFMVTFPYGYHAGFNHGFNCAEAINFATPRWIDYGKMASQCSCGESTVTFSMDPFVRIVQPESYELWKHRQDLAIVEHTEPRVAESQELSNWRDDIVLRRAALGLRLLPNLTAQCPTQPVSSGHCYNPKGCGTDAVPGSAFQSSAYHTQTQSLTLGMSARVLLPSTGSWGSGRGRGRGQGQGRGCSRGRGHGCCTRELGTEEPTVQPASKRRLLMGTRSRAQGHRPQLPLANDLMTNLSL

SEQ ID NO: 13-20 - skipped (8 entries)

SEQ ID NO: 21 - DNA, 73 bp, Homo sapiens
ggccggctggggttcctggggatgggatttgcttcctgtcacaaatcacattgccagggatttccaaccgacc

SEQ ID NO: 22 - DNA, 97 bp, Homo sapiens
ctcaggtgctctggctgcttgggttcctggcatgctgatttgtgacttaagattaaaatcacattgccagggattaccacgcaaccacgaccttggc

SEQ ID NO: 23 - DNA, 90 bp, Homo sapiens
ccgggcccctgtgagcatcttaccggacagtgctggatttcccagcttgactctaacactgtctggtaacgatgttcaaaggtgacccgc

SEQ ID NO: 24 - DNA, 95 bp, Homo sapiens
ccagctcgggcagccgtggccatcttactgggcagcattggatggagtcaggtctctaatactgcctggtaatgatgacggcggagccctgcacg

SEQ ID NO: 25 - DNA, 68 bp, Homo sapiens
ccctcgtcttacccagcagtgtttgggtgcggttgggagtctctaatactgccgggtaatgatggagg

SEQ ID NO: 26 - DNA, 102 bp, Homo sapiens
ggtcctctgactctcttcggtgacgggtattcttgggtggataatacggattacgttgttattgcttaagaatacgcgtagtcgaggagagtaccagcggca

SEQ ID NO: 27 - DNA, 20 bp, Homo sapiens
ctttactcagtacaacatac

SEQ ID NO: 28 - protein, 349 aa, Homo sapiens
ASESETLNPSARIMTFYPTMEEFRNFSRYIAYIESQGAHRAGLAKVVPPKEWKPRASYDDIDDLVIPAPIQQLVTGQSGLFTQYNIQKKAMTVREFRKIANSDKYCTPRYSEFEELERKYWKNLTFNPPIYGADVNGTLYEKHVDEWNIGRLRTILDLVEKESGITIEGVNTPYLYFGMWKTSFAWHTEDMDLYSINYLHFGEPKSWYSVPPEHGKRLERLAKGFFPGSAQSCEAFLRHKMTLISPLMLKKYGIPFDKVTQEAGEFMITFPYGYHAGFNHGFNCAESTNFATRRWIEYGKQAVLCSCRKDMVKISMDVFVRKFQPERYKLWKAGKDNTVIDHTLPTPEA

SEQ ID NO: 29 - protein, 342 aa, Bacillus subtilis
  MOD_RES (1)..(1): Any amino acid
  MOD_RES (13)..(13): Any amino acid
  MOD_RES (30)..(30): Any amino acid
  MOD_RES (76)..(76): Any amino acid
  MOD_RES (186)..(186): Any amino acid
  MOD_RES (227)..(227): Any amino acid
  MOD_RES (260)..(260): Any amino acid
XGSDKIHHHHHHXSAVTESVLESIISPVTXSEFLEEYWPVKPLVARGEVERFTSIPGFEKVRTLENVLAIYNNPVXVVGDAVIEESEGITDRFLVSPAEALEWYEKGAALEFDFTDLFIPQVRRWIEKLKAELRLPAGTSSKAIVYAAKNGGGFKAHFDAYTNLIFQIQGEKTWKLAKNENVSNPXQHYDLSEAPYYPDDLQSYWKGDPPKEDLPDAEIVNLTPGTXLYLPRGLWHSTKSDQATLALNITFGQPAWLDLXLAALRKKLISDNRFRELAVNHQSLHESSKSELNGYLESLIQTLSENAETLTPEQIFQSQDSDFDPYQSTQLVFRQLLTSYKF

SEQ ID NO: 30 - protein, 424 aa, Salmonella enterica
SNAMTTPFTHETLPADPKAAIRQMKQALRAQIGDVQAVFDRLSATIAARVAEINDLKAQGQPVWPIIPFSELAMGNISDATRAEVKRRGCAVIKGHFPREQALAWDQSMLDYLDKNHFDEVYKGPGDNFFGTLSASRPEIYPVYWSQAQMQARQSEEMALAQSFLNRLWQVEHDGKRWFNPDISIIYPDRIRRRPPGTTSKGLGAHTDSGALERWLLPAYQQVFASVFNGNVEQYDPWNAAHRTDVEEYTVDNTTKCSVFRTFQGWTALSDMLPGQGLLHVVPIPEAMAYILLRPLLDDVPEDELCGVAPGRVLPISEQWHPLLMAALTSIPPLEAGDSVWWHCDVIHSVAPVENQQGWGNVMYIPAAPMCEKNLAYARKVKAALETGASPGDFPREDYETTWEGRFTLRDLNIHGKRALGIDV

SEQ ID NO: 31 - protein, 330 aa, Arabidopsis thaliana
MAELLLVETPIPQQKHYESKPFPAVISPPSASIPIPALSLPLFTQTIKTQKHYLDSLLHESGAVLFRGFPVNSADDFNDVVEAFGFDELPYVGGAAPRTSVVGRVFTANESPPDQKIPFHHEMAQVREFPSKLFFYCEIEPKCGGETPIVLSHVVYERMKDKHPEFVQRLEEHGLLYVRVLGEDDDPSSPIGRGWKSTFLTHDKNLAEQRAVDLGMKLEWTEDGGAKTVMGPIPAIKYDESRNRKVWFNSMVAAYTGWEDKRNDPRKAVTFGDGKPLPADIVHDCLRILEEECVAVPWQRGDVLLIDNWAVLHSRRPFDPPRRVLASLCK

SEQ ID NO: 32 - protein, 283 aa, Escherichia coli
MSERLSITPLGPYIGAQISGADLTRPLSDNQFEQLYHAVLRHQVVFLRDQAITPQQQRALAQRFGELHIHPVYPHAEGVDEIIVLDTHNDNPPDNDNWHTDVTFIETPPAGAILAAKELPSTGGDTLWTSGIAAYEALSVPFRQLLSGLRAEHDFRKSFPEYKYRKTEEEHQRWREAVAKNPPLLHPVVRTHPVSGKQALFVNEGFTTRIVDVSEKESEALLSFLFAHITKPEFQVRWRWQPNDIAIWDNRVTQHYANADYLPQRRIMHRATILGDKPFYRAG

SEQ ID NO: 33 - protein, 357 aa, Streptomyces coelicolor
MKHHHHHHHSDYDIPTTENLYFQGSAANAAGPASRYDVTLDQSDAELVEEIAWKLATQATGRPDDAEWVEAARNAWHAWPATLRRDLAGFRRDSGPDGAIVLRGLPVDSMGLPPTPRVNGSVQREASLGAAVLLMTACGLGDPGAFLPEKNGALVQDVVPVPGMEEFQGNAGSTLLTFHNENAFHEHRPDFVMLLCLRADPTGRAGLRTACVRRVLPLLSDSTVDALWAPEFRTAPPPSFQLSGPEEAPAPVLLGDRSDPDLRVDLAATEPVTERAAEALRELQAHFDATAVTHRLLPGELAIVDNRVTVHGRTEFTPRYDGTDRWLQRTFVLTDLRRSRAMRPADGYVLGAAPQPA

SEQ ID NO: 34 - protein, 325 aa, Escherichia coli
MNALTAVHNNAVDSGQDYSGFTLTPSAQSPRLLELTFTEQTTKQFLEQVAEWPVQALEYKSFLRFRVGKILDDLCANQLQPLLLKTLLNRAEGALLINAVGIDDVAQADEMVKLATAVAHLIGRSNFDAMSGQYYARFVVKNVDNSDSYLRQPHRVMELHNDGTYVEEITDYVLMMKIDEQNMQGGNSLLLHLDDWEHLDHYFRHPLARRPMRFAAPPSKNVSKDVFHPVFDVDQQGRPVMRYIDQFVQPKDFEEGVWLSELSDAIETSKGILSVPVPVGKFLLINNLFWLHGRDRFTPHPDLRRELMRQRGYFAYATHHYQTHQ

SEQ ID NO: 35 - protein, 277 aa, Geobacillus kaustophilus
  MOD_RES (1)..(1): Any amino acid
  MOD_RES (15)..(15): Any amino acid
  MOD_RES (43)..(43): Any amino acid
  MOD_RES (91)..(91): Any amino acid
  MOD_RES (124)..(124): Any amino acid
  MOD_RES (137)..(137): Any amino acid
  MOD_RES (169)..(169): Any amino acid
  MOD_RES (172)..(172): Any amino acid
  MOD_RES (257)..(257): Any amino acid
XAIQRRIRRVKTVQXTTNSPIHRSGSVLEPGNWQEYDPFLLLXEDIFERGTFDVHPHRGIETVTYVISGELEHFDSKAGHSTLGPGDVQWXTAGRGVVHKEDPASGSTVHSLQLWVNLPSAYKXTEPRYQNLRSKDXPVRKEEGATIRVFSGSSKGVKAPTKNIVPVTXVEXIVEPGTTVVQDLPGHYNGFLYILEGSGVFGADNIEGKAGQALFFSRHNRGEETELNVTAREKLRLLLYAGEPVNEPVVAYGPFVXNTPEQIREAIRDYQEGRFGR

SEQ ID NO: 36 - protein, 324 aa, Streptomyces clavuligerus
MTSVDCTAYGPELRALAARLPRTPRADLYAFLDAAHTAAASLPGALATALDTFNAEGSEDGHLLLRGLPVEADADLPTTPSSTPAPEDRSLLTMEAMLGLVGRRLGLHTGYRELRSGTVYHDVYPSPGAHHLSSETSETLLEFHTEMAYHRLQPNYVMLACSRADHERTAATLVASVRKALPLLDERTRARLLDRRMPCCVDVAFRGGVDDPGAIAQVKPLYGDADDPFLGYDRELLAPEDPADKEAVAALSKALDEVTEAVYLEPGDLLIVDNFRTTHARTPFSPRWDGKDRWLHRVYIRTDRNGQLSGGERAGDVVAFTPRG

SEQ ID NO: 37 - protein, 273 aa, Pectobacterium carotovorum
  MOD_RES (1)..(1): Any amino acid
  MOD_RES (11)..(11): Any amino acid
  MOD_RES (37)..(37): Any amino acid
  MOD_RES (142)..(142): Any amino acid
  MOD_RES (188)..(188): Any amino acid
  MOD_RES (244)..(244): Any amino acid
XSEIVKFNPVXASGFGAYIDHRDFLEAKTETIKNLLXRQGFVVVKNLDIDSDTFRDIYSAYGTIVEYADEKIGVGFGYRDTLKLEGEKGKIVTGRGQLPFHADGGLLLSQVDQVFLYAAEIKNVKFRGATTVCDHALACQEXPAHLLRVLEEETFEVRVLERGYYVDVSPDGWFKVPVFTDLGWVRKXLIYFPFDEGQPASWEPRIVGFTDHETQAFFQELGAFLKQPRYYYKHFWEDGDLLIXDNRRVIHEREEFNDDDIVRRLYRGQTADI

SEQ ID NO: 38 - protein, 354 aa, Escherichia coli
  MOD_RES (1)..(1): Any amino acid
  MOD_RES (13)..(13): Any amino acid
  MOD_RES (108)..(108): Any amino acid
  MOD_RES (143)..(143): Any amino acid
  MOD_RES (211)..(211): Any amino acid
  MOD_RES (253)..(253): Any amino acid
  MOD_RES (265)..(265): Any amino acid
  MOD_RES (268)..(268): Any amino acid
XGSSHHHHHHGSXTDNNQNSREQFYQHISGQNLTPLWESLHHLVPKTPNANCAPAYWNYQEIRPLLLESGGLIGAKEAVRRVLVLENPALRGQSSITATLYAGLQLIXPGEVAPSHRHNQSALRFIVEGKGAFTAVDGERTPXNEGDFILTPQWRWHDHGNPGDEPVIWLDGLDLPLVNILGCGFAEDYPEEQQPVTRKEGDYLPRYAANXLPLRHQTGNSSPIFNYRYDRSREVLHDLTRLGDADEWDGYKXRYVNPVTGGYPXPSXGAFLQLLPKGFASRVARTTDSTIYHVVEGSGQVIIGNETFSFSAKDIFVVPTWHGVSFQTTQDSVLFSFSDRPVQEALGLFREARY

SEQ ID NO: 39 - protein, 216 aa, Oceanicola granulosus
MSLAQIKSLFATRLYHAPLSEHGPALDPAEFAASCYSIAEDDDAGQEWCEREGYPGYTSYASLTDLPWRFPIFADLVKSLDAHVAAFAEDLEFELDGKALRLEDIWINILPEGGVHGSHIHPHSVISGTTYVAMPEGTSALKLEDPRLPFMMAAPTRRKGAREELRTFRSVAPKVGDVLLWESWLRHEVPMNMAEEDRISVSFNYAWGEGHHHHHH

SEQ ID NO: 40 - protein, 394 aa, Ruegeria pomeroyi
MGSSHHHHHHSSGLVPRGSHMAPTEIKPEDDILGRARVRDTPELEAYYDDLAKIETGALWTVANDIEPWEPTPKSAPVHWKWSDLRREVLRAIDLVRPEDAGRRVVYLRNPQRKDVSAACGWLFSGIQTMKAGERAGAHRHAASALRFIMEGSGAYTIVDGHKVELGANDFVLTPNGTWHEHGILESGTECIWQDGLDIPLTNCLEANFYEVHPNDYQTTDIPLNDSPLTYGGPALLPQLDKWDKPYSPLLKYSWEPTYEALLNYAKASDGSPYDGLILRYTNPQTGGHPMLTMGASMQMLRPGEHTKAHRHTGNVIYNVAKGQGYSIVGGKRFDWSEHDIFCVPAWTWHEHCNTQERDDACLFSFNDFPVMEKLGFWAEQALEDNGGHQIVAD